(19) United States
(12) Patent Application Publication
(10) Pub. No.: US 2022/0111065 A1
(43) Pub. Date: Apr. 14, 2022

(54) MOLECULAR ADJUVANT

(71) Applicants: ADC THERAPEUTICS SA, Epalinges (CH); MEDIMMUNE LIMITED, Cambridge (GB)

(72) Inventors: Patrick Hendrikus Cornelis VAN BERKEL, London (GB); Jay Marshall FEINGOLD, Murray Hill, NJ (US); Jens WUERTHNER, Epalinges (CH); James ADAMS, Murray Hill, NJ (US)

(21) Appl. No.: 17/057,486

(22) PCT Filed: May 22, 2019
(86) PCT No.: PCT/EP2019/063262

§ 371 Date: Nov. 20, 2020

(30) Foreign Application Priority Data
May 23, 2018 (GB) 1808507.6
Aug. 10, 2018 (GB) 1813067.4
Nov. 7, 2018 (GB) 1818152.9

(51) Int. Cl. A61K 47/68; A61K 47/54; A61K 47/55; A61K 45/06; A61K 35/17; A61K 35/15; A61K 39/39; A61K 39/00; A61P 35/00

(52) U.S. Cl. CPC A61K 47/6849; A61K 47/545; A61K 47/55; A61K 45/06; A61P 35/00; A61K 35/15; A61K 39/39; A61K 39/0011; A61K 35/17

(57) ABSTRACT

The present disclosure relates to therapies for the treatment of a disorders characterized by a disorder-associated antigen (DAA); vaccination methods are disclosed. In particular, the disclosure describes anti-CD25 ADC molecular adjuvants for use in inducing or enhancing a subject's immune response against a DAA, allowing for treatment of the disorder characterized by the DAA. Also disclosed are dosage regiments for the treatment of solid tumours with an AThe present disclosure relates to the treatment of pathological conditions, such as cancer, with anti-CD25 ADCs.

This application claims priority from: United Kingdom application GB1808507.6, filed on 23 May 2018; United Kingdom application GB1813067.4, filed on 10 Aug. 2018; and United Kingdom application GB1818152.9, filed on 7 Nov. 2018. The disclosure of these three priority document is incorporated by reference into the present application for all purposes.

### FIELD

The present disclosure relates to therapies for the treatment of a disorders characterized by a disorder-associated antigen (DAA); vaccination methods are disclosed. In particular, the disclosure describes a molecular adjuvant for use in inducing or enhancing a subject's immune response against a DAA, allowing for treatment of the disorder characterized by the DAA.

### BACKGROUND

Vaccines

A vaccine is a biological preparation that induces acquired immunity to a particular disease, providing protection (prophylactic vaccine) or aiding treatment (therapeutic vaccine).

Vaccines typically have a basic composition that includes two principal components: one or more antigens derived from a causal agent (such as a neoplastic cell or a pathogenic microorganism), responsible for the specificity, and an adjuvant to enhance the immunogenicity and protective efficacy of the vaccine (see Schijns et al. 2011, Exp. Rev. Vac. 10:4, pp. 539-550).

Over several decades, studies of vaccines have evolved towards the search for well-characterized and highly-purified antigens, such as recombinant proteins and peptides; over the last several years, this search has included the introduction of defined, potent and safer adjuvants for a new generation of vaccines (Garcon et al. 2017, Hum. Vac. & Imm. 13:1, pp. 19-33). Aluminum salts were the first substances used as vaccine adjuvants, and they were the only approved adjuvants in preventive human vaccines for decades. However, owing to their relatively weak capacity to induce cellular immunity and an emerging debate regarding possible links between aluminum and certain severe post-vaccination reactions (Batista-Duharte et al., Toxicol. Lett. 10 (2) (2011) 97-105; Bonam et al., Trends Pharmacol. Sci. (2017)) the search for alternative adjuvants has become an area of intense investigation. For many years, the search for new adjuvants has been active but empirical, and as a consequence a growing number of new substances with documented adjuvant activity have been reported in the literature. However, owing to safety concerns, very few adjuvants, such as MF59 and ASO3, have been licensed for use in human vaccines (Garcon et al. 2017, ibid.; Bonam et al., 2017, ibid.). For this reason, there has been an increasing trend towards the rational design of vaccines using defined molecules with well-characterized cellular and molecular mechanisms of action (so-called “Molecular adjuvants”).

Modulating Regulatory Immune Cells

One of the current directions of the molecular adjuvant approach is the targeting of immune regulatory networks (Davies et al., Methods Mol. Biol. 1494 (2017) 107-125). Growing knowledge of the immune system and how it is regulated has shown that there a number of sub-populations of immune cells which have a regulatory function in modulating immune system responses. For example, myeloid-derived suppressor cells (MDSCs) and mesenchymal stromal cells (MSCs) have both been reported to exert negative regulation of immune responses, including effector T-cell responses, and implicated in assisting tumour growth (Bianchi et al. 2011, Histol Histopathol. July; 26(7):941-51). Similarly, Type II NKT cells have been reported as having an important role in down-regulating tumour immunosurveillance in a number of tissues (Terabe et al., Nat Immunol 2000; 1:515-20; Terabe et al., J Exp Med 2005; 202:1627-33). Finally, some studies have shown that depletion of Tregs prior to vaccination in murine models can enhance immune responses to certain vaccines, suggesting that Tregs can interfere with the generation of vaccine-induced immunity (Klages et al., Cancer Res. 70 (20) (2010) 7788-7799; Fisher et al. Immun. Inflamm. Dis. 5 (1) (2016) 16-28).

Molecular Adjuvants

Accordingly, a number of different treatment strategies targeted at immune regulation have been proposed or examined for impact on clinical outcome. Supported by growing knowledge of the immune system, many of these strategies utilise molecular adjuvants to improve vaccine efficacy. These molecular adjuvants are typically defined molecules with characterized cellular and molecular mechanisms that interact with the immune system with the aim of improving vaccination-associated clinical outcomes. Example treatment strategies include: metronomic chemotherapy, CD25 antibodies, CTLA4 antibodies, anti-GITR antibodies, anti-OX40, PD1 pathway modulation, Indoleamine 2,3-dioxygenase inhibitors, anti-LAG3, CCR4 antagonists, anti-FOXP3, blockade of TGFβ, Listeriolysin O, blockade of adenosine-mediated immune suppression, anti-angiogenic molecules, folate receptor 4 antibodies, nicotinamide adenine dinucleotide, TLR modulation, and ICOS antibodies (reviewed in Batista-Duharte et al., Pharmacological Research 129 (2018) 237-250).

However, a number of studies have identified cancer models where Treg modulation has no benefit (murine pancreatic carcinoma model—Keenan et al., Gastroenterology 146 (7) (2014) 1784-1794) or highlighted the risks of regulatory immune cell modulation such as disruption of immune tolerance, and induction of inflammatory disorders or autoimmune processes (Bayry et al., Virus disease 25 (1) (2014) 18-25; van Elsas et al., Exp. Med. 194(4) (2001) 481-489). These risks are in part due to the fact that the highly complex mechanisms that control regulatory immune cells are not yet well-defined (Berod et al., Microbiol. Biotechnol. 5 (2) (2012)260-269), and in part due the use of modulators that target Treg signal molecules (such as receptors or other proteins) that form parts of signalling pathways common to both Tregs and effector T cells (Teffs). Certainly, Tregs and activated T-cells share several features and receptors, which explains the weak selectivity of some immunomodulators intended for specific Treg inhibition (Pere et al., Oncoimmunology 1 (3) (2012) 326-333; Ustun et al., Blood 118 (19) (2011) 5084-5095).

As a consequence, more recent studies have proposed increasingly focussed strategies designed to inhibit regulatory immune cell function rather than their deletion by using combined checkpoint inhibition to improve efficacy and safety (Pere et al. ibid.; Ustun et al. ibid.; Wang et al., Cell Res. 27 (1) (2017) 11-37); specific and effective molecular adjuvants are an essential part of these strategies.

### SUMMARY

The present authors have studied the effects of administering CD25-ADCs in a number of different disease models, including models with CD25-ve tumor target cells. Their observations indicated an efficacy beyond what was expected for either of direct target cell killing by the ADC combined with the so-called ‘bystander effect’ indirect cell killing, as described in WO/2016/083468. Building on these observations, the present authors reasoned that increased efficacy of ADCx25 arose from targeted cell-killing of AD25+ve regulatory immune cells, such as Tregs. That is, the present authors have determined that the CD25-ADCs described herein have applications as powerful and specific molecular adjuvants.

The therapies described herein include those that induce or enhance a subject's immune response. In particular, in certain aspects the therapies include treating a disorder by inducing or enhancing the immune response of a subject against an antigen associated with the disorder. In some aspects the therapies described herein enhance or induce an immune response by targeting immune regulatory cells with a CD25-ADC. The targeting of immune regulatory cells in this manner allows for a reduction in the negative regulation of the subject's immune responses to an existing or newly presented antigen.

In some aspects the therapies described herein enhance or induce an immune response by directly killing target cells through cytotoxic ADC binding to the target cells and/or, through a ‘bystander effect’, indirectly killing target cells in the proximity of cells that are directly bound by the cytotoxic ADC (see, for example, WO/2016/083468). Without wishing to be bound by theory, it is believed that this killing of target cells causes the release of target antigens, ‘stranger signals’, ‘neo-epitopes’, and/or ‘danger signals’ into the extracellular environment where they can interact with and further stimulate a subject's immune system (see, for example, Virgil EJC Schijns & Ed C Lavelle (2011) Expert Review of Vaccines, 10:4, 539-550).

Thus, in one aspect, the present disclosure provides a method of inducing or enhancing an immune response in a subject, the method comprising the step of administering a CD25-ADC to the subject. The immune response may be disorder-associated antigen (DAA) specific immune response, such as a CD8+ T cell response, a CD4+ T cell response, an antibody response, or a memory cell response.

In another aspect, the present disclosure provides a method of treating or preventing a disorder a subject, wherein the disorder is characterized by a disorder-associated antigen (DAA), the method comprising administering a CD25-ADC to the subject.

In some embodiments the CD25-ADC is administered in combination with a DAA. The DAA may be administered as part of a vaccine composition, optionally wherein the CD25-ADC is also part of the same vaccine composition.

The CD25-ADC may administered in combination with the DAA or vaccine compostion, although they may be administered comcomitantly. Preferably, the CD25-ADC is administered before the DAA or vaccine composition. The immune-suppressive activity or size of a population of regulatory immune cells in the subject may be reduced by at least 90% before the DAA or vaccine composition is administered. Preferably the regulatory immune cells are Treg cells.

The DAA may be tumour-associated antigen (TAA) or a pathogen-associated antigen (PAA). The PAA is derived from a pathogen selected from the group consisting of a virus, bacterium, fungus, protozoa, parasite, prion protein, or protein aggregate.

In some cases the subject has, is suspected of having, has been diagnosed with, or is at risk of, a disorder characterized by a disorder-associated antigen (DAA). In some cases the subject has been selected for treatment on the basis that the subject has, is suspected of having, has been diagnosed with, or is at risk of, a disorder characterized by a disorder-associated antigen (DAA).

The disorder may be a proliferative disorder such as cancer.

In some cases, the proliferative disorder or cancer is a solid tumour, which may comprise or consist of CD25-ve neoplastic cells. The solid tumour may be associated with CD25+ve infiltrating cells, in some cases high levels of CD25+ve infiltrating cells. In some cases the solid tumour is selected from the group consisting of pancreatic cancer, breast cancer (including triple negative breast cancer), colorectal cancer, gastric and oesophageal cancer, melanoma, non-small cell lung cancer, ovarian cancer, hepatocellular carcinoma, renal cell carcinoma, bladder, and head and neck cancer.

In some cases the proliferative disorder or cancer is lymphoma or leukaemia, such as Hodgkin's Lymphoma; non-Hodgkin's, including diffuse large B-cell lymphoma (DLBCL), follicular lymphoma, (FL), Mantle Cell lymphoma (MCL), chronic lymphatic lymphoma (CLL) Marginal Zone B-cell lymphoma (MZBL); and leukemias, including Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph-ALL). The proliferative disorder or cancermay be associated with elevated levels of regulatory immune cells, such as Treg cells.

The subject may be selected for treatment on the basis that the subject has received an adoptive cell transfer, such as a bone marrow transplant. The adoptive cell transfer may be an autologous cell transfer or an allogenic cell transfer, and may be a stem cell transfer and/or an immune cell transfer. In some cases, the subject received the adoptive cell transfer at least 3 months prior to the administration of the CD25-ADC, such as at least 6 months, at least 12 months, at least 18 months, or at least 24 months prior to the administration of the CD25-ADC.

The CD25-ADC may administered in combination with a cell therapy. Preferably, the CD25-ADC is administered before the cell therapy, such as at least 7 days before the cell therapy. The immune-suppressive activity or size of a population of regulatory immune cells in the subject may be reduced by at least 90% before the cell therapy is administered. Preferably the regulatory immune cells are Treg cells. The cell therapy may comprise the administration of autologous cells, allogenic cells, stem cells, and/or immune cells.

Preferably, the administered cells are immune cells such as T-cells, Natural Killer (NK) cells, Natural Killer T-cell (NKT), Lymphokine-activated Killer (LAK) cells, or macrophages. The immune cells express a chimeric antigen receptor (CAR). In some embodiments the immune cells are CAR T-cells, such as a 1^{st} generation CAR T-cell, a 2^{nd} generation CAR T-cell, a 3^{rd} generation CAR T-cell, a 4^{th} generation CAR T-cell, a TRUCK, a smart CAR, or an iCAR.

In another aspect, the disclosure also provides an antibody-drug conjugate compound as disclosed herein for use in a method of treatment as disclosed herein.

In another aspect, the disclosure also provides a composition or pharmaceutical composition comprising an antibody-drug conjugate compound as disclosed herein for use in a method of treatment as disclosed herein.

In another aspect, the disclosure also provides a use of an antibody-drug conjugate compound as disclosed herein in the preparation of a medicament for use in a method of treatments as disclosed herein.

### DETAILED DESCRIPTION

The present authors have studied the effects of administering the CD25-ADC, ADCx25, in a number of different disease models. Their observations indicated an efficacy of treatment beyond what was expected for a combination of direct target cell killing by the ADC combined with the so-called ‘bystander effect’ indirect cell killing, as described in WO/2016/083468. Building on these observations, the present authors reasoned that increased efficacy of ADCx25 arose from targeted cell-killing of AD25+ve regulatory immune cells, such as Tregs. That is, the present authors have determined that the CD25-ADCs described herein have applications as powerful and specific molecular adjuvants.

Accordingly, in one aspect the present disclosure provides a method of inducing or enhancing an immune response in a subject, the method comprising the step of administering a CD25-ADC to the subject.

In another aspect, the present disclosure provides a method of treating or preventing a disorder a subject, wherein the disorder is characterized by a disorder-associated antigen (DAA), the method comprising administering a CD25-ADC to the subject.

Inducing or Enhancing an Immune Response

The present disclosure provides method of inducing or enhancing an immune response in a subject.

Preferably the immune response is a disorder-associated antigen (DAA) specific immune response. That is, the induced or enhanced immune response is elicited by and/or targeted to the DAA.

For example, in some embodiments the DAA-specific immune response is the activation and/or proliferation of CD8+ve T-cells (commonly referred to as T-Helper cells, or Th cells). DAA-specific Th cells will express a T-cell receptor (TCR) that specifically binds the DAA.

In some embodiments the DAA-specific immune response is the activation and/or proliferation of CD4+ve T-cells (commonly referred to as cytotoxic T-cells, or T_{c} cells). DAA-specific T_{c} cells will express a T-cell receptor (TCR) that specifically binds the DAA.

In some embodiments the DAA-specific immune response is the activation and/or proliferation of B-cells. DAA-specific B-cells will express a B-cell receptor (BCR) and/or secrete antibodies that specifically bind the DAA.

In some embodiments the DAA-specific immune response is the secretion of antibodies that specifically bind the DAA.

In some embodiments the DAA-specific immune response is the generation of memory cells. The memory cells may be memory T-cells or memory B-cells. The memory T-cells may be memory Tₕ-cells or memory T_{c}-cells. DAA-specific memory cells will express a surface receptor that specifically binds the DAA.

The term ‘inducing or enhancing an immune response’ as used herein refers to creating, or increasing the level of, the relevant immune response through administration of the CD25-ADC. The enhancement may be relative to, for example, a control subject or population of subjects who have not received the CD25-ADC.

In some embodiments the existence or level of the immune response may be assessed by measuring the titer of a specific cell or molecule in a sample taken from a subject. For example, the existence or level of a DAA-specific T_{c}-cell response can be assessed by identifying and counting the relevant cells in a sample of peripheral blood taken from a subject.

In some embodiments, induction of an immune response means that following administration of the CD25-ADC the level of immune response is increased from am undetectable to a detectable level. In some embodiments an immune response is deemed to have ben induced if administration of the CD25-ADC is effective in preventing, ameliorating and/or treating a disorder characterized by a disorder-associated antigen (DAA). Prevention encompasses inhibiting or reducing the spread of the disorder or inhibiting or reducing the onset, development or progression of one or more of the symptoms associated with the disorder. Amelioration as used in herein may refer to the reduction of visible or perceptible disorder symptoms, or any other measurable manifestation of the disorder.

In some embodiments, enhancement of an immune response means that following administration of the CD25-ADC the level of the immune response is increased by at least 10%, such as by at least 20%, at least 30%, at least 40%, at least 40%, at least 50%, at least 60%, at least 70%, at least 80%, at least 90%, at least 100%, at least 150%, at least 200%, at least 300%, at least 400%, or at least 500%.

In some embodiments the level of immune response is assessed by determining the number of activated CD4+ve T-cell cells. In some embodiments the level of immune response is assessed by determining the size of certain cell populations, such as NK cells, monocyes, or dendritic cells). In some embodiments the level of immune response is assessed by determining the titre of a specific antibody or antibodies, for example the titre of an antibody or antibodies specific for a DAA.

In some embodiments the immune response is induced or enhanced by administering the CD25-ADC to the subject in combination with a second immunostimulatory agent. For example, the CD25-ADC may be administered to the subject in combination with a CD3/DAA bispecific T-cell engager (BiTE), an anti-CD47 therapeutic, a PD-1 inhibitor, a PDL-1 inhibitor, a GITR agonist, an OX40 agonist, or a CTLA-1 antagonist, In some embodiments the second immunostimulatory agent used as a monotherapy does not induce a significant immune response in the subject.

Treating Disorders Characterised by a DAA

The present disclosure provides a method of treating or preventing a disorder a subject, wherein the disorder is characterized by a disorder-associated antigen (DAA).

Disorders characterised by a DAA include proliferative diseases such as cancer, wherein neoplastic cells express one or more antigen that is either (i) not expressed on non-neoplastic cells, or more usually (ii) expressed at ha higher level on neoplastic cells than on non-neoplastic cells. Examples of this type of disorder include most prostate cancer s (DAA=PSMA) and some breast cancers (DAA=HER2).

Disorders characterised by a DAA also include diseases caused by a pathogen, wherein the pathogen is (or expresses) an antigen. Examples of this type of disorder include Human papillomavirus (HPV; DAA(s)=capsid protein(s) and some breast cancers (DAA=HER2).

A more detailed discussion of disorders characterised by a DAA, along with a selection of example DAAs can be found herein below in the section entitled “Treated disorders”.

Without wishing to be bound by theory, the efficacy of the treatment methods described herein is believed to arise from the induction and/or enhancement of the subject's immune response against the DAA. This induction and/or enhancement is believed to be due to the administration of the CD25-ADC reducing the immune-suppressive activity of a population of immune regulatory cells (such as CD25+ve T_{reg} cells). The reduction in regulatory-cell immune-suppression allows for the induction and/or enhancement of the immune response against the DAA.

Treg Cells

Tregs are identified by surface expression of CD4+ve/CD25^{high}/CD127^{low/-} [33] and form two main subsets: natural Tregs (nTregs),which are thymus-derived, and induced, adaptive or peripheral Tregs (pTregs), which are derived from naive CD4+ T cells under a variety of conditions (Curotto de Lafaille et al., Immunity 30 (2009)626-635-).

Tregs have immunosuppressive activity, with the more important effector T cells (Teffs) that are suppressed by Tregs being Th1 (control of infections and tumors), Th2 (effectors against extra-cellular parasites, including helminths), Th17 (play an important role in pathogen clearance at mucosal surfaces, are effective against pathogenic fungi and are also involved in different inflammatory process) and CTLs (cytotoxic T cells). Treg-mediated suppression can be accomplished in two ways:by cell-cell contact or by means of soluble mediators and cytokines (paracrine signaling). Several mechanisms have been identified as involved in the immunosuppressive function of Tregs, including:
(1) inhibition by immunoregulatory cytokines such as TGFβ, IL-10,and IL-35;
(2) cytolysis of effector cells by producing granzyme and perforin;
(3) metabolic interruption, including inhibition of proliferative response via the IL-2 receptor, cAMP-mediated metabolic inhibition, tryptophane depletion and immunomodulation mediated by the A2 adenosine receptor or kynurenine; and
(4) interactions with dendritic cells that modulates their function and maturation (Batista-Duharte et al. 2018, ibid.; Arce-Sillas, et al., J. Immunol. Res. 2016, 1720827).

Administration of CD25-ADC without DAA

In some embodiments the DAA is not administered in combination with the DAA. This may be the case in embodiments where, for example, the DAA is already present in the subject when the CD25-ADC is administered. For example, in some embodiments the subject may already have a proliferative disease characterised by a neoplasm whose cells express the DAA. In these cases the subject's immune response against the DAA may be suppressed or attenuated by a population of immune regulatory cells either globally, or in the neoplasm microenvironment.

For example, as discussed in more detail herein, a number of proliferative disorders have been reported to be associated with an increased number of immune regulatory cells such as T_{reg} cells. Without wishing to be bound by theory, it is believed that reducing the immune-suppressive activity of immune regulatory cells allows for the effective ‘unmasking’ of the DAA-expressing neoplastic cells and their attack by the subject's immune system.

Accordingly, through stimulation of the subject's immune system the administration of the CD25-ADC allows for the effective treatment of a broad range of disorders.

Administration of CD25-ADC in Combination with DAA In some embodiments the DAA is administered in combination with a CD25-ADC.

In embodiments where the DAA is administered to the subject, the term “DAA” encompasses both, (1) the antigen itself in the form it is recognised by the subject immune system, and (2) molecules which stimulate an immune response specific to the DAA. For example, for the HIV core protein p24, the p24 polypeptide itself is a DAA of type (1). An immune response against the p24 protein may also be generated by administering to the subject a suitable vaccine vector containing a nucleotide encoding the p24 protein; accordingly, the nucleotide encoding p24 is a DAA of type (2).

In some embodiments the DAA is a “self” antigen. For example, many tumour associated antigens (TAAs) are self antigens which are expressed at higher levels on neoplastic cells.

In some embodiments the DAA is a “non-self” antigen. For example, many antigens expressed by pathogenic microorganisms are non-self antigens.

Vaccine Compositions

A DAA may be administered as part of a vaccine composition.

Preferably the vaccine composition comprises a therapeutically effective amount of the DAA. Therapeutically effective amount refers to an amount of DAA that is effective for preventing, ameliorating and/or treating a disorder characterized by the DAA. Prevention encompasses inhibiting or reducing the spread of the disorder or inhibiting or reducing the onset, development or progression of one or more of the symptoms associated with the disorder. Amelioration as used in herein may refer to the reduction of visible or perceptible disorder symptoms or any other measurable manifestation of the disorder.

Examples of suitable types of vaccine composition include:
vectored vaccines, such as viral vectored vaccine, bacterial vectored vaccine, or plasmid DNA;
non-vectored vaccines, such as those comprising a naked protein DAA.

The term “vectored vaccines” is well known in the art and includes plasmid DNA, recombinants of poxviruses such as MV A, replicating vaccinia, fowlpox, avipox, also of adenoviruses including nonhuman primate adenoviruses, of alphaviruses, of vesicular stomatitis virus, and bacterial vectors such as Salmonella, Shigella and BCG.

The vectored vaccine may contain, or contain a nucleic acid encoding, a recombinant protein DAA. The recombinant DAA may be expressed in a viral vector.

Examples of viral vectors include vaccinia virus vectors such as MVA or NYVAC, avipox vectors such as fowlpox or canarypox (eg. ALVAC), vectors based on herpes virus, and vectors based on Venezuelan equine encephalitis virus (VEE).

Examples of bacterial vectors include recombinant BCG and recombinant Salmonella and Salmonella transformed with plasmid DNA.

Examples of non-vectored vaccines include carrier molecules such as lipid-tailed peptides known as lipopeptides, peptides fused to carrier proteins such as KLH either as fusion proteins or by chemical linkage, and antigens modified with a targeting tag, for example C3d or C4b binding protein. Alternatively, naked antigens may be administered.

In certain embodiments, the vaccine compositions further comprise one or more adjuvants. Adjuvants are known in the art to further increase the immune response to an applied antigenic determinant. The terms “adjuvant” and “immune stimulant” are used interchangeably herein, and are defined as one or more substances that cause stimulation of the immune system. In this context, an adjuvant is used to enhance an immune response to a DAA.

Examples of suitable adjuvants include aluminium salts such as aluminium hydroxide and/or aluminium phosphate; oil-emulsion compositions (or oil-in-water compositions), including squalene-water emulsions, such as MF59 (see e.g. WO 90/14837); saponin formulations, such as for example QS21 and Immunostimulating Complexes (ISCOMS) (see e.g. U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g. antibodies or fragments thereof (e.g. directed against the antigen itself or CD1a, CD3, CD7, CD80) and ligands to receptors (e.g. CD40L, GMCSF, GCSF, etc), which stimulate immune response upon interaction with recipient cells.

In certain embodiments the vaccine compositions comprise aluminium as an adjuvant, e.g. in the form of aluminium hydroxide, aluminium phosphate, aluminium potassium phosphate, or combinations thereof, in concentrations of 0.05-5 mg, e.g. from 0.075-1.0 mg, of aluminium content per dose.

Further examples of adjuvants are discussed in He et al. 2015, Hum. Vac. & Imm. 11:2, pp. 477-488; Garcon et al. 2017, Hum. Vac. & Imm. 13:1, pp. 19-33; Schijns et al. 2011, Exp. Rev. Vac. 10:4, pp. 539-550.

Administration of the vaccine compositions can be performed using standard routes of administration. Non-limiting embodiments include parenteral administration, such as intradermal, intramuscular, subcutaneous, transcutaneous, or mucosal administration, e.g. intranasal, oral, and the like. In one embodiment a composition is administered by intramuscular injection. The skilled person knows the various possibilities to administer a composition, e.g. a vaccine in order to induce an immune response to the antigen(s) in the vaccine. In certain embodiments, a composition is administered intramuscularly.

The DAA or vaccine compositions may be administered, either as prime, or as boost, in a homologous or heterologous prime-boost regimen. If a boosting vaccination is performed, typically, such a boosting vaccination will be administered to the same subject at a time between one week and one year, preferably between two weeks and four months, after administering the composition to the subject for the first time (which is in such cases referred to as ‘priming vaccination’). In certain embodiments, the administration comprises a prime and at least one booster administration.

Sequence of Administration

The DAA may be administered to the subject before the CD25-ADC, concomitantly with the CD25-ADC, or after the CD25-ADC

Preferably, the CD25-ADC is administered before the DAA. For example, the CD25-ADC may be administered 1 hour, 2 hours, 6 hours, 12 hours, or 24 hours before the DAA. The CD25-ADC may be administered 1 day, 2 days, 3 days, 4 days, 5 days, 6 days, or 7 days before the DAA. Preferably the CD25-ADC is administered at least 1 day before the DAA, even more preferably at least 2 days before the DAA.

In some embodiments the immune-suppressive activity of a population of immune regulatory cells is reduced before the DAA is administered to the subject. In some embodiments the reduction in the immune-suppressive activity of the immune regulatory cell population is achieved by killing a proportion of the population. In some embodiments the reduction in the immune-suppressive activity of the immune regulatory cell population is achieved by inhibiting the immune-suppressive activity of a proportion of the regulatory cell population without killing the cells.

The immune regulatory cells may be myeloid-derived suppressor cells (MDSCs), mesenchymal stromal cells (MSCs), Type II NKT cells, Treg cells, or any other immune regulatory immune cells as defined herein.

Preferably the immune regulatory cells whose immune-suppressive activity is reduced are Treg cells. The term “Treg” cells as used herein refers to regulatory T-cells. This cell population may be identified by the following pattern of surface-marker expression: CD4+ve, CD25^{high}, CD127^{low/-} (see Yu et al. 2012, Inflammation 35(6), pp. 1773-1780).

In some embodiments the immune-suppressive activity of a population of immune regulatory cells is reduced by at least 10%, at least 20%, at least 30%, at least 40%, at least 50%, at least 60%, at least 70%, at least 70%, at least 90%, at least 95%, or at least 98% before the DAA is administered to the subject. Preferably, the reduction is measured relative to levels in the same subject prior to the administration of the CD25-ADC.

In some embodiments the immune-suppressive activity of a population of immune regulatory cells is assessed by measuring the level of inhibitory cytokines such as IL-10, TGFβ, or IL-35 (see Bettini et al., Current opinion in immunology. 2009; 21(6):612-618). In some embodiments the immune-suppressive activity of a population of immune regulatory cells is assessed by measuring the expression of specific genes such as LAYN, MADEH1, or CCR8 (see de Simone et al., Immunity. 2016 Nov. 15; 45(5): 1135-1147).

In some embodiments the size of a population of immune regulatory cells is reduced by at least 10%, at least 20%, at least 30%, at least 40%, at least 50%, at least 60%, at least 70%, at least 70%, at least 90%, at least 95%, or at least 98% before the DAA is administered to the subject. Preferably, the reduction in population size is measured relative to levels in the same subject prior to the administration of the CD25-ADC.

In some embodiments the population of immune regulatory cells is measured systemically, for example by using FACS on a representative sample such as whole blood, bone-marrow, lymph-nodes, spleen, peyer-plaques, or tonsils. In some embodiments the population of immune regulatory cells is measured locally, for example in a sample taken from a tumour or the tumour microenvironment. The local population of immune regulatory cells may be measured by, for example, FACS, immunohisochemistry or immunofluorescence of tissue sections. Alternatively, techniques such as RNAscope® may be used on tissue sections to quantify immune regulatory cells in biopsies. Local measurement of cell population is preferred in situations where local measurement is possible (for example, for solid tumours).

In some embodiments the CD25-ADC is administered concomitantly with the DAA.

In some embodiments, the dose of CD25-ADC administered is about 20 μg/kg to 80 μg/kg, such as about 20, 25, 30, 35, 40, 45, 50, 55, 60, 65, 70, 75, or 80 μg/kg.

CD25

The type I transmembrane protein CD25 is present on activated T- and B-cells, some thymocytes, myeloid precursors, and oligodendrocytes. On activated T-cells, it forms heterodimers with the beta- and gamma subunits (CD122 and CD132), thus comprising the high-affinity receptor for IL-2. This ligand represents a survival factor for activated T-cells, as removal of IL-2 leads to immediate death of these cells.

In case of B-cells, CD25 is physiologically expressed in early developmental stages of late pro-B and pre-B cells. Malignancies arising from this stage of B-cell differentiation may thus also express CD25. Mast cell lesions are also positive for CD25 which is thus considered as a key diagnostic criterion for determination of systemic mastocytosis. In Hodgkin lymphomas, CD25 is reported to be not expressed in Hodgkin-/Reed-Sternberg cells in nodular lymphocyte predominance Hodgkin lymphoma (NLPHL), whereas the same cell type expresses CD25 at varying levels in classical Hodgkin’ lymphomas of mixed cellularity type. The general expression levels are reported to be lower than in tumor infiltrating lymphocytes (TILs), which may result in problems demonstrating CD25 tumor cells in these cases (Levi et al., Merz et al, 1995).

Expression of the target antigen has also been reported for several B- and T-cell-derived subtypes of non-Hodgkin-lymphomas, i.e. B-cell chronic lymphatic leukemia, hairy cell leukemia, small cell lymphocytic lymphoma/chronic lymphocytic leukemia as well as adult T-cell leukemia/lymphoma and anaplastic large cell lymphoma.

CD25 may be localised to the membrane, with some expression observed in the cytoplasm. Soluble CD25 may also be observed outside of cells, such as in serum.

Antibody Therapy

Antibody therapy has been established for the targeted treatment of subjects with cancer, immunological and angiogenic disorders (Carter, P. (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumour cells in the treatment of cancer, targets delivery of the drug moiety to tumours, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert J. (2005) Current Opin. in Pharmacol. 5:543-549; Hamann P. (2005) Expert Opin. Ther. Patents 15(9):1087-1103; Payne, G. (2003) Cancer Cell 3:207-212; Trail et al (2003) Cancer Immunol. Immunother. 52:328-337; Syrigos and Epenetos (1999) Anticancer Research 19:605-614).

CD25 ADCs

As used herein, the term “CD25-ADC” refers to an ADC in which the antibody component is an anti-CD25 antibody. In a preferred aspect, the CD25-ADC has the structure defined in the following paragraphs:

1. A conjugate of formula L—(D^{L})ₚ, where D^{L} is of formula I or II:

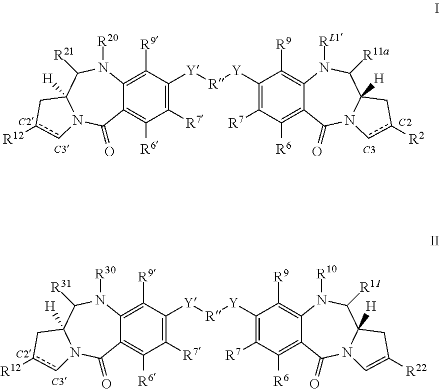

wherein:

L is an antibody (Ab) which is an antibody that binds to CD25;
when there is a double bond present between C2′ and C3′, R¹² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

(id)

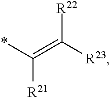

wherein each of R²¹, R²² and R²³ are independently selected from H, C₁₋₃ saturated aikyl, C₂₋₃ alkenyl, C₂₋₃ alkynyl and cyclopropyl, where the total number of carbon atoms in the R¹² group is no more than 5;

(ie)

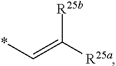

wherein one of R^{25a} and R^{25b} is H and the other is selected from: phenyl, which phenyl is optionally substituted by a group selected from halo, methyl, methoxy; pyridyl; and thiophenyl; and

(if)

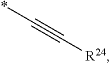

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl; when there is a single bond present between C2′ and C3′,

R¹² is

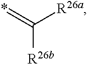

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;

R⁶ and R⁹ are independently selected from H, R, OH, OR, SH, SR, NH₂, NHR, NRR′, nitro, Me₃Sn and halo;

where R and R′ are independently selected from optionally substituted C₁₋₁₂ alkyl, C₃₋₂₀ heterocyclyl and C-20 aryl groups;

R⁷ is selected from H, R, OH, OR, SH, SR, NH₂, NHR, NHRR′, nitro, Me₃Sn and halo;

R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;

Y and Y′ are selected from O, S, or NH;

R^{6′}, R^{7′}, R^{9′} are selected from the same groups as R⁶, R⁷ and R⁹ respectively;

[Formula I]

R^{L1′} is a linker for connection to the antibody (Ab);

R^{11a} is selected from OH, OR^{A}, where R^{A} is C₁₋₄ alkyl, and SO_{z}M, where z is 2 or 3 and M is a monovalent pharmaceutically acceptable cation;

R²⁰ and R²¹ either together form a double bond between the nitrogen and carbon atoms to which they are bound or;

R²⁰ is selected from H and R^{C}, where R^{C} is a capping group;

R²¹ is selected from OH, OR^{A} and SO_{z}M;

when there is a double bond present between C2 and C3, R² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

(id)

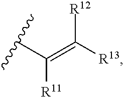

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

(ie)

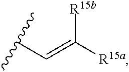

wherein one of R^{15a} and R^{15b} is H and the other is selected from: phenyl, which phenyl is optionally substituted by a group selected from halo, methyl, methoxy; pyridyl; and thiophenyl; and

(if)

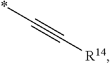

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2 and C3,

R² is

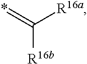

where R^{16a} and R_{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

[Formula II]

R²² is of formula IIIa, formula IIIb or formula IIIc:

(a)

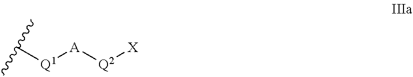

where A is a C₅₋₇ aryl group, and either

(i) Q¹ is a single bond, and Q² is selected from a single bond and —Z—(CH₂)ₙ—, where Z is selected from a single bond, O, S and NH and n is from 1 to 3; or

(ii) Q¹ is —CH═CH—, and Q² is a single bond;

(b)

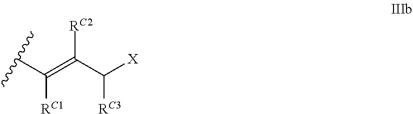

where;

R^{C1}, R^{C2} and R^{C3} are independently selected from H and unsubstituted C₁₋₂ alkyl;

(c)

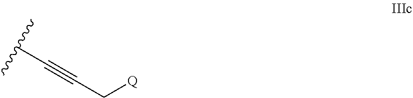

where Q is selected from O—R^{L2′}, S—R^{L2′} and NR^{N}—R^{L2′} and R_{N} is selected from H, methyl and ethyl

X is selected from the group comprising: O—R^{L2′}, S—R^{L2′}, CO₂—R^{L2′}, CO—R^{L2′}, NH—C(═O)—R^{L2′} NHNH—R^{L2′}, CONHNH—R^{L2′},

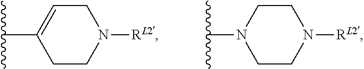

NR^{N}R^{L2′}, wherein RN is selected from the group comprising H and C₁₋₄ alkyl;

R^{L2′} is a linker for connection to the antibody (Ab);

R¹⁰ and R¹¹ either together form a double bond between the nitrogen and carbon atoms to which they are bound or;

R¹⁰ is H and R¹¹ is selected from OH, OR^{A} and SO_{z}M;

R³⁰ and R³¹ either together form a double bond between the nitrogen and carbon atoms to which they are bound or;

R³⁰ is H and R³¹ is selected from OH, OR^{A} and SO_{z}M.

2. The conjugate according to statement 1, wherein the conjugate is not:

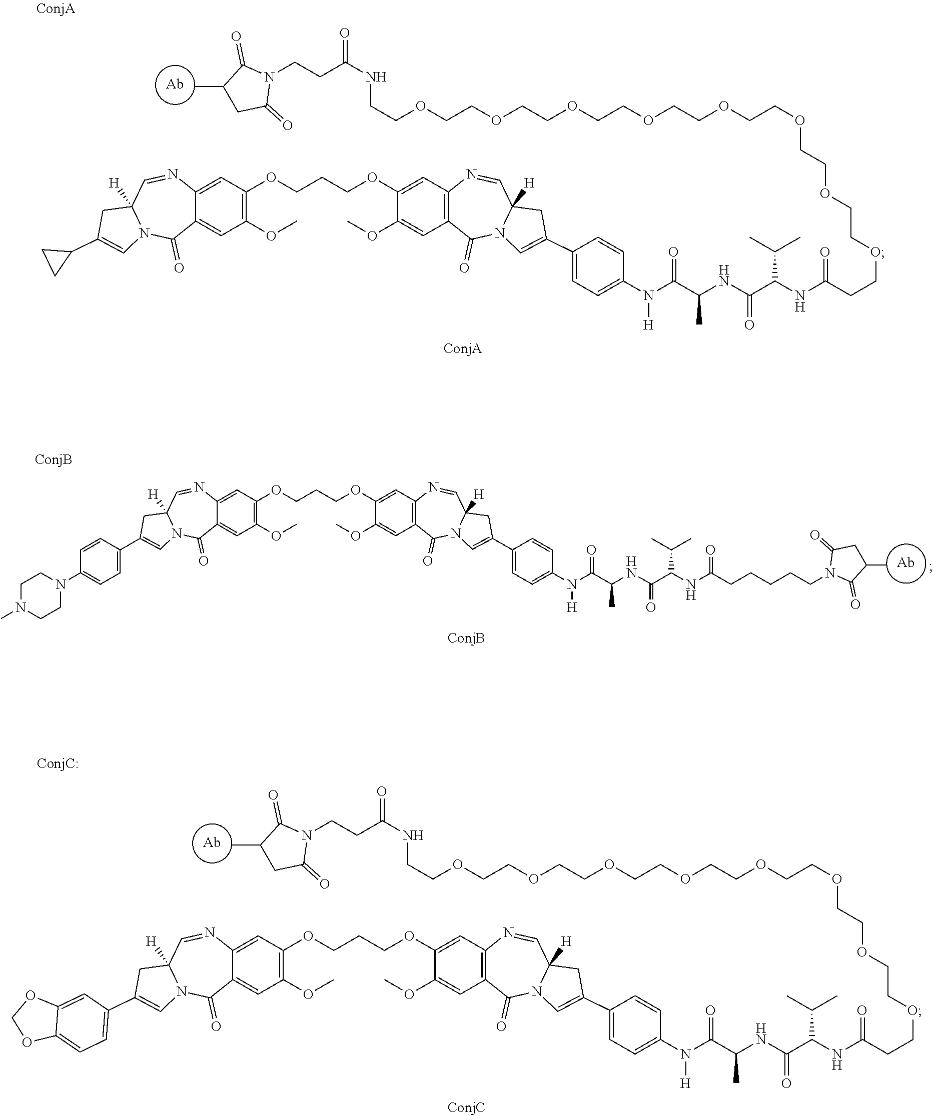

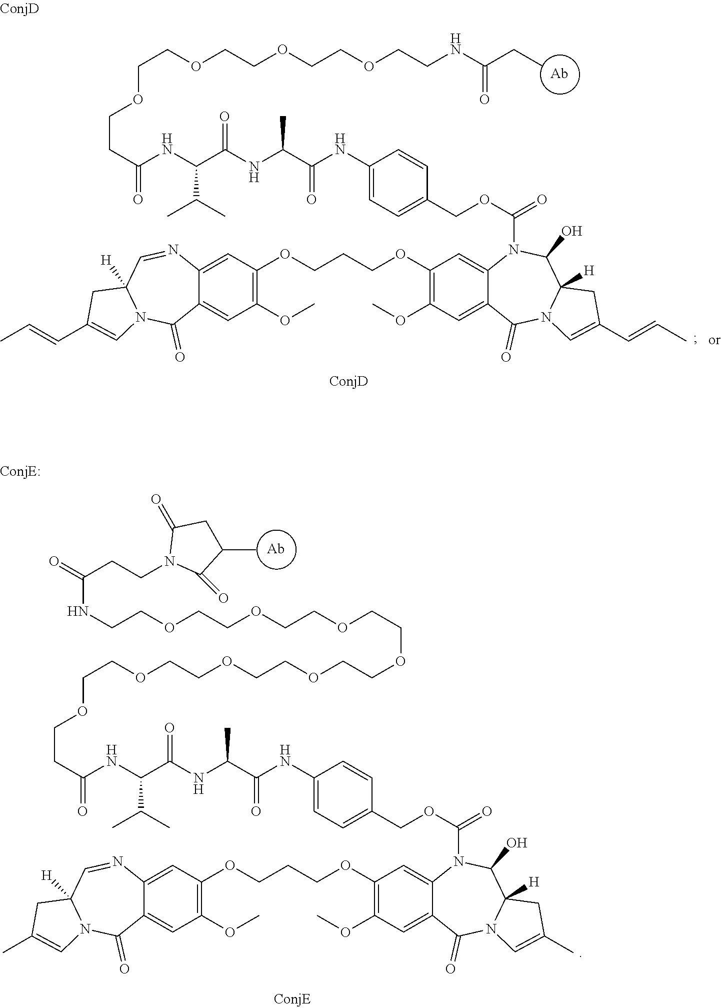

3. The conjugate according to either statement 1 or statement 2, wherein R⁷ is selected from H, OH and OR.

4. The conjugate according to statement 3, wherein R⁷ is a C₁₋₄ alkyloxy group.

5. The conjugate according to any one of statements 1 to 4, wherein Y is O.

6. The conjugate according to any one of the preceding statements, wherein R″ is C₃₋₇ alkylene.

7. The conjugate according to any one of statements 1 to 6, wherein R⁹ is H.

8. The conjugate according to any one of statements 1 to 7, wherein R⁶ is selected from H and halo.

9. The conjugate according to any one of statements 1 to 8, wherein there is a double bond between C2′ and C3′, and R¹² is a C₅₋₇ aryl group.

10. The conjugate according to statement 9, wherein R¹² is phenyl.

11. The conjugate according to any one of statements 1 to 8, wherein there is a double bond between C2′ and C3′, and R¹² is a C₃₋₁₀ aryl group.

12. The conjugate according to any one of statements 9 to 11, wherein R¹² bears one to three substituent groups.

13. The conjugate according to any one of statements 9 to 12, wherein the substituents are selected from methoxy, ethoxy, fluoro, chloro, cyano, bis-oxy-methylene, methyl-piperazinyl, morpholino and methyl-thiophenyl.

14. The conjugate according to any one of statements 1 to 8, wherein there is a double bond between C2′ and C3′, and R¹² is a C₁₋₅ saturated aliphatic alkyl group.

15. A compound according to statement 16, wherein R¹² is methyl, ethyl or propyl.

16. The conjugate according to any one of statements 1 to 8, wherein there is a double bond between C2′ and C3′, and R¹² is a C₃₋₆ saturated cycloalkyl group.

17. The conjugate according to statement 16, wherein R¹² is cyclopropyl.

18. The conjugate according to any one of statements 1 to 8, wherein there is a double bond between C2′ and C3′, and R¹² is a group of formula:

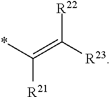

19. The conjugate according to statement 18, wherein the total number of carbon atoms in the R¹² group is no more than 4.

20. The conjugate according to statement 19, wherein the total number of carbon atoms in the R¹² group is no more than 3.

21. The conjugate according to any one of statements 18 to 20, wherein one of R²¹, R²² and R²³ is H, with the other two groups being selected from H, C₁₋₃ saturated alkyl, C₂₋₃ alkenyl, C₂₋₃ alkynyl and cyclopropyl.

22. The conjugate according to any one of statements 18 to 20, wherein two of R²¹, R²² and R²³ are H, with the other group being selected from H, C₁₋₃ saturated alkyl, C₂₋₃ alkenyl, C₂₋₃ alkynyl and cyclopropyl.

23. The conjugate according to any one of statements 1 to 8, wherein there is a double bond between C2′ and C3′, and R¹² is a group of formula:

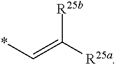

24. The conjugate according to statement 23, wherein R¹² is the group:

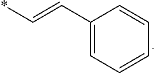 [SMILES: */C=C/c1ccccc1]

25. The conjugate according to any one of statements 1 to 8, wherein there is a double bond between C2′ and C3′, and R¹² is a group of formula:

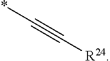

26. The conjugate according to statement 25, wherein R²⁴ is selected from H, methyl, ethyl, ethenyl and ethynyl.

27. The conjugate according to statement 26, wherein R²⁴ is selected from H and methyl.

28. The conjugate according to any one of statements 1 to 8, wherein there is a single bond between C2′ and C3′, R¹² is

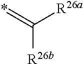

and R^{26a} and R^{26b} are both H.

29. The conjugate according to any one of statements 1 to 8, wherein there is a single bond between C2′ and C3′, R¹² is

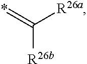

and R^{26a} and R^{26b} are both methyl.

30. The conjugate according to any one of statements 1 to 8, wherein there is a single bond between C2′ and C3′, R¹² is

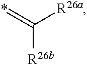

one of R^{26a} and R^{26b} is H, and the other is selected from C₁₋₄ saturated alkyl, C₂₋₃ alkenyl, which alkyl and alkenyl groups are optionally substituted.

[Formula I]

31. The conjugate according to any one of statements 1 to 30, wherein there is a double bond between C2 and C3, and R² is a C₅₋₇ aryl group.

32. The conjugate according to statement 31, wherein R² is phenyl.

33. The conjugate according to any one of statements 1 to 30, wherein there is a double bond between C2 and C3, and R¹ is a C₈₋₁₀ aryl group.

34. A compound according to any one of statements 31 to 33, wherein R² bears one to three substituent groups.

35. The conjugate according to any one of statements 31 to 34, wherein the substituents are selected from methoxy, ethoxy, fluoro, chloro, cyano, bis-oxy-methylene, methyl-piperazinyl, morpholino and methyl-thiophenyl.

36. The conjugate according to any one of statements 1 to 30, wherein there is a double bond between C2 and C3, and R² is a C₁₋₅ saturated aliphatic alkyl group.

37. The conjugate according to statement 36, wherein R² is methyl, ethyl or propyl.

38. The conjugate according to any one of statements 1 to 30, wherein there is a double bond between C2 and C3, and R² is a C₃₋₆ saturated cycloalkyl group.

39. The conjugate according to statement 38, wherein R² is cyclopropyl.

40. The conjugate according to any one of statements 1 to 30, wherein there is a double bond between C2 and C3, and R² is a group of formula:

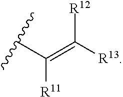

41. The conjugate according to statement 40, wherein the total number of carbon atoms in the R² group is no more than 4.

42. The conjugate according to statement 41, wherein the total number of carbon atoms in the R² group is no more than 3.

43. The conjugate according to any one of statements 40 to 42, wherein one of R¹¹, R¹² and R¹³ is H, with the other two groups being selected from H, C₁₋₃ saturated alkyl, C₂₋₃ alkenyl, C₂₋₃ alkynyl and cyclopropyl.

44. The conjugate according to any one of statements 40 to 42, wherein two of R¹¹, R¹² and R¹³ are H, with the other group being selected from H, C₁₋₃ saturated alkyl, C₂₋₃ alkenyl, C₂₋₃ alkynyl and cyclopropyl.

45. The conjugate according to any one of statements 1 to 30, wherein there is a double bond between C2 and C3, and R² is a group of formula:

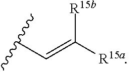

46. The conjugate according to statement 45, wherein R² is the group:

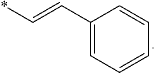 [SMILES: */C=C/c1ccccc1]

47. The conjugate according to any one of statements 1 to 30, wherein there is a double bond between C2 and C3, and R² is a group of formula:

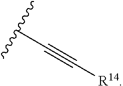

48. The conjugate according to statement 48, wherein R¹⁴ is selected from H, methyl, ethyl, ethenyl and ethynyl.

49. The conjugate according to statement 48, wherein R¹⁴ is selected from H and methyl.

50. The conjugate according to any one of statements 1 to 30, wherein there is a single bond between C2 and C3, R² is

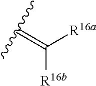

and R^{16a} and R^{16b} are both H.

51. The conjugate according to any one of statements 1 to 30, wherein there is a single bond between C2 and C3, R² is

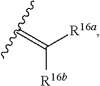

and R^{16a} and R^{16b} are both methyl.

52. The conjugate according to any one of statements 1 to 30, wherein there is a single bond between C2 and C3, R² is

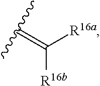

one of R^{16a} and R^{16b} is H, and the other is selected from C₁₋₄ saturated alkyl, C₂₋₃ alkenyl, which alkyl and alkenyl groups are optionally substituted.

53. The conjugate according to any one of statements 1 to 52, wherein R^{11a} is OH.

54. The conjugate according to any one of statements 1 to 53, wherein R²¹ is OH.

55. The conjugate according to any one of statements 1 to 53, wherein R²¹ is OMe.

56. The conjugate according to any one of statements 1 to 55, wherein R²⁰ is H.

57. The conjugate according to any one of statements 1 to 55, wherein R²⁰ is R^{C}.

58. The conjugate according to statement 57, wherein R^{C} is selected from the group consisting of: Alloc, Fmoc, Boc, Troc, Teoc, Psec, Cbz and PNZ.

60. The conjugate according to statement 57, wherein R^{C} is a group:

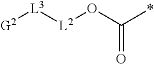

where the asterisk indicates the point of attachment to the N10 position, G² is a terminating group, L³ is a covalent bond or a cleavable linker L¹, L² is a covalent bond or together with OC(=O) forms a self-immolative linker.

61. The conjugate according to statement 60, wherein G² is Ac or Moc or is selected from the group consisting of: Alloc, Fmoc, Boc, Troc, Teoc, Psec, Cbz and PNZ.

62. The conjugate according to any one of statements 1 to 53, wherein R²⁰ and R²¹ together form a double bond between the nitrogen and carbon atoms to which they are bound.

[Formula II]

63. The conjugate according to any one of statements 1 to 30, wherein R²² is of formula IIIa, and A is phenyl.

64. The conjugate according to any one of statements 1 to 30 and statement 63, wherein R²² is of formula IIa, and Q¹ is a single bond.

65. The conjugate according to statement 63, wherein Q² is a single bond.

66. The conjugate according to statement 63, wherein Q² is —Z—(CH₂)ₙ—, Z is O or S and n is 1 or 2.

67. The conjugate according any one of statements 1 to 30 and statement 63, wherein R²² is of formula IIIa, and Q¹ is —CH═CH—.

68. The conjugate according to any one of statements 1 to 30, wherein R²² is of formula IIIb,

and R^{C1}, R^{C2} and R^{C3} are independently selected from H and methyl.

69. The conjugate according to statement 68, wherein R^{C1}, R^{C2} and R^{C3} are all H.

70. The conjugate according to statement 68, wherein R^{C1}, R^{C2} and R^{C3} are all methyl.

71. The conjugate according to any one of statements 1 to 30 and statements 63 to 70, wherein R²² is of formula IIIa or formula IIIb and X is selected from O—R^{L2′}, S—R^{L2′}, CO₂—R^{L2′}, —N—C(═O)—R^{L2′} and NH—R^{L2′}.

72. The conjugate according to statement 71, wherein X is NH—R^{L2′}.

73. The conjugate according to any one of statements 1 to 30, wherein R²² is of formula IIIc, and Q is NR^{N}—R^{L2′}.

74. The conjugate according to statement 73, wherein RN is H or methyl.

75. The conjugate according to any one of statements 1 to 30, wherein R²² is of formula IIIc, and Q is O—R^{L2′} or S—R^{L2′}.

76. The conjugate according to any one of statements 1 to 30 and statements 63 to 75, wherein R¹¹ is OH.

77. The conjugate according to any one of statements 1 to 30 and statements 63 to 75, wherein R¹¹ is OMe.

78. The conjugate according to any one of statements 1 to 30 and statements 63 to 77, wherein R¹⁰ is H.

79. The conjugate according to any one of statements 1 to 30 and statements 63 to 75, wherein R¹⁰ and R¹¹ together form a double bond between the nitrogen and carbon atoms to which they are bound.

80. The conjugate according to any one of statements 1 to 30 and statements 63 to 79, wherein R³¹ is OH.

81. The conjugate according to any one of statements 1 to 30 and statements 63 to 79, wherein R³¹ is OMe.

82. The conjugate according to any one of statements 1 to 30 and statements 63 to 81, wherein R³⁰ is H.

83. The conjugate according to any one of statements 1 to 30 and statements 63 to 79, wherein R³⁰ and R³¹ together form a double bond between the nitrogen and carbon atoms to which they are bound.

84. The conjugate according to any one of statements 1 to 83, wherein R^{6′}, R^{7′}, R^{9′}, and Y′ are the same as R⁶, R⁷, R⁹, and Y.

85. The conjugate according to any one of statements 1 to 84 wherein, wherein L-R^{L1′} or L-R^{L2′} is a group:

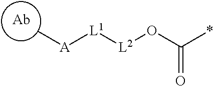

where the asterisk indicates the point of attachment to the PBD, Ab is the antibody, L¹ is a cleavable linker, A is a connecting group connecting L¹ to the antibody, L² is a covalent bond or together with —OC(═O)— forms a self-immolative linker.

86. The conjugate of statement 85, wherein L¹ is enzyme cleavable.

87. The conjugate of statement 85 or statement 86, wherein L¹ comprises a contiguous sequence of amino acids.

88. The conjugate of statement 87, wherein L¹ comprises a dipeptide and the group —X₁—X₂— in dipeptide, —NH—X₁-X₂—CO—, is selected from:
-Phe-Lys-,
-Val-Ala-,
-Val-Lys-,
-Ala-Lys-,
-Val-Cit-,
-Phe-Cit-,
-Leu-Cit-,
-Ile-Cit-,
-Phe-Arg-,
-Trp-Cit-.

89. The conjugate according to statement 88, wherein the group —X₁—X₂— in dipeptide, —NH—X₁-X₂—CO—, is selected from:
-Phe-Lys-,
-Val-Ala-,
-Val-Lys-,
-Ala-Lys-,
-Val-Cit-.

90. The conjugate according to statement 89, wherein the group —X₁—X₂— in dipeptide, —NH—X₁-X₂—CO—, is -Phe-Lys-, -Val-Ala- or -Val-Cit-.

91. The conjugate according to any one of statements 88 to 90, wherein the group X₂—CO— is connected to L².

92. The conjugate according to any one of statements 88 to 91, wherein the group NH—X₁— is connected to A.

93. The conjugate according to any one of statements 88 to 92, wherein L² together with OC(=O) forms a self-immolative linker.

94. The conjugate according to statement 93, wherein C(═O)O and L² together form the group:

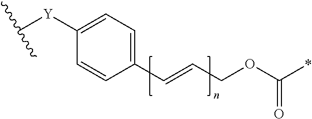

where the asterisk indicates the point of attachment to the PBD, the wavy line indicates the point of attachment to the linker L¹, Y is NH, O, C(═O)NH or C(═O)O, and n is 0 to 3.

95. The conjugate according to statement 94, wherein Y is NH.

96. The conjugate according to statement 94 or statement 95, wherein n is 0.

97. The conjugate according to statement 95, wherein L¹ and L² together with —OC(═O)-comprise a group selected from:

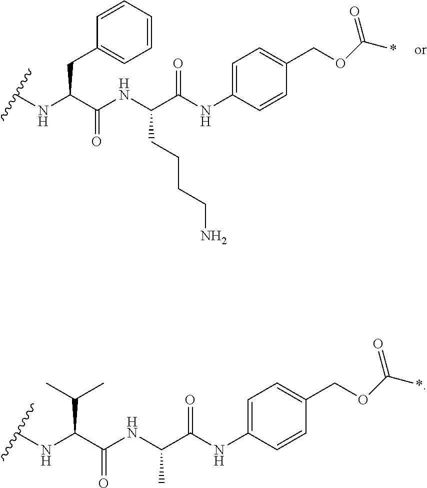

where the asterisk indicates the point of attachment to the PBD, and the wavy line indicates the point of attachment to the remaining portion of the linker L¹ or the point of attachment to A.

98. The conjugate according to statement 97, wherein the wavy line indicates the point of attachment to A.

99. The conjugate according to any one of statements 85 to 98, wherein A is:

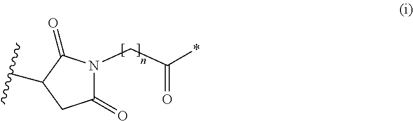

where the asterisk indicates the point of attachment to L¹, the wavy line indicates the point of attachment to the antibody, and n is 0 to 6; or

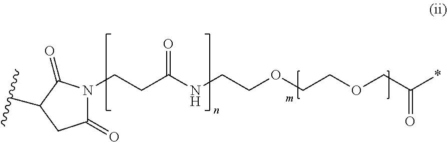

where the asterisk indicates the point of attachment to L¹, the wavy line indicates the point of attachment to the antibody, n is 0 or 1, and m is 0 to 30.

100. A conjugate according to statement 1 of formula ConjA:

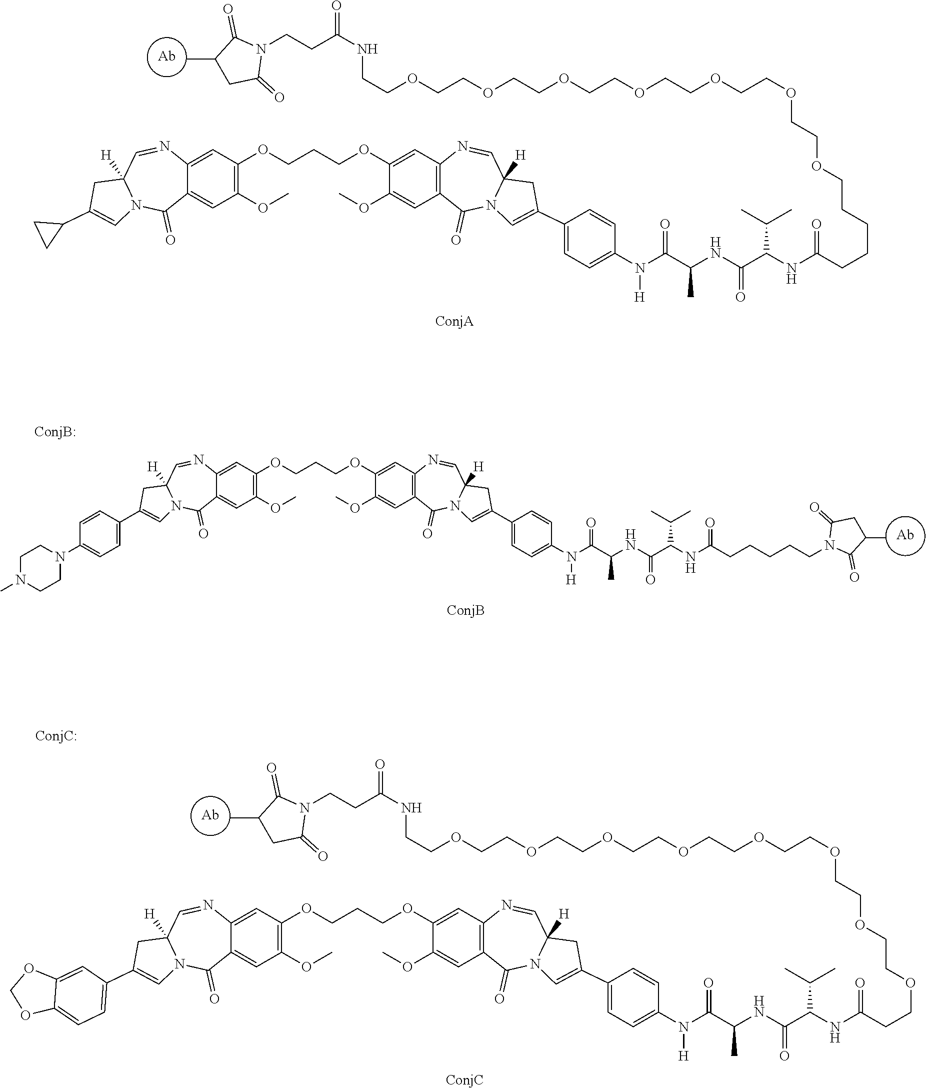

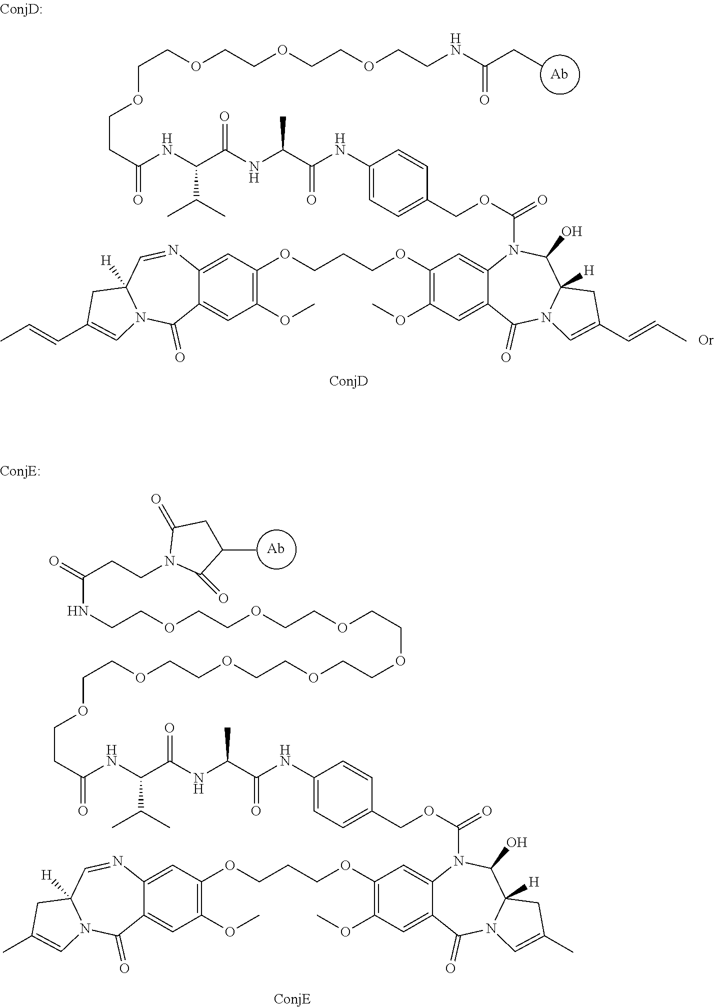

101. The conjugate according to any one of statements 1 to 100 wherein the antibody comprises:
a VH domain having a VH CDR1 with the amino acid sequence of SEQ ID NO.3, a VH CDR2 with the amino acid sequence of SEQ ID NO.4, and a VH CDR3 with the amino acid sequence of SEQ ID NO.5.

102. The conjugate according to any one of statements 1 to 101 wherein the antibody comprises a VH domain having the sequence according to SEQ ID NO. 1.

103. The conjugate according to any one of statements 1 to 102 wherein the antibody comprises:
a VL domain comprising a VL CDR1 with the amino acid sequence of SEQ ID NO.6, a VL CDR2 with the amino acid sequence of SEQ ID NO.7, and a VL CDR3 with the amino acid sequence of SEQ ID NO.8.

104. The conjugate according to any one of statements 1 to 103 wherein the antibody comprises a VL domain having the sequence according to SEQ ID NO. 2.

105. The conjugate according to any one of statements 1 to 103 wherein the antibody in an intact antibody.

106. The conjugate according to any one of statements 1 to 105 wherein the antibody is humanised, deimmunised or resurfaced.

107. The conjugate according to any one of statements 1 to 104 wherein the antibody is a fully human monoclonal IgG1 antibody, preferably IgG1,K.

108. The conjugate according to any one of statements 1 to 107 wherein the drug loading (p) of drugs (D) to antibody (Ab) is an integer from 1 to about 8.

109. The conjugate according to statement 108, wherein p is 1, 2, 3, or 4.

110. The conjugate according to statement 108 comprising a mixture of the antibody-drug conjugate compounds, wherein the average drug loading per antibody in the mixture of antibody-drug conjugate compounds is about 2 to about 5.

The definition of the terms used in the above statement are as defined in WO2014/057119.

Preferred CD25-ADC Embodiments

The term anti-CD25-ADC may include any embodiment described in WO 2014/057119. In particular, in preferred embodiments the ADC has the chemical structure:

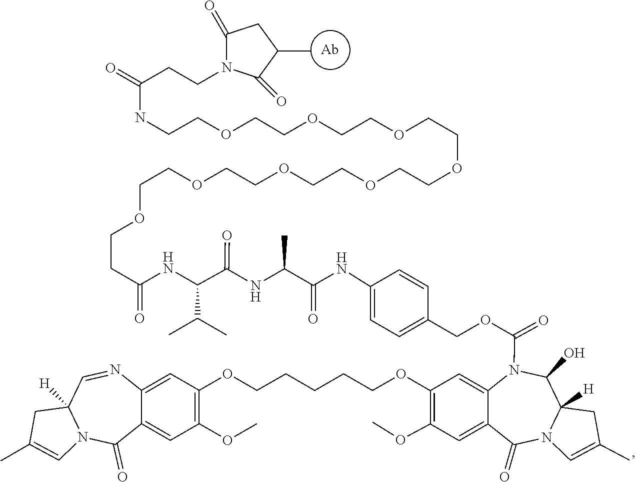

where the Ab is a CD25 antibody, and the DAR is between 1 and 8.

The antibody may comprise a VH domain comprising a VH CDR1 with the amino acid sequence of SEQ ID NO.3, a VH CDR2 with the amino acid sequence of SEQ ID NO.4, and a VH CDR3 with the amino acid sequence of SEQ ID NO.5.

In some aspects the antibody component of the anti-CD25-ADC is an antibody comprising: a VH domain comprising a VH CDR1 with the amino acid sequence of SEQ ID NO.3, a VH CDR2 with the amino acid sequence of SEQ ID NO.4, and a VH CDR3 with the amino acid sequence of SEQ ID NO.5. In some embodiments the antibody comprises a VH domain having the sequence according to SEQ ID NO. 1.

The antibody may further comprise: a VL domain comprising a VL CDR1 with the amino acid sequence of SEQ ID NO.6, a VL CDR2 with the amino acid sequence of SEQ ID NO.7, and a VL CDR3 with the amino acid sequence of SEQ ID NO.8. In some embodiments the antibody further comprises a VL domain having the sequence according to SEQ ID NO. 2.

In some embodiments the antibody comprises a VH domain and a VL domain, the VH and VL domains having the sequences of SEQ ID NO. 1 paired with SEQ ID NO. 2.

The VH and VL domain(s) may pair so as to form an antibody antigen binding site that binds CD25.

In preferred embodiments the antibody is an intact antibody comprising a VH domain and a VL domain, the VH and VL domains having sequences of SEQ ID NO. 1 and SEQ ID NO. 2.

In some embodiments the antibody is a fully human monoclonal IgG1 antibody, preferably IgG1,κ.

In some embodiments the antibody is the AB12 antibody described in WO 2004/045512 (Genmab A/S).

In an aspect the antibody is an antibody as described herein which has been modified (or further modified) as described below. In some embodiments the antibody is a humanised, deimmunised or resurfaced version of an antibody disclosed herein.

The most preferred anti-CD25-ADCs for use with the aspects of the present disclosure is ADCX25/ADCT-301/Camidanlumab Tesirine; the structure of ADCx25 is described herein below.

ADCx25

ADCx25 is an antibody drug conjugate composed of a human antibody against human CD25 attached to a pyrrolobenzodiazepine (PBD) warhead via a cleavable linker. The mechanism of action of ADCX25 depends on CD25 binding. The CD25 specific antibody targets the antibody drug conjugate (ADC) to cells expressing CD25. Upon binding, the ADC internalizes and is transported to the lysosome, where the protease sensitive linker is cleaved and free PBD dimer is released inside the target cell. The released PBD dimer inhibits transcription in a sequence-selective manner, due either to direct inhibition of RNA polymerase or inhibition of the interaction of associated transcription factors. The PBD dimer produces covalent crosslinks that do not distort the DNA double helix and which are not recognized by nucleotide excision repair factors, allowing for a longer effective period (Hartley 2011).

It has the chemical structure:

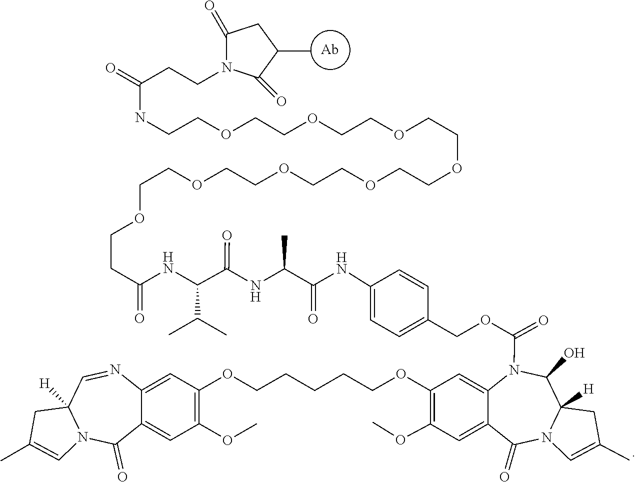

Ab represents Antibody AB12 (fully human monoclonal IgG1, K antibody with the VH and VL sequences SEQ ID NO. 1 and SEQ ID NO. 2, respectively, also known as HuMax-TAC). It is synthesised as described in WO 2014/057119 (Conj AB12-E) and typically has a DAR (Drug to Antibody Ratio) of 2.0+/−0.3.

CD25 Binding

The “first target protein” (FTP) as used herein is preferably CD25.

As used herein, “binds CD25” is used to mean the antibody binds CD25 with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds CD25 with an association constant (Kₐ) at least 2, 3, 4, 5, 10, 20, 50, 100, 200, 500, 1000, 2000, 5000, 104, 10⁵ or 10⁶-fold higher than the antibody's association constant for BSA, when measured at physiological conditions. The antibodies of the disclosure can bind CD25 with a high affinity. For example, in some embodiments the antibody can bind CD25 with a K_{D} equal to or less than about 10⁻⁶ M, such as equal to or less than one of 1×10⁻⁶, 10⁻⁷, 10⁻⁸, 10⁻⁹, 10⁻¹⁰, 10⁻¹¹, 10⁻¹², 10⁻¹³ or 10⁻¹⁴.

In some embodiments, CD25 polypeptide corresponds to Genbank accession no. NP_000408, version no. NP_000408.1 GI:4557667, record update date: Sep. 9, 2012 04:59 PM. In one embodiment, the nucleic acid encoding CD25 polypeptide corresponds to Genbank accession no. NM_000417, version no. NM_000417.2 GI:269973860, record update date: Sep. 9, 2012 04:59 PM. In some embodiments, CD25 polypeptide corresponds to Uniprot/Swiss-Prot accession No. P01589.

Therapeutic Uses of CD25 ADCs

The efficacy of an Antibody Drug Conjugate comprising an anti-CD25 antibody (herein termed a CD25-ADC) in the treatment of, for example, cancer is known—see, for example, WO2014/057119, WO2016/083468, and WO2016/166341.

Combination with Cell Therapy

In an aspect, the therapies described herein are combined with cell therapy, such as CAR T-cell therapy. Without wishing to be bound by theory, in this aspect the administration of a CD25-ADC is used to reduce the immune-suppressive activity of a population of immune regulatory cells, to allow for greater efficacy of a cell therapy administered in combination with the CD25-ADC.

Preferably, the CD25-ADC is administered before the cell therapy. For example, the CD25-ADC may be administered 1 hour, 2 hours, 6 hours, 12 hours, or 24 hours before the cell therapy. The CD25-ADC may be administered 1 day, 2 days, 3 days, 4 days, 5 days, 6 days, 7 days, 8 days, 9 days, 10 days, 11 days, 12 days, 13 days, or 14 days before the cell therapy.

Preferably the gap between CD25-ADC and cell therapy administrations is sufficient for the systemic level of CD25-ADC to drop to 25% or less of the administered dose (i.e. 2 half-lives of the CD25-ADC) before the cell therapy is administered. This is so the cells administered during the cell therapy are not exposed to high levels of the CD25-ADC. Typically, the CD25-ADC is administered at least 7 days before the cell therapy.

In some embodiments the immune-suppressive activity of a population of immune regulatory cells is reduced before the cell therapy is administered to the subject. In some embodiments the reduction in the immune-suppressive activity of the immune regulatory cell population is achieved by killing a proportion of the population. In some embodiments the reduction in the immune-suppressive activity of the immune regulatory cell population is achieved by inhibiting the immune-suppressive activity of a proportion of the regulatory cell population without filling the cells.

The immune regulatory cells may be myeloid-derived suppressor cells (MDSCs), mesenchymal stromal cells (MSCs), Type II NKT cells, Treg cells, or any other immune regulatory immune cells as defined herein.

Preferably the immune regulatory cells whose immune-suppressive activity is reduced are Treg cells. The term “Treg” cells as used herein refers to regulatory T-cells. This cell population may be identified by the following pattern of surface-marker expression: CD4+ve, CD25^{high} CD127^{llow/-} (see Yu et al. 2012, Inflammation 35(6), pp. 1773-1780.

In some embodiments the immune-suppressive activity of a population of immune regulatory cells is reduced by at least 10%, at least 20%, at least 30%, at least 40%, at least 50%, at least 60%, at least 70%, at least 70%, at least 90%, at least 95%, or at least 98% before the cell therapy is administered to the subject. Preferably, the reduction is measured relative to levels in the same subject prior to the administration of the CD25-ADC.

In some embodiments the immune-suppressive activity of a population of immune regulatory cells is assessed by measuring the level of inhibitory cytokines such as IL-10, TGFβ, or IL-35 (see Bettini et al., Current opinion in immunology. 2009; 21(6):612-618). In some embodiments the immune-suppressive activity of a population of immune regulatory cells is assessed by measuring the expression of specific genes such as LAYN, MADEH1, or CCR8 (see de Simone et al., Immunity. 2016 Nov. 15; 45(5): 1135-1147).

In some embodiments the size of a population of immune regulatory cells is reduced by at least 10%, at least 20%, at least 30%, at least 40%, at least 50%, at least 60%, at least 70%, at least 70%, at least 90%, at least 95%, or at least 98% before the cell therapy is administered to the subject. Preferably, the reduction in population size is measured relative to levels in the same subject prior to the administration of the CD25-ADC.

In some embodiments the population of immune regulatory cells is measured systemically, for example by using FACS on a representative sample such as whole blood. In some embodiments the population of immune regulatory cells is measured locally, for example in a sample taken from a tumour or the tumour microenvironment. The local population of immune regulatory cells may be measured by, for example, immunofluorescence of tissue sections.

In some embodiments the CD25-ADC is administered concomitantly with the cell therapy.

In some embodiments the cell therapy comprises the administration of autologous cells. In some embodiments the cell therapy comprises the administration of allogenic cells.

In some embodiments the cell therapy comprises the administration of stem cells.

In some embodiments the cell therapy comprises the administration of immune cells. The immune cells may be cytotoxic effector cells. The immune cells may be T-cells, Natural Killer (NK) cells, Natural Killer T-cell (NKT), Lymphokine-activated Killer (LAK) cells, or macrophages.

In preferred embodiments, the immune cells are ‘CAR immune cells’, as defined herein below.

CAR Immune Cells

Conventional immune cells may be genetically modified such that they express a CAR as described and referenced herein below. An immune cells so modified is described herein as “CAR immune cell” and is suitable for use in the cell therapies as described herein.

CARs may be expressed in a number of different immune cells. Suitable immune cells for expressing a CAR include T-cells—such as cytotoxic T-cells and helper T-cells—as well as Natural Killer (NK) cells.

In preferred cases, the CAR immune cell is a T-cell. In these cases, the CAR expressing T-cell is termed here in as a “CAR T-cell”.

CARs

As used herein, the term Chimeric antigen receptors (CARs) has its normal meaning in the art, including The current state of the art with respect to CARs is reviewed in for example: Hartmann, J., et al., EMBO Mol. Med., 2017, Vol. 9, Issue 9, pp. 1183-1197//Brudno, JN. & Kochendorfer, J N., 2016, Blood, vol. 127, no. 26, pp. 3321-3330//Zhang and Xu, J. Hematology & Oncology, 2017, 10:1, DOI 10.1186/s13045-016-0379-6//references cited therein.

Briefly, all CARs are composed of an extracellular binding domain, a hinge region, a transmembrane domain, and one or more intracellular signaling domains. Single-chain variable fragments (scFvs) derived from tumor antigen-reactive antibodies are typically used as extracellular binding domains. All CARs harbor the CD3epsilon chain domain as the intracellular signaling domain.

Like antibodies, the extracellular binding domain of a CAR is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple CARs. Each CAR that specifically binds to a different epitope has a different structure. Thus, one antigen may have more than one corresponding CAR. A CAR may comprise a full-length immunoglobulin molecule or an immunologically active portion of a full-length immunoglobulin molecule, i.e., a molecule that contains an antigen binding site that immunospecifically binds an antigen of a target of interest or part thereof (eg. An scFv), such targets including but not limited to, cancer cells.

In addition to the CD3 epsilon chain, second- and third-generation CARs also contain one or more co-stimulatory domains, like CD28 and/or 4-1 BB. These co-stimulatory domains improve proliferation, cytokine secretion, resistance to apoptosis, and in vivo persistence of cells expressing the CARs. Third-generation CARs exhibit improved effector functions and in vivo persistence as compared to second-generation CARs.

Fourth-generation CARs, so-called TRUCKs or armored CARs, combine the expression of a second-generation CAR with factors that enhance anti-tumoral activity, such as cytokines, costimulatory ligands, or enzymes that degrade the extracellular matrix of solid tumors (Chmielewski & Abken, 2015, Expert Opin Biol Ther 15, pp. 1145-1154).

Further varieties of CARs exist with enhanced safety features. These include so-called smart T cells which are either equipped with a suicide gene or include synthetic control devices are under non-clinical and clinical investigation (Zhang & Xu, ibid.).

Combination with Checkpoint Inhibitors

In an aspect, the CD25-ADCs described herein are administered in combination with a checkpoint inhibitor.

The CD25-ADC may be administered before the checkpoint inhibitor, simultaneous with the checkpoint inhibitor, or after the checkpoint inhibitor.

The checkpoint inhibitor may be, for example, a PD1 antagonist, a PD-L1 antagonist, a GITR agonist, an OX40 agonist, or a CTLA-4 antagonist

PD1 Antagonists

Programmed death receptor I (PD1) is an immune-inhibitory receptor that is primarily expressed on activated T and B cells. Interaction with its ligands has been shown to attenuate T-cell responses both in vitro and in vivo. Blockade of the interaction between PD1 and one of its ligands, PD-L1, has been shown to enhance tumor-specific CD8+ T-cell immunity and may therefore be helpful in clearance of tumor cells by the immune system.

PD1 (encoded by the gene Pdcdl) is an Immunoglobulin superfamily member related to CD28, and CTLA-4. PD1 has been shown to negatively regulate antigen receptor signalling upon engagement of its ligands (PD-L1 and/or PD-L2). The structure of murine PD1 has been solved as well as the co-crystal structure of mouse PD1 with human PD-L1 (Zhang, X., et al., (2004) Immunity 20: 337-347; Lin, et al., (2008) Proc. Natl. Acad. Sci. USA 105: 30I I-6). PD1 and like family members are type I transmembrane glycoproteins containing an Ig Variable-type (V-type) domain responsible for ligand binding and a cytoplasmic tail that is responsible for the binding of signaling molecules. The cytoplasmic tail of PD1 contains two tyrosine-based signaling motifs, an ITIM (immunoreceptor tyrosine-based inhibition motif) and an ITSM (immunoreceptor tyrosine-based switch motif).

In humans, expression of PD1 (on tumor infiltrating lymphocytes) and/or PD-L1 (on tumor cells) has been found in a number of primary tumor biopsies assessed by immunohistochemistry. Such tissues include cancers of the lung, liver, ovary, cervix, skin, colon, glioma, bladder, breast, kidney, esophagus, stomach, oral squamous cell, urothelial cell, and pancreas as well as tumors of the head and neck (Brown, J. A., et al., (2003) J Immunol. I 70: I257-I266; Dong H., et al., (2002) Nat. Med. 8: 793-800; Wintterle, et al., (2003) Cancer Res. 63: 7462-7467; Strome, S. E., et al., (2003) Cancer Res. 63: 650I-6505; Thompson, R. H., et al., (2006) Cancer Res. 66: 3381-5; Thompson, et al., (2007) Clin. Cancer Res. 13: I 757-6I; Nomi, T., et al., (2007) Clin. Cancer Res. 13: 2I5I-7). More strikingly, PD-ligand expression on tumor cells has been correlated to poor prognosis of cancer patients across multiple tumor types (reviewed in Okazaki and Honjo, (2007) Int. Immunol. 19: 813-824).

To date, numerous studies have shown that interaction of PD1 with its ligands (PD-L1 and PD-L2) leads to the inhibition of lymphocyte proliferation in vitro and in vivo. Blockade of the PD1/PD-L1 interaction could lead to enhanced tumor-specific T-cell immunity and therefore be helpful in clearance of tumor cells by the immune system. To address this issue, a number of studies were performed. In a murine model of aggressive pancreatic cancer (Nomi, T., et al. (2007) Clin. Cancer Res. 13: 2151-2157), the therapeutic efficacy of PD1/PD-L1 blockade was demonstrated. Administration of either PD1 or PD-L1 directed antibody significantly inhibited tumor growth. Antibody blockade effectively promoted tumor reactive CD8+ T cell infiltration into the tumor resulting in the up-regulation of anti-tumor effectors including IFN gamma, granzyme Band perforin. Additionally, the authors showed that PD1 blockade can be effectively combined with chemotherapy to yield a synergistic effect. In another study, using a model of squamous cell carcinoma in mice, antibody blockade of PD1 or PD-L1 significantly inhibited tumor growth (Tsushima, F., et al., (2006) Oral Oneal. 42: 268-274).

“PD1 antagonist” means any chemical compound or biological molecule that stimulates an immune reaction through inhibition of PD1 signalling.

To examine the extent of enhancement of, e.g., PD1 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) with PD1 inhibitors is advantageous, because on the one hand, the ADC will directly kill the FTP positive tumor cells, while on the other hand the PD1 inhibitor will engage the patient's own immune system to eliminate the cancer cells. Next to FTP(+) tumor cells, FTP negative tumor cells in close proximity to FTP(+) tumor cells will potentially be killed by the bystander mechanism of the PBD-dimer released after cell kill of CD25(+) cells. Hence, the ADC will directly kill the tumor cells.

The resulting release of tumor associated antigens from cells that are killed with the PBD dimer will trigger the immune system, which will be further enhanced by the use of programmed cell death protein 1 (PD1) inhibitors, expressed on a large proportion of tumour infiltrating lymphocytes (TILs) from many different tumour types. Blockade of the PD1 pathway may enhance antitumour immune responses against the antigens released from the tumors killed by the ADC by diminishing the number and/or suppressive activity of intratumoral TReg cells.

The major function of PD1 is to limit the activity of T-cells at the time of an anti-inflammatory response to infection and to limit autoimmunity. PD1 expression is induced when T-cells become activated, and binding of one of its own ligands inhibits kinases involved in T-cell activation. Hence, in the tumor environment this may translate into a major immune resistance, because many tumours are highly infiltrated with TReg cells that probably further suppress effector immune responses. This resistance mechanism is alleviated by the use of PD1 inhibitors in combination with the ADC.

PD1 antagonists suitable for use as secondary agents in the present disclosure include:
a) a PD1 antagonist which inhibits the binding of PD1 to its ligand binding partners.
b) a PD1 antagonist which inhibits the binding of PD1 to PD-L1.
c) a PD1 antagonist which inhibits the binding of PD-1 to PDL2.
d) a PD1 antagonist which inhibits the binding of PD-1 to both PDLI and PDL2.
e) a PD1 antagonist of parts (a) to (d) which is an antibody.

Specific PD1 antagonists suitable for use as secondary agents in the present disclosure include:
a) pembrolizumab (brand name Keytruda)
   i. CAS Number→1374853-91-4
      (see http://www.cas.org/content/chemical-substances/faqs)
   ii. NCBI Pubchem reference→254741536
      (see https://pubchem.ncbi.nlm.nih.gov/)
   iii. DrugBank reference→DB09037
      (see https://www.drugbank.ca/)
   iv. Unique Ingredient Identifier (UNII)→DPT003T46P
   (see http://www.fda.gov/Forlndustry/DataStandards/SubstanceRegistration System-UniqueIngredientIdentifierUNII/default.htm)
b) nivolumab (brand name Opdivo)
   i. CAS Number→946414-94-4
      (see http://www.cas.org/content/chemical-substances/faqs)
   ii. DrugBank reference→DB09035
      (see https://www.drugbank.ca/)
c) MED10680 (formerly AMP-514)
   As described in WO2014/055648, WO2015/042246, WO2016/127052, WO2017/004016, WO2012/145493, U.S. Pat. No. 8,609,089, WO2016/007235, WO2016/011160; Int. J. Mol. Sci. 2016 July; 17(7): 1151, doi: 10.3390/ijms17071151; and Drug Discov Today, 2015 September; 20(9):1127-34. doi: 10.1016/j.drudis.2015.07.003.
   See also clinical trials NCT02271945 and NCT02013804 at https://clinicaltrials.gov/ct2/home
d) PDR001 (spartalizumab)
   i. CAS Number→1935694-88-4
      (see http://www.cas.org/content/chemical-substances/faqs)
   ii. Unique Ingredient Identifier (UNII)→QOG25L6Z8Z
      (see http://www.fda.gov/Forlndustry/DataStandards/SubstanceRegistration System-UniquelngredientldentifierUNll/default.htm)
      As described in WO2016/007235 and WO2016/011160
      NCI thesaurus code→C121625
      (see https://ncit.nci.nih.gov/ncitbrowser/)
e) Camrelizumab [INCSHR-1210] (Incyte)
   i. CAS Number→1798286-48-2
      (see http://www.cas.org/content/chemical-substances/faqs)
   ii. Unique Ingredient Identifier (UNII)→73096E137E
      (see

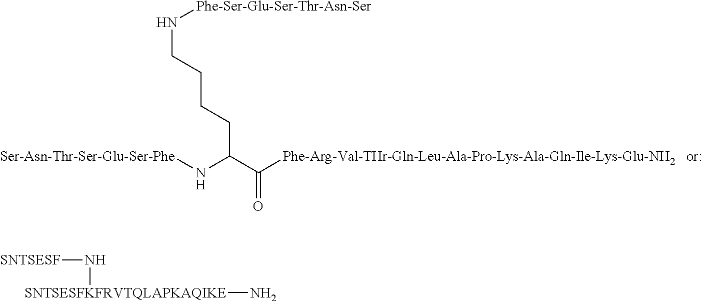

http://www.fda.gov/Forlndustry/DataStandards/SubstanceRegistration System-UniquelngredientldentifierUNll/default.htm)
f) AUNP12 (peptide) (Aurigene/PierreFabre)
   i. Disclosed in WO2011/161699 as SEQ ID NO:49 a.k.a. “compound 8”, see Example 2 on page 77 of the A2 publication of WO2011/161699.
   ii. CAS Number→1353563-85-5 (see http://www.cas.org/content/chemical-substances/faqs)
g) Pidilizumab (CT-01 1)
   i. CAS Number→1036730-42-3
      (see http://www.cas.org/content/chemical-substances/faqs)
   ii. Unique Ingredient Identifier (UNII)→B932PAQ1BQ
      (see http://www.fda.gov/Forlndustry/DataStandards/SubstanceRegistration System-UniquelngredientldentifierUNll/default.htm)
h) Cemiplimab (formerly REGN-2810, SAR-439684)
   i. CAS Number→1801342-60-8
      (see http://www.cas.org/content/chemical-substances/faqs)
   ii. Unique Ingredient Identifier (UNII)→6QVL057INT
      (see http://www.fda.gov/Forlndustry/DataStandards/SubstanceRegistration System-UniqueIngredientIdentifierUNII/default.htm)
      As described in WO2016/007235
      NCI thesaurus code→C121540
      (see https://ncit.nci.nih.gov/ncitbrowser/)
i) BGB-A317 (Tislelizumab)
   i. As described in U.S. Pat. No. 9,834,606 B2
   ii. See clinical trial NCT03209973 (https://clinicaltrials.gov/)
   iii. NCI thesaurus code C121775
      (see https://ncit.nci.nih.gov/ncitbrowser/)
j) BGB-108
   See WO2016/000619 and U.S. Pat. No. 8,735,553
k) AMP-224
   see clinical trial NCT02298946, https://clinicaltrials.gov/ct2/home

In some embodiments, PD1 polypeptide corresponds to Genbank accession no. AAC51773, version no. AAC51773.1, record update date: Jun. 23, 2010 09:24 AM. In one embodiment, the nucleic acid encoding PD1 polypeptide corresponds to Genbank accession no. U64863, version no. U64863.1, record update date: Jun. 23, 2010 09:24 AM. In some embodiments, PD1 polypeptide corresponds to Uniprot/Swiss-Prot accession No. Q15116.

PD-L1 Antagonists

“PD-L1 antagonist” means any chemical compound or biological molecule that stimulates an immune reaction through inhibition of PD-L1 signalling.

To examine the extent of enhancement of, e.g., PD-L1 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with PD-L1 inhibitors is advantageous because, on the one hand, the ADC will directly kill the FTP positive tumor cells while, on the other hand, the PD-L1 inhibitor will engage the patient's own immune system to eliminate the cancer cells.

Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor cells. The resulting release of tumor associated antigens from cells that are killed with the PBD dimer will trigger the immune system, which will be further enhanced by the use of programmed cell death protein 1 ligand inhibitors (PD-L1, aka B7-H1 or CD274).

PD-L1 is commonly upregulated on the tumour cell surface from many different human tumours. Interfering with the PD1 ligand expressed on the tumor will avoid the immune inhibition in the tumor microenvironment and therefore blockade of the PD1 pathway using PDL1 inhibitors may enhance antitumour immune responses against the antigens released from the tumors killed by the ADC.

Combining an ADC, which targets a first target protein (FTP) with PD1 inhibitors is advantageous, because on the one hand, the ADC will directly kill the FTP positive tumor cells, while on the other hand the PD1 inhibitor will engage the patient's own immune system to eliminate the cancer cells. Next to FTP(+) tumor cells, FTP negative tumor cells in close proximity to FTP(+) tumor cells will potentially be killed by the bystander mechanism of the PBD-dimer released after cell kill of CD19(+) or CD22 (+) cells. Hence, the ADC will directly kill the tumor cells.

PD-L1 antagonists suitable for use as secondary agents in the present disclosure include PD-L1 antagonists that:
(a) are PD-L1 binding antagonists;
(b) inhibit the binding of PD-L1 to PD1;
(c) inhibit the binding of PD-L1 to B7-1;
(d) inhibit the binding of PD-L1 to both PD1 and B7-1;
(e) are anti-PD-L1 antibodies.

Specific PD-L1 antagonists suitable for use as secondary agents in the present disclosure include:
a) atezolizumab (MPDL3280A, brand name Tecentriq)
   i. CAS Number→1380723-44-3
      (see http://www.cas.org/content/chemical-substances/faqs)
   ii. DrugBank reference→DB11595
      (see https://www.drugbank.ca/)
   iii. Unique Ingredient Identifier (UNII)→52CM10WC3Y
      (see http://www.fda.gov/Forlndustry/DataStandards/SubstanceRegistration System-UniqueIngredientIdentifierUNII/default.htm)
b) BMS-936559/MDX-1105
   I. CAS Number→1422185-22-5
      (see http://www.cas.org/content/chemical-substances/fags)
   II. see clinical trial NCT02028403, https://clinicaltrials.gov/ct2/home
   Ill. See WO2007/005874 for antibody sequences, in particular the:
      i. Antibody having:

a. VH CDR1 = DYGFS
b. VH CDR2 = WITAYNGNTNYAQKLQG
c. VH CDR3 = DYFYGMDV
d. VL CDR1 = RASQSVSSYLV
e. VL CDR2 = DASNRAT
f. VL CDR3 = QQRSNWPRT

ii. Antibody having:

a. VH CDR1 = TYAIS
b. VH CDR2 = GIIPIFGKAHYAQKFQG
c. VH CDR3 = KFHFVSGSPFGMDV
d. VL CDR1 = RASQSVSSYLA
e. VL CDR2 = DASNRAT
f. VL CDR3 = QQRSNWPT

iii. Antibody having:

a. VH CDR1 = SYDVH
b. VH CDR2 = WLHADTGITKFSQKFQG
c. VH CDR3 = ERIQLWFDY
d. VL CDR1 = RASQGISSWLA
e. VL CDR2 = AASSLQS
f. VL CDR3 = QQYNSYPYT

c) durvalumab/MED14736
i. CAS Number→1428935-60-7
   (see http://www.cas.org/content/chemical-substances/fags)
ii. Unique Ingredient Identifier (UNII)→28×28×90 KV
   (see http://www.fda.gov/Forlndustry/DataStandards/SubstanceRegistration System-UniquelngredientldentifierUNII/default.htm)

iii. VH sequence
EVQLVESGGGLVQPGGSLRLSCAASGFTFSRYWMSWVRQA
PGKGLEWVANIKQDGSEKYYVDSVKGRFTISRDNAKNSLY
LQMNSLRAEDTAVYYCAREGGWFGELAFDYWGQGTLVTVS
S
iv. VL sequence
EIVLTQSPGTLSLSPGERATLSCRASQRVSSSYLAWYQQKP
GQAPRLLIYDASSRATGIPDRFSGSGSGTDFTLTISRLEPE
DFAVYYCQQYGSLPWTFGQGTKVEIK

d) Avelumab/MSB0010718C
   i. CAS Number→1537032-82-8
      (see http://www.cas.org/content/chemical-substances/faqs)
   ii. Unique Ingredient Identifier (UNII)→KXG2PJ5511
      (see http://www.fda.gov/Forlndustry/DataStandards/SubstanceRegistration System-UniqueIngredientIdentifierUNII/default.htm)

In some embodiments, PD-L1 polypeptide corresponds to Genbank accession no. AAF25807, version no. AAF25807.1, record update date: Mar. 10, 2010 10:14 PM. In one embodiment, the nucleic acid encoding PD1 polypeptide corresponds to Genbank accession no. AF177937, version no. AF177937.1, record update date: Mar. 10, 2010 10:14 PM. In some embodiments, PD1 polypeptide corresponds to Uniprot/Swiss-Prot accession No. Q9NZQ7.

GITR Agonists

The term “glucocorticoid-induced TNF receptor” (abbreviated herein as “GITR”), also known as TNF receptor superfamily 18 (TNFRSF18, CD357), TEASR, and 312C2, as used herein, refers to a member of the tumor necrosis factor/nerve growth factor receptor family. GITR is a 241 amino acid type I transmembrane protein characterized by three cysteine pseudo-repeats in the extracellular domain and specifically protects T-cell receptorinduced apoptosis, although it does not protect cells from other apoptotic signals, including Fas triggering, dexamethasone treatment, or UV irradiation (Nocentini, G., et al. (1997) Proc. Natl. Acad. Sci. USA 94:6216-622).

GITR activation increases resistance to tumors and viral infections, is involved in autoimmune/inflammatory processes and regulates leukocyte extravasation (Nocentini supra; Cuzzocrea, et al. (2004) J Leukoc. Biol. 76:933-940; Shevach, et al. (2006) Nat. Rev. Immunol. 6:613-618; Cuzzocrea, et al. (2006) J Immunol. I 77:631-641; and Cuzzocrea, et al. (2007) FASEB J 21:1 I 7-129). In tumor mouse models, agonist GITR antibody, DTA-I, was combined with an antagonist CTLA-4 antibody, and showed synergistic results in complete tumor regression of advanced stage tumors in some test group mice (Ko, et al. (2005) J Exp. Med. 7:885-891).

The nucleic acid and amino acid sequences of human GITR (hGITR), of which there are three splice variants, are known and can be found in, for example GenBank Accession Nos. gi:40354198 [NM_005092.3], gi:23238190 [NM_004195.3], gi:23238193 [NM_148901.1], and gi:23238196 [NM_148902.1].

“GITR agonist” means any chemical compound or biological molecule that stimulates an immune reaction through activation of GITR signalling. Also contemplated are soluble GITR-L proteins, a GITR binding partner.

To examine the extent of enhancement of, e.g., GITR activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with GITR agonists is advantageous, because on the one hand the ADC will directly kill the FTP positive tumor cells, while on the other hand the GITR agonist will engage the patient's own immune system to eliminate the cancer cells. Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor. The resulting release of tumor associated antigens from cells killed with the PBD dimer will trigger the immune system, which will be further enhanced by the use of a GITR agonist.

GITR (Glucocorticoid-Induced TNFR-Related protein) is expressed transiently on activated T-cells and expressed constitutively at high levels on T-regs with further induction following activation. GITR ligation via its ligand GITRL stimulates both proliferation and function of both effector and regulatory CD4+ T cells. This promotes T-cell survival, and differentiation into effector cells, while abrogating suppression. Therefore it will be beneficial to target a FTP(+) tumor with the ADC, causing the antigenic cell death, while the GITR agonist induces a stronger, durable immune response.

Specific GITR agonists suitable for use as secondary agents in the present disclosure include:
a) MED11873, a GITR ligand fusion protein developed by MedImmune
   See WO2016/196792, US20160304607
   NCI thesaurus code→C124651
      (see https://ncit.nci.nih.gov/ncitbrowser)
   See also clinical trial NCT023126110 at https://clinicaltrials.gov/ct2/home
   See Tigue N J, Bamber L, Andrews J, et al. MED11873, a potent, stabilized hexameric agonist of human GITR with regulatory T-cell targeting potential. Oncoimmunology. 2017; 6(3):e1280645. doi:10.1080/2162402X.2017.1280645.
b) INCAGN1876, is an agonist antibody targeting the glucocorticoid-induced TNFR-related protein, or GITR. Discovered during a collaboration with Ludwig Cancer Research. INCAGN1876 is being co-developed with
   See clinical trials NCT02583165 and NCT03277352 at https://clinicaltrials.gov/ct2/home
c) TRX518, a humanized agylcosylated (Fc disabled) IgG1 anti-GITR mAb with immune-modulating activity developed by Leap Therapeutics
   See WO2006/105021 for sequences 58, 60-63; and EP2175884 sequences 1-7:

VL comprising the sequence
(CDR underline):
EIVMTQSPATLSVSPGERATLSCKASQNVGTNVAWYQQKPGQ
APRLLIYSASYRYSGIPARFSGSGSGTEFTLTISSLQSEDFA
VYYCQQYNTDPLTFGGGTKVEIK
VH comprising the sequence
(CDR underline):
QVTLRESGPALVKPTQTLTLTCTFSGFSLSTSGMGVGWIRQPPG
KALEWLAHIWWDDDKYY [symbol] PSLKSRLTISKDTSKNQVVLTMTNMD
PVDTATYYCARTRRYFPFAYWGQGTLVTVS
QVTLRESGPALVKPTQTLTLTCTFSGFSLSTSGMGVGWIRQPPG
KALEWLAHIWWDDDKYY [symbol] PSLKSRLTISKDTSKNQVVLTMTNM
DPVDTATYYCARTRRYFPFAYWGQGTLVTVS

See clinical trials NCT01239134 and NCT02628574 at https://clinicaltrials.gov/ct2/home
NCI thesaurus code→C95023
(see https://ncit.nci.nih.gov/ncitbrowser)
d) GWN323, an anti-GITR agonistic monoclonal antibody, which activates GITRs found on multiple types of T-cells. GWN323 is developed by Novartis
   See WO2016/196792
   NCI thesaurus code→C128028
      (see https://ncit.nci.nih.gov/ncitbrowser)
   See clinical trial NCT02740270 at https://clinicaltrials.gov/ct2/home
e) MK-1248, a humanized IgG4 anti-human glucocorticoid-induced tumor necrosis factor receptor (GITR) agonistic monoclonal antibody (MoAb) with significantly reduced effector function
   See clinical trial NCT02553499 at https://clinicaltrials.gov/ct2/home
   MK-1248 has the same CDR as MK4166 (see Sukumar et al., Cancer Res. 2017)
f) MK-4166, a humanized IgG1 anti-human glucocorticoid-induced tumor necrosis factor receptor (GITR) agonistic monoclonal antibody (MoAb) with potential immunomodulating activity (see Sukumar et al., Cancer Res. 2017).
   See clinical trial NCT02132754 at https://clinicaltrials.gov/ct2/home
   See Sukumar, et al., (2017), Cancer Research. 77. canres.1439.2016. 10.1158/0008-5472.CAN-16-1439.
   NCI thesaurus code C116065
   (see https://ncit.nci.nih.gov/ncitbrowser/)
g) BMS-986156, An anti-human glucocorticoid-induced tumor necrosis factor receptor (GITR; tumor necrosis factor superfamily member 18; TNFRSF18; CD357) agonistic monoclonal antibody
   See clinical trial NCT02598960 at https://clinicaltrials.gov/ct2/home
   NCI thesaurus code C132267
   (see https://ncit.nci.nih.gov/ncitbrowser/)

Sequences of agonist anti-GITR antibodies are provided in WO2011/028683 and WO2006/105021.

In some embodiments, GITR polypeptide corresponds to Genbank accession no. AAD22635, version no. AAD22635.1, record update date: Mar. 10, 2010 09:42 PM. In one embodiment, the nucleic acid encoding GITR polypeptide corresponds to Genbank accession no. AF125304, version no. AF125304.1, record update date: Mar. 10, 2010 09:42 PM. In some embodiments, GITR polypeptide corresponds to Uniprot/Swiss-Prot accession No. Q9Y5U5.

OX40 Agonists

OX40 (CD134; TNFRSF4) is a member of the TNFR super-family and is expressed by CD4 and CD8 T cells during antigen-specific priming. OX40 expression is largely transient following TCR/CD3 cross-linking, and by the presence of inflammatory cytokines. In the absence of activating signals, relatively few mature T cell subsets express OX40 at biologically relevant levels. Generating optimal “killer” CD8 T cell responses requires T cell receptor activation plus co-stimulation, which can be provided through ligation of OX40 using a OX40 agonist. This activating mechanism augments T cell differentiation and cytolytic function leading to enhanced anti-tumor immunity. Therefore it will be beneficial to target a FTP(+) tumor with the ADC, causing the antigenic cell death, while the OX40 agonist induces a stronger, durable immune response.

The OX40 agonist may be selected from the group consisting of an OX40 agonist antibody, an OX40L agonist fragment, an OX40 oligomeric receptor, and an OX40 immunoadhesin. In some embodiments, the OX40 binding agonist is a trimeric OX40L-Fc protein.

In some embodiments, the OX40 binding agonist is an OX40L agonist fragment comprising one or more extracellular domains of OX40L. In some embodiments, the OX40 binding agonist is an OX40 agonist antibody that binds human OX40. In some embodiments, the OX40 agonist antibody depletes cells that express human OX40. In some embodiments, the OX40 agonist antibody depletes cells that express human OX40 in vitro. In some embodiments, the cells are CD4+ effector T cells. In some embodiments, the cells are Treg cells. In some embodiments, the depleting is by ADCC and/or phagocytosis. In some embodiments, the depleting is by ADCC. In some embodiments, the OX40 agonist antibody binds human OX40 with an affinity of less than or equal to about 1 nM. In some embodiments, the OX40 agonist antibody increases CD4+ effector T cell proliferation and/or increasing cytokine production by the CD4+ effector T cell as compared to proliferation and/or cytokine production prior to treatment with anti-human OX40 agonist antibody. In some embodiments, the cytokine is gamma interferon. In some embodiments, the OX40 agonist antibody increases memory T cell proliferation and/or increasing cytokine production by the memory cell. In some embodiments, the cytokine is gamma interferon. In some embodiments, the OX40 agonist antibody inhibits Treg function. In some embodiments, the OX40 agonist antibody inhibits Treg suppression of effector T cell function. In some embodiments, effector T cell function is effector T cell proliferation and/or cytokine production. In some embodiments, the effector T cell is a CD4+ effector T cell. In some embodiments, the OX40 agonist antibody increases OX40 signal transduction in a target cell that expresses OX40. In some embodiments, OX40 signal transduction is detected by monitoring NFkB downstream signalling.

“OX40 agonist” means any chemical compound or biological molecule that stimulates an immune reaction through iactivation of OX40 signalling.

To examine the extent of enhancement of, e.g., OX40 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with OX40 agonists is advantageous, because on the one hand the ADC will directly kill the FTP positive tumor cells, while on the other hand the OX40 agonist will engage the patient's own immune system to eliminate the cancer cells. Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor. The resulting release of tumor associated antigens from cells killed with the PBD dimer will trigger the immune system, which will be further enhanced by the use of a OX40 agonist.

Specific OX40 agonists suitable for use as secondary agents in the present disclosure include:
a) MED10562 (aka Tavolixizumab, Tavolimab)
   i. CAS Number→1635395-25-3
      (see http://www.cas.org/content/chemical-substances/fags)
   ii. Unique Ingredient Identifier (UNII)→4LU9B48U4D
      (see http://www.fda.gov/ForIndustry/DataStandards/SubstanceRegistration System-UniquelngredientidentifierUNII/default.htm)
      See clinical trial NCT02318394 at https://clinicaltrials.gov/ct2/home
      As described in WO2015/095423, WO2015/153514, WO2016/073380 & WO2016/081384
      NCI thesaurus code→C120041
      (see https://ncit.nci.nih.gov/ncitbrowser/)

Heavy Chain sequence:
QVQLQESGPGLVKPSQTLSLTCAVYGGSFSSGYWN
WIRKHPGKGLEYIGYISYNGITYHNPSLKSRITIN
RDTSKNQYSLQLNSVTPEDTAVYYCARYKYDYDGG
HAMDYWGQGTLVTVSSASTKGPSVFPLAPSSKSTS
GGTAALGCLVKDYFPEPVTVSWNSGALTSGVHTFP
AVLQSSGLYSLSSVVTVPSSSLGTQTYICNVNHKP
SNTKVDKRVEPKSCDKTHTCPPCPAPELLGGPSVF
LFPPKPKDTLMISRTPEVTCWVDVSHEDPEVKFNV
VYVDGVEVHNAKTKPREEQYNSTYRVVSVLTVLHQ
DWLNGKEYKCKVSNKALPAPIEKTISKAKGQPREP
QVYTLPPSREEMTKNQVSLTCLVKGFYPSDIAVEW
ESNGQPENNYKTTPPVLDSDGSFFLYSKLTVDKSR
WQQGNVFSCSVMHEALHNHYTQKSLSLSPG
Light chain sequence:
DIQMTQSPSSLSASVGDRVTITCRASQDISNYLNW
YQQKPGKAPKLLIYYTSKLHSGVPSRFSGSGSGTD
YTLTISSLQPEDFATYYCQQGSALPWTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC

b) MED16383 (Efizonerimod alfa)
   i. CAS Number→1635395-27-5
      (see http://www.cas.org/content/chemical-substances/fags)
   ii. Unique Ingredient Identifier (UNII)→1 MH7C2X8KE
      (see http://www.fda.gov/ForIndustry/DataStandards/SubstanceRegistration System-UniqueIngredientIdentifierUNII/default.htm)
      See clinical trial NCT02221960 at https://clinicaltrials.gov/ct2/home
      As described in WO2015/095423, WO2016/081384, and WO2016/189124
      NCI thesaurus code→C118282
      (see https://ncit.nci.nih.gov/ncitbrowser/)

Amino acid sequence (Seq ID no. 17
from W02016/189124):
ESKYGPPCPPCPAPEFLGGPSVFLFPPKPKDTLMIS
RTPEVTCVVVDVSQEDPEVQFNWYVDGVEVHNAKT
KPREEQFNSTYRVVSVLTVLHQDWLNGKEYKCKVS
NKGLPSSIEKTISKAKGQPREPQVYTLPPSQEEMT
KNQVSLTCLVKGFYPSDIAVEWESNGQPENNYKTT
PPVLDSDGSFFLYSRLTVDKSRWQEGNVFSCSVMH
EALHNHYTQKSLSLSLGKDQDKIEALSSKVQQLER
SIGLKDLAMADLEQKVLEMEASTQVSHRYPRIQSI
KVQFTEYKKEKGFILTSQKEDEIMKVQNNSVIINC
DGFYLISLKGYFSQEVNISLHYQKDEEPLFQLKKV
RSVNSLMVASLTYKDKVYLNVTTDNTSLDDFHVNG
GELILIHQNPGEFCVL

c) MOXR0916 (also known as RG7888, Pogalizumab), a humanized anti-OX40 monoclonal antibody
   i. CAS Number→1638935-72-4
      (see http://www.cas.org/content/chemical-substances/faqs)
   ii. Unique Ingredient Identifier (UNII)→C78148TF1D
      (see http://www.fda.gov/ForIndustry/DataStandards/SubstanceRegistration System-UniqueIngredientIdentifierUNII/default.htm)
   iii. NCI thesaurus code→C121376
      (see https://ncit.nci.nih.gov/ncitbrowser/)
d) OX40mAb24 (9B12)
   i. OX40mAb24 is a humanised version of 9B12. 9B12 is a murine IgGI, anti-OX40 mAb directed against the extracellular domain of human OX40 (CD134) (Weinberg, A. D., et al. J Immunother 29, 575-585 (2006)).
   ii. See WO2016/057667 Seq ID no.59 for OX40mAb24 VH sequence, no.29 for VL sequence (no.32 is an alternative VL):

VH sequence
QVQLQESGPGLVKPSQTLSLTCAVYGGSFSSGYWN
WIRKHPGKGLEYIGYISYNGITYHNPSLKSRITIN
RDTSKNQYSLQLNSVTPEDTAVYYCARYKYDYDGG
HAMDYWGQGTLVTVSS
VL sequence
DIQMTQSPSSLSASVGDRVTITCRASQDISNYLNW
YQQKPGKAPKLLIYYTSKLHSGVPSRFSGSGSGTD
YTLTISSLQPEDFATYYCQQGSALPWTFGQGTKVE
IK

e) INCAGN1949
   i. See Gonzalez et al. 2016, DOI: 10.1158/1538-7445.AM2016-3204
   ii. See clinical trial NCT02923349 at https://clinicaltrials.gov/ct2/home
   iii. Antibody sequences are disclosed in WO2016/179517 A1:
      i. In particular, an antibody comprising the sequences:

VH CDR1 → GSAMH
VH CDR2 → RIRSKANSYATAYAASVKG
VH CDR3 → GIYDSSGYDY
VL CDR1 → RSSQSLLHSNGYNYLD
VL CDR2 → LGSNRAS
VL CDR3 → MQALQTPLT

ii. Such as, an antibody comprising the sequences:

VH→
EVQLVESGGGLVQPGGSLKLSCAASGFTFSGSAMHWVRQA
SGKGLEVVVGRIRSKANSYATAYAASVKGRFTISRDDSKN
TAYLQMNSLKTEDTAVYYCTSGIYDSSGYDYWGQGTLVTV
SS
VL→
DIVMTQSPLSLPVTPGEPASISCRSSQSLLHSNGYNYLDW
YLQKPGQSPQLLIYLGSNRASGVPDRFSGSGSGTDFTLKI
SRVEAEDVGVYYCMQALQTPLTFGGGTKVEIK

g) GSK3174998, a humanized IgG1 agonistic anti-OX40 monoclonal antibody (mAb)
   See clinical trial NCT02528357 at https://clinicaltrials.gov/ct2/home
h) PF-04518600 (PF-8600) is an investigational, fully human, monoclonal antibody (mAb) that targets OX40 protein
   See patent WO 2017/130076 A1
   See clinical trial NCT02315066 at https://clinicaltrials.gov/ct2/home—NCI thesaurus code→C121927
      (see https://ncit.nci.nih.gov/ncitbrowser/)

In some embodiments, OX40 polypeptide corresponds to Genbank accession no. CAA53576, version no. CAA53576.1, record update date: Feb. 2, 2011 10:10 AM. In one embodiment, the nucleic acid encoding OX40 polypeptide corresponds to Genbank accession no. X75962, version no. X75962.1, record update date: Feb. 2, 2011 10:10 AM. In some embodiments, OX40 polypeptide corresponds to Uniprot/Swiss-Prot accession No. P43489.

CTLA Antagonist

CTLA4 (CD152) is expressed on activated T cells and serves as a co-inhibitor to keep T cell responses in check following CD28-mediated T cell activation. CTLA4 is believed to regulate the amplitude of the early activation of naive and memory T cells following TCR engagement and to be part of a central inhibitory pathway that affects both antitumor immunity and autoimmunity. CTLA4 is expressed exclusively on T cells, and the expression of its ligands CD80 (B7.1) and CD86 (B7.2), is largely restricted to antigen-presenting cells, T cells, and other immune mediating cells. Antagonistic anti-CTLA4 antibodies that block the CTLA4 signalling pathway have been reported to enhance T cell activation. One such antibody, ipilimumab, was approved by the FDA in 2011 for the treatment of metastatic melanoma. Another anti-CTLA4 antibody, tremelimumab, was tested in phase Ill trials for the treatment of advanced melanoma, but did not significantly increase the overall survival of patients compared to the standard of care (temozolomide or dacarbazine) at that time.

“CTLA4 agonist” means any chemical compound or biological molecule that stimulates an immune reaction through inhibition of CTLA4 signalling.

To examine the extent of enhancement of, e.g., CTLA4 activity, samples or assays comprising a given, e.g., protein, gene, cell, or organism, are treated with a potential activating or inhibiting agent and are compared to control samples treated with an inactive control molecule. Control samples are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Combining an ADC, which targets a first target protein (FTP) positive lymphomas and leukemias with CTLA4 inhibitors is advantageous, because on the one hand, the ADC will directly kill the FTP positive tumor cells, while on the other hand the CTLA4 inhibitor will engage the patient's own immune system to eliminate the cancer cells. Next to FTP(+) tumor cells, target negative tumor cells in close proximity to FTP(+) tumor cells will potentially be killed by the bystander mechanism of the PBD-dimer released after cell kill of FTP(+) cells. Hence, the ADC will directly kill the tumor. The resulting release of tumor associated antigens from cells killed with the PBD dimer will trigger the immune system, which will be further enhanced by the use of CTLA4 inhibitors expressed on a large proportion of tumour infiltrating lymphocytes (TILs) from many different tumour types.

The major function of CTLA4 (CD152) is to regulate the amplitude of the early stages of T cell activation, and as such it counteracts the activity of the T cell co-stimulatory receptor, CD28, In the tumor microenvironment. Blockade of the CTLA4 pathway may therefore enhance enhancement of effector CD4+ T cell activity, while it inhibits TReg cell-dependent immunosuppression. Therefore it will be beneficial to target a FTP(+) tumor with the ADC, causing the antigenic cell death, while the CTLA4 blockade induces a stronger immune, durable response.

Specific CTLA4 antagonists suitable for use as secondary agents in the present disclosure include:
a) ipilimumab
   i. CAS Number→477202-00-9
      (see http://www.cas.org/content/chemical-substances/faqs)
   ii. Unique Ingredient Identifier (UNII)→6T8C155666
      (see http://www.fda.gov/ForIndustry/DataStandards/SubstanceRegistration System-UnigueIngredientIdentifierUNII/default.htm)
b) Tremelimumab
   i. CAS Number→745013-59-6
      (see http://www.cas.org/content/chemical-substances/faqs)
   ii. Unique Ingredient Identifier (UNII)→QEN1X95CIX
      (see http://www.fda.gov/Forlndustry/DataStandards/SubstanceRegistration System-UniqueIngredientIdentifierUNII/default.htm)

iii. VH sequence
[SEQ ID NO. 1]
GVVQPGRSLRLSCAASGFTFSSYGMHWVRQAPGKGLEWVA
VIWYDGSNKYYADSVKGRFTISRDNSKNTLYLQMNSLRAE
DTAVYYCARDPRGATLYYYYYGMDVWGQGTTVTVSSASTK
GPSVFPLAPCSRSTSESTAALGCLVKDYFPEPVTVSWNSG
ALTSGVH
iv. VL sequence
[SEQ ID NO. 2]
PSSLSASVGDRVTITCRASQSINSYLDWYQQKPGKAPKLL
IYAASSLQSGVPSRFSGSGSGTDFTLTISSLQPEDFATYY
CQQYYSTPFTFGPGTKVEIKRTVAAPSVFIFPPSDEQLKS
GTASVVCLLNNFYPREAKV

In some embodiments, CTLA polypeptide corresponds to Genbank accession no. AAL07473, version no. AAL07473.1, record update date: Mar. 11, 2010 01:28 AM. In one embodiment, the nucleic acid encoding CTLA4 polypeptide corresponds to Genbank accession no. AF414120, version no. AF414120.1, record update date: Mar. 11, 2010 01:28 AM. In some embodiments, OX40 polypeptide corresponds to Uniprot/Swiss-Prot accession No. P16410.

Combination with Radiotherapy

The therapies described herein include those that induce or enhance a subject's immune response by, for example, targeting immune regulatory cells such as Treg cells with a CD25-ADC. The targeting of immune regulatory cells in this manner allows for a reduction in the negative regulation of the subject's immune responses to an existing or newly presented antigen.

The therapies described herein may therefore be advantageously combined with other therapies which induce or enhance a subject's immune response. In this context, preclinical and clinical data indicate that combination of CD25-ADC administration with radiotherapy will lead to significant clinical benefits.

The advantage is derived from the potential of radiotherapy to convert immunologically ‘cold’ tumors into ‘hot’ tumors by a combination of distinct mechanisms including: (a) increasing tumor immunogenicity via the upregulation of antigenic expression, antigen processing, major histocompatibility molecules, and costimulatory signals; (b) overcoming an immunosuppressive tumor microenvironment by shifting the cytokine balance in favor of immunostimulation (e.g. by increasing the production of immunostimulatory cytokines); (c) recruiting antigen-presenting and immune effector cells to the tumor microenvironment (see Ko et al., Ther Adv Med Oncol 2018, Vol. 10: 1-11, DOI: 10.1177/1758834018768240). The effect of this immunological conversion of tumours is then amplifies by the immune regulatory cell depletion resulting from CD25-ADC administration. Consistent with this, relapse and regrowth of tumours following radiotherapy and PD1 blockade is associated with Treg repopulation of the tumour microenvironment (Oweida et al., Clin Cancer Res Jul. 24 2018 DOI: 10.1158/1078-0432.CCR-18-1038).

Accordingly, in an aspect the CD25-ADCs described herein are administered in combination with radiotherapy.

As used herein, the terms “radiation therapy” or “radiotherapy” may refer to the medical use of ionizing radiation as part of cancer treatment to control or eradicate malignant cells. Radiotherapy may be used for curative, adjuvant, or palliative treatment. Suitable types of radiotherapy include conventional external beam radiotherapy, stereotactic radiation therapy (e.g., Axesse, Cyberknife, Gamma Knife, Novalis, Primatom, Synergy, X-Knife, TomoTherapy or Trilogy), Intensity-Modulated Radiation Therapy, particle therapy (e.g., proton therapy), brachytherapy, delivery of radioisotopes, intraoperative radiotherapy, Auger therapy, Volumetric modulated arc therapy (VMAT), Virtual simulation, 3-dimensional conformal radiation therapy, and intensity-modulated radiation therapy.

In one embodiment, radiatiotherapy uses high-energy radiation to shrink tumors and kill cancer cells. The radiation may be, for example, X-rays, gamma rays, or charged particles. Modes of cell killing through radiation include DNA damage either directly or by creating free radicals within cells that in turn damage DNA.

Radiation may be delivered by a machine outside the body (external-beam radiation therapy), or may come from radioactive material placed in the body near cancer cells (internal radiation therapy, also called brachy therapy). In one example of systemic radiation therapy, radioactive substances, such as radioactive iodine, are used which travel in the blood to kill cancer cells.

Preferably, the radiotherapy is administered in a regime designed to minimize any immunosuppressive effects of the radiation. For example, preclinical evidence indicates high radiation doses above 12-18 Gy result in an attenuation of tumor immunogenicity (Vanpouille-Box C., et al., Nat Commun 2017; 8: 15618). In addition, it is known that circulating lymphocytes are particularly radiosensitive (see Yovino S., et al., Cancer Invest 2013; 31: 140-144); this indicates radiotherapy regimes aimed at stimulating an anti-tumour immune response should aim to mimimise both (1) the amount of vasculature exposed in each treatment, and (2) the number of exposures in the treatment regime.

Radiation dosages may be fractionated and administered in sequence; for example, on consecutive days until the total desired radiation dose is delivered.

The CD25-ADC may be administered before the radiotherapy, simultaneous with the radiotherapy, or after the radiotherapy. Preferably the CD25-ADC is administered after the radiotherapy, for example the same day or the day after the completion of a radiotherapy dose.

Treated Disorders

The therapies described herein include those that induce or enhance a subject's immune response. In particular, in certain aspects the therapies include treating a disorder by inducing or enhancing the immune response of a subject against an antigen associated with the disorder.

In some aspects the therapies described herein enhance or induce an immune response by targeting immune regulatory cells with an antibody conjugated, i.e. covalently attached by a linker, to a PBD drug moiety, i.e. toxin. When the drug is not conjugated to an antibody, the PBD drug has a cytotoxic effect. The biological activity of the PBD drug moiety is thus modulated by conjugation to an antibody. The antibody-drug conjugates (ADC) of the disclosure selectively deliver an effective dose of a cytotoxic agent to the targeted tissue whereby greater selectivity, i.e. a lower efficacious dose, may be achieved. The targeting of immune regulatory cells in this manner allows for a reduction in the negative regulation of the subject's immune responses to an existing or newly presented antigen.

The methods described herein may be used in combination with other immune response stimulating agents in order to further enhance and/or induce an immune response. This approach is expected to have utility in, for example, highly immunosuppressive circumstances that are not overcome through use of a single immunostimulating agnet/method.

For example, molcules such as CD3/DAA bi-specific T-cell engagers (BiTEs) function to direct cytotoxic T-cells' cell-killing activity against target cells bearing a DAA. BiTEs therefore stimulate an immune reaction against DAA bearing cells (see Zimmerman et al., International Immunology, Volume 27, Issue 1, January 2015, Pages 31-37). A well known example of a BiTE is Blinatumomab—a CD3/CD19 BiTE used to treat CD19+ve B-cell linage cancers such as CLL and ALL (see Robinson et al. Blood 2018:blood-2018-02-830992).

However, the immune reaction stimulated by a BiTE may still be suppressed by, for example: (1) high levels of immune suppressive cells (see Ellerman, Methods, Volume 154, 1 Feb. 2019, Pages 102-117), and/or (2) activation of immune regulatory cells by the BiTE itself (see Koristka et al. 2015, Oncoimmunology. 2015 March; 4(3): e994441). Accordingly, the methods for reducing the immune-suppressive activity of a population of immune regulatory cells described herein may be usefully combined with, for example, BiTEs to further enhance the immune response against DAA bearing target cells. Such a combination will have particular utility in patient populations where the efficacy of a first immune stimulatory agent/method (eg. BiTE) is inhibited ro reduced by the immune-suppressive activity of a population of CD25+ve immune regulatory cells such as Tregs.

In some aspects the therapies described herein enhance or induce an immune response by directly killing target cells through cytotoxic ADC binding to the target cells and/or, through a ‘bystander effect’, indirectly killing target cells in the proximity of cells that are directly bound by the cytotoxic ADC (see, for example, WO/2016/083468). The killing of target cells causes the release of target antigens, ‘stranger signals’, and/or ‘danger signals’ into the extracellular environment where they can interact with and stimulate a subject's immune system (see, for example, Virgil E J C Schijns & Ed C Lavelle (2011) Expert Review of Vaccines, 10:4, 539-550).

Thus, in one aspect, the present disclosure provides a method of inducing or enhancing an immune response in a subject, the method comprising the step of administering a CD25-ADC to the subject. The induction or enhancement of immune response may be due to the reduction in the immune-suppressive activity of an immune regulatory cell population, as defined herein.

In one aspect, the present disclosure provides a method of treating or preventing a disorder a subject, wherein the disorder is characterized by a disorder-associated antigen (DAA), the method comprising administering a CD25-ADC to the subject.

In some aspects the CD25-ADC is administered in combination with a disorder-associated antigen (DAA). This may be done with a view to inducing or enhancing an immune response against the DAA and so treating the disorder associated with the co-administered DAA. The DAA may be a protein, polypeptide, peptide, peptide mimetic, nucleic acid encoding a protein, polypeptide, peptide, peptide mimetic, sugar, oligiosaccharide, lipid, phospholipid, liposaccharide, or lipoprotein. Typically the DAA is a cell-surface antigen, meaning that in its normal pathogenic context it is found on the surface of a cell or pathogen such that is accessible to cells and molecules of a subject's immune system.

In a further aspect there is also provided a CD25-ADC as described herein for use in inducing or enhancing an immune response in a subject, or for use in treating or preventing a disorder a subject wherein the disorder is characterized by a disorder-associated antigen (DAA). Another aspect of the present disclosure provides the use of a CD25-ADC as described herein in the manufacture of a medicament for inducing or enhancing an immune response in a subject, or for treating or preventing a disorder a subject wherein the disorder is characterized by a disorder-associated antigen (DAA).

One of ordinary skill in the art is readily able to determine whether or not a candidate therapy treats a particular disorder characterized by a disorder-associated antigen (DAA). For example, assays which may conveniently be used to assess the activity offered by a particular compound are described below.

The therapies described herein may be used to treat a proliferative disorder. The term “proliferative disorder” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis. Cancers of interest include, but are not limited to, leukemias and ovarian cancers.

Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Proliferative disorders of interest include, but are not limited to, Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph-ALL) [Fielding A., Haematologica. 2010 January; 95(1): 8-12].

Proliferative disorders of particular interest include those associated with elevated numbers of regulatory immune cells, such as Treg cells. These include chronic lymphatic lymphoma (CLL), T-cell Acute Lymphoblastic Leukaemia (T-ALL), and B-cell non-Hodgkin's Lymphoma, such as Acute Myeloid Leukaemia (AML) [Niedzwiecki et al., J. Immun. R., Vol. 2018, Article ID 1292404].

Classical Hodgkins lymphoma includes the subtypes nodular sclerosing, lymphocyte predominant, lymphocyte depleted and mixed cellularity. The Hodgkins lymphoma subtype may not be defined. In certain aspects, the patients tested according to the methods here have Hodgkins lymphoma of the nodular sclerosing and mixed cellularity subtypes.

The proliferative disease may be characterised by the presence of a neoplasm comprising both CD25+ve and CD25−ve cells.

The proliferative disease may be characterised by the presence of a neoplasm composed of CD25−ve neoplastic cells, optionally wherein the CD25−ve neoplastic cells are associated with CD25+ve non-neoplastic cells such as CD25+ve Tregs.

The target neoplasm or neoplastic cells may be all or part of a solid tumour.

Solid tumors may be neoplasms, including non-haematological cancers, comprising or composed of CD25+ve neoplastic cells. Solid tumors may be neoplasms infiltrated with CD25+ve cells, such as CD25+ve Tregs; such solid tumours may lack expression of CD25 (that is, comprise or be composed of CD25-ve neoplastic cells).

For example, the solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer (including triple negative breast cancer), colorectal cancer, gastric and oesophageal cancer, melanoma, non-small cell lung cancer, ovarian cancer, hepatocellular carcinoma, renal cell carcinoma, bladder, and head and neck cancer.

The solid tumour may be a tumour with low levels of infiltrating T-cells, such as infiltrating regulatory T-cells.

Less preferably, the solid tumour may be a tumour that is not associated or infiltrated with CD25+ve cells, such as CD25+ve Tregs.

In some embodiments the high/low/no infiltrating T-cell status of a tumour is determined by measuring the ratio of T-regulatory cells/T-effector using, for example, FACS analusis of T-cells in a sample. In some embodiments the level of infiltrating T-cells is determined to be ‘high’ if the ratio of T-regulatory cells/T-effector is at least 20. In some embodiments the level of infiltrating T-cells is determined to be ‘low’ if the ratio of T-regulatory cells/T-effector is less than 20.

The neoplasm or neoplastic cells may be all or part of an established tumour. An ‘established tumour’ as described herein may be, for example, a tumour such as a solid tumour diagnosed or identified in a naïve subject.

In some cases the naïve subject is a subject that has not yet been treated to reduce the immune-suppressive activity of an immune regulatory cell population, as defined herein; for example; treated with an anti-CD25 antibody or a CD25-ADC. In some cases the naïve subject is a subject that has not yet been treated with ADCx25, as defined herein.

The neoplasm or neoplastic cells may be a circulating tumour or circulating tumour cells (CTC; Gupta et al. 2006, Cell. 127 (4): 679-95; Rack et al., 2014. Journal of the National Cancer Institute. 106 (5)). The CTCs may be, or comprise, metastatic cells (i.e. CTCs capable of establishing metastatic tumours in a subject).

It is contemplated that the therapies of the present disclosure may be used to treat various proliferative disorders. Exemplary conditions or hyperproliferative disorders include benign or malignant tumors; leukemia, haematological, and lymphoid malignancies. Others include neuronal, glial, astrocytal, hypothalamic, glandular, macrophagal, epithelial, stromal, blastocoelic, inflammatory, angiogenic and immunologic, including autoimmune disorders and graft-versus-host disease (GVHD).

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

It is contemplated that the therapies of the present disclosure may be used to treat any proliferative disorder that is characterized by a tumor-associated antigen (TAA). Typically, a TAA is an antigen that is either: (1) expressed only on neoplastic cells, or (2) expressed at higher levels by neoplastic cells as compared to non-neoplastic (i.e. normal cells). Often a TAA will be an antigen present on the surface of a neoplastic cell.

Tumour Associated Antigens

In some aspects, the TAA is selected from the group consisting of:

(1) BMPR1B (bone morphogenetic protein receptor-type IB)

Nucleotide

Genbank accession no. NM_001203

Genbank version no. NM_001203.2 GI:169790809

Genbank record update date: Sep. 23, 2012 02:06 PM

Polypeptide

Genbank accession no. NP_001194

Genbank version no. NP_001194.1 GI:4502431

Genbank record update date: Sep. 23, 2012 02:06 PM

### CROSS-REFERENCES

ten Dijke, P., et al Science 264 (5155): 101-104 (1994), Oncogene 14 10 (11):1377-1382 (1997)); WO2004/063362 (Claim 2); WO2003/042661 (Claim 12); US2003/134790-A1 (Page 38-39); WO2002/102235 (Claim 13; Page 296); WO2003/055443 (Page 91-92); WO2002/99122 (Example 2; Page 528-530); WO2003/029421 (Claim 6); WO2003/024392 (Claim 2; FIG. 112); WO2002/98358 (Claim 1; Page 183); WO2002/54940 (Page 100-101); WO2002/59377(Page 349-350); WO2002/30268 (Claim 27; Page 376); 15 WO2001/48204 (Example; FIG. 4); NP_001194 bone morphogenetic protein receptor, type IB/pid=NP_001194.1.; MIM:603248; AY065994

(2) E16 (LA T1, SLC7A5)

Nucleotide

Genbank accession no. NM_003486

Genbank version no. NM_003486.5 GI:71979931

Genbank record update date: Jun. 27, 2012 12:06 PM

Polypeptide

Genbank accession no. NP_003477

Genbank version no. NP_003477.4 GI:71979932

Genbank record update date: Jun. 27, 2012 12:06 PM

### CROSS REFERENCES

Biochem. Biophys. Res. Commun. 255 (2), 283-288 (1999), Nature 395 (6699):288-291 (1998), Gaugitsch, H. W., et 20 al (1992) J. Biol. Chem. 267 (16):11267-11273); WO2004/048938 (Example 2); WO2004/032842 (Example IV); WO2003/042661 (Claim 12); WO2003/016475 (Claim 1); WO2002/78524 (Example 2); WO2002/99074 (Claim 19; Page 127-129); WO2002/86443 (Claim 27; Pages 222, 393); WO2003/003906 (Claim 10; Page 293); WO2002/64798 (Claim 33; Page 93-95); WO2000/14228 (Claim 5; Page 133-136); US2003/224454 (FIG. 3); 25 WO2003/025138 (Claim 12; Page 150); NP_003477 solute carrier family 7 (cationic amino acid transporter, y+system), member 5/pid=NP_003477.3—Homo sapiens; MIM:600182; NM_015923.

(3) STEAP1 (Six Transmembrane Epithelial Antigen of Prostate)

Nucleotide

Genbank accession no. NM_012449

Genbank version no. NM_012449.2 GI:22027487

Genbank record update date: Sep. 9, 2012 02:57 PM

Polypeptide

Genbank accession no. NP_036581

Genbank version no. NP_036581.1 GI:9558759

Genbank record update date: Sep. 9, 2012 02:57 PM

### CROSS REFERENCES

Cancer Res. 61 (15), 5857-5860 (2001), Hubert, R. S., et al (1999) Proc. Natl. Acad. Sci. U.S.A. 96 (25):14523-14528); WO2004/065577 (Claim 6); WO2004/027049 (FIG. 1L); EP1394274 (Example 11); WO2004/016225 (Claim 2); WO2003/042661 (Claim 12); US2003/157089 (Example 5); US2003/185830 (Example 5); US2003/064397 (FIG. 2); WO2002/89747 (Example 5; Page 618-619); WO2003/022995 (Example 9; FIG. 13A, Example 53; Page 173, Example 2; FIG. 2A); six transmembrane epithelial antigen of the prostate; MIM:604415.

(4) 0772P (CA125, MUC16)

Nucleotide

Genbank accession no. AF361486

Genbank version no. AF361486.3 GI:34501466

Genbank record update date: Mar. 11, 2010 07:56 AM

Polypeptide

Genbank accession no. AAK74120

Genbank version no. AAK74120.3 GI:34501467

Genbank record update date: Mar. 11, 2010 07:56 AM

### CROSS REFERENCES

J. Biol. Chem. 276 (29):27371-27375 (2001)); WO2004/045553 (Claim 14); WO2002/92836 (Claim 6; FIG. 12); WO2002/83866 (Claim 15; Page 116-121); US2003/124140 (Example 16); GI:34501467;

(5) MPF (MPF, MSLN, SMR, Megakaryocyte Potentiating Factor, Mesothelin)

Nucleotide

Genbank accession no. NM_005823

Genbank version no. NM_005823.5 GI:293651528

Genbank record update date: Sep. 2, 2012 01:47 PM

Polypeptide

Genbank accession no. NP_005814

Genbank version no. NP_005814.2 GI:53988378

Genbank record update date: Sep. 2, 2012 01:47 PM

### CROSS REFERENCES

Yamaguchi, N., et al Biol. Chem. 269 (2), 805-808 (1994), Proc. Natl. Acad. Sci. U.S.A. 96 (20):11531-11536 (1999), Proc. Natl. Acad. Sci. U.S.A. 93 10 (1):136-140 (1996), J. Biol. Chem. 270 (37):21984-21990 (1995)); WO2003/101283 (Claim 14); (WO2002/102235 (Claim 13; Page 287-288); WO2002/101075 (Claim 4; Page 308-309); WO2002/71928 (Page 320-321); WO94/10312 (Page 52-57); IM:601051.

(6) Napi3b (NAPI-3B, NPTIIb, SLC34A2, solute carrier family 34 (sodium phosphate), member 2, type II sodium-dependent phosphate transporter 3b)

Nucleotide

Genbank accession no. NM_006424

Genbank version no. NM_006424.2 GI:110611905

Genbank record update date: Jul. 22, 2012 03:39 PM

Polypeptide

Genbank accession no. NP_006415

Genbank version no. NP_006415.2 GI:110611906

Genbank record update date: Jul. 22, 2012 03:39 PM

### CROSS REFERENCES

J. Biol. Chem. 277 (22):19665-19672 (2002), Genomics 62 (2):281-284 (1999), Feild, J. A., et al (1999) Biochem. Biophys. Res. Commun. 258 (3):578-582); WO2004/022778 (Claim 2); EP1394274 (Example 11); WO2002/102235 (Claim 13; Page 20 326); EP0875569 (Claim 1; Page 17-19); WO2001/57188 (Claim 20; Page 329); WO2004/032842 (Example IV); WO2001/75177 (Claim 24; Page 139-140); MIM:604217.

(7) Sema 5b (FLJ10372, KIAA1445, Mm.42015, SEMA5B, SEMAG, Semaphorin 5b Hlog, 25 sema domain, seven thrombospondin repeats (type 1 and type 1-like), transmembrane domain (TM) and short cytoplasmic domain, (semaphorin) 5B)

Nucleotide

Genbank accession no. AB040878

Genbank version no. AB040878.1 GI:7959148

Genbank record update date: Aug. 2, 2006 05:40 PM

Polypeptide

Genbank accession no. BAA95969

Genbank version no. BAA95969.1 GI:7959149

Genbank record update date: Aug. 2, 2006 05:40 PM

### CROSS REFERENCES

Nagase T., et al (2000) DNA Res. 7 (2):143-150); WO2004/000997 (Claim 1); WO2003/003984 (Claim 1); WO2002/06339 (Claim 1; Page 50); WO2001/88133 (Claim 1; Page 41-43, 48-58); WO2003/054152 (Claim 20); WO2003/101400 (Claim 11); Accession: 30 Q9P283; Genew; HGNC:10737

(8) PSCA hlg (2700050C12Rik, C530008016Rik, RIKEN cDNA 2700050C12, RIKEN cDNA 2700050C12 gene)

Nucleotide

Genbank accession no. AY358628

Genbank version no. AY358628.1 G1:37182377

Genbank record update date: Dec. 1, 2009 04:15 AM

Polypeptide

Genbank accession no. AAQ88991

Genbank version no. AAQ88991.1 GI:37182378

Genbank record update date: Dec. 1, 2009 04:15 AM

### CROSS REFERENCES

Ross et al (2002) Cancer Res. 62:2546-2553; US2003/129192 (Claim 2); US2004/044180 (Claim 12); US2004/044179 35 (Claim 11); US2003/096961 (Claim 11); US2003/232056 (Example 5); WO2003/105758 16 (Claim 12); US2003/206918 (Example 5); EP1347046 (Claim 1); WO2003/025148 (Claim 20); GI:37182378.

(9) ETBR (Endothelin type B receptor)

Nucleotide

Genbank accession no. AY275463

Genbank version no. AY275463.1 GI:30526094

Genbank record update date: Mar. 11, 2010 02:26 AM

Polypeptide

Genbank accession no. AAP32295

Genbank version no. AAP32295.1 GI:30526095

Genbank record update date: Mar. 11, 2010 02:26 AM

### CROSS REFERENCES

Nakamuta M., et al Biochem. Biophys. Res. Commun. 177, 34-39, 1991; Ogawa Y., et al Biochem. Biophys. Res. Commun. 178, 248-255, 1991; Arai H., et al Jpn. Circ. J. 56, 1303-1307, 1992; Arai H., et al J. Biol. Chem. 268, 3463-3470, 1993; Sakamoto A., Yanagisawa M., et al Biochem. Biophys. Res. Commun. 178, 656-663, 1991; Elshourbagy N. A., et al J. Biol. Chem. 268, 3873-3879, 1993; Haendler B., et al J. Cardiovasc. Pharmacol. 20, s1-S4, 1992; Tsutsumi M., et al Gene 228, 43-49, 1999; Strausberg R. L., et al Proc. Natl. Acad. Sci. U.S.A. 99, 16899-16903, 2002; Bourgeois C., et al J. Clin. Endocrinol. Metab. 82, 3116-3123, 1997; Okamoto Y., et al Biol. Chem. 272, 21589-21596, 1997; Verheij J.B., et al Am. J. Med. Genet. 108, 223-225, 2002; Hofstra R. M. W., et al Eur. J. Hum. Genet. 5, 180-185, 1997; Puffenberger E.G., et al Cell 79, 1257-1266, 1994; Attie T., et al, Hum. Mol. Genet. 4, 2407-15 2409, 1995; Auricchio A., et al Hum. Mol. Genet. 5:351-354, 1996; Amiel J., et al Hum. Mol. Genet. 5, 355-357, 1996; Hofstra R. M. W., et al Nat. Genet. 12, 445-447, 1996; Svensson P. J., et al Hum. Genet. 103, 145-148, 1998; Fuchs S., et al Mol. Med. 7, 115-124, 2001; Pingault V., et al (2002) Hum. Genet. 111, 198-206; WO2004/045516 (Claim 1); WO2004/048938 (Example 2); WO2004/040000 (Claim 151); WO2003/087768 (Claim 1); 20 WO2003/016475 (Claim 1); WO2003/016475 (Claim 1); WO2002/61087 (FIG. 1); WO2003/016494 (FIG. 6); WO2003/025138 (Claim 12; Page 144); WO2001/98351 (Claim 1; Page 124-125); EP0522868 (Claim 8; FIG. 2); WO2001/77172 (Claim 1; Page 297-299); US2003/109676; U.S. Pat. No. 6,518,404 (FIG. 3); U.S. Pat. No. 5,773,223 (Claim 1a; Col 31-34); WO2004/001004.

(10) MSG783 (RNF124, hypothetical protein FLJ20315)

Nucleotide

Genbank accession no. NM_017763

Genbank version no. NM_017763.4 GI:167830482

Genbank record update date: Jul. 22, 2012 12:34 AM

Polypeptide

Genbank accession no. NP_060233

Genbank version no. NP_060233.3 GI:56711322

Genbank record update date: Jul. 22, 2012 12:34 AM

### CROSS REFERENCES

WO2003/104275 (Claim 1); WO2004/046342 (Example 2); WO2003/042661 (Claim 12); WO2003/083074 (Claim 14; Page 61); WO2003/018621 (Claim 1); WO2003/024392 (Claim 2; FIG. 93); WO2001/66689 (Example 6); LocusID:54894.

(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein)

Nucleotide

Genbank accession no. AF455138

Genbank version no. AF455138.1 GI:22655487

Genbank record update date: Mar. 11, 2010 01:54 AM

Polypeptide

Genbank accession no. AAN04080

Genbank version no. AAN04080.1 GI:22655488

Genbank record update date: Mar. 11, 2010 01:54 AM

### CROSS REFERENCES

Lab. Invest. 82 (11):1573-1582 (2002)); WO2003/087306; US2003/064397 (Claim 1; FIG. 1); WO2002/72596 (Claim 13; Page 54-55); WO2001/72962 (Claim 1; FIG. 4B); 35 WO2003/104270 (Claim 11); WO2003/104270 (Claim 16); US2004/005598 (Claim 22); WO2003/042661 (Claim 12); US2003/060612 (Claim 12; FIG. 10); WO2002/26822 (Claim 23; FIG. 2); WO2002/16429 (Claim 12; FIG. 10); GI:22655488.

(12) TrpM4 (BR22450, FLJ20041, TRPM4, TRPM4B, transient receptor potential cation 5 channel, subfamily M, member 4)

Nucleotide

Genbank accession no. NM_017636

Genbank version no. NM_017636.3 GI:304766649

Genbank record update date: Jun. 29, 2012 11:27 AM

Polypeptide

Genbank accession no. NP_060106

Genbank version no. NP_060106.2 GI:21314671

Genbank record update date: Jun. 29, 2012 11:27 AM

### CROSS REFERENCES

Xu, X. Z., et al Proc. Natl. Acad. Sci. U.S.A. 98 (19):10692-10697 (2001), Cell 109 (3):397-407 (2002), J. Biol. Chem. 278 (33):30813-30820 (2003)); US2003/143557 (Claim 4); WO2000/40614 (Claim 14; Page 100-103); WO2002/10382 (Claim 1; FIG. 9A); WO2003/042661 (Claim 12); WO2002/30268 (Claim 27; Page 391); US2003/219806 (Claim 4); WO2001/62794 (Claim 10 14; FIG. 1A-D); MIM:606936.

(13) CRIPTO (CR, CR1, CRGF, CRIPTO, TDGF1, teratocarcinoma-derived growth factor)

Nucleotide

Genbank accession no. NM_003212

Genbank version no. NM_003212.3 GI:292494881

Genbank record update date: Sep. 23, 2012 02:27 PM

Polypeptide

Genbank accession no. NP_003203

Genbank version no. NP_003203.1 GI:4507425

Genbank record update date: Sep. 23, 2012 02:27 PM

### CROSS REFERENCES

Ciccodicola, A., et al EMBO J. 8 (7):1987-1991 (1989), Am. J. Hum. Genet. 49 (3):555-565 (1991)); US2003/224411 (Claim 1); WO2003/083041 (Example 1); WO2003/034984 (Claim 12); WO2002/88170 (Claim 2; Page 52-53); WO2003/024392 (Claim 2; FIG. 58); WO2002/16413 (Claim 1; Page 94-95, 105); WO2002/22808 (Claim 2; FIG. 1); U.S. Pat. No. 5,854,399 (Example 2; Col 17-18); U.S. Pat. No. 5,792,616 (FIG. 2); MIM:187395.

(14) CD21 (CR2 (Complement receptor 2) or C3DR (C3d/Epstein Barr virus receptor) or Hs.73792)

Nucleotide

Genbank accession no M26004

Genbank version no. M26004.1 GI:181939

Genbank record update date: Jun. 23, 2010 08:47 AM

Polypeptide

Genbank accession no. AAA35786

Genbank version no. AAA35786.1 GI:181940

Genbank record update date: Jun. 23, 2010 08:47 AM

### CROSS REFERENCES

Fujisaku et al (1989) J. Biol. Chem. 264 (4):2118-2125); Weis J. J., et al J. Exp. Med. 167, 1047-1066, 1988; Moore M., et al Proc. Natl. Acad. Sci. U.S.A. 84, 9194-9198, 1987; Barel M., et al Mol. Immunol. 35, 1025-1031, 1998; Weis J. J., et al Proc. Natl. Acad. Sci. U.S.A. 83, 5639-5643, 1986; Sinha S. K., et al (1993) J. Immunol. 150, 5311-5320; WO2004/045520 (Example 4); US2004/005538 (Example 1); WO2003/062401 (Claim 9); WO2004/045520 (Example 4); WO91/02536 (FIGS. 9.1-9.9); WO2004/020595 (Claim 1); Accession: P20023; Q13866; Q14212; EMBL; M26004; AAA35786.1.

(15) CD79b (CD79B, CD79β, IGb (immunoglobulin-associated beta), B29)

Nucleotide

Genbank accession no NM_000626

Genbank version no. NM_000626.2 GI:90193589

Genbank record update date: Jun. 26, 2012 01:53 PM

Polypeptide

Genbank accession no. NP_000617

Genbank version no. NP_000617.1 GI:11038674

Genbank record update date: Jun. 26, 2012 01:53 PM

### CROSS REFERENCES

Proc. Natl. Acad. Sci. U.S.A. (2003) 100 (7):4126-4131, Blood (2002) 100 (9):3068-3076, Muller et al (1992) Eur. J. Immunol. 22 (6):1621-1625); WO2004/016225 (claim 2, FIG. 140); WO2003/087768, US2004/101874 (claim 1, page 102); WO2003/062401 (claim 9); WO2002/78524 (Example 2); US2002/150573 (claim 35 5, page 15); U.S. Pat. No. 5,644,033; WO2003/048202 (claim 1, pages 306 and 309); WO 99/58658, U.S. Pat. No. 6,534,482 (claim 13, FIG. 17A/B); WO2000/55351 (claim 11, pages 1145-1146); MIM:147245

(16) FcRH2 (IFGP4, IRTA4, SPAP1A (SH2 domain containing phosphatase anchor protein 5 1a), SPAP1B, SPAP1C)

Nucleotide

Genbank accession no NM_030764

Genbank version no. NM_030764.3 GI:227430280

Genbank record update date: Jun. 30, 2012 12:30 AM

Polypeptide

Genbank accession no. NP_110391

Genbank version no. NP_110391.2 GI:19923629

Genbank record update date: Jun. 30, 2012 12:30 AM

### CROSS REFERENCES

AY358130); Genome Res. 13 (10):2265-2270 (2003), Immunogenetics 54 (2):87-95 (2002), Blood 99 (8):2662-2669 (2002), Proc. Natl. Acad. Sci. U.S.A. 98 (17):9772-9777 (2001), Xu, M. J., et al (2001) Biochem. Biophys. Res. Commun. 280 (3):768-775; WO2004/016225 (Claim 2); WO2003/077836; WO2001/38490 (Claim 5; FIG. 18D-1-18D-2); WO2003/097803 (Claim 12); 10 WO2003/089624 (Claim 25); MIM:606509.

(17) HER2 (ErbB2)

Nucleotide

Genbank accession no M11730

Genbank version no. M11730.1 GI:183986

Genbank record update date: Jun. 23, 2010 08:47 AM

Polypeptide

Genbank accession no. AAA75493

Genbank version no. AAA75493.1 GI:306840

Genbank record update date: Jun. 23, 2010 08:47 AM

### CROSS REFERENCES

Coussens L., et al Science (1985) 230(4730):1132-1139); Yamamoto T., et al Nature 319, 230-234, 1986; Semba K., et al Proc. Natl. Acad. Sci. U.S.A. 82, 6497-6501, 1985; Swiercz J. M., et al J. Cell Biol. 165, 869-15 880, 2004; Kuhns J. J., et al J. Biol. Chem. 274, 36422-36427, 1999; Cho H.-S., et al Nature 421, 756-760, 2003; Ehsani A., et al (1993) Genomics 15, 426-429; WO2004/048938 (Example 2); WO2004/027049 (FIG. 11); WO2004/009622; WO2003/081210; WO2003/089904 (Claim 9); WO2003/016475 (Claim 1); US2003/118592; WO2003/008537 (Claim 1); WO2003/055439 (Claim 29; FIG. 1A-B); WO2003/025228 (Claim 37; FIG. 5C); 20 WO2002/22636 (Example 13; Page 95-107); WO2002/12341 (Claim 68; FIG. 7); WO2002/13847 (Page 71-74); WO2002/14503 (Page 114-117); WO2001/53463 (Claim 2; Page 41-46); WO2001/41787 (Page 15); WO2000/44899 (Claim 52; FIG. 7); WO2000/20579 (Claim 3; FIG. 2); U.S. Pat. No. 5,869,445 (Claim 3; Col 31-38); WO9630514 (Claim 2; Page 56-61); EP1439393 (Claim 7); WO2004/043361 (Claim 7); WO2004/022709; WO2001/00244 25 (Example 3; FIG. 4); Accession: P04626; EMBL; M11767; AAA35808.1. EMBL; M11761; AAA35808.1

Antibodies

Abbott: US20110177095
For example, an antibody comprising CDRs having overall at least 80% sequence identity to CDRs having amino acid sequences of SEQ ID NO:3 (CDR-H1), SEQ ID NO:4 (CDR-H2), SEQ ID NO:5 (CDR-H3), SEQ ID NO:104 and/or SEQ ID NO:6 (CDR-L1), SEQ ID NO:7 (CDR-L2), and SEQ ID NO:8 (CDR-L3), wherein the anti-HER2 antibody or anti-HER2 binding fragment has reduced immunogenicity as compared to an antibody having a VH of SEQ ID NO:1 and a VL of SEQ ID NO:2.

Biogen: US20100119511
For example, ATCC accession numbers: PTA-10355, PTA-10356, PTA-10357, PTA-10358
For example, a purified antibody molecule that binds to HER2 comprising a all six CDR's from an antibody selected from the group consisting of BIIB71F10 (SEQ ID NOs:11, 13), B11B69A09 (SEQ ID NOs:15, 17); BIIB67F10 (SEQ ID NOs:19, 21); BIIB67F11 (SEQ ID NOs:23, 25), B11B66A12 (SEQ ID NOs:27, 29), BIIB66C01 (SEQ ID NOs:31, 33), BIIB65C10 (SEQ ID NOs:35, 37), B11B65H09 (SEQ ID NOs:39, 41) and B11B65B03 (SEQ ID NOs:43, 45), or CDRs which are identical or which have no more than two alterations from said CDRs.

Herceptin (Genentech)—U.S. Pat. No. 6,054,297; ATCC accession no. CRL-10463 (Genentech)

Pertuzumab (Genentech)
US20110117097
   for example, see SEQ IDs No. 15&16, SEQ IDs No. 17&18, SEQ IDs No. 23&24 & ATCC accession numbers HB-12215, HB-12216, CRL 10463, HB-12697.
US20090285837
US20090202546
   for example, ATCC accession numbers: HB-12215, HB-12216, CRL 10463, H B-12698.
US20060088523
   for example, ATCC accession numbers: HB-12215, HB-12216
   for example, an antibody comprising the variable light and variable heavy amino acid sequences in SEQ ID Nos. 3 and 4, respectively.
   for example, an antibody comprising a light chain amino acid sequence selected from SEQ ID No. 15 and 23, and a heavy chain amino acid sequence selected from SEQ ID No. 16 and 24
US20060018899
   for example, ATCC accession numbers: (7C2) HB-12215, (7F3) HB-12216, (4D5) CRL-10463, (2C4) HB-12697.
   for example, an antibody comprising the amino acid sequence in SEQ ID No. 23, or a deamidated and/or oxidized variant thereof.
US2011/0159014
   for example, an antibody having a light chain variable domain comprising the hypervariable regions of SEQ ID NO: 1”.
   For example, an antibody having a heavy chain variable domain comprising the hypervariable regions of SEQ ID NO: 2.
US20090187007

Glycotope: TrasGEX antibody http://www.glycotope.com/pipeline
For example, see International Joint Cancer Institute and Changhai Hospital Cancer Cent: HMTI-Fc Ab—Gao J., et al BMB Rep. 2009 Oct. 31; 42(10):636-41.

Symphogen: US20110217305
Union Stem Cell &Gene Engineering, China—Liu H Q., et al Xi Bao Yu Fen Zi Mian YiXue Za Zhi. 2010 May; 26(5):456-8.

(18) NCA (CEACAM6)

Nucleotide

Genbank accession no M18728

Genbank version no. M18728.1 GI:189084

Genbank record update date: Jun. 23, 2010 08:48 AM

Polypeptide

Genbank accession no. AAA59907

Genbank version no. AAA59907.1 GI:189085

Genbank record update date: Jun. 23, 2010 08:48 AM

### CROSS REFERENCES

Barnett T., et al Genomics 3, 59-66, 1988; Tawaragi Y., et al Biochem. Biophys. Res. Commun. 150, 89-96, 1988; Strausberg R. L., et al Proc. Natl. Acad. Sci. U.S.A. 99:16899-16903, 2002; WO2004/063709; EP1439393 (Claim 7); WO2004/044178 (Example 4); WO2004/031238; WO2003/042661 (Claim 12); WO2002/78524 (Example 2); WO2002/86443 (Claim 27; Page 427); WO2002/60317 (Claim 2); Accession: P40199; Q14920; EMBL; M29541; AAA59915.1. EMBL; M18728.

(19) MDP (DPEP1)

Nucleotide

Genbank accession no BC017023

Genbank version no. BC017023.1 GI:16877538

Genbank record update date: Mar. 6, 2012 01:00 PM

Polypeptide

Genbank accession no. AAH17023

Genbank version no. AAH17023.1 GI:16877539

Genbank record update date: Mar. 6, 2012 01:00 PM

### CROSS REFERENCES

Proc. Natl. Acad. Sci. U.S.A. 99 (26):16899-16903 (2002)); WO2003/016475 (Claim 1); WO2002/64798 (Claim 33; Page 85-87); JP05003790 (FIG. 6-8); WO99/46284 (FIG. 9); MIM:179780.

(20) IL20R-alpha (IL20Ra, ZCYTOR7)

Nucleotide

Genbank accession no AF184971

Genbank version no. AF184971.1 GI:6013324

Genbank record update date: Mar. 10, 2010 10:00 PM

Polypeptide

Genbank accession no. AAF01320

Genbank version no. AAF01320.1 GI:6013325

Genbank record update date: Mar. 10, 2010 10:00 PM

### CROSS REFERENCES

Clark H.F., et al Genome Res. 13, 2265-2270, 2003; Mungall A. J., et al Nature 425, 805-811, 2003; Blumberg H., et al Cell 104, 9-19, 2001; Dumoutier L., et al J. Immunol. 167, 3545-3549, 2001; Parrish-Novak J., et al J. Biol. Chem. 277, 47517-47523, 2002; Pletnev S., et al (2003) 10 Biochemistry 42:12617-12624; Sheikh F., et al (2004) J. Immunol. 172, 2006-2010; EP1394274 (Example 11); US2004/005320 (Example 5); WO2003/029262 (Page 74-75); WO2003/002717 (Claim 2; Page 63); WO2002/22153 (Page 45-47); US2002/042366 (Page 20-21); WO2001/46261 (Page 57-59); WO2001/46232 (Page 63-65); WO98/37193 (Claim 1; Page 55-59); Accession: Q9UHF4; Q6UWA9; Q96SH8; EMBL; AF184971; AAF01320.1.

(21) Brevican (BCAN, BEHAB)

Nucleotide

Genbank accession no AF229053

Genbank version no. AF229053.1 GI:10798902

Genbank record update date: Mar. 11, 2010 12:58 AM

Polypeptide

Genbank accession no. AAG23135

Genbank version no. AAG23135.1 GI:10798903

Genbank record update date: Mar. 11, 2010 12:58 AM

### CROSS REFERENCES

Gary S.C., et al Gene 256, 139-147, 2000; Clark H.F., et al Genome Res. 13, 2265-2270, 2003; Strausberg R. L., et al Proc. Natl. Acad. Sci. U.S.A. 99, 16899-16903, 2002; US2003/186372 (Claim 11); US2003/186373 (Claim 11); US2003/119131 (Claim 1; FIG. 52); US2003/119122 (Claim 1; 20 FIG. 52); US2003/119126 (Claim 1); US2003/119121 (Claim 1; FIG. 52); US2003/119129 (Claim 1); US2003/119130 (Claim 1); US2003/119128 (Claim 1; FIG. 52); US2003/119125 (Claim 1); WO2003/016475 (Claim 1); WO2002/02634 (Claim 1)

(22) EphB2R (DRT, ERK, Hek5, EPHT3, Tyro5)

Nucleotide

Genbank accession no NM_004442

Genbank version no. NM_004442.6 GI:111118979

Genbank record update date: Sep. 8, 2012 04:43 PM

Polypeptide

Genbank accession no. NP_004433

Genbank version no. NP_004433.2 GI:21396504

Genbank record update date: Sep. 8, 2012 04:43 PM

### CROSS REFERENCES

Chan, J. and Watt, V. M., Oncogene 6 (6), 1057-1061 (1991) Oncogene 10 (5):897-905 (1995), Annu. Rev. Neurosci. 21:309-345 (1998), Int. Rev. Cytol. 196:177-244 (2000)); WO2003042661 (Claim 12); WO200053216 (Claim 1; Page 41); WO2004065576 (Claim 1); WO2004020583 (Claim 9); WO2003004529 (Page 128-132); WO200053216 (Claim 1; Page 42); MIM:600997.

(23) ASLG659 (B7h)

Nucleotide

Genbank accession no. AX092328

Genbank version no. AX092328.1 GI:13444478

Genbank record update date: Jan. 26, 2011 07:37 AM

### CROSS REFERENCES

US2004/0101899 (Claim 2); WO2003104399 (Claim 11); WO2004000221 (FIG. 3); US2003/165504 (Claim 1); US2003/124140 (Example 2); US2003/065143 (FIG. 60); WO2002/102235 (Claim 13; Page 299); US2003/091580 (Example 2); WO2002/10187 (Claim 6; FIG. 10); WO2001/94641 (Claim 12; FIG. 7b); WO2002/02624 (Claim 13; FIG. 1A-1B); US2002/034749 (Claim 54; Page 45-46); WO2002/06317 (Example 2; Page 320-321, Claim 34; Page 321-322); WO2002/71928 (Page 468-469); WO2002/02587 (Example 1; FIG. 1); WO2001/40269 (Example 3; Pages 190-192); WO2000/36107 (Example 2; Page 205-207); WO2004/053079 (Claim 12); WO2003/004989 (Claim 1); WO2002/71928 (Page 233-234, 452-453); WO 01/16318.

(24) PSCA (Prostate stem cell antigen precursor)

Nucleotide

Genbank accession no AJ297436

Genbank version no. AJ297436.1 GI:9367211

Genbank record update date: Feb. 1, 2011 11:25 AM

Polypeptide

Genbank accession no. CAB97347

Genbank version no. CAB97347.1 GI:9367212

Genbank record update date: Feb. 1, 2011 11:25 AM

### CROSS REFERENCES

Reiter R. E., et al Proc. Natl. Acad. Sci. U.S.A. 95, 1735-1740, 1998; Gu Z., et al Oncogene 19, 1288-1296, 2000; Biochem. Biophys. Res. Commun. (2000) 275(3):783-788; WO2004/022709; EP1394274 (Example 11); US2004/018553 (Claim 17); WO2003/008537 (Claim 1); WO2002/81646 (Claim 1; Page 164); WO2003/003906 (Claim 10; Page 288); WO2001/40309 (Example 1; FIG. 17); US2001/055751 (Example 1; FIG. 1b); WO2000/32752 (Claim 18; FIG. 1); WO98/51805 (Claim 17; Page 97); WO98/51824 (Claim 10; Page 94); WO98/40403 (Claim 2; FIG. 1B); Accession: 043653; EMBL; AF043498; AAC39607.1

(25) GEDA

Nucleotide

Genbank accession no AY260763

Genbank version no. AY260763.1 GI:30102448

Genbank record update date: Mar. 11, 2010 02:24 AM

Polypeptide

Genbank accession no. AAP14954

Genbank version no. AAP14954.1 GI:30102449

Genbank record update date: Mar. 11, 2010 02:24 AM

### CROSS REFERENCES

AP14954 lipoma HMGIC fusion-partnerlike protein/pid=AAP14954.1—Homo sapiens (human); WO2003/054152 (Claim 20); WO2003/000842 (Claim 1); WO2003/023013 (Example 3, Claim 20); US2003/194704 (Claim 45); GI:30102449;

(26) BAFF-R (B cell—activating factor receptor, BLyS receptor 3, BR3)

Nucleotide

Genbank accession no AF116456

Genbank version no. AF116456.1 GI:4585274

Genbank record update date: Mar. 10, 2010 09:44 PM

Polypeptide

Genbank accession no. AAD25356

Genbank version no. AAD25356.1 GI:4585275

Genbank record update date: Mar. 10, 2010 09:44 PM

### CROSS REFERENCES

BAFF receptor/pid=NP_443177.1—Homo sapiens: Thompson, J. S., et al Science 293 (5537), 2108-2111 (2001); WO2004/058309; WO2004/011611; WO2003/045422 (Example; Page 32-33); WO2003/014294 (Claim 35; FIG. 6B); WO2003/035846 (Claim 70; Page 615-616); WO2002/94852 (Col 136-137); WO2002/38766 25 (Claim 3; Page 133); WO2002/24909 (Example 3; FIG. 3); MIM:606269; NP_443177.1; NM_052945_1; AF132600

(27) CD22 (B-cell receptor CD22-B isoform, BL-CAM, Lyb-8, Lyb8, SIGLEC-2, FLJ22814)

Nucleotide

Genbank accession no AK026467

Genbank version no. AK026467.1 GI:10439337

Genbank record update date: Sep. 11, 2006 11:24 PM

Polypeptide

Genbank accession no. BAB15489

Genbank version no. BAB15489.1 GI:10439338

Genbank record update date: Sep. 11, 2006 11:24 PM

### CROSS REFERENCES

Wilson et al (1991) J. Exp. Med. 173:137-146; 30 WO2003/072036 (Claim 1; FIG. 1); IM:107266; NP_001762.1; NM_001771_1.

(27a) CD22 (CD22 molecule)

Nucleotide

Genbank accession no X52785

Genbank version no. X52785.1 GI:29778

Genbank record update date: Feb. 2, 2011 10:09 AM

Polypeptide

Genbank accession no. CAA36988

Genbank version no. CAA36988.1 GI:29779

Genbank record update date: Feb. 2, 2011 10:09 AM

### CROSS REFERENCES

Stamenkovic I. et al., Nature 345 (6270), 74-77 (1990)??

Other Information

Official Symbol: CD22

Other Aliases: SIGLEC-2, SIGLEC2

Other Designations: B-cell receptor CD22; B-lymphocyte cell adhesion molecule; BL-CAM; CD22 antigen; T-cell surface antigen Leu-14; sialic acid binding Ig-like lectin 2; sialic acid-binding Ig-like lectin 2

Antibodies

G5/44 (Inotuzumab): DiJoseph J F., et al Cancer Immunol Immunother. 2005 January; 54(1):11-24.

Epratuzumab-Goldenberg D M., et al Expert Rev Anticancer Ther. 6(10): 1341-53, 2006.

(28) CD79a (CD79A, CD79alpha), immunoglobulin-associated alpha, a B cell-specific protein that covalently interacts with Ig beta (CD79B) and forms a complex on the surface with Ig M 35 molecules, transduces a signal involved in B-cell differentiation), pl: 4.84, MW: 25028 TM: 2

[P] Gene Chromosome: 19q13.2).

Nucleotide

Genbank accession no NM_001783

Genbank version no. NM_001783.3 GI:90193587

Genbank record update date: Jun. 26, 2012 01:48 PM

Polypeptide

Genbank accession no. NP_001774

Genbank version no. NP_001774.1 GI:4502685

Genbank record update date: Jun. 26, 2012 01:48 PM

### CROSS REFERENCES

WO2003/088808, US2003/0228319; WO2003/062401 (claim 9); US2002/150573 (claim 4, pages 13-14); WO99/58658 (claim 13, FIG. 16); WO92/07574 (FIG. 1); U.S. Pat. No. 5,644,033; Ha et al (1992) J. Immunol. 148(5):1526-1531; Müller et al (1992) Eur. J. Immunol.. 22:1621-1625; Hashimoto et al (1994) Immunogenetics 40(4):287-295; Preud'homme et al (1992) Clin. Exp. 5 Immunol. 90(1):141-146; Yu et al (1992) J. Immunol. 148(2) 633-637; Sakaguchi et al (1988) EMBO J. 7(11):3457-3464

(29) CXCR5 (Burkitt's lymphoma receptor 1, a G protein-coupled receptor that is activated by the CXCL13 chemokine, functions in lymphocyte migration and humoral defense, plays a 10 role in HIV-2 infection and perhaps development of AIDS, lymphoma, myeloma, and leukemia); 372 aa, pl: 8.54 MW: 41959 TM: 7 [P] Gene Chromosome: 11q23.3,

Nucleotide

Genbank accession no NM_001716

Genbank version no. NM_001716.4 GI:342307092

Genbank record update date: Sep. 30, 2012 01:49 PM

Polypeptide

Genbank accession no. NP_001707

Genbank version no. NP_001707.1 GI:4502415

Genbank record update date: Sep. 30, 2012 01:49 PM

### CROSS REFERENCES

WO2004/040000; WO2004/015426; US2003/105292 (Example 2); U.S. Pat. No. 6,555,339 (Example 2); WO2002/61087 (FIG. 1); WO2001/57188 (Claim 20, page 269); WO2001/72830 (pages 12-13); WO2000/22129 (Example 1, pages 152-153, 15 Example 2, pages 254-256); WO99/28468 (claim 1, page 38); U.S. Pat. No. 5,440,021 (Example 2, col 49-52); WO94/28931 (pages 56-58); WO92/17497 (claim 7, FIG. 5); Dobner et al (1992) Eur. J. Immunol. 22:2795-2799; Barella et al (1995) Biochem. J. 309:773-779

(30) HLA-DOB (Beta subunit of MHC class II molecule (Ia antigen) that binds peptides and 20 presents them to CD4+ T lymphocytes); 273 aa, pl: 6.56, MW: 30820.TM: 1 [P] Gene Chromosome: 6p21.3)

Nucleotide

Genbank accession no NM_002120

Genbank version no. NM_002120.3 GI:118402587

Genbank record update date: Sep. 8, 2012 04:46 PM

Polypeptide

Genbank accession no. NP_002111

Genbank version no. NP_002111.1 GI:4504403

Genbank record update date: Sep. 8, 2012 04:46 PM

### CROSS REFERENCES

Tonnelle et al (1985) EMBO J. 4(11):2839-2847; Jonsson et al (1989) Immunogenetics 29(6):411-413; Beck et al (1992) J. Mol. Biol. 228:433-441; Strausberg et al (2002) Proc. Natl. Acad. Sci USA 99:16899-16903; Servenius et al (1987) J. Biol. Chem. 262:8759-8766; Beck et al (1996) J. Mol. Biol. 25 255:1-13; Naruse et al (2002) Tissue Antigens 59:512-519; WO99/58658 (claim 13, FIG. 15); U.S. Pat. No. 6,153,408 (Col 35-38); U.S. Pat. No. 5,976,551 (col 168-170); U.S. Pat. No. 6,011,146 (col 145-146); Kasahara et al (1989) Immunogenetics 30(1):66-68; Larhammar et al (1985) J. Biol. Chem. 260(26):14111-14119

(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pl: 7.63, MW: 47206 TM: 1 [P] Gene Chromosome: 17p13.3).

Nucleotide

Genbank accession no NM_002561

Genbank version no. NM_002561.3 GI:325197202

Genbank record update date: Jun. 27, 2012 12:41 AM

Polypeptide

Genbank accession no. NP_002552

Genbank version no. NP_002552.2 GI:28416933

Genbank record update date: Jun. 27, 2012 12:41 AM

### CROSS REFERENCES

Le et al (1997) FEBS Lett. 418(1-2):195-199; WO2004/047749; WO2003/072035 (claim 10); Touchman et al (2000) Genome Res. 10:165-173; WO2002/22660 (claim 20); WO2003/093444 (claim 1); WO2003/087768 (claim 1); WO2003/029277 (page 82)

(32) CD72 (B-cell differentiation antigen CD72, Lyb-2); 359 aa, pl: 8.66, MW: 40225, TM: 1 5 [P] Gene Chromosome: 9p13.3).

Nucleotide

Genbank accession no NM_001782

Genbank version no. NM_001782.2 GI:194018444

Genbank record update date: Jun. 26, 2012 01:43 PM

Polypeptide

Genbank accession no. NP_001773

Genbank version no. NP_001773.1 GI:4502683

Genbank record update date: Jun. 26, 2012 01:43 PM

### CROSS REFERENCES

WO2004042346 (claim 65); WO2003/026493 (pages 51-52, 57-58); WO2000/75655 (pages 105-106); Von Hoegen et al (1990) J. Immunol. 144(12):4870-4877; Strausberg et al (2002) Proc. Natl. Acad. Sci USA 99:16899-16903.

(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis); 661 aa, pl: 6.20, MW: 74147 TM: 1 [P] Gene Chromosome: 5q12).

Nucleotide

Genbank accession no NM_005582

Genbank version no. NM_005582.2 GI:167555126

Genbank record update date: Sep. 2, 2012 01:50 PM

Polypeptide

Genbank accession no. NP_005573

Genbank version no. NP_005573.2 GI:167555127

Genbank record update date: Sep. 2, 2012 01:50 PM

### CROSS REFERENCES

US2002/193567; WO97/07198 (claim 11, pages 39-42); Miura et al (1996) 15 Genomics 38(3):299-304; Miura et al (1998) Blood 92:2815-2822; WO2003/083047; WO97/44452 (claim 8, pages 57-61); WO2000/12130 (pages 24-26).

(34) FcRH1 (Fc receptor-like protein 1, a putative receptor for the immunoglobulin Fc domain that contains C2 type Ig-like and ITAM domains, may have a role in B-lymphocyte 20 differentiation); 429 aa, pl: 5.28, MW: 46925 TM: 1 [P] Gene Chromosome: 1q21-1q22)

Nucleotide

Genbank accession no NM_052938

Genbank version no. NM_052938.4 GI:226958543

Genbank record update date: Sep. 2, 2012 01:43 PM

Polypeptide

Genbank accession no. NP_443170

Genbank version no. NP_443170.1 GI:16418419

Genbank record update date: Sep. 2, 2012 01:43 PM

### CROSS REFERENCES

WO2003/077836; WO2001/38490 (claim 6, FIG. 18E-1-18-E-2); Davis et al (2001) Proc. Natl. Acad. Sci USA 98(17):9772-9777; WO2003/089624 (claim 8); EP1347046 (claim 1); WO2003/089624 (claim 7).

(35) IRTA2 (Immunoglobulin superfamily receptor translocation associated 2, a putative immunoreceptor with possible roles in B cell development and lymphomagenesis; deregulation of the gene by translocation occurs in some B cell malignancies); 977 aa, pl: 6.88, MW: 106468, TM: 1 [P] Gene Chromosome: 1q21)

Nucleotide

Genbank accession no AF343662

Genbank version no. AF343662.1 GI:13591709

Genbank record update date: Mar. 11, 2010 01:16 AM

Polypeptide

Genbank accession no. AAK31325

Genbank version no. AAK31325.1 GI:13591710

Genbank record update date: Mar. 11, 2010 01:16 AM

### CROSS REFERENCES

AF343663, AF343664, AF343665, AF369794, AF397453, AK090423, AK090475, AL834187, AY358085; Mouse:AK089756, AY158090, AY506558; NP_112571.1; WO2003/024392 (claim 2, FIG. 97); Nakayama et al (2000) Biochem. Biophys. Res. Commun. 277(1):124-127; WO2003/077836; WO2001/38490 (claim 3, FIG. 18B-1-18B-2).

(36) TENB2 (TMEFF2, tomoregulin, TPEF, HPP1, TR, putative transmembrane 35 proteoglycan, related to the EGF/heregulin family of growth factors and follistatin); 374 aa)

Nucleotide

Genbank accession no AF179274

Genbank version no. AF179274.2 GI:12280939

Genbank record update date: Mar. 11, 2010 01:05 AM

Polypeptide

Genbank accession no. AAD55776

Genbank version no. AAD55776.2 GI:12280940

Genbank record update date: Mar. 11, 2010 01:05 AM

### CROSS REFERENCES

NCBI Accession: AAD55776, AAF91397, AAG49451, NCBI RefSeq: NP_057276; NCBI Gene: 23671; OMIM: 605734; SwissProt Q9UIK5; AY358907, CAF85723, CQ782436; WO2004/074320; JP2004113151; WO2003/042661; WO2003/009814; EP1295944 (pages 69-70); WO2002/30268 (page 329); WO2001/90304; US2004/249130; US2004/022727; WO2004/063355; US2004/197325; US2003/232350; 5 US2004/005563; US2003/124579; Horie et al (2000) Genomics 67:146-152; Uchida et al (1999) Biochem. Biophys. Res. Commun. 266:593-602; Liang et al (2000) Cancer Res. 60:4907-12; Glynne-Jones et al (2001) Int J Cancer. October 15; 94(2):178-84.

(37) PSMA-FOLH1 (Folate hydrolase (prostate-specific membrane antigen) 1)

Nucleotide

Genbank accession no M99487

Genbank version no. M99487.1 GI:190663

Genbank record update date: Jun. 23, 2010 08:48 AM

Polypeptide

Genbank accession no. AAA60209

Genbank version no. AAA60209.1 GI:190664

Genbank record update date: Jun. 23, 2010 08:48 AM

### CROSS REFERENCES

Israeli R. S., et al Cancer Res. 53 (2), 227-230 (1993)

Other Information

Official Symbol: FOLH1

Other Aliases: GIG27, FGCP, FOLH, GCP2, GCPII, NAALAD1, NAALAdase, PSM, PSMA, mGCP

Other Designations: N-acetylated alpha-linked acidic dipeptidase 1; N-acetylated-alpha-linked acidic dipeptidase I; NAALADase I; cell growth-inhibiting gene 27 protein; folylpoly-gamma-glutamate carboxypeptidase; glutamate carboxylase II; glutamate carboxypeptidase 2; glutamate carboxypeptidase II; membrane glutamate carboxypeptidase; prostate specific membrane antigen variant F; pteroylpoly-gamma-glutamate carboxypeptidase

Antibodies

U.S. Pat. No. 7,666,425:

Antibodies produces by Hybridomas having the following ATCC references:ATCC accession No. HB-12101, ATCC accession No. HB-12109, ATCC accession No. HB-12127 and ATCC accession No. HB-12126.

Proscan: a monoclonal antibody selected from the group consisting of 8H12, 3E11, 17G1, 29B4, 30C1 and 20F2 (U.S. Pat. No. 7,811,564; Moffett S., et al Hybridoma (Larchmt). 2007 December; 26(6):363-72).

Cytogen: monoclonal antibodies 7E11-C5 (ATCC accession No. HB 10494) and 9H10-A4 (ATCC accession No. HB11430)—U.S. Pat. No. 5,763,202

GlycoMimetics: NUH2—ATCC accession No. HB 9762 (U.S. Pat. No. 7,135,301)

Human Genome Science: HPRAJ70—ATCC accession No. 97131 (U.S. Pat. No. 6,824,993); Amino acid sequence encoded by the cDNA clone (HPRAJ70) deposited as American Type Culture Collection (“ATCC”) Deposit No. 97131

Medarex: Anti-PSMA antibodies that lack fucosyl residues—U.S. Pat. No. 7,875,278

Mouse anti-PSMA antibodies include the 3F5.4G6, 3D7.1.1, 4E10-1.14, 3E11, 4D8, 3E6, 3C9, 2C7, 1G3, 3C4, 3C6, 4D4, 1G9, 5C8B9, 3G6, 4C8B9, and monoclonal antibodies. Hybridomas secreting 3F5.4G6, 3D7.1.1, 4E10-1.14, 3E11, 4D8, 3E6, 3C9, 2C7, 1G3, 3C4, 3C6, 4D4, 1 G9, 5C8B9, 3G6 or 4C8B9 have been publicly deposited and are described in U.S. Pat. No. 6,159,508. Relevant hybridomas have been publicly deposited and are described in U.S. Pat. No. 6,107,090. Moreover, humanized anti-PSMA antibodies, including a humanized version of J591, are described in further detail in PCT Publication WO 02/098897.

Other mouse anti-human PSMA antibodies have been described in the art, such as mAb 107-1A4 (Wang, S. et al. (2001) Int. J. Cancer 92:871-876) and mAb 2C9 (Kato, K. et al. (2003) Int. J. Urol. 10:439-444).

Examples of human anti-PSMA monoclonal antibodies include the 4A3, 7F12, 8C12, 8A11, 16F9, 2A10, 2C6, 2F5 and 1C3 antibodies, isolated and structurally characterized as originally described in PCT Publications WO 01/09192 and WO 03/064606 and in U.S. Provisional Application Ser. No. 60/654,125, entitled “Human Monoclonal Antibodies to Prostate Specific Membrane Antigen (PSMA)”, filed on Feb. 18, 2005. The V.sub.H amino acid sequences of 4A3, 7F12, 8C12, 8A11, 16F9, 2A10, 2C6, 2F5 and 1C3 are shown in SEQ ID NOs: 1-9, respectively. The V.sub.L amino acid sequences of 4A3, 7F12, 8C12, 8A11, 16F9, 2A10, 2C6, 2F5 and 1C3 are shown in SEQ ID NOs: 10-18, respectively.

Other human anti-PSMA antibodies include the antibodies disclosed in PCT Publication WO 03/034903 and US Application No. 2004/0033229.

NW Biotherapeutics: A hybridoma cell line selected from the group consisting of 3F5.4G6 having ATCC accession number HB12060, 3D7-1.1. having ATCC accession number HB12309, 4E10-1.14 having ATCC accession number HB12310, 3E11 (ATCC HB12488), 4D8 (ATCC HB12487), 3E6 (ATCC HB12486), 3C9 (ATCC HB12484), 2C7 (ATCC HB12490), 1G3 (ATCC HB12489), 3C4 (ATCC HB12494), 3C6 (ATCC HB12491), 4D4 (ATCC HB12493), 1G9 (ATCC HB12495), 5C8B9 (ATCC HB12492) and 3G6 (ATCC HB12485)—see U.S. Pat. No. 6,150,508

PSMA Development Company/Progenics/Cytogen—Seattle Genetics: mAb 3.9, produced by the hybridoma deposited under ATCC Accession No. PTA-3258 or mAb 10.3, produced by the hybridoma deposited under ATCC Accession No. PTA-3347—U.S. Pat. No. 7,850,971

PSMA Development Company—Compositions of PSMA antibodies (US 20080286284, Table 1)
This application is a divisional of U.S. patent application Ser. No. 10/395,894, filed on Mar. 21, 2003 (U.S. Pat. No. 7,850,971)

University Hospital Freiburg, Germany—mAbs 3/A12, 3/E7, and 3/F11 (Wolf P., et al Prostate. 2010 Apr. 1; 70(5):562-9).

(38) SST (Somatostatin Receptor; note that there are 5 subtypes)

(38.1) SSTR2 (Somatostatin receptor 2)

Nucleotide

Genbank accession no NM_001050

Genbank version no. NM_001050.2 GI:44890054

Genbank record update date: Aug. 19, 2012 01:37 PM

Polypeptide

Genbank accession no. NP_001041

Genbank version no. NP_001041.1 GI:4557859

Genbank record update date: Aug. 19, 2012 01:37 PM

### CROSS REFERENCES

Yamada Y., et al Proc. Natl. Acad. Sci. U.S.A. 89 (1), 251-255 (1992); Susini C., et al Ann Oncol. 2006 December; 17(12):1733-42

Other Information

Official Symbol: SSTR2

Other Designations: SRIF-1; SS2R; somatostatin receptor type 2

(38.2) SSTR5 (Somatostatin receptor 5)

Nucleotide

Genbank accession no D16827

Genbank version no. D16827.1 GI:487683

Genbank record update date: Aug. 1, 2006 12:45 PM

Polypeptide

Genbank accession no. BAA04107

Genbank version no. BAA04107.1 GI:487684

Genbank record update date: Aug. 1, 2006 12:45 PM

### CROSS REFERENCES

Yamada, Y., et al Biochem. Biophys. Res. Commun. 195 (2), 844-852 (1993)

Other Information

Official Symbol: SSTR5

Other Aliases: SS-5-R

Other Designations: Somatostatin receptor subtype 5; somatostatin receptor type 5

(38.3) SSTR1

(38.4) SSTR3

(38.5) SSTR4

AvB6—Both Subunits (39+40)

(39) ITGAV (Integrin, alpha V;

Nucleotide

Genbank accession no M14648 J02826 M18365

Genbank version no. M14648.1 GI:340306

Genbank record update date: Jun. 23, 2010 08:56 AM

Polypeptide

Genbank accession no. AAA36808

Genbank version no. AAA36808.1 GI:340307

Genbank record update date: Jun. 23, 2010 08:56 AM

### CROSS REFERENCES

Suzuki S., et al Proc. Natl. Acad. Sci. U.S.A. 83 (22), 8614-8618 (1986)

Other Information

Official Symbol: ITGAV

Other Aliases: CD51, MSK8, VNRA, VTNR

Other Designations: antigen identified by monoclonal antibody L230; integrin alpha-V; integrin alphaVbeta3; integrin, alpha V (vitronectin receptor, alpha polypeptide, antigen CD51); vitronectin receptor subunit alpha

(40) ITGB6 (Integrin, beta 6)

Nucleotide

Genbank accession no NM_000888

Genbank version no. NM_000888.3 GI:9966771

Genbank record update date: Jun. 27, 2012 12:46 AM

Polypeptide

Genbank accession no. NP_000879

Genbank version no. NP_000879.2 GI:9625002

Genbank record update date: Jun. 27, 2012 12:46 AM

### CROSS REFERENCES

Sheppard D. J., et al Biol. Chem. 265 (20), 11502-11507 (1990)

Other Information

Official Symbol: ITGB6

Other Designations: integrin beta-6

Antibodies

Biogen: U.S. Pat. No. 7,943,742—Hybridoma clones 6.3G9 and 6.8G6 were deposited with the ATCC, accession numbers ATCC PTA-3649 and -3645, respectively.

Biogen: U.S. Pat. No. 7,465,449—In some embodiments, the antibody comprises the same heavy and light chain polypeptide sequences as an antibody produced by hybridoma 6.1A8, 6.3G9, 6.8G6, 6.2B1, 6.2B10, 6.2A1, 6.2E5, 7.1G10, 7.7G5, or 7.1C5.

Centocor (J&J): U.S. Pat. Nos. 7,550,142; 7,163,681
For example in U.S. Pat. No. 7,550,142—an antibody having human heavy chain and human light chain variable regions comprising the amino acid sequences shown in SEQ ID NO: 7 and SEQ ID NO: 8.

Seattle Genetics: 15H3 (Ryan M C., et al Cancer Res Apr. 15, 2012; 72(8 Supplement): 4630)

(41) CEACAM5 (Carcinoembryonic antigen-related cell adhesion molecule 5)

Nucleotide

Genbank accession no M17303

Genbank version no. M17303.1 GI:178676

Genbank record update date: Jun. 23, 2010 08:47 AM

Polypeptide

Genbank accession no. AAB59513

Genbank version no. AAB59513.1 GI:178677

Genbank record update date: Jun. 23, 2010 08:47 AM

### CROSS REFERENCES

Beauchemin N., et al Mol. Cell. Biol. 7 (9), 3221-3230 (1987)

Other Information

Official Symbol: CEACAM5

Other Aliases: CD66e, CEA

Other Designations: meconium antigen 100

Antibodies

AstraZeneca-Medlmmune:US 20100330103; US20080057063;
US20020142359
   for example an antibody having complementarity determining regions (CDRs) with the following sequences: heavy chain; CDR1—DNYMH, CDR2—WIDPENGDTE YAPKFRG, CDR3—LIYAGYLAMD Y; and light chain CDR1—SASSSVTYMH, CDR2—STSNLAS, CDR3—QQRSTYPLT.
   Hybridoma 806.077 deposited as European Collection of Cell Cultures (ECACC) deposit no. 96022936.

Research Corporation Technologies, Inc.:U.S. Pat. No. 5,047,507

Bayer Corporation: U.S. Pat. No. 6,013,772

BioAlliance: U.S. Pat. Nos. 7,982,017; 7,674,605
U.S. Pat. No. 7,674,605
   an antibody comprising the heavy chain variable region sequence from the amino acid sequence of SEQ ID NO: 1, and the light chain variable region sequence from the amino acid sequence of SEQ ID NO:2.
   an antibody comprising the heavy chain variable region sequence from the amino acid sequence of SEQ ID NO:5, and the light chain variable region sequence from the amino acid sequence of SEQ ID NO:6.

Celltech Therapeutics Limited: U.S. Pat. No. 5,877,293

The Dow Chemical Company: U.S. Pat. Nos. 5,472,693; 6,417,337; 6,333,405
U.S. Pat. No. 5,472,693—for example, ATCC No. CRL-11215
U.S. Pat. No. 6,417,337—for example, ATCC CRL-12208
U.S. Pat. No. 6,333,405—for example, ATCC CRL-12208

Immunomedics, Inc: U.S. Pat. Nos. 7,534,431; 7,230,084; 7,300,644; 6,730,300;
US20110189085
   an antibody having CDRs of the light chain variable region comprise: CDR1 comprises KASQDVGTSVA (SEQ ID NO: 20); CDR2 comprises WTSTRHT (SEQ ID NO: 21); and CDR3 comprises QQYSLYRS (SEQ ID NO: 22);
   and the CDRs of the heavy chain variable region of said anti-CEA antibody comprise: CDR1 comprises TYWMS (SEQ ID NO: 23); CDR2 comprises EIHPDSSTINYAPSLKD (SEQ ID NO: 24); and CDR3 comprises LYFGFPWFAY (SEQ ID NO: 25).
US20100221175; US20090092598; US20070202044; US20110064653; US20090185974; US20080069775.

(42) MET (met proto-oncogene; hepatocyte growth factor receptor)

Nucleotide

Genbank accession no M35073

Genbank version no. M35073.1 GI:187553

Genbank record update date: Mar. 6, 2012 11:12 AM

Polypeptide

Genbank accession no. AAA59589

Genbank version no. AAA59589.1 GI:553531

Genbank record update date: Mar. 6, 2012 11:12 AM

### CROSS REFERENCES

Dean M., et al Nature 318 (6044), 385-388 (1985)

Other Information

Official Symbol: MET

Other Aliases: AUTS9, HGFR, RCCP2, c-Met

Other Designations: HGF receptor; HGF/SF receptor; SF receptor; hepatocyte growth factor receptor; met proto-oncogene tyrosine kinase; proto-oncogene c-Met; scatter factor receptor; tyrosine-protein kinase Met

Antibodies

Abgenix/Pfizer: US20100040629
for example, the antibody produced by hybridoma 13.3.2 having American Type Culture Collection (ATCC) accession number PTA-5026; the antibody produced by hybridoma 9.1.2 having ATCC accession number PTA-5027; the antibody produced by hybridoma 8.70.2 having ATCC accession number PTA-5028; or the antibody produced by hybridoma 6.90.3 having ATCC accession number PTA-5029.

Amgen/Pfizer: US20050054019
for example, an antibody comprising a heavy chain having the amino acid sequences set forth in SEQ ID NO: 2 where X2 is glutamate and X4 is serine and a light chain having the amino acid sequence set forth in SEQ ID NO: 4 where X8 is alanine, without the signal sequences; an antibody comprising a heavy chain having the amino acid sequences set forth in SEQ ID NO: 6 and a light chain having the amino acid sequence set forth in SEQ ID NO: 8, without the signal sequences; an antibody comprising a heavy chain having the amino acid sequences set forth in SEQ ID NO: 10 and a light chain having the amino acid sequence set forth in SEQ ID NO: 12, without the signal sequences; or an antibody comprising a heavy chain having the amino acid sequences set forth in SEQ ID NO: 14 and a light chain having the amino acid sequence set forth in SEQ ID NO: 16, without the signal sequences.

Agouron Pharmaceuticals (Now Pfizer): US20060035907

Eli Lilly: US20100129369

Genentech: U.S. Pat. No. 5,686,292; US20100028337; US20100016241; US20070129301; US20070098707; US20070092520, US20060270594; US20060134104; US20060035278; US20050233960; US20050037431
U.S. Pat. No. 5,686,292—for example, ATCC HB-11894 and ATCC HB-11895
US 20100016241—for example, ATCC HB-11894 (hybridoma 1A3.3.13) or HB-11895 (hybridoma 5D5.11.6)

National Defense Medical Center, Taiwan: Lu R M., et al Biomaterials. 2011 April; 32(12):3265-74.

Novartis: US20090175860
for example, an antibody comprising the sequences of CDR1, CDR2 and CDR3 of heavy chain 4687, wherein the sequences of CDR1, CDR2, and CDR3 of heavy chain 4687 are residues 26-35, 50-65, and 98-102, respectively, of SEQ ID NO: 58; and the sequences of CDR1, CDR2, and CDR3 of light chain 5097, wherein the sequences of CDR1, CDR2, and CDR3 of light chain 5097 are residues 24-39,55-61, and 94-100 of SEQ ID NO: 37.

Pharmacia Corporation: US20040166544

Pierre Fabre: US20110239316, US20110097262, US20100115639

Sumsung: US 20110129481—for example a monoclonal antibody produced from a hybridoma cell having accession number KCLRF-BP-00219 or accession number of KCLRF-BP-00223.

Samsung: US 20110104176—for example an antibody produced by a hybridoma cell having Accession Number: KCLRF-BP-00220.

University of Turin Medical School: DN-30 Pacchiana G., et al J Biol Chem. 2010 Nov. 12; 285(46):36149-57

Van Andel Research Institute: Jiao Y., et al Mol Biotechnol. 2005 September; 31(1):41-54.

(43) MUC1 (Mucin 1, cell surface associated)

Nucleotide

Genbank accession no J05581

Genbank version no. J05581.1 GI:188869

Genbank record update date: Jun. 23, 2010 08:48 AM

Polypeptide

Genbank accession no. AAA59876

Genbank version no. AAA59876.1 GI:188870

Genbank record update date: Jun. 23, 2010 08:48 AM

### CROSS REFERENCES

Gendler S. J., et al J. Biol. Chem. 265 (25), 15286-15293 (1990)

Other Information

Official Symbol: MUC1

Other Aliases: RP11-263K19.2, CD227, EMA, H23AG, KL-6, MAM6, MUC-1, MUC-1/SEC, MUC-1/X, MUC1/ZD, PEM, PEMT, PUM

Other Designations: DF3 antigen; H23 antigen; breast carcinoma-associated antigen DF3; carcinoma-associated mucin; episialin; krebs von den Lungen-6; mucin 1, transmembrane; mucin-1; peanut-reactive urinary mucin; polymorphic epithelial mucin; tumor associated epithelial mucin; tumor-associated epithelial membrane antigen; tumor-associated mucin

Antibodies

AltaRex—Quest Pharma Tech: U.S. Pat. No. 6,716,966—for example an Alt-1 antibody produced by the hybridoma ATCC No PTA-975.

AltaRex—Quest Pharma Tech: U.S. Pat. No. 7,147,850

CRT: 5E5—Sorensen A L., et al Glycobiology vol. 16 no. 2 pp. 96-107, 2006; HMFG2—Burchell J., et al Cancer Res., 47, 5476-5482 (1987)

Glycotope GT-MAB: GT-MAB 2.5-GEX (Website: http://www.glycotope.com/pipeline/pankomab-gex)

Immunogen: U.S. Pat. No. 7,202,346
for example, antibody MJ-170: hybridoma cell line MJ-170 ATCC accession no. PTA-5286Monoclonal antibody MJ-171: hybridoma cell line MJ-171 ATCC accession no. PTA-5287; monoclonal antibody MJ-172: hybridoma cell line MJ-172 ATCC accession no. PTA-5288; or monoclonal antibody MJ-173: hybridoma cell line MJ-173 ATCC accession no. PTA-5302

Immunomedics: U.S. Pat. No. 6,653,104

Ramot Tel Aviv Uni: U.S. Pat. No. 7,897,351

Regents Uni. CA: U.S. Pat. No. 7,183,388; US20040005647; US20030077676.

Roche GlycArt: U.S. Pat. No. 8,021,856

Russian National Cancer Research Center: Imuteran-Ivanov P K., et al Biotechnol J. 2007 July; 2(7):863-70

Technische Univ Braunschweig: (IIB6, HT186-B7, HT186-D11, HT186-G2, HT200-3A-C1, HT220-M-D1, HT220-M-G8)—Thie H., et al PLoS One. 2011 Jan. 14; 6(1):e15921

(44) CA9 (Carbonic anhydrase IX)

Nucleotide

Genbank accession no. X66839

Genbank version no. X66839.1 GI:1000701

Genbank record update date: Feb. 2, 2011 10:15 AM

Polypeptide

Genbank accession no. CAA47315

Genbank version no. CAA47315.1 GI:1000702

Genbank record update date: Feb. 2, 2011 10:15 AM

### CROSS REFERENCES

Pastorek J., et al Oncogene 9 (10), 2877-2888 (1994)

Other Information

Official Symbol: CA9

Other Aliases: CAIX, MN

Other Designations: CA-IX; P54/58N; RCC-associated antigen G250; RCC-associated protein G250; carbonate dehydratase IX; carbonic anhydrase 9; carbonic dehydratase; membrane antigen MN; pMW1; renal cell carcinoma-associated antigen G250

Antibodies

Abgenix/Amgen: US20040018198

Affibody: Anti-CAIX Affibody molecules (http://www.affibody.com/en/Product-Portfolio/Pipeline/)

Bayer: U.S. Pat. No. 7,462,696

Bayer/Morphosys: 3ee9 mAb—Petrul H M., et al Mol Cancer Ther. 2012 February; 11(2):340-9

Harvard Medical School: Antibodies G10, G36, G37, G39, G45, G57, G106, G119, G6, G27, G40 and G125. Xu C., et al PLoS One. 2010 Mar. 10; 5(3):e9625

Institute of Virology, Slovak Academy of Sciences (Bayer)—U.S. Pat. No. 5,955,075
for example, M75—ATCC Accession No. HB 11128 or MN12—ATCC Accession No. HB 11647

Institute of Virology, Slovak Academy of Sciences: U.S. Pat. No. 7,816,493
for example the M75 monoclonal antibody that is secreted from the hybridoma VU-M75, which was deposited at the American Type Culture Collection under ATCC No. HB 11128; or the V/10 monoclonal antibody secreted from the hybridoma V/10-VU, which was deposited at the International Depository Authority of the Belgian Coordinated Collection of Microorganisms (BCCM) at the Laboratorium voor Moleculaire Bioloqie-Plasmidencollectie (LMBP) at the Universeit Gent in Gent, Belgium, under Accession No. LMBP 6009CB.

Institute of Virology, Slovak Academy of Sciences US20080177046; US20080176310; US20080176258; US20050031623

Novartis: US20090252738

Wilex: U.S. Pat. No. 7,691,375—for example the antibody produced by the hybridoma cell line DSM ASC 2526.

Wilex: US20110123537; Rencarex: Kennett R H., et al Curr Opin Mol Ther. 2003 February; 5(1):70-5

Xencor: US20090162382

(45) EGFRvIII (Epidermal growth factor receptor (EGFR), transcript variant 3,

Nucleotide

Genbank accession no. NM_201283

Genbank version no. NM_201283.1 GI:41327733

Genbank record update date: Sep. 30, 2012 01:47 PM

Polypeptide

Genbank accession no. NP_958440

Genbank version no. NP_958440.1 GI:41327734

Genbank record update date: Sep. 30, 2012 01:47 PM

### CROSS-REFERENCES

Batra S K., et al Cell Growth Differ 1995; 6:1251-1259.

Antibodies:

U.S. Pat. Nos. 7,628,986 and 7,736,644 (Amgen)
For example, a heavy chain variable region amino acid sequence selected from the group consisting of SEQ ID NO: 142 and variants & a light chain variable region amino acid sequence selected from the group consisting of: SEQ ID NO: 144 and variants.

US20100111979 (Amgen)
For example, an antibody comprising a heavy chain amino acid sequence comprising:
CDR1 consisting of a sequence selected from the group consisting of the amino acid sequences for the CDR1 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17);
CDR2 consisting of a sequence selected from the group consisting of the amino acid sequences for the CDR2 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17); and
CDR3 consisting of a sequence selected from the group consisting of the amino acid sequences for the CDR3 region of antibodies 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).

US20090240038 (Amgen)
For example, an antibody having at least one of the heavy or light chain polypeptides comprises an amino acid sequence that is at least 90% identical to the amino acid sequence selected from the group consisting of: SEQ ID NO: 2, SEQ ID NO: 19, SEQ ID NO: 142, SEQ ID NO: 144, and any combination thereof.

US20090175887 (Amgen)
For example, an antibody having a heavy chain amino acid sequence selected from the group consisting of the heavy chain amino acid sequence of antibody 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).

US20090156790 (Amgen)
For example, antibody having heavy chain polypeptide and a light chain polypeptide, wherein at least one of the heavy or light chain polypeptides comprises an amino acid sequence that is at least 90% identical to the amino acid sequence selected from the group consisting of: SEQ ID NO: 2, SEQ ID NO: 19, SEQ ID NO: 142, SEQ ID NO: 144, and any combination thereof.

US20090155282, US20050059087 and US20050053608 (Amgen)
For example, an antibody heavy chain amino acid sequence selected from the group consisting of the heavy chain amino acid sequence of antibody 13.1.2 (SEQ ID NO: 138), 131 (SEQ ID NO: 2), 170 (SEQ ID NO: 4), 150 (SEQ ID NO: 5), 095 (SEQ ID NO: 7), 250 (SEQ ID NO: 9), 139 (SEQ ID NO: 10), 211 (SEQ ID NO: 12), 124 (SEQ ID NO: 13), 318 (SEQ ID NO: 15), 342 (SEQ ID NO: 16), and 333 (SEQ ID NO: 17).

MR1-1 (U.S. Pat. No. 7,129,332; Duke)
For example, a variant antibody having the sequence of SEQ ID NO.18 with the substitutions S98P-T99Y in the CDR3 VH, and F92W in CDR3 VL.

L8A4, H10, Y10 (Wikstrand C J., et al Cancer Res. 1995 Jul. 15; 55(14):3140-8; Duke)

US20090311803 (Harvard University)
For example, SEQ ID NO:9 for antibody heavy chain variable region, and SEQ ID NO: 3 for light chain variable region amino acid sequences

US20070274991 (EMD72000, also known as matuzumab; Harvard University)
For example, SEQ ID NOs: 3 & 9 for light chain and heavy chain respectively

U.S. Pat. No. 6,129,915 (Schering)
For example, SEQ. ID NOs: 1, 2, 3, 4, 5 and 6.

mAb CH12—Wang H., et al FASEB J. 2012 January; 26(1):73-80 (Shanghai Cancer Institute).

RAbDMvIII—Gupta P., et al BMC Biotechnol. 2010 Oct. 7; 10:72 (Stanford University Medical Center).

mAb Ua30—Ohman L., et al Tumour Biol. 2002 March-April; 23(2):61-9 (Uppsala University).

Han D G., et al Nan Fang Yi Ke Da Xue Xue Bao. 2010 January; 30(1):25-9 (Xi'an Jiaotong University).

(46) CD33 (CD33 molecule)

Nucleotide

Genbank accession no. M_23197

Genbank version no. NM_23197.1 GI:180097

Genbank record update date: Jun. 23, 2010 08:47 AM

Polypeptide

Genbank accession no. AAA51948

Genbank version no. AAA51948.1 GI:188098

Genbank record update date: Jun. 23, 2010 08:47 AM

### CROSS-REFERENCES

Simmons D., et al J. Immunol. 141 (8), 2797-2800 (1988)

Other Information

Official Symbol: CD33

Other Aliases: SIGLEC-3, SIGLEC3, p67

Other Designations: CD33 antigen (gp67); gp67; myeloid cell surface antigen CD33; sialic acid binding Ig-like lectin 3; sialic acid-binding Ig-like lectin

Antibodies

H195 (Lintuzumab)—Raza A., et al Leuk Lymphoma. 2009 August; 50(8):1336-44; U.S. Pat. No. 6,759,045 (Seattle Genetics/Immunomedics)

mAb OKT9: Sutherland, D. R. et al. Proc Natl Acad Sci USA 78(7): 4515-4519 1981, Schneider, C., et al J Biol Chem 257, 8516-8522 (1982)

mAb E6: Hoogenboom, H. R., et al J Immunol 144, 3211-3217 (1990)

U.S. Pat. No. 6,590,088 (Human Genome Sciences)
For example, SEQ ID NOs: 1 and 2 and ATCC accession no. 97521

U.S. Pat. No. 7,557,189 (Immunogen)
For example, an antibody or fragment thereof comprising a heavy chain variable region which comprises three CDRs having the amino acid sequences of SEQ ID NOs:1-3 and a light chain variable region comprising three CDRs having the amino acid sequences of SEQ ID NOs:4-6.

(47) CD19 (CD19 molecule)

Nucleotide

Genbank accession no. NM_001178098

Genbank version no. NM_001178098.1 GI:296010920

Genbank record update date: Sep. 10, 2012 12:43 AM

Polypeptide

Genbank accession no. NP_001171569

Genbank version no. NP_001171569.1 GI:296010921

Genbank record update date: Sep. 10, 2012 12:43 AM

### CROSS-REFERENCES

Tedder T F., et al J. Immunol. 143 (2): 712-7 (1989)

Other Information

Official Symbol: CD19

Other Aliases: B4, CVID3

Other Designations: B-lymphocyte antigen CD19; B-lymphocyte surface antigen B4; T-cell surface antigen Leu-12; differentiation antigen CD19

Antibodies

Immunogen: HuB4-Al-Katib A M., et al Clin Cancer Res. 2009 Jun. 15; 15(12):4038-45.

4G7: Kügler M., et al Protein Eng Des Sel. 2009 March; 22(3):135-47
For example, sequences in FIG. 3 of of Knappik, A. et al. J Mol Biol 2000 February; 296(1):57-86

AstraZeneca/MedImmune: MEDI-551—Herbst R., et al J Pharmacol Exp Ther. 2010 October; 335(1):213-22

Glenmark Pharmaceuticals: GBR-401—Hou S., et al Mol Cancer Ther November 2011 10 (Meeting Abstract Supplement) C164

U.S. Pat. No. 7,109,304 (Immunomedics)
For example, an antibody comprising the sequence of hA19Vk (SEQ ID NO:7) and the sequence of hA19VH (SEQ ID NO:10)

U.S. Pat. No. 7,902,338 (Immunomedics)
For example, an antibody or antigen-binding fragment thereof that comprises the light chain complementarity determining region CDR sequences CDR1 of SEQ ID NO: 16 (KASQSVDYDGDSYLN); CDR2 of SEQ ID NO: 17 (DASNLVS); and CDR3 of SEQ ID NO: 18 (QQSTEDPWT) and the heavy chain CDR sequences CDR1 of SEQ ID NO: 19 (SYWMN); CDR2 of SEQ ID NO: 20 (QIWPGDGDTNYNGKFKG) and CDR3 of SEQ ID NO: 21 (RETTTVGRYYYAMDY) and also comprises human antibody framework (FR) and constant region sequences with one or more framework region amino acid residues substituted from the corresponding framework region sequences of the parent murine antibody, and wherein said substituted FR residues comprise the substitution of serine for phenylalanine at Kabat residue 91 of the heavy chain variable region.

Medarex: MDX-1342—Cardarelli P M., et al Cancer Immunol Immunother. 2010 February; 59(2):257-65.

MorphoSys/Xencor: MOR-208/XmAb-5574—Zalevsky J., et al Blood. 2009 Apr. 16; 113(16):3735-43

U.S. Pat. No. 7,968,687 (Seattle Genetics)
An antibody or antigen-binding fragment comprising a heavy chain variable domain comprising the amino acid sequence of SEQ ID NO:9 and a light chain variable domain comprising the amino acid sequence of SEQ ID NO: 24.

4G7 chim—Lang P., et al Blood. 2004 May 15; 103(10):3982-5 (University of Tubingen)
For example, FIG. 6 and SEQ ID No: 80 of US20120082664

Zhejiang University School of Medicine: 2E8—Zhang J., et al J Drug Target. 2010 November; 18(9):675-8

(48) IL2RA (Interleukin 2 receptor, alpha); NCBI Reference Sequence: NM_000417.2);

Nucleotide

Genbank accession no. NM_000417

Genbank version no. NM_000417.2 GI:269973860

Genbank record update date: Sep. 9, 2012 04:59 PM

Polypeptide

Genbank accession no. NP_000408

Genbank version no. NP_000408.1 GI:4557667

Genbank record update date: Sep. 9, 2012 04:59 PM

### CROSS-REFERENCES

Kuziel W. A., et al J. Invest. Dermatol. 94 (6 SUPPL), 27S-32S (1990)

Other Information

Official Symbol: IL2RA

Other Aliases: RP11-536K7.1, CD25, IDDM10, IL2R, TCGFR

Other Designations: FIL-2 receptor subunit alpha; IL-2-RA; IL-2R subunit alpha; IL2-RA; TAC antigen; interleukin-2 receptor subunit alpha; p55

Antibodies

U.S. Pat. No. 6,383,487 (Novartis/UCL: Baxilisimab [Simulect])

U.S. Pat. No. 6,521,230 (Novartis/UCL: Baxilisimab [Simulect])
For example, an antibody having an antigen binding site comprises at least one domain which comprises CDR1 having the amino acid sequence in SEQ. ID. NO: 7, CDR2 having the amino acid sequence in SEQ. ID. NO: 8, and CDR3 chaving the amino acid sequence in SEQ. ID. NO: 9; or said CDR1, CDR2 and CDR3 taken in sequence as a whole comprise an amino acid sequence which is at least 90% identical to SEQ. ID. NOs: 7, 8 and 9 taken in sequence as a whole.

Daclizumab—Rech A J., et al Ann N Y Acad Sci. 2009 September; 1174:99-106 (Roche)

(49) AXL (AXL receptor tyrosine kinase)

Nucleotide

Genbank accession no. M76125

Genbank version no. M76125.1 GI:292869

Genbank record update date: Jun. 23, 2010 08:53 AM

Polypeptide

Genbank accession no. AAA61243

Genbank version no. AAA61243.1 GI:29870

Genbank record update date: Jun. 23, 2010 08:53 AM

### CROSS-REFERENCES

O'Bryan J. P., et al Mol. Cell. Biol. 11 (10), 5016-5031 (1991); Bergsagel P. L., et al J. Immunol. 148 (2), 590-596 (1992)

Other Information

Official Symbol: AXL

Other Aliases: JTK11, UFO

Other Designations: AXL oncogene; AXL transforming sequence/gene; oncogene AXL; tyrosine-protein kinase receptor UFO

Antibodies

YW327.6S2—Ye X., et al Oncogene. 2010 Sep. 23; 29(38):5254-64. (Genentech)

BergenBio: BGB324 (http://www.bergenbio.com/BGB324)

(50) CD30—TNFRSF8 (Tumor necrosis factor receptor superfamily, member 8)

Nucleotide

Genbank accession no. M83554

Genbank version no. M83554.1 GI:180095

Genbank record update date: Jun. 23, 2010 08:53 AM

Polypeptide

Genbank accession no. AAA51947

Genbank version no. AAA51947.1 GI:180096

Genbank record update date: Jun. 23, 2010 08:53 AM

### CROSS-REFERENCES

Durkop H., et al Cell 68 (3), 421-427 (1992)

Other Information

Official Symbol: TNFRSF8

Other Aliases: CD30, D1S166E, Ki-1

Other Designations: CD30L receptor; Ki-1 antigen; cytokine receptor CD30; lymphocyte activation antigen CD30; tumor necrosis factor receptor superfamily member 8

(51) BCMA (B-cell maturation antigen)—TNFRSF17 (Tumor necrosis factor receptor superfamily, member 17)

Nucleotide

Genbank accession no. Z29574

Genbank version no. Z29574.1 GI:471244

Genbank record update date: Feb. 2, 2011 10:40 AM

Polypeptide

Genbank accession no. CAA82690

Genbank version no. CAA82690.1 GI:471245

Genbank record update date: Feb. 2, 2011 10:40 AM

### CROSS-REFERENCES

Laabi Y., et al Nucleic Acids Res. 22 (7), 1147-1154 (1994)

Other Information

Official Symbol: TNFRSF17

Other Aliases: BCM, BCMA, CD269

Other Designations: B cell maturation antigen; B-cell maturation factor; B-cell maturation protein; tumor necrosis factor receptor superfamily member 17

(52) CT Ags—CTA (Cancer Testis Antigens)

### CROSS-REFERENCES

Fratta E., et al. Mol Oncol. 2011 April; 5(2):164-82; Lim S H., at al Am J Blood Res. 2012; 2(1):29-35.

(53) CD174 (Lewis Y)—FUT3 (fucosyltransferase 3 (galactoside 3(4)-L-fucosyltransferase, Lewis blood group)

Nucleotide

Genbank accession no. NM000149

Genbank version no. NM000149.3 GI:148277008

Genbank record update date: Jun. 26, 2012 04:49 PM

Polypeptide

Genbank accession no. NP_000140

Genbank version no. NP_000140.1 GI:4503809

Genbank record update date: Jun. 26, 2012 04:49 PM

### CROSS-REFERENCES

Kukowska-Latallo, J. F., et al Genes Dev. 4 (8), 1288-1303 (1990)

Other Information

Official Symbol: FUT3

Other Aliases: CD174, FT3B, FucT-III, LE, Les

Other Designations: Lewis FT; alpha-(1,3/1,4)-fucosyltransferase; blood group Lewis alpha-4-fucosyltransferase; fucosyltransferase Ill; galactoside 3(4)-L-fucosyltransferase

(54) CLEC14A (C-type lectin domain family 14, member A; Genbank accession no. NM175060)

Nucleotide

Genbank accession no. NM175060

Genbank version no. NM175060.2 GI:371123930

Genbank record update date: Apr. 1, 2012 03:34 PM

Polypeptide

Genbank accession no. NP_778230

Genbank version no. NP_778230.1 GI:28269707

Genbank record update date: Apr. 1, 2012 03:34 PM

Other Information

Official Symbol: CLEC14A

Other Aliases: UNQ236/PRO269, C14orf27, CEG1, EGFR-5

Other Designations: C-type lectin domain family 14 member A; CIECT and EGF-like domain containing protein; epidermal growth factor receptor 5

(55) GRP78—HSPA5 (heat shock 70 kDa protein 5 (glucose-regulated protein, 78 kDa)

Nucleotide

Genbank accession no. NM005347

Genbank version no. NM005347.4 GI:305855105

Genbank record update date: Sep. 30, 2012 01:42 PM

Polypeptide

Genbank accession no. NP_005338

Genbank version no. NP_005338.1 GI:16507237

Genbank record update date: Sep. 30, 2012 01:42 PM

### CROSS-REFERENCES

Ting J., et al DNA 7 (4), 275-286 (1988)

Other Information

Official Symbol: HSPA5

Other Aliases: BIP, GRP78, MIF2

Other Designations: 78 kDa glucose-regulated protein; endoplasmic reticulum lumenal Ca(2+)-binding protein grp78; immunoglobulin heavy chain-binding protein

(56) CD70 (CD70 molecule) L08096

Nucleotide

Genbank accession no. L08096

Genbank version no. L08096.1 GI:307127

Genbank record update date: Jun. 23, 2010 08:54 AM

Polypeptide

Genbank accession no. AAA36175

Genbank version no. AAA36175.1 GI:307128

Genbank record update date: Jun. 23, 2010 08:54 AM

### CROSS-REFERENCES

Goodwin R. G., et al Cell 73 (3), 447-456 (1993)

Other Information

Official Symbol: CD70

Other Aliases: CD27L, CD27LG, TNFSF7

Other Designations: CD27 ligand; CD27-L; CD70 antigen; Ki-24 antigen; surface antigen CD70; tumor necrosis factor (ligand) superfamily, member 7; tumor necrosis factor ligand superfamily member 7

Antibodies

MDX-1411 against CD70 (Medarex)

h1F6 (Oflazoglu, E., et al, Clin Cancer Res. 2008 Oct. 1; 14(19):6171-80; Seattle Genetics)
For example, see US20060083736 SEQ ID NOs: 1, 2, 11 and 12 and FIG. 1.

(57) Stem Cell specific antigens. For example:
5T4 (see entry (63) below)
CD25 (see entry (48) above)
CD32
   Polypeptide
      Genbank accession no. ABK42161
      Genbank version no. ABK42161.1 GI:117616286
      Genbank record update date: Jul. 25, 2007 03:00 PM
LGR5/GPR49
   Nucleotide
      Genbank accession no. NM_003667
      Genbank version no. NM_003667.2 GI:24475886
      Genbank record update date: Jul. 22, 2012 03:38 PM
   Polypeptide
      Genbank accession no. NP_003658
      Genbank version no. NP_003658.1 GI:4504379
      Genbank record update date: Jul. 22, 2012 03:38 PM
Prominin/CD133
   Nucleotide
      Genbank accession no. NM_006017
      Genbank version no. NM_006017.2 GI:224994187
      Genbank record update date: Sep. 30, 2012 01:47 PM
   Polypeptide
      Genbank accession no. NP_006008
      Genbank version no. NP_006008.1 GI:5174387
      Genbank record update date: Sep. 30, 2012 01:47 PM

(58) ASG-5

### CROSS-REFERENCES

(Smith L. M., et. al AACR 2010 Annual Meeting (abstract #2590); Gudas J. M., et. al. AACR 2010 Annual Meeting (abstract #4393)

Antibodies

Anti-AGS-5 Antibody: M6.131 (Smith, L. M., et. al AACR 2010 Annual Meeting (abstract #2590)

(59) ENPP3 (Ectonucleotide pyrophosphatase/phosphodiesterase 3)

Nucleotide

Genbank accession no. AF005632

Genbank version no. AF005632.2 GI:4432589

Genbank record update date: Mar. 10, 2010 09:41 PM

Polypeptide

Genbank accession no. AAC51813

Genbank version no. AAC51813.1 GI:2465540

Genbank record update date: Mar. 10, 2010 09:41 PM

### CROSS-REFERENCES

Jin-Hua P., et al Genomics 45 (2), 412-415 (1997)

Other Information

Official Symbol: ENPP3

Other Aliases: RP5-988G15.3, B10, CD203c, NPP3, PD-IBETA, PDNP3

Other Designations: E-NPP 3; dJ1005H11.3 (phosphodiesterase 1/nucleotide pyrophosphatase 3); dJ914N13.3 (phosphodiesterase 1/nucleotide pyrophosphatase 3); ectonucleotide pyrophosphatase/phosphodiesterase family member 3; gp130RB13-6; phosphodiesterase I beta; phosphodiesterase 1/nucleotide pyrophosphatase 3; phosphodiesterase-1 beta

(60) PRR4 (Pro/ine rich 4 (Iacrimal))

Nucleotide

Genbank accession no. NM_007244

Genbank version no. NM_007244.2 GI:154448885

Genbank record update date: Jun. 28, 2012 12:39 PM

Polypeptide

Genbank accession no. NP_009175

Genbank version no. NP_009175.2 GI:154448886

Genbank record update date: Jun. 28, 2012 12:39 PM

### CROSS-REFERENCES

Dickinson D. P., et al Invest. Ophthalmol. Vis. Sci. 36 (10), 2020-2031 (1995)

Other Information

Official Symbol: PRR4

Other Aliases: LPRP, PROL4

Other Designations: lacrimal proline-rich protein; nasopharyngeal carcinoma-associated proline-rich protein 4; proline-rich polypeptide 4; proline-rich protein 4

(61) GCC—GUCY2C (guanylate cyclase 2C (heat stable enterotoxin receptor)

Nucleotide

Genbank accession no. NM_004963

Genbank version no. NM_004963.3 GI:222080082

Genbank record update date: Sep. 2, 2012 01:50 PM

Polypeptide

Genbank accession no. NP_004954

Genbank version no. NP_004954.2 GI:222080083

Genbank record update date: Sep. 2, 2012 01:50 PM

### CROSS-REFERENCES

De Sauvage F. J., et al J. Biol. Chem. 266 (27), 17912-17918 (1991); Singh S., et al Biochem. Biophys. Res. Commun. 179 (3), 1455-1463 (1991)

Other Information

Official Symbol: GUCY2C

Other Aliases: DIAR6, GUC2C, MUCIL, STAR

Other Designations: GC-C; STA receptor; guanylyl cyclase C; hSTAR; heat-stable enterotoxin receptor; intestinal guanylate cyclase

(62) Liv-1—SLC39A6 (Solute carrier family 39 (zinc transporter), member 6)

Nucleotide

Genbank accession no. U41060

Genbank version no. U41060.2 GI:12711792

Genbank record update date: Nov. 30, 2009 04:35 PM

Polypeptide

Genbank accession no. AAA96258

Genbank version no. AAA96258.2 GI:12711793

Genbank record update date: Nov. 30, 2009 04:35 PM

### CROSS-REFERENCES

Taylor K M., et al Biochim Biophys Acta. 2003 Apr. 1; 1611(1-2):16-30

Other Information

Official Symbol: SLC39A6

Other Aliases: LIV-1

Other Designations: LIV-1 protein, estrogen regulated; ZIP-6; estrogen-regulated protein LIV-1; solute carrier family 39 (metal ion transporter), member 6; solute carrier family 39 member 6; zinc transporter ZIP6; zrt- and Irt-like protein 6

(63) 5T4, Trophoblast glycoprotein, TPBG-TPBG (trophoblast glycoprotein)

Nucleotide

Genbank accession no. AJ012159

Genbank version no. AJ012159.1 GI:3805946

Genbank record update date: Feb. 1, 2011 10:27 AM

Polypeptide

Genbank accession no. CAA09930

Genbank version no. CAA09930.1 GI:3805947

Genbank record update date: Feb. 1, 2011 10:27 AM

### CROSS-REFERENCES

King K. W., et al Biochim. Biophys. Acta 1445 (3), 257-270 (1999)

Other Information
Official Symbol: TPBG
Other Aliases: 5T4, 5T4AG, M6P1
Other Designations: 5T4 oncofetal antigen; 5T4 oncofetal trophoblast glycoprotein; 5T4 oncotrophoblast glycoprotein

(64) CD56—NCMA1 (Neural cell adhesion molecule 1)

Nucleotide

Genbank accession no. NM_000615

Genbank version no. NM_000615.6 GI:336285433

Genbank record update date: Sep. 23, 2012 02:32 PM

Polypeptide

Genbank accession no. NP_000606

Genbank version no. NP_000606.3 GI:94420689

Genbank record update date: Sep. 23, 2012 02:32 PM

### CROSS-REFERENCES

Dickson, G., et al, Cell 50 (7), 1119-1130 (1987)

Other Information

Official Symbol: NCAM1

Other Aliases: CD56, MSK39, NCAM

Other Designations: antigen recognized by monoclonal antibody 5.1H11; neural cell adhesion molecule, NCAM

Antibodies

Immunogen: HuN901 (Smith S V., et al Curr Opin Mol Ther. 2005 August; 7(4):394-401)
For example, see humanized from murine N901 antibody. See FIGS. 1b and 1e of Roguska, M. A., et al. Proc Natl Acad Sci USA February 1994; 91:969-973.

(65) CanAg (Tumor associated antigen CA242)

### CROSS-REFERENCES

Haglund C., et al Br J Cancer 60:845-851, 1989; Baeckstrom D., et al J Biol Chem 266:21537-21547, 1991

Antibodies

huC242 (Tolcher A W et al., J Clin Oncol. 2003 Jan. 15; 21(2):211-22; Immunogen)
For example, see US20080138898A1 SEQ ID NO: 1 and 2

(66) FOLR1 (Folate Receptor 1)

Nucleotide

Genbank accession no. J05013

Genbank version no. J05013.1 GI:182417

Genbank record update date: Jun. 23, 2010 08:47 AM

Polypeptide

Genbank accession no. AAA35823

Genbank version no. AAA35823.1 GI:182418

Genbank record update date: Jun. 23, 2010 08:47 AM

### CROSS-REFERENCES

Elwood P. C., et al J. Biol. Chem. 264 (25), 14893-14901 (1989)

Other Information

Official Symbol: FOLR1

Other Aliases: FBP, FOLR

Other Designations: FR-alpha; KB cells FBP; adult folate-binding protein; folate binding protein; folate receptor alpha; folate receptor, adult; ovarian tumor-associated antigen MOv18

Antibodies

M9346A—Whiteman K R., et al Cancer Res Apr. 15, 2012; 72(8 Supplement): 4628 (Immunogen)

(67) GPNMB (Glycoprotein (transmembrane) nmb)

Nucleotide

Genbank accession no. X76534

Genbank version no. X76534.1 GI:666042

Genbank record update date: Feb. 2, 2011 10:10 AM

Polypeptide

Genbank accession no. CAA54044

Genbank version no. CAA54044.1 GI:666043

Genbank record update date: Feb. 2, 2011 10:10 AM

### CROSS-REFERENCES

Weterman M. A., et al Int. J. Cancer 60 (1), 73-81 (1995)

Other Information

Official Symbol: GPNMB

Other Aliases: UNQ1725/PRO9925, HGFIN, NMB

Other Designations: glycoprotein NMB; glycoprotein nmb-like protein; osteoactivin; transmembrane glycoprotein HGFIN; transmembrane glycoprotein NMB

Antibodies

Celldex Therapeutics: CR011 (Tse K F., et al Clin Cancer Res. 2006 Feb. 15; 12(4):1373-82)
For example, see EP1827492B1 SEQ ID NO: 22, 24, 26, 31, 33 and 35

(68) TIM-1—HAVCR1 (Hepatitis A virus cellular receptor 1)

Nucleotide

Genbank accession no. AF043724

Genbank version no. AF043724.1 GI:2827453

Genbank record update date: Mar. 10, 2010 06:24 PM

Polypeptide

Genbank accession no. AAC39862

Genbank version no. AAC39862.1 GI:2827454

Genbank record update date: Mar. 10, 2010 06:24 PM

### CROSS-REFERENCES

Feigelstock D., et al J. Virol. 72 (8), 6621-6628 (1998)

Other Information

Official Symbol: HAVCR1

Other Aliases: HAVCR, HAVCR-1, KIM-1, KIM1, TIM, TIM-1, TIM1, TIMD-1, TIMD1

Other Designations: T cell immunoglobin domain and mucin domain protein 1; T-cell membrane protein 1; kidney injury molecule 1

(69) RG-1/Prostate tumor target Mindin—Mindin/RG-1

### CROSS-REFERENCES

Parry R., et al Cancer Res. 2005 Sep. 15; 65(18):8397-405

(70) B7-H4—VTCN1 (V-set domain containing T cell activation inhibitor 1

Nucleotide

Genbank accession no. BX648021

Genbank version no. BX648021.1 GI:34367180

Genbank record update date: Feb. 2, 2011 08:40 AM

### CROSS-REFERENCES

Sica G L., et al Immunity. 2003 June; 18(6):849-61

Other Information

Official Symbol: VTCN1

OtherAliases: RP11-229A19.4, B7-H4, B7H4, B7S1, B7X, B7h.5, PRO1291, VCTN1

Other Designations: B7 family member, H4; B7 superfamily member 1; T cell costimulatory molecule B7x; T-cell costimulatory molecule B7x; V-set domain-containing T-cell activation inhibitor 1; immune costimulatory protein B7-H4

(71) PTK7 (PTK7 protein tyrosine kinase 7)

Nucleotide

Genbank accession no. AF447176

Genbank version no. AF447176.1 GI:17432420

Genbank record update date: Nov. 28, 2008 01:51 PM

Polypeptide

Genbank accession no. AAL39062

Genbank version no. AAL39062.1 GI:17432421

Genbank record update date: Nov. 28, 2008 01:51 PM

### CROSS-REFERENCES

Park S. K., et al J. Biochem. 119 (2), 235-239 (1996)

Other Information

Official Symbol: PTK7

Other Aliases: CCK-4, CCK4

Other Designations: colon carcinoma kinase 4; inactive tyrosine-protein kinase 7; pseudo tyrosine kinase receptor 7; tyrosine-protein kinase-like 7

(72) CD37 (CD37 molecule)

Nucleotide

Genbank accession no. NM_001040031

Genbank version no. NM_001040031.1 GI:91807109

Genbank record update date: Jul. 29, 2012 02:08 PM

Polypeptide

Genbank accession no. NP_001035120

Genbank version no. NP_001035120.1 GI:91807110

Genbank record update date: Jul. 29, 2012 02:08 PM

### CROSS-REFERENCES

Schwartz-Albiez R., et al J. Immunol. 140 (3), 905-914 (1988)

Other Information

Official Symbol: CD37

Other Aliases: GP52-40, TSPAN26

Other Designations: CD37 antigen; cell differentiation antigen 37; leukocyte antigen CD37; leukocyte surface antigen CD37; tetraspanin-26; tspan-26

Antibodies

Boehringer Ingelheim: mAb 37.1 (Heider K H., et al Blood. 2011 Oct. 13; 118(15):4159-68)

Trubion: CD37-SMIP (G28-1 scFv-Ig) ((Zhao X., et al Blood. 2007; 110: 2569-2577)
For example, see US20110171208A1 SEQ ID NO: 253

Immunogen: K7153A (Deckert J., et al Cancer Res Apr. 15, 2012; 72(8 Supplement): 4625)

(73) CD138—SDC1 (syndecan 1)

Nucleotide

Genbank accession no. AJ551176

Genbank version no. AJ551176.1 GI:29243141

Genbank record update date: Feb. 1, 2011 12:09 PM

Polypeptide

Genbank accession no. CAD80245

Genbank version no. CAD80245.1 GI:29243142

Genbank record update date: Feb. 1, 2011 12:09 PM

### CROSS-REFERENCES

O'Connell F P., et al Am J Clin Pathol. 2004 February; 121(2):254-63

Other Information

Official Symbol: SDC1

Other Aliases: CD138, SDC, SYND1, syndecan

Other Designations: CD138 antigen; heparan sulfate proteoglycan fibroblast growth factor receptor; syndecan proteoglycan 1; syndecan-1

Antibodies

Biotest: chimerized MAb (nBT062)—(Jagannath S., et al PosterASH#3060, 2010; WIPO Patent Application WO/2010/128087)
For example, see US20090232810 SEQ ID NO: 1 and 2

Immunogen: B-B4 (Tassone P., et al Blood 104_3688-3696)
For example, see US20090175863A1 SEQ ID NO: 1 and 2

(74) CD74 (CD74 molecule, major histocompatibility complex, class II invariant chain)

Nucleotide

Genbank accession no. NM_004355

Genbank version no. NM_004355.1 GI:343403784

Genbank record update date: Sep. 23, 2012 02:30 PM

Polypeptide

Genbank accession no. NP_004346

Genbank version no. NP_004346.1 GI:10835071

Genbank record update date: Sep. 23, 2012 02:30 PM

### CROSS-REFERENCES

Kudo, J., et al Nucleic Acids Res. 13 (24), 8827-8841 (1985)

Other Information

Official Symbol: CD74

Other Aliases: DHLAG, HLADG, II, Ia-GAMMA

Other Designations: CD74 antigen (invariant polypeptide of major histocompatibility complex, class II antigen-associated); HLA class II histocompatibility antigen gamma chain; HLA-DR antigens-associated invariant chain; HLA-DR-gamma; Ia-associated invariant chain; MHC HLA-DR gamma chain; gamma chain of class II antigens; p33

Antibodies

Immunomedics: hLL1 (Milatuzumab,)—Berkova Z., et al Expert Opin Investig Drugs. 2010 January; 19(1):141-9)
For example, see US20040115193 SEQ ID NOs: 19, 20, 21, 22, 23 and 24

Genmab: HuMax-CD74 (see website)

(75) Claudins—CLs (Claudins)

### CROSS-REFERENCES

Offner S., et al Cancer Immunol Immunother. 2005 May; 54(5):431-45, Suzuki H., et al Ann N Y Acad Sci. 2012 July; 1258:65-70)

In humans, 24 members of the family have been described—see literature reference.

(76) EGFR (Epidermal growth factor receptor)

Nucleotide

Genbank accession no. NM_005228

Genbank version no. NM_005228.3 GI:41927737

Genbank record update date: Sep. 30, 2012 01:47 PM

Polypeptide

Genbank accession no. NP_005219

Genbank version no. NP_005219.2 GI:29725609

Genbank record update date: Sep. 30, 2012 01:47 PM

### CROSS-REFERENCES

Dhomen N S., et al Crit Rev Oncog. 2012; 17(1):31-50

Other Information

Official Symbol: EGFR

Other Aliases: ERBB, ERBB1, HER1, PIG61, mENA

Other Designations: avian erythroblastic leukemia viral (v-erb-b) oncogene homolog; cell growth inhibiting protein 40; cell proliferation-inducing protein 61; proto-oncogene c-ErbB-1; receptor tyrosine-protein kinase erbB-1

Antibodies

BMS: Cetuximab (Erbitux)—Broadbridge V T., et al Expert Rev Anticancer Ther. 2012 May; 12(5):555-65.
For example, see U.S. Pat. No. 6,217,866—ATTC deposit No. 9764.

Amgen: Panitumumab (Vectibix)—Argiles G., et al Future Oncol. 2012 April; 8(4):373-89
For example, see U.S. Pat. No. 6,235,883 SEQ ID NOs: 23-38.

Genmab: Zalutumumab—Rivera F., et al Expert Opin Biol Ther. 2009 May; 9(5):667-74.

YM Biosciences: Nimotuzumab—Ramakrishnan M S., et al MAbs. 2009 January-February; 1(1):41-8.
For example, see U.S. Pat. No. 5,891,996 SEQ ID NOs: 27-34.

(77) Her3 (ErbB3)—ERBB3 (v-erb-b2 erythroblastic leukemia viral oncogene homolog 3 (avian))

Nucleotide

Genbank accession no. M34309

Genbank version no. M34309.1 GI:183990

Genbank record update date: Jun. 23, 2010 08:47 PM

Polypeptide

Genbank accession no. AAA35979

Genbank version no. AAA35979.1 GI:306841

Genbank record update date: Jun. 23, 2010 08:47 PM

### CROSS-REFERENCES

Plowman, G. D., et al., Proc. Natl. Acad. Sci. U.S.A. 87 (13), 4905-4909 (1990)

Other Information

Official Symbol: ERBB3

Other Aliases: ErbB-3, HER3, LCCS2, MDA-BF-1, c-erbB-3, c-erbB3, erbB3-S, p180-ErbB3, p45-sErbB3, p85-sErbB3

Other Designations: proto-oncogene-like protein c-ErbB-3; receptor tyrosine-protein kinase erbB-3; tyrosine kinase-type cell surface receptor HER3

Antibodies

Merimack Pharma: MM-121 (Schoeberl B., et al Cancer Res. 2010 Mar. 15; 70(6):2485-2494)
For example, see US2011028129 SEQ ID NOs: 1, 2, 3, 4, 5, 6, 7 and 8.

(78) RON—MST1R (macrophage stimulating 1 receptor (c-met-related tyrosine kinase))

Nucleotide

Genbank accession no. X70040

Genbank version no. X70040.1 GI:36109

Genbank record update date: Feb. 2, 2011 10:17 PM

Polypeptide

Genbank accession no. CAA49634

Genbank version no. CAA49634.1 GI:36110

Genbank record update date: Feb. 2, 2011 10:17 PM

### CROSS-REFERENCES

Ronsin C., et al Oncogene 8 (5), 1195-1202 (1993)

Other Information

Official Symbol: MST1R

Other Aliases: CD136, CDw136, PTK8, RON

Other Designations: MSP receptor; MST1R variant RON30; MST1R variant RON62; PTK8 protein tyrosine kinase 8; RON variant E2E3; c-met-related tyrosine kinase; macrophage-stimulating protein receptor; p185-Ron; soluble RON variant 1; soluble RON variant 2; soluble RON variant 3; soluble RONvariant 4

(79) EPHA2 (EPH receptor A2)

Nucleotide

Genbank accession no. BC037166

Genbank version no. BC037166.2 GI:33879863

Genbank record update date: Mar. 6, 2012 01:59 PM

Polypeptide

Genbank accession no. AAH37166

Genbank version no. AAH37166.1 GI:22713539

Genbank record update date: Mar. 6, 2012 01:59 PM

### CROSS-REFERENCES

Strausberg R. L., et al Proc. Natl. Acad. Sci. U.S.A. 99 (26), 16899-16903 (2002)

Other Information

Official Symbol: EPHA2

Other Aliases: ARCC2, CTPA, CTPP1, ECK

Other Designations: ephrin type-A receptor 2; epithelial cell receptor protein tyrosine kinase; soluble EPHA2 variant 1; tyrosine-protein kinase receptor ECK

Antibodies

Medimmune: 1C1 (Lee J W., et al Clin Cancer Res. 2010 May 1; 16(9):2562-2570)
For example, see US20090304721A1 FIGS. 7 and 8.

(80) CD20—MS4A1 (membrane-spanning 4-domains, subfamily A, member 1)

Nucleotide

Genbank accession no. M27394

Genbank version no. M27394.1 GI:179307

Genbank record update date: Nov. 30, 2009 11:16 AM

Polypeptide

Genbank accession no. AAA35581

Genbank version no. AAA35581.1 GI:179308

Genbank record update date: Nov. 30, 2009 11:16 AM

### CROSS-REFERENCES

Tedder T. F., et al Proc. Natl. Acad. Sci. U.S.A. 85 (1), 208-212 (1988)

Other Information

Official Symbol: MS4A1

Other Aliases: B1, Bp35, CD20, CVID5, LEU-16, MS4A2, S7

Other Designations: B-lymphocyte antigen CD20; B-lymphocyte cell-surface antigen B1; CD20 antigen; CD20 receptor; leukocyte surface antigen Leu-16

Antibodies

Genentech/Roche: Rituximab—Abdulla N E., et al BioDrugs. 2012 Apr. 1; 26(2):71-82.
For example, see U.S. Pat. No. 5,736,137, ATCC deposit No. HB-69119.

GSK/Genmab: Ofatumumab—Nightingale G., et al Ann Pharmacother. 2011 October; 45(10):1248-55.
For example, see US20090169550A1 SEQ ID NOs: 2, 4 and 5.

Immunomedics: Veltuzumab—Goldenberg D M., et al Leuk Lymphoma. 2010 May; 51(5):747-55.
For example, see U.S. Pat. No. 7,919,273B2 SEQ ID NOs: 1, 2, 3, 4, 5 and 6.

(81) Tenascin C—TNC (Tenascin C)

Nucleotide

Genbank accession no. NM_002160

Genbank version no. NM_002160.3 GI:340745336

Genbank record update date: Sep. 23, 2012 02:33 PM

Polypeptide

Genbank accession no. NP_002151

Genbank version no. NP_002151.2 GI:153946395

Genbank record update date: Sep. 23, 2012 02:33 PM

### CROSS-REFERENCES

Nies D. E., et al J. Biol. Chem. 266 (5), 2818-2823 (1991); Siri A., et al Nucleic Acids Res. 19 (3), 525-531 (1991)

Other Information

Official Symbol: TNC

Other Aliases: 150-225, GMEM, GP, HXB, JI, TN, TN-C

Other Designations: GP 150-225; cytotactin; glioma-associated-extracellular matrix antigen; hexabrachion (tenascin); myotendinous antigen; neuronectin; tenascin; tenascin-C isoform 14/AD1/16

Antibodies

Philogen: G11 (von Lukowicz T., et al J Nucl Med. 2007 April; 48(4):582-7) and F16 (Pedretti M., et al Lung Cancer. 2009 April; 64(1):28-33)
For example, see U.S. Pat. No. 7,968,685 SEQ ID NOs: 29, 35, 45 and 47.

(82) FAP (Fibroblast activation protein, alpha)

Nucleotide

Genbank accession no. U09278

Genbank version no. U09278.1 GI:1888315

Genbank record update date: Jun. 23, 2010 09:22 AM

Polypeptide

Genbank accession no. AAB49652

Genbank version no. AAB49652.1 GI:1888316

Genbank record update date: Jun. 23, 2010 09:22 AM

### CROSS-REFERENCES

Scanlan, M. J., et al Proc. Natl. Acad. Sci. U.S.A. 91 (12), 5657-5661 (1994)

Other Information

Official Symbol: FAP

Other Aliases: DPPIV, FAPA

Other Designations: 170 kDa melanoma membrane-bound gelatinase; integral membrane serine protease; seprase

(83) DKK-1 (Dickkopf 1 homolog (Xenopus laevis)

Nucleotide

Genbank accession no. NM_012242

Genbank version no. NM_012242.2 GI:61676924

Genbank record update date: Sep. 30, 2012 01:48 PM

Polypeptide

Genbank accession no. NP_036374

Genbank version no. NP_036374.1 GI:7110719

Genbank record update date: Sep. 30, 2012 01:48 PM

### CROSS-REFERENCES

Fedi P. et al J. Biol. Chem. 274 (27), 19465-19472 (1999)

Other Information

Official Symbol: DKK1

Other Aliases: UNQ492/PRO1008, DKK-1, SK

Other Designations: dickkopf related protein-1; dickkopf-1 like; dickkopf-like protein 1; dickkopf-related protein 1; hDkk-1

Antibodies

Novartis: BHQ880 (Fulciniti M., et al Blood. 2009 Jul. 9; 114(2):371-379)
For example, see US20120052070A1 SEQ ID NOs: 100 and 108.

(84) CD52 (CD52 molecule)

Nucleotide

Genbank accession no. NM_001803

Genbank version no. NM_001803.3 GI:1519245483

Genbank record update date: May 1, 2019 02:13 AM

Polypeptide

Genbank accession no. NP_001794

Genbank version no. NP_001794.2 GI:68342030

Genbank record update date: May 1, 2019 02:13 AM

### CROSS-REFERENCES

Xia M. Q., et al Eur. J. Immunol. 21 (7), 1677-1684 (1991)

Other Information

Official Symbol: CD52

Other Aliases: CDW52

Other Designations: CAMPATH-1 antigen; CD52 antigen (CAMPATH-1 antigen); CDW52 antigen (CAMPATH-1 antigen); cambridge pathology 1 antigen; epididymal secretory protein E5; he5; human epididymis-specific protein 5

Antibodies

Alemtuzumab (Campath)—Skoetz N., et al Cochrane Database Syst Rev. 2012 Feb. 15; 2:CD008078.
For example, see Drugbank Acc. No. DB00087 (BIOD00109, BTDO0109)

(85) CS1—SLAMF7 (SLAM family member 7)

Nucleotide

Genbank accession no. NM_021181

Genbank version no. NM_021181.3 GI:1993571

Genbank record update date: Jun. 29, 2012 11:24 AM

Polypeptide

Genbank accession no. NP_067004

Genbank version no. NP_067004.3 GI:19923572

Genbank record update date: Jun. 29, 2012 11:24 AM

### CROSS-REFERENCES

Boles K. S., et al Immunogenetics 52 (3-4), 302-307 (2001)

Other Information

Official Symbol: SLAMF7

Other Aliases: UNQ576/PRO1138, 19A, CD319, CRACC, CS1

Other Designations: 19A24 protein; CD2 subset 1; CD2-like receptor activating cytotoxic cells; CD2-like receptor-activating cytotoxic cells; membrane protein FOAP-12; novel LY9 (lymphocyte antigen 9) like protein; protein 19A

Antibodies

BMS: elotuzumab/HuLuc63 (Benson D M., et al J Clin Oncol. 2012 Jun. 1; 30(16):2013-2015)
For example, see US20110206701 SEQ ID NOs: 9, 10, 11, 12, 13, 14, 15 and 16.

(86) Endoglin—ENG (Endoglin)

Nucleotide

Genbank accession no. AF035753

Genbank version no. AF035753.1 GI:3452260

Genbank record update date: Mar. 10, 2010 06:36 PM

Polypeptide

Genbank accession no. AAC32802

Genbank version no. AAC32802.1 GI:3452261

Genbank record update date: Mar. 10, 2010 06:36 PM

### CROSS-REFERENCES

Rius C., et al Blood 92 (12), 4677-4690 (1998)

Official Symbol: ENG

Other Information

Other Aliases: RP11-228B15.2, CD105, END, HHT1, ORW, ORW1

Other Designations: CD105 antigen

(87) Annexin A1—ANXA1 (Annexin A1)

Nucleotide

Genbank accession no. X05908

Genbank version no. X05908.1 GI:34387

Genbank record update date: Feb. 2, 2011 10:02 AM

Polypeptide

Genbank accession no. CAA29338

Genbank version no. CAA29338.1 GI:34388

Genbank record update date: Feb. 2, 2011 10:02 AM

### CROSS-REFERENCES

Wallner B. P., et al Nature 320 (6057), 77-81 (1986)

Other Information

Official Symbol: ANXA1

Other Aliases: RP11-71A24.1, ANX1, LPC1

Other Designations: annexin I (lipocortin I); annexin-1; calpactin II; calpactin-2; chromobindin-9; lipocortin I; p35; phospholipase A2 inhibitory protein

(88) V-CAM (CD106)—VCAM1 (Vascular cell adhesion molecule 1)

Nucleotide

Genbank accession no. M60335

Genbank version no. M60335.1 GI:340193

Genbank record update date: Jun. 23, 2010 08:56 AM

Polypeptide

Genbank accession no. AAA61269

Genbank version no. AAA61269.1 GI:340194

Genbank record update date: Jun. 23, 2010 08:56 AM

### CROSS-REFERENCES

Hession C., et al J. Biol. Chem. 266 (11), 6682-6685 (1991)

Other Information

Official Symbol VCAM1

Other Aliases: CD106, INCAM-100

Other Designations: CD106 antigen; vascular cell adhesion protein 1

(89) DLK-1 (Protein delta homolog 1)

Nucleotide

Genbank accession no. Z12172

Genbank version no. Z12172.1

Genbank record update date: Feb. 2, 2011 10:34 AM

Polypeptide

Genbank accession no. CAA78163

Genbank version no. CAA78163.1

Genbank record update date: Feb. 2, 2011 10:34 AM

### CROSS-REFERENCES

Laborda J. et al., J. Biol. Chem. 268:3817-3820(1993)

Other Information

Official Symbol DLK-1

Other Aliases: pG2

Other Designations: cleaved into Fetal antigen 1, FA1

(90) KAAG1 (Kidney-associated antigen 1)

Nucleotide

Genbank accession no. AF181720

Genbank version no. AF181720.1

Genbank record update date: Jul. 26, 2016 05:57 AM

Polypeptide

Genbank accession no. AAF23611

Genbank version no. AAF23611.1

Genbank record update date: Jul. 26, 2016 05:57 AM

### CROSS-REFERENCES

Van den Eynde B. J. et al., J. Exp. Med. 190:1793-1800(1999)

Other Information

Official Symbol KAAG1

Other Aliases: RU2 antisense gene protein

Pathogen Associated Antigens

The therapies described herein may be used to treat a pathogen-associated disorder. The term “pathogen-associated disorder” pertains disorder caused by, exacerbated-by, or associated with a pathogen. In some embodiments the pathogen-associated disorder is viral, bacterial, fungal, protozoan, parasitical, prion, or protein aggregate; that is, in some embodiments the disorder is associated with a virus, bacterium, fungus, protozoa, parasite, prion protein, or protein aggregate.

It is contemplated that the therapies of the present disclosure may be used to treat any pathogen-associated disorder that is characterized by a pathogen-associated antigen (PAA). Typically, a PAA is an antigen that is: (1) expressed by, or present on, the pathogen, and (2) not expressed by, or present in, the treated subject. Often a PAA will be an antigen present on the surface of a pathogen.

In some embodiments the pathogen-associated disorder is a viral disorder. For example, in some embodiments the pathogen-associated disorder is selected from the group consisting of Adenovirus infection, HIV/AIDS, Alphavirus Encephalitis, Arenavirus, Argentine hemorrhagic fever, Arthropod-borne viral encephalitis, Avian Influenza, Bolivian Hemorrhagic Fever, Borna disease, Chickenpox, Chikungunya, Coxsackievirus Infection, Crimean-Congo Hemorrhagic Fever, Cytomegalovirus infection, Dengue fever, Eastern equine encephalitis, Ebola, Echovirus Infection, Epstein-Barr Virus infection, Epstein-Barr Virus Related Tumours, Fifth Disease, Filovirus, Flavivirus, German measles, Hand, foot & mouth disease, Hemorrhagic fever with renal syndrome, Herpes Virus (Herpesviridae) infection, Herpes Simplex Virus Infection, Herpes Zoster Virus, Human Papilloma Virus Associated Epidermal Lesions, Human Papilloma Virus related Cervical Cancer, Mononucleosis, Influenza, Japanese Encephalitis, Kaposi Sarcoma, Korean Hemorrhagic Fever, Kyasanur Forest Disease, Lassa Fever, Lymphocytic choriomeningitis, Marburg Virus Disease, Measles, Molluscum Contagiosum, Mumps, Murray Valley encephalitis, Norwalk Virus related Diarrhea, Omsk hemorrhagic fever, Orthomyxoviruses, Parainfluenza Virus Infection, Paramyxovirus, Parvovirus B19 Infection, Picornavirus, Poxviruses, Rabies, Respiratory syncytial virus infection, Rift Valley Fever, Rotavirus diarrhea, Rubella, Rubeola, Smallpox, St. Louis Encephalitis, Tick-borne Encephalitis, Varicella, Variola, Venezuelan equine encephalitis, Viral hemorrhagic fevers, Western equine encephalitis, West Nile Virus disease Yellow Fever, and Zika

In some embodiments the PAA is derived from a virus. For example, in some embodiments the PAA is selected from the group consisting of Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirusalitis, GB virus C/Hepatitis G virus Pegivirus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus O, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

In some embodiments the PAA is a viral antigen. For example, in some embodiments the PAA is selected from the group consisting of the antigens having the following unique and stable UniProt entry identifier (see https://www.uniprot.org/help/entrv name): Q69549, Q07852, Q2HR82, Q7T928, P0C742, A0A0P0EFP7, Q80908, Q98839, Q9QJ49, P06427, P68564, Q5GFC6, Q3KSQ7, A0A0P0ERX0, PODJZ1, Q98223, P03193, P04016, P04364, E1U5P1, P14353, A0A0P0ELC3, Q9PX75, Q7T5D1, F5HG51, P17384, Q9QR71, E1U5N7, P29170, A0A0N716U3, P03285, Q98226, P89471, P27225, Q3KSP4, Q5VHB0, P03493, B5SNS7, P20879, Q98200, P52540, Q07857, Q3KSP5, Q5GFC1, P0C723, B5SNR2, P09295, Q2KS45, F5HINO, P21735, P20540, Q76DN6, P21043, B5SNS8, P03286, Q2KS32, Q9WF19, P22422, P35986, Q8BEL8, P33861, B5SNRO, P09731, Q9PWVO, P33828, P24836, Q1HVC3, Q76RB7, P0C733, B5SNS9, F5HDD3, Q2KS76, QOR5R3, P26555, Q8AZJ5, Q76RD5, Q3KSS5, B5SNS4, P09730, Q9PX47, P03086, P50782, P0C726, A9J188, Q1HVD3, B5SNT9, Q7M6G6, 11V199, P21097, P36825, P29996, A9J194, P0C722, B5SNS6, P09721, Q2TQY6, Q89187, P17387, P33803, Q77DT3, Q1HVF8, A9JOQ5, P09720, Q3S8C7, P20988, F5HAY6, P33042, Q6J3L8, Q1HVG1, A9JOW5, P09716, Q6QCM3, P20992, P03105, P03263, Q8QMU7, P68599, A9J1E2, F5H9N9, Q6QCP6, P68614, P36741, Q1HVD1, Q81971, P21041, A9J1J8, P21045, Q77JF8, P33843, Q9JH45, P03177, Q8QMX2, P24937, A9J1G6, P21099, Q6QCK2, P21057, P50798, P20843, 057247, Q81871, A9J1E9, P21100, Q6QCQ0, P33851, P03101, P33041, Q8QN10, P03277, A9JOX4, Q80923, Q6QCV8, P68618, P50822, P89446, C3V9V5, P03276, A9J141, P36723, Q6QCP2, P21065, P50800, Q3KSQ2, Q75Q70, P0C738, A9JOZ6, P26547, Q6QCM2, Q76QZ9, Q07861, P09250, Q91W62, P21036, A9JOS4, P17382, Q6QCWO, P21074, P50788, P21028, B9A5A8, P33859, A9JOV2, P36725, Q6QCL6, P68597, P36736, P18095, B9A5C6, P03284, A9JOW7, P06927, Q98222, P33835, Q80912, Q73367, B9A5D6, 012792, A9JOR2, P03118, Q806A8, P33836, P68320, P04591, Q3KSQ4, Q9DUC4, A9JON9, P04015, Q98261, P33837, P22425, P20874, Q4TUF4, P36716, A9J170, P27221, Q98216, PODJY9, Q98157, P08666, Q5VHB9, P21089, A9J138, P06463, Q98317, POCK28, P35256, P28864, Q5GFC8, Q9PX71, A9JOX6, PODODO, Q805Y6, Q6TUQ9, Q05128, P10189, Q8BEL5, Q9PX43, A9J107, Q80948, Q7T5D7, P33840, F5HC97, P52450, Q5VHC3, P21000, A9J1M0, Q45UF8, Q98315, P33842, Q9QJ40, P20978, Q5VHC6, P21001, A9J1K3, Q73369, Q98319, POCK20, Q9ENL1, F5HIG1, Q76RC6, Q9QJ58, A9J1R0, 070901, Q98275, P33847, Q89865, P10192, B9A5C1, P21115, A9J1T7, P60170, Q98311, P60672, Q77375, P89431, B9A5E0, P03224, A9J152, Q01351, Q76SZ3, P68616, Q9WT46, P52375, B9A5D2, P68345, A9J1U3, P52516, Q98210, P33854, P60505, P52451, B9A5A7, P03199, A9J101, P24435, Q98256, Q89183, Q9WT54, P04850, B9A5B0, Q3KST3, A9J1L5, Q9QJ28, Q98188, P21060, F5HCP3, Q6WB98, B9A5D9, P03197, A9JOU9, Q01352, Q76SZ4, P33832, P16773, 036634, B9A5D8, Q3KST5, A9J127, P52444, Q806B9, P21061, Q6SW10, P26629, Q3KSQ9, P30117, A9J1J4, P52445, Q7T5E1, P33855, F5HGQ8, Q77372, B9A5C5, Q3KSU2, A9J1K6, P20511, Q98294, P21069, F5H9Z4, P10211, B9A5B7, Q1HVH3, A9J136, P20523, Q805Y9, P21070, F5HAM0, P21022, Q5GFC2, P0C737, A9JOW9, P21085, Q806B8, P21066, P16741, P32991, E1U5P5, P03210, A9JOR8, P21119, Q89900, P21063, A8W995, P03347, Q5GFA9, P03206, A9J1K5, P21121, 11V175, P20990, P68620, P04592, E1U5P9, P21044, A9J114, P20542, 11V198, P33838, Q9QJ45, Q8JSZ3, Q8BEL7, P21104, A9J1E5, P20559, Q2KS4I, POCK27, Q69569, PODOEO, B9A5A6, P33797, A9JOP1, P68491, Q6JGG8, P20991, Q6SW14, P68762, B9A5B8, P12537, A9JOV4, P20551, Q2KS68, Q89489, P89436, P11235, K7ZLNO, P03274, A9J161, P20510, Q82009, P20535, P52463, P21030, Q3KSRO, P0C717, A9J1L4, P68485, Q4JEP8, P33816, Q9QJ33, P36320, Q6TV17, P30119, A9JOR6, P20512, Q82005, P33848, P52464, P0C763, B9A5B2, P0C734, A9J112, P68487, Q6QC16, P33850, F5HF47, Q69091, F8S4P1, P34016, A9JOT9, P20513, Q77JF7, P33852, P16821, P04488, C9EA16, 057211, A9J103, Q9DUC3, Q6QCL5, P33853, F5H8Q3, P89475, Q5VHA2, P33065, A9J148, Q9WGZ1, Q6QCL3, P33876, P16756, P68324, E1U5N3, P36713, A9J110, P68475, Q6QCT9, P21071, P16766, F5HAK9, Q6QCL1, 070739, A9J1V5, P68477, Q6QCQ2, P21067, Q6SW63, Q9QOU6, Q67721, Q9QU30, A9JOXO, P20547, Q6QCJ5, P33856, F5HAE6, P16827, Q6QCT4, P03207, A9J123, P20548, Q6X674, P21068, P52548, P52376, Q6QCQ1, Q3KSS8, A9JOU1, P20553, Q6QCP5, Q89186, Q06092, Q2HR92, Q6QCJ9, P21106, A9JOU3, P68626, Q6QCM5, P68595, P16798, P09300, Q3KSU9, P21090, B8XTP8, P20526, Q775H9, P33794, P08560, P90489, Q3KSVO, P21039, A9J1G1, P21120, Q6QCN1, P68594, F5HFB4, P03179, Q3KSU6, P34013, A9J1J5, P20527, Q6QCU3, P20534, Q6SW55, Q3KSV4, Q9QCF1, P34014, A9J1L3, P20530, Q6QCN2, P33839, P16813, Q1HVJO, Q98198, P33860, A9J114, P21123, Q6QCN5, P20994, P09288, Q6WB99, Q98255, P21042, A9J134, P20552, Q98213, Q07032, P16731, PODJX6, Q98274, Q9WSV7, A9JOQ7, F5HET4, Q98215, P21114, Q9WT06, Q3KSR5, Q7T5C1, P04133, A9J129, Q69548, Q98280, P68632, P52472, F5HF49, Q805Z9, Q9PZTO, A9JOW3, Q9QJ50, Q98197, P21096, P16762, P24935, Q98236, P12538, A9JOT7, F5H8R0, Q98259, P33849, P16787, P52448, Q98282, P21040, A9JOX5, Q69550, Q98248, P20993, Q6SWA4, Q2HR95, 040975, P20508, A9J1N4, P90463, Q98273, P68592, Q6SW89, Q1HVE7, Q98244, P03279, A9J1M1, Q9QJ46, Q98193, P33827, P16786, P16789, Q806C1, P36712, A9J1NO, Q69558, Q98283, P21058, Q6SW82, P04294, Q98278, P35988, A9JOX2, Q9QJ43, Q98307, P21064, P16775, P59632, Q7T5D6, P03281, A9J1R4, Q9QJ41, Q98196, P33844, P17146, P16728, Q806C0, P24447, A9J1F0, Q69559, Q7T5C5, P20985, Q6SWC3, F5HGN8, Q98271, P52346, A9J117, P52529, Q7T5C4, P33833, P16833, P09291, Q98302, P09253, A9J1T0, P03293, Q7T5E9, P20987, P16721, F5HE74, Q8V727, P03195, A9J1V3, Q7TLC7, Q7T5D4, PODJZ0, P10186, Q69551, Q7T400, Q3KST7, A9J1J3, PODKB2, Q805Z8, P33841, Q20MD0, Q89882, Q98175, P89469, A9JOU5, Q9ENK7, Q7T5E8, P20989, Q20MD6, F5HHS3, Q806A4, Q2HRC6, A9J1R6, A5HBD5, Q89478, Q98300, Q9WF16, P16845, Q806C2, P21050, A9JOY6, P35258, Q9YIE0, P20995, Q9WF14, P16849, Q98272, P68449, A9J1L0, Q9ENL4, Q6RK96, Q89485, Q9WF20, P17148, Q98301, P33822, A9J1F7, Q91N14, Q9PWU3, P20996, Q98172, P16822, Q98237, P21081, A9J0Q2, Q89843, Q5EY84, P20983, Q98281, P52545, Q98310, P21080, A9J132, P20882, Q4JEP6, P33858, Q98203, Q2HRB2, Q98227, P33862, A9J1E3, POC1C6, Q9PX68, P33857, Q98293, Q69554, Q7T5E6, P14351, A9J172, P03290, Q2KS40, P09279, Q98322, Q9WT45, Q7T401, P14075, A9J150, P03294, Q2KS44, P14348, Q98297, P24437, Q98284, Q0R5Q9, A9J1N6, P27557, Q6RK77, Q76QZ8, Q98312, P16801, Q806C4, P03383, A9J1C9, P03107, 11V197, P20531, Q87644, Q6SW48, Q805Z1, P04581, A9J1M3, Q05138, Q5EY48, P20842, Q98204, F5HFG3, Q89811, Q77377, A9J1I0, P03099, Q2KS77, Q5MQDO, Q98219, P16814, Q1WDN6, P20872, A9J1F2, P36753, Q2KS62, P03083, Q98328, P16797, Q806B4, P33064, A9J116, P36755, Q6RK85, A5HBD7, Q98206, F5HFJ8, Q98289, P03255, A9J112, P06794, Q9PX39, P20880, Q98205, P24439, Q85288, P03259, A9J1P3, P06418, 11V161, Q73370, Q6TVS6, P16781, Q7T5D2, P03243, A9J157, P32990, Q77SK4, P04611, Q6TVL3, P16847, Q806A5, P04491, A9JOZ4, P36735, Q9WF18, P04608, Q6TVMO, POCK56, Q98270, P36705, A9JOX9, P33814, Q7ZBU5, P14079, Q6TVT7, P16719, Q98309, P0DJX0, A9J1D8, P25486, Q76630, F5HF23, Q6TVJ4, Q6SW79, Q7T5C2, Q3KSU3, A9J1D1, Q02515, 11V8P0, P04487, Q6TVJ8, P28282, Q98212, P03211, A9JOP3, P27558, Q6TUQ1, F7V996, Q6TVT5, P16816, Q98316, Q3KSS4, A9JOS7, Q80918, Q6TUQ0, P68608, Q6TVK9, P16746, Q98269, Q69022, Q80945, P06918, Q6TUP1, P33805, Q6TVL6, Q6SWC6, A7KCN3, Q1HVG4, Q89184, Q80946, Q6TUS9, P15423, Q6QCV5, P16776, Q98286, P68634, Q07045, Q80953, Q6TUY4, P24940, Q6TVN7, P16737, Q8V721, P06498, 013311, Q5MQC7, DOZ5U4, P21077, Q6TVK0, P16828, Q805Y1, P36707, Q20MC9, Q6Q1R9, DOZ5T3, P21062, Q6TVS8, P16829, Q7T5C8, P68978, Q83977, P35260, DOZ5S5, P24933, Q6TVK5, P16738, Q9YIW0, P36709, Q20MD1, Q91N13, DOZ5T4, P68340, Q6TVJ0, P16770, Q98202, P21047, Q20MC7, P69481, DOZ5U8, PODOE5, Q6TVR8, P28275, Q98321, P33819, Q83976, P52439, Q9PWU2, P36714, Q6TVU9, P32988, Q805Z3, P06497, Q9WF13, P52440, DOZ5U9, P22112, Q6TVU1, P04413, Q7T5C6, P03242, G8H3V2, Q9YJJ8, DOZ5U3, P21132, Q6TVK6, P09722, Q806A6, P03191, Q80924, P05956, DOZSU1, P20841, Q6TVI8, Q6SW00, Q98279, POC6Z1, D6NGF9, P69726, DOZ5R9, P33831, Q6TVM5, F5HDK1, Q805Y0, F5HGH5, 11V8N7, P05954, Q80929, Q6Q1S2, Q6TVK4, P69332, Q98186, Q82040, 11V8N8. In each of the above Uniprot records, the sequence of the protein antigen relevant for the present disclosure is version 1, as designated in the Uniprot record by the record number followed by “−1”. This sequence designation is unique and unchaging. For example, in the Uniprot record 11V8N8 the the sequence of the protein antigen relevant for the present disclosure is designated in the Uniprot record as “I1V8N8-1”.

In some embodiments the pathogen-associated disorder is a bacterial disorder. For example, in some embodiments the pathogen-associated disorder is selected from the group consisting of Actinomycosis, Acute prostatitis, Anaerobic infection, Bacillary peliosis, Bacteremia, Bacterial pneumonia, Bacteroides ureolyticus, Baggio-Yoshinari syndrome, Barcoo fever, Bartonellosis, Biliary fever, Botryomycosis, Bovine campylobacteriosis, Brazilian purpuric fever, Brazilian Purpuric Fever, Brodie abscess, Burkholderia cepacia complex, Buruli ulcer, Campylobacteriosis, Capnocytophaga canimorsus, Cariogram, Carrion's disease, Chlamydia suis, Cholera, bacterial prostatitis, osteomyelitis, endodontic lesions, bovine pleuropneumonia, dermatitis, Diphtheria, Diphtheritic stomatitis, Epidural abscess, Epiglottitis, Erysipelas, Legionaires' disease, Far East scarlet-like fever, Fitz-Hugh-Curtis syndrome, Foot rot, Gardnerella vaginalis, Garre's sclerosing osteomyelitis, Granuloma inguinale, Haemophilus meningitis, monocytotropic ehrlichiosis, Lemierre's syndrome, Leprosy, Listeriosis, Lyme disease, Meningococcal disease, Mycobacterium avium-intracellulare infection, Necrotizing fasciitis, Nocardiosis, Noma, Omphalitis, Orbital cellulitis, Periodontal abscess, Periorbital cellulitis, Peritonsillar abscess, Pott disease, Proctitis, Pseudomonas infection, Psittacosis, Pyaemia, Pyomyositis, Q fever, Retropharyngeal abscess, Salmonellosis, Serratia infection, Shigellosis, Southern tick-associated rash illness, Staphylococcal scalded skin syndrome, brucellosis, Syphilis, Tetanus, Toxic shock syndrome, Trench fever, Tropical ulcer, Tubo-ovarian abscess, Ureaplasma urealyticum infection, Tuberculosis, Vertebral osteomyelitis, Waterhouse-Friderichsen syndrome, Whooping cough, Xanthogranulomatous osteomyelitis, and Yersiniosis.

In some embodiments the PAA is derived from a bacterium. For example, in some embodiments the PAA is iderived from a bacterium selected from the group consisting of Acetobacter aurantius, Acinetobacter baumannii, Actinomyces israelii, Agrobacterium radiobacter, Agrobacterium tumefaciens, Anaplasma sp. inc. Anaplasma phagocytophilum, Azorhizobium caulinodans, Azotobacter vinelandii, Bacillus, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus Thuringiensis, Bacteroides sp. inc Bacteroides fragilis, Bacteroides gingivalis, Bacteroides melaninogenicus, Prevotella melaninogenica, Bartonella sp. inc. Bartonella henselae, Bartonella Quintana, Bordetella sp. inc. Bordetella bronchiseptica, Bordetella pertussis, Borrelia burgdorferi, Brucella sp. inc. Brucella abortus, Brucella melitensis, Brucella suis, Burkholderia sp. inc. Burkholderia mallei, Burkholderia pseudomallei, Burkholderia cepacia, Calymmatobacterium granulomatis, Campylobacter sp. inc. Campylobacter coli, Campylobacter fetus, Campylobacter jejuni, Campylobacter pylori, Chlamydia sp. inc. Chlamydia trachomatis, Chlamydophila sp. inc. Chlamydophila pneumoniae, Chlamydophila psittaci, Clostridium sp. inc. Clostridium botulinum, Clostridium difficile, Clostridium perfringens, Clostridium tetani, Corynebacterium sp. inc. Corynebacterium diphtheriae, Corynebacterium fusiforme, Coxiella burnetii, Ehrlichia chaffeensis, Enterobacter cloacae, Enterococcus sp. inc. Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus sp. inc. Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus sp. inc. Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium sp. inc. Mycobacterium avium, Mycobacterium bovis, Mycobacterium diphtheria, Mycobacterium intracellulare, Mycobacterium leprae, Mycobacterium lepraemurium, Mycobacterium phlei, Mycobacterium smegmatis, Mycobacterium tuberculosis, Mycoplasma sp. inc. Mycoplasma fermentans, Mycoplasma genitalium, Mycoplasma hominis, Mycoplasma penetrans, Mycoplasma pneumoniae, Neisseria sp. inc. Neisseria gonorrhoeae, Neisseria meningitides, Pasteurella sp. inc. Pasteurella multocida, Pasteurella tularensis, Peptostreptococcus, Porphyromonas gingivalis, Prevotella melaninogenica, Pseudomonas aeruginosa, Rhizobium radiobacter, Rickettsia sp. inc. Rickettsia prowazekii, Rickettsia psittaci, Rickettsia quintana, Rickettsia rickettsia, Rickettsia trachomae, Rochalimaea sp. inc. Rochalimaea henselae, Rochalimaea quintana, Rothia dentocariosa, Salmonella sp. inc. Salmonella enteritidis, Salmonella typhi, Salmonella typhimurium, Serratia marcescens, Shigella dysenteriae, Spirillum volutans, Staphylococcus sp. inc. Staphylococcus aureus, Staphylococcus epidermidis, Stenotrophomonas maltophilia, Streptococcus sp. inc. Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema sp. inc. Treponema pallidum, Treponema denticola, Vibrio sp. inc. Vibrio cholerae, Vibrio comma, Vibrio parahaemolyticus, Vibrio vulnificus, Wolbachia, Yersinia sp. inc. Yersinia enterocolitica, Yersinia pestis, and Yersinia pseudotuberculosis.

In some embodiments the PAA is a bacterial antigen. For example, in some embodiments the PAA is selected from the group consisting of the antigens having the following unique and stable UniProt entry identifier (see https://www.uniprot.org/help/entrv name): P13423, Q9S5G6, Q7CCJ3, Q9L6R5, Q00473, C6DHE3, P9WIQ6, P37669, P42382, P31494, Q9AIX9, P9WL65, B1JS09, P9WLS9, Q83F12, Q2FDM1, P39180, P58237, P21979, Q9L6Q8, Q9HUF7, POAG01, L8FKX3, B1LLW8, P26950, P45680, Q9AJ64, 053379, Q56978, QOSYY3, P62605, Q2FUX3, P9WGU1, P72139, Q07297, Q57HS2, P26403, B4TNU6, P0A569, Q1R4E1, QOPAS1, C3PCX2, Q9AJ75, POAB35, P9WG11, P27833, P11089, Q6GDG4, P9WQP1, P9WJA3, Q7A3D7, B4SZ40, P26406, A8G841, PODOA7, B5QVI4, P18194, Q73DZ5, BORVK4, P0AB37, P9WGU0, B7MH57, Q06968, Q6G636, P9WIR7, Q9ZGM0, Q6GDN3, POAAA8, P37746, QOSYY4, P9WJD7, B5XYX5, P0C109, Q63GDO, 067998, P33792, QOP9X8, Q1CNJ1, Q06970, Q31SH3, P9WMK1, P60665, Q6G6A8, B7NTE8, Q07024, P9WLS5, P9WNK4, P27828, POA3P1, Q7A6D2, Q04974, Q04976, A1VZQ4, A4TRB6, Q06982, Q3YTH5, P9WPE7, Q4A122, Q79ZY3, A8A6P9, Q48476, P19478, 032629, B5BIU1, POA3P2, A8ZOC1, 066256, 031822, Q6F7F9, B5BIU3, Q06983, P18012, P9WK61, Q05852, Q5HDQ9, A9R8J0, Q06951, P18164, P44935, Q7MYM6, E4QEX4, B9DQ98, P16665, Q8FVC3, Q57254, Q66G07, P02975, Q6G6A5, P9WQN9, Q5HD54, Q8NV83, B1LLW7, Q50864, Q92BW7, Q83AF7, B4F1W8, POCOJ7, Q5HQE7, P37917, Q83BT6, P9WMK0, POAC79, E3PPC4, P18014, J714B7, Q6GDU6, Q53587, Q04866, P52642, Q48754, P14727, B5QV16, Q9LBG3, P31495, Q83F59, P9WN15, P50927, POAC78, POCK93, B2TT54, P9WQP3, Q5HLD1, P31784, Q6CZF1, Q8Z514, P9WM17, 030620, B7UNB2, P11657, PODJH1, P07643, Q6U113, P50928, A7FD62, P11312, Q03947, POC2T2, Q8CR67, Q5HCY4, 033789, P37748, Q83AX3, P68641, A1AHW3, Q9KKA3, Q6G2B2, P29721, Q9RNC7, Q84168, B7M5E2, P04740, Q54150, P9WQB1, P77682, P16952, POAG02, P0A236, Q83A32, P21762, Q1CNJ0, Q2YLR6, Q9S3N1, P19649, F7YJG6, Q1RGK9, C4ZZ62, P02973, P9W155, P9WPE5, P43413, Q7BQ98, A9MXG6, Q56083, Q2YIU6, P26276, Q8FBP8, P05430, Q811A1, P29720, Q9R2W4, P29697, B1LLW6, P33781, A7X6T9, P9WNK5, Q9S5G3, P46861, B5BIU2, Q00329, Q579D9, POCOB3, P27835, POCL66, P54166, P29724, Q04972, POA3N4, A8A6Q0, Q06971, Q7A377, P9WK45, Q06963, Q83D73, Q8Z396, POA1P4, Q9ZDHO, P27951, 033953, 031178, A6U0C2, P38369, Q45207, Q9F9F2, Q1CNI9, Q06974, Q8VSC3, Q06277, Q7DBF3, Q9HTB6, A7FD61, P27243, P43112, P45430, B614D1, Q8KQL6, Q2F160, P37761, G3XD61, Q52764, B11WA5, P06179, P18011, B5XK69, Q8DR60, P55293, P43109, P37742, Q45214, Q661N6, B7LU62, P9WIG7, A5IRJ3, P55294, P37749, Q9KH57, Q9L6R7, POA2K7, Q03946, P9WMU1, Q5LGZ8, P37745, P43110, P37755, P0A687, Q9X6B1, P95730, P96989, Q8CPR3, P26391, P37750, Q04102, B614D2, P0AEP6, Q2FDT8, Q53020, Q9S5G4, P37763, Q9RND2, P26405, P9WIG4, Q63K34, P37792, Q83EK8, AOR9F0, BORVLO, P71241, POA5B8, B5FN86, P25733, Q2FV52, P29723, P9WG64, Q46769, P21876, P26404, P9WIG5, P26949, B5EZ53, PODJ01, B7H9Q4, POC7J0, P26388, P50929, Q8ZAE1, Q3KMQ9, Q2YWD9, P9WJD9, P9WGM7, P61888, P29228, Q48478, P9WMU0, Q02192, POAG00, P18195, Q651A4, P37780, Q9XC60, P50930, B7LU61, POCOZ7, Q5HCY1, P02974, Q9L5X1, P31782, Q45215, Q05347, Q03084, Q63K37, B5FN87, PODH58, Q8NXC3, Q51832, Q8KIU8, P16624, A6TGI3, Q5ZXP3, Q6GDN1, Q49575, Q9L5X0, P9WGE7, Q45212, P37781, G3XD23, Q9X6B2, B5QV15, P15921, Q81YW9, P26392, PODMP6, P50931, COQ3A6, P16625, P60157, Q8FDQ2, PODF61, P23504, P11000, P0A235, B2TU00, P26948, C3LHC1, POCL67, C1EWE6, P37759, P67066, POA3N5, Q8Z395, P48219, Q5HL49, Q53047, POA4G3, Q53781, POA5N3, P37784, Q8Z389, Q63K35, B7JNE4, B7J1T8, A8FED1, Q6E7F4, Q9L6R4, Q7PA29, B5L3X1, P48214, Q8CMZ9, P33406, P31304, Q2G2J2, P37751, P26395, Q8ZAE4, QOSNDO, G3XD94, Q9HUG6, P22940, Q9S642, P67067, P22882, B5L3F2, G4WJD4, Q4AOG5, POCOJ8, P42363, Q6G723, P36667, Q05342, Q1CBP2, Q45010, P31496, Q83BSO, P37916, P37777, Q3YVHO, 052972, A8ACU6, P24301, A71Y64, PODJ00, Q9L5W9, Q2G2U9, Q4KXC9, P26471, A8ACU5, Q9ZLT1, P37918, P80369, 032270, P26394, A9R819, AOQU51, B7MH58, P12834, Q2YZ63, P26493, PODF60, A6QES8, P43111, Q48479, A7ZTZ6, P55980, Q02938, P20148, P17915, Q2SYI1, Q8XAS7, P9WQP0, B7L952, P69050, POA3L9, Q81QT1, P9WIN9, P13367, Q8KQC3, Q50863, B1JPZ9, A1VYV6, Q04973, P85410, Q2MGH6, Q7VJ79, P37457, 006052, B1IWA6, P24428, B1MHR6, Q9ABRO, Q07408, P80582, P29230, Q56902, B7MR14, P26926, Q04975, P13415, Q8A2Z5, P57041, B7L953, Q05861, P35272, P33548, J9W7B2, Q8P120, POA5Q3, P31781, Q49536, Q56903, A8ACU7, P94851, Q4L524, 006653, B1V8K7, E6MXWO, Q8XAQ3, P46729, B7NF99, POA2U4, AOAOH3MGR5, Q9AHT6, P9WNF5, P37779, Q49537, Q2FE11, B7M5E0, Q83ES6, B7HU46, P9W141, 030405, Q52657, Q8FBP9, P46730, Q1R4E2, Q326Z6, AOAOH3M3S8, Q99ZN9, P42364, P61887, Q49538, Q2FVC1, A7MQI6, P9WNB1, A9VSQ8, P9WHZ3, Q9A9H3, Q08137, P56258, P46731, B2VG51, Q03945, P9WK44, PODC90, Q9KIJ3, P55254, 085341, Q9ZB73, B7UNB3, P9WNQ7, Q2YWW6, Q6GDU9, Q826C5, P9WP33, B1XAG6, P46732, Q7MYM5, P65645, 007750, Q5XC63, Q9CFZ5, P26393, PODMP7, Q79FB3, A8A6P8, P9WNR7, Q6G167, Q6G613, Q06952, P9WQB0, A9R8J1, P12809, A9MJ16, Q9LAB5, Q49803, Q49588, POA4G4, BORVK9, P26389, Q83AH2, B5YY43, P9WNK3, Q49WE6, Q8NUV4, Q49VP4, Q00488, B5EZ52, P58248, Q57HS3, Q8X8T3, A5U990, P9WGT7, P31305, P44914, Q5JBG6, Q9KW51, B11WA7, PODOA6, B71W03, P18159, Q2FE05, P13719, A9MJ17, A1KJU9, B4TB26, Q51473, P9WK60, P46024, POA4V7, P37760, Q58YV9, Q99RE2, B2K060, P0A567, A7GKYO, Q2YW66, Q99RD4, P0C934, B5RFR1, P21160, B4SZ39, P72138, P24094, P29725, Q49771, POC8S3, Q9HZ76, Q7A3K7, A1AHW4, P54925, A7XOP5, Q5HD61, Q6G6H5, P19993, Q8XAR8, Q50397, Q5PKK8, Q9ZGM1, Q9ZJ31, 031357, P9WIN6, P37778, A8G840, Q8CN38, Q6CZF0, P9WP32, Q2FZP7, P41484, Q3S2Y1, P24093, Q5PKK9, POA4V5, BORVK6, Q83ELO, Q72U69, P0C223, P9WIN7, P37744, Q31UJ1, Q4L9R5, Q8FBP7, P9WNZ7, A6QFM7, P9WIM6, P77293, P43313, B5EZ54, POC2T1, Q329X2, Q7A3J9, T2GP47, Q45011, Q5XDI6, P55253, Q329X1, Q9ZCX6, Q7MYM4, POC1D7, Q99V75, P9WIM7, Q9JRN5, 068770, B5RFQ9, Q2YKY9, Q83IX6, Q9CCP6, P9WK74, Q83CY8, Q8P280, POC7J4, Q83PH8, Q7DBF7, B1JPF1, P0A647, Q6GARO, Q50703, Q51366, P9WNB3, B4TB27, P19361, P29229, A5U2B3, POA5J1, POA3U8, POA4G2, P55257, A9MXG7, Q79FU3, B4TB25, P40136, Q3S2Y2, Q6FFS6, Q56598, Q93GW2, B7MR15, P14062, P21875, Q01411, A0A0H3M9Z0, P17953, P9WNF3, P27830, B7UNB1, Q5HLD2, C6DHE2, Q9ZKJ5, Q4L8Y7, P9WIB5, Q56623, P19421, C4ZZ61, P9WQP2, Q45206, 085342, P31502, P25393, P0A669, 066251, C0Q3A8, Q49WH7, B4TNU5, P9WPS9, Q9ZH99, Q9A1S2, Q57301, P35635, B1JPZ8, Q06947, Q45209, P37753, P9WK65, P9WPE4, 052957, Q52938, A7ZTZ8, P9WIB4, Q8ZAE3, Q06972, A9WNAO, Q56127, Q06953, 088005, P37458, P31951, Q8FBQ3, P62586, AOQWU8, P15599, Q05868, P18481, B2TTZ8, Q79FW5, POAAA7, Q06981, Q48485, PODMDO, P37747, G4WJD3, Q1CBP1, A5U3Q3, A1JI79, P42216, P9WK75, 034194, P9WQN6, Q9JRN7, Q31UI9, P9WJQ1, A7MQI7, P15488, Q48481, P0DC91, P37785, P19422, B2K059, Q05862, A7FD60, P42217, POAOVO, P25734, P9WQN7, 083346, Q329X3, P9WJY7, A7ZTZ7, P11933, Q5L7M8, Q8DPY9, P68667, P60533, Q8ZAF1, 052956, Q8ZAF0, P0C7U7, P46733, POA5P3, P0A619, Q83DJ3, A1AHW2, POA5P9, Q8XAQ5, P04953, Q56872, Q2YKV1, Q2G1T6, 034191, Q6G2B4, P9WQN8, B7L8D5, A9R9H4, P9WK55, POA5N9, P9WG65, Q5HLV2, QOSYY5, P9W101, B5YY42, P19528, Q7BJX9, Q53107, Q2YW63, D3QY10, Q6G2B3, A1KQD8, B3FN88, A4TSQ1, P42218, P15598, P08180, Q4UNEO, Q1CBP0, P9WHW9, B5RFR0, Q00045, Q89ZXO, Q00474, Q8NUU9, P26879, Q9R3F2, Q83CP9, Q8Z397, Q9X6BO, Q7BHI8, P23033, Q03155, Q9AJ83, B614D0, POA5Q7, B2TTZ9, Q00046, A4Q8F7, P61711, Q45208, POA521, Q6FYW8, P94217, A8G842, P69957, P18010, P26194, 051401, Q52658, Q8ZAF2, Q81U45, Q31UJ0, P57039, Q8X7P7, P26397, Q47592, P9WPE6, P46815, P38016, B1XAG4, P69958, POCAX7, 032606, P58827, Q9AJ37, B7LU63, 033075, Q3YVG9, Q83E37, 031314, P26398, Q2SYH7, Q05433, AOR006, Q83E09, Q83IX5, P21207, P9WIP0, P9WIR0, P80069, Q9AJ79, QOTAS9, P9WIM8, P26880, P02970, 030511, P14168, G3XD01, P24251, B4TNU7, Q8VQ99, C6DHE4, P23995, P9WIP1, P9WIR1, P46842, Q9AJ77, Q8Z386, P9WIM9, P26881, P14190, C5WO22, P26402, P27829, POABW7, B7NTE9, P31631, Q66G05, P69960, POA5Q5, P9WIR3, P9WIR6, Q9AJ80, A9MJ15, P0C873, Q7ARC3, P14191, P18009, Q99191, P27832, P9WPH9, C4ZZ60, Q7A423, P27836, P15917, B2RHG2, P25732, Q48919, Q9AJ82, B5FN88, P68588, P37919, Q6E7F2, D2AJUO, P26400, B2K058, P9WJD8, B7MH59, Q6GED5, B7NFAO, P42502, B2RHG4, P9WIR2, Q9F9L1, Q9AJ63, Q5PKK7, P69966, P9WIZ7, P21982, A6QK59, Q50862, Q8XAQ8, P9WNK7, P45341, Q2FV55, POAC80, P23994, P9WIN8, P80200, Q9ZJD1, Q92JP8, Q8DVU8, POA5Y3, Q68X15, P52616, P99160, Q03584, B5YY41, POA565, QOTAT1, Q9KJT6, Q077R2, P42213, Q83A83, P55746, P56876, Q4UNI5, Q1RJI4, P9WGT6, Q8YD01, P13253, P60158, Q83DP8, B7M5E1, P9WNK6, Q3YVG8, Q97PA9, Q57HS4, Q47334, Q03490, P16626, P37915, Q9ZD49, Q92ID4, Q81TU1, P9WKW3, P11764, Q4L980, Q56128, B1XAG5, Q6E7F1, B7NTE7, Q83AY0, A4WG18, Q8X8T4, P46841, P60532, Q04971, Q9AJ81, Q4UM04, P69965, P44067, O51941, Q5HCQ9, P26401, Q0TAT0, P9WIQ7, B7N288, P36429, B4SZ38, Q9S5G5, Q7MXKO, P07889, B9J2U2, Q92JF7, P13422, P31522, Q83CZ8, Q06969, Q7A2K6, Q48475, A4TRB5, AOAOH2VDN9, B7NF98, Q53782, A9MXG5, Q01410, Q48899, P09239, Q6HNU4, Q3L8P3, Q83DN9, P68589, P34001, Q06973, Q79ZW0, Q03583, Q66G06, P9WNK2, Q1R4E3, Q7A2K8, Q6CZF2, Q9ZN40, 085343, P42384, Q5HH69, P0C1U6, P0AB36, P31527, Q48456, P05431, P18013, P26396, Q05032, P51836, Q9CNG8, Q99RX4, Q8Z388, P65301, A0Q4N6, Q00330, P76372, POA5P5, A4TRB4, P0A519, and P37741. In each of the above Uniprot records, the sequence of the protein antigen relevant for the present disclosure is version 1, as designated in the Uniprot record by the record number followed by “−1”. This sequence designation is unique and unchaging. For example, in the Uniprot record P37741 the the sequence of the protein antigen relevant for the present disclosure is designated in the Uniprot record as “P37741-1”.

In some embodiments the pathogen-associated disorder is a fungal disorder. For example, in some embodiments the pathogen-associated disorder is selected from the group consisting of Aspergillosis, Candidiasis, Coccidioidomycosis, Cryptococcus gattii infection, onychomycosis, microsporidiosis, mucormycosis, pneumocystis pneumonia, sporotrichosis, blastomycosis, Candida auris infection, Cryptococcus neoformans infection, fungal eye infection including keratitis and endophthalmitis, Histoplasmosis, mycetoma, dermatophytosis inc. tinea pedis tinea corporis, and talaromycosis.

In some embodiments the PAA is derived from a fungus. For example, in some embodiments the PAA is selected from the group consisting of Aspergillus sp., Candida sp. including C. albicans and C. auris, Coccidioides sp., Cryptococcus gattii, Pneumocystis jirovecii, Sporothrix sp., Blastomyces sp., Cryptococcus neoformans, Histoplasma sp., Talaromyces sp. including T. marneffei, Anncaliia algerae, A. connori, A. vesicularum, Encephalitozoon cuniculi, E. hellem, E. intestinalis, Enterocytozoon bieneusi, Microsporidium ceylonensis, M. africanum, Nosema ocularum, Pleistophora sp., Trachipleistophora hominis, T. anthropophthera, Vittaforma corneae, and Tubulinosema acridophagus.

In some embodiments the PAA is a fungal antigen. For example, in some embodiments the PAA is selected from the group consisting of the antigens having the following unique and stable UniProt entry identifier (see https://www.uniprot.org/help/entrv name): P87020, P43588, Q9UQY2, A6ZTR3, C5P230, Q1E3R8, B3STN5, Q4WMJ7, Q03392, Q08723, O13834, O94275, P20967, B2ZRS9, Q9XZV1, E9RCR4, P15873, Q9P7S2, P53549, E9CX44, O74531, P53119, Q95WA3, Q4WMJ1, POCB51, Q12377, P33298, P46589, 074783, P40985, Q95WA4, Q4WMJ9, Q59L12, P38204, 042931, Q1E8D2, Q09855, P24814, P00016, Q4WMJ8, G1UB63, Q03280, P41836, 074623, P87060, A4GYZO, P39940, Q4WMJ0, Q5AOX8, Q9UT05, 014250, 060022, Q5AB48, E9R9Y3, POCH07, Q9P3U4, Q59XX2, POCX83, 059770, Q9USX1, Q5F2J0, P46984, POCH09, P38199, P50142, O74440, Q10329, Q00746, O60182, Q4WMJ5, P38764, Q9USS7, Q00022, Q9US13, Q9UT97, P06104, Q5ABZ2, Q92211, P40016, Q1K9C4, Q03834, Q04062, P38624, O00103, POC7N7, Q04638, P38766, O60106, P25847, P87048, P25451, O00102, O74111, Q9US46, P35178, P38109, P25336, POCH06, P23724, P21734, Q8X082, Q5ALO3, P05759, Q10435, P32454, POCH08, Q12417, P52492, Q3E833, P40034, Q06103, P38202, P36049, QOC9L7, P21242, P15731, O14170, P36132, P53953, O14099, P53874, QOC9L6, Q9USQ9, POCG72, P25375, P38203, P38200, P34247, 042721, Q05583, P50086, Q08562, O94579, O74349, P52286, P38201, POCW95, P85437, P40303, Q09765, Q09682, Q04781, Q9Y709, 094264, Q12018, Q03071, 043063, P23566, 094393, P35728, P40482, Q10311, P38205, F2Z266, P25043, P38820, P40555, C5P3X6, P32565, 074549, 013959, Q5AMT2, Q01939, P0CG63, Q92462, P53152, Q12250, 013685, P38630, 043069, P40302, 042646, Q09841, P29469, P38886, P22515, P38629, P38862, P30657, Q02159, P30656, P39014, P41878, P46595, Q08273, P53323, 014126, 094609, P33297, P33310, P50524, P33296, P23639, Q01532, P36612, P52490, P40327, P33311, P32496, Q99344, P32379, P07267, P23638, Q9P7R4, P33299, Q2UNX8, P20606, P52491, 074445, P00729, P18239, P28263, 094517, Q8TGEO, 094672, 074810, 074894, 013807, P37898, 013731, P21243, P36113, 074873, P15732, Q9UTG2, QOC9L4, 074762, P19812, Q09720, POCW94, Q9UU15, Q07963, 014326, QOC9L5, 042897, P33202, P22141, O74983, P15303, P54860, 075004, 013790, Q10335, 060152, P30655, Q8TG42, POC8R3, Q5AJCO, Q03705, P15646, P40825, Q9C1X4, Q9Y7T8, Q9HFP8, Q01475, Q9UTN8, O94444, and Q9Y818. In each of the above Uniprot records, the sequence of the protein antigen relevant for the present disclosure is version 1, as designated in the Uniprot record by the record number followed by “−1”. This sequence designation is unique and unchaging. For example, in the Uniprot record Q9Y818 the the sequence of the protein antigen relevant for the present disclosure is designated in the Uniprot record as “Q9Y818-1”.

In some embodiments the pathogen-associated disorder is a protozoan disorder. For example, in some embodiments the pathogen-associated disorder is selected from the group consisting of Acanthamoeba keratitis, African trypanosomiasis, Avian malaria, Babesiosis, Besnoitiosis, Blastocystosis, Chagas disease, Cryptosporidiosis, Cyclosporiasis, Dientamoebiasis, Giardiasis, Histomoniasis, Malaria, Premunity, Sappinia amoebic encephalitis, Surra, Toxoplasmosis, Trichomoniasis, and Trypanosomiasis.

In some embodiments the PAA is derived from a protozoa. For example, in some embodiments the PAA is selected from the group consisting of Acanthamoeba, Trypanosoma sp. including T. brucei, T. cruzi, and T. evansi, Plasmodium sp. including P. falciparum, P. vivax, P. ovale, P. malariae, P. knowlesi, P. relictum, P. anasum, and P. gallinaceum, Hemoproteus sp., Babesia sp., Besnoitia sp., Blastocystis sp., Cryptosporidium sp. including C. parvum and C. hominis, Cyclospora cayetanensis, Dientamoeba fragilis, Giardia so. including G. lamblia and G. intestinalis, Histomonas meleagridis, Sappinia sp. including S. diploidea and S. pedata, Toxoplasma gondii, and Trichomonas vaginalis.

In some embodiments the PAA is a protozoan antigen. For example, in some embodiments the PAA is selected from the group consisting of the antigens having the following unique and stable UniProt entry identifier (see https://www.uniprot.org/help/entry name): A0A074T1L1, A0A0S4KF97, A0A0S4JQZ4, A0A0S4JAS1, A0A0S4J2H8, A0A0S4J8R3, A0A0S4IP92, A0A0S4IVI6, A0A074SMM4, A0A0S4JU95, A0A0S4JUU2, A0A0S4J2G0, A0A0S4JKP3, A0A0S4JNU2, A0A0S4J4S8, A0A0S4J1D6, A0A0S4JJ69, A0A0S4JQW3, A0A0S4KGR9, A0A0S4JOSO, A0A0S4IJQ6, A0A0S4J021, A0A0S4JLK8, A0A0S4JET1, A0A0S4IM54, A0A0S4JTM6, A0A0S4JEP5, A0A0S4IXR8, A0A0S4J1A6, A0A0S4IT85, A0A0S4JB95, A0A0S4IP11, A0A0S4JG58, A0A0S4J014, A0A0S4JAG3, A0A0S4JBV9, A0A0S4IMDO, A0A0S4KGC5, A0A0S4IKB9, A0A0S4JMF9, A0A0S4JJX3, A0A0S4IVI8, A0A074TOF7, A0A0S4KMS7, A0A0S4IQ67, A0A0S4JAF7, A0A0S4J3C2, A0A0S4J897, A0A0S4J998, A0A0S4JAD5, A0A074SUM1, A0A0S4ITE7, A0A0S4IJN2, A0A0S4KHF9, A0A0S4J6W7, A0A0S4J872, A0A0S4JS05, A0A0S4JQZO, A0A074TVK6, B6DTN7, A0A0S4KLY4, A0A0S4JS11, A0A0S4J353, A0A0S4IXE4, A0A0S4J7G3, A0A0S4J4W4, A0A074TOB9, A0A0S4IXG7, A0A0S4IVU4, A0A0S4JB29, A0A0S4IIQ2, A0A0S4JB24, A0A0S4J5S1, A0A0S4IZD9, A0A074TOJ3, A0A0S4JL29, A0A0S4IR72, A0A0S4JB58, A0A0S4JB16, A0A0S4IL58, A0A074STGO, A0A0S4JJQ5, A0A074T2W9, A0A0S4JBM4, A0A0S4IXS4, A0A0S4JXN2, A0A0S4ILC9, A0A0S4IW93, A0A0S4JIN3, A0A0S4KK21, A0A074TOJ7, A0A0S4JD35, A0A0S4IS00, A0A0S4JBS2, A0A0S4J5K3, A0A0S4J654, A0A0S4IJSO, A0A0S4J299, A0A074TCC9, A0A0S4J4B4, A0A0S4JON5, A0A0S4IRK3, A0A0S4JNX5, A0A0S4J9S4, A0A0S4JUB6, A0A0S4JDR2, A0A074T113, A0A0S4J954, A0A0S4KLL4, A0A0S4KEG2, A0A0S4J8T9, A0A0S4JJVO, A0A0S4JF82, A0A0S4J7H1, A0A074TWU5, A0A0S4JF41, A0A0S4JFAO, A0A0S4JS89, A0A0S4JE28, A0A0S4KFY6, A0A0S4J5C1, A0A0S4J603, A0A074T241, A0A0S4IL47, A0A0S4J985, A0A0S4IT79, A0A0S4JHS5, A0A0S4JCG7, A0A0S4KQ64, A0A0S4JQ27, A0A074SL18, A0A0S4J840, A0A0S4JIC5, A0A0S4KEN7, A0A0S4IPW7, A0A0S4IR61, A0A0S4IS67, A0A0S4IMD6, A0A074SV31, A0A0S4JEJ4, A0A0S4KIF1, A0A0S4JD68, A0A0S4JLCO, A0A0S4JTS4, A0A0S4JNW2, A0A0S4JRC8, A0A074SKW9, A0A0S4IZX6, A0A0S4JHEO, A0A0S4JT29, A0A0S4JSD2, A0A0S4J100, A0A0S4J3U1, A0A0S4IMY9, A0A0S4JMU5, A0A0S4J9N3, A0A0S4IVH1, A0A0S4JQW5, A0A0S4IS12, A0A0S4JAQ6, A0A0S4JC49, A0A0S4JDY8, A0A0S4JN05, A0A0S4ILK6, A0A0S4JHG5, A0A0S4KGV4, A0A0S4J515, A0A0S4IQA7, A0A0S4IV27, A0A0S4JAM9, A0A0S4JJG5, A0A0S4JQB6, A0A0S4JLH4, A0A0S4IVN7, A0A0S4JEZ5, A0A0S4IM08, A0A0S4IH88, A0A0S4JLB2, A0A0S4JEE7, A0A0S4IVX6, A0A0S4IU55, A0A0S4IN83, A0A0S4IZ03, A0A0S4IVV6, A0A0S4KEL3, A0A0S4ISG8, A0A0S4JV10, A0A0S4IIK7, A0A0S4JC54, A0A0S4IKB3, A0A0S4J7S2, A0A0S4JD74, A0A0S4JQ06, A0A0S4J746, A0A0S4JA77, A0A0S4JF29, A0A0S4JAE3, A0A0S4JFY5, A0A0S4JMQ7, A0A0S4J7Q5, A0A0S4J1U3, A0A0S4KK93, A0A0S4IHU9, A0A0S4J2L4, A0A0S4ITD5, A0A0S4JTQ7, A0A0S4JP32, A0A0S4IWS5, A0A0S4JB17, A0A0S4ILT2, A0A0S4JLL8, A0A0S4JDS1, A0A0S4JBL4, A0A0S4JU85, A0A0S4JRD7, A0A0S4J2K2, A0A0S4IU91, A0A0S4IXT3, A0A0S4KJT6, A0A0S4JFC9, A0A0S4JNK9, A0A0S4JV99, A0A0S4JVN7, A0A0S4J113, A0A0S4JAQ3, A0A0S4IXN5, A0A0S4ILX2, A0A0S4IQE4, A0A0S4KF94, A0A0S4JEZ2, A0A0S4IK14, A0A0S4KEC2, A0A0S4JE78, A0A0S4IND6, A0A0S4J3T7, A0A0S4JTK3, A0A0S4JJFO, A0A0S4IMBO, A0A0S4JL12, A0A0S4IQP3, A0A0S4JV52, A0A0S4ITN5, A0A0S4J2M0, A0A0S4IX99, A0A0S4J6B1, A0A0S4JOU7, A0A0S4IMZO, A0A0S4J7H0, A0A0S4JF05, A0A0S4JGN7, A0A0S4KK37, A0A0S4JDTO, A0A0S4JS26, A0A0S4JD14, A0A0S4ITR1, A0A0S4JOH7, A0A0S4J260, A0A0S4JWD6, A0A0S4J8Q8, A0A0S4JE04, A0A0S4JNM8, A0A0S4J7L5, A0A0S4JP43, A0A0S4J3A2, A0A0S4IXY3, A0A0S4J419, A0A0S4JOZO, A0A0S4IY44, A0A0S4JRN5, A0A0S4KHD7, A0A0S4JOFO, A0A0S4J4L7, A0A0S4J539, A0A0S4J3Q1, A0A0S4J542, A0A0S4IL46, A0A0S4KL26, A0A0S4JEG7, A0A0S4JVS2, A0A0S4KP55, A0A0S4JOG9, A0A0S4JJ63, A0A0S4IP14, A0A0S4J8P6, A0A0S4J2R4, A0A0S4J761, A0A0S4IJN8, A0A0S4JSG5, A0A0S4IP99, A0A0S4JHM1, A0A0S4IN27, A0A0S4KEC0, A0A0S4JSC5, A0A0S4J4G2, A0A0S4JAN4, A0A0S4JEP2, A0A0S4J9M5, A0A0S4ISU4, A0A0S4J604, A0A0S4ILR7, A0A0S4JGL5, A0A0S4JOKO, A0A0S4JSB8, A0A0S4JLZO, A0A0S4JOQ7, A0A0S4IVU5, A0A0S4J1U5, A0A0S4IQP4, A0A0S4IT62, A0A0S4JUR5, A0A0S4IU73, A0A0S4J6BO, A0A0S4JCX5, A0A0S4JCT9, A0A0S4J2U8, A0A0S4KJA7, A0A0S4J8U4, A0A0S4JS12, A0A0S4JWB3, A0A0S4ISN8, A0A0S4JDX7, A0A0S4J592, A0A0S4IU40, A0A0S4IJ71, A0A0S4J3B3, A0A0S4JCC6, A0A0S4JAU0, A0A0S4J360, A0A0S4JG36, A0A0S4JNL2, A0A0S4IWB9, A0A0S4J805, A0A0S4JMGO, A0A0S4JG17, A0A0S4J206, A0A0S4J4C3, A0A0S4ILW5, A0A0S4KIB1, A0A0S4JZB5, A0A0S4IR85, A0A0S4IVQ8, A0A0S4JSH3, A0A0S4KIR5, A0A0S4JK76, A0A0S4IZC7, A0A0S4JSA1, A0A0S4JJG7, A0A0S4IMS3, A0A0S4IY39, A0A0S4IWU0, A0A0S4J4P0, A0A0S4JEG9, A0A0S4IW61, A0A0S4ITN3, A0A0S4J2P7, A0A0S4IS4I, A0A0S4KMV2, A0A0S4J278, A0A0S4JVZ3, A0A0S4JU20, A0A0S4JPM5, A0A0S4IRQ2, A0A0S4JTY6, A0A0S4J814, A0A0S4J2V2, A0A0S4IVC7, A0A0S4IZW2, A0A0S4JH12, A0A0S4JU35, A0A0S4J6M1, A0A0S4JMU7, A0A0S4JLM8, A0A0S4JMV1, A0A0S4JEK1, A0A0S4J5X0, A0A0S4JL76, A0A0S4JJI3, A0A0S4JPN6, A0A0S4J254, A0A0S4IQ10, A0A0S4KK75, A0A0S4JEXO, A0A0S4JEH8, A0A0S4IV96, A0A0S4JFV3, A0A0S4IZX5, A0A0S4KIT5, A0A0S4JAW7, A0A0S4IIS0, A0A0S4J5A0, A0A0S4KHE4, and A0A0S4IUK8. In each of the above Uniprot records, the sequence of the protein antigen relevant for the present disclosure is version 1, as designated in the Uniprot record by the record number followed by “−1”. This sequence designation is unique and unchaging. For example, in the Uniprot record A0A0S4IUK8 the the sequence of the protein antigen relevant for the present disclosure is designated in the Uniprot record as “A0A0S4IUK8-1”.

In some embodiments the pathogen-associated disorder is a parasitical disorder. For example, in some embodiments the pathogen-associated disorder is selected from the group consisting of Echinococcosis, Amebiasis, Ancylostomiasis, Angiostrongyliasis, Anisakiasis, Ascariasis, Balantidiasis, Granulomatous Amebic Encephalitis, Baylisascariasis, Schistosomiasis, Capillariasis, Clonorchiasis, Cysticercosis, Diphyllobothriasis, Filariasis, Enterobiasis, Fascioliasis, Fasciolopsiasis, gnathostomiasis, Heterophyiasis, Hymenolepiasis, Leishmaniasis, Opisthorchiasis, Loiasis, Onchocerciasis, Paragonimiasis, Sarcocystosis, Taeniasis, Toxocariasis, Trichinellosis, and Trichuriasis.

In some embodiments the PAA is derived from a parasite. For example, in some embodiments the PAA is selected from the group consisting of Echinococcus sp. including E. granulosus and E. multilocularis, Entamoeba histolytica, Ancylostoma sp. including A. brazilense, A. caninum, and A. ceylanicum, Uncinaria stenocephala, Angiostrongylus sp. including Angiostrongylus cantonensis, Ascaris sp. including A. lumbricoides and A. suum, Balantidium coli, Balamuthia mandrillaris, Baylisascaris procyonis, Schistosoma sp. including S. mansoni, S. haematobium, and S. japonicum, Capillaria hepatica, Capillaria philippinensis, Clonorchis sp., Taenia sp. including T. solium, T. saginata, and T. asiatica, Diphyllobothrium sp. including Diphyllobothrium latum, Dipylidium sp., Dracunculus medinensis, Enterobius vermicularis, Fasciola sp. including Fasciola hepatica, Fasciolopsis buski, Gnathostoma sp., Heterophyes heterophyes, Naegleria fowleri, Onchocerca volvulus, Paragonimus sp., Sarcocystis sp., Strongyloides sp., Toxocara canis, Toxocara cati, and Trichinella sp..

In some embodiments the PAA is a parasitical antigen. For example, in some embodiments the PAA is selected from the group consisting of the antigens having the following unique and stable UniProt entry identifier (see https://www.uniprot.org/help/entry name): B6KAMO, P90661, Q81E47, P14593, P18269, Q4U9M9, P23253, P13826, P50498, P04934, P13828, P13814, P50490, P19597, P02897, P02890, P09792, Q02752, P18270, P06914, P15714, P08569, P68874, P06016, P08515, Q8MZJ8, P15964, P08676, P22622, P08677, Q25540, P02892, P87020, P42665, P18271, P13815, P20287, 096175, P35661, P08418, P29030, P62884, P67877, Q07828, P50491, C6KTB7, P04926, P02894, Q9TY95, Q5F2J0, Q03400, P08675, Q95WA4, Q9F9F2, P35666, Q9AJ37, Q81E95, P09841, Q95WA3, P04922, P50492, P08672, Q05870, P05691, Q25619, P26332, Q03994, P08673, P19260, P06015, P31008, P17503, P16445, P02893, P67878, P15744, Q9XZV1, P08307, P21849, P32072, P69192, P22545, Q815D2, P08674, P14223, Q4UNEO, P61074, Q4FX73, P22621, P26694, P54190, Q25306, P13399, Q01443, P02898, Q4UM04, P81860, Q81KW2, P26624, P06915, P50489, Q4UN15, Q7KQM2, P02891, B6KV60, Q03110, P21303, P62883, B3STN5, and P23093. In each of the above Uniprot records, the sequence of the protein antigen relevant for the present disclosure is version 1, as designated in the Uniprot record by the record number followed by “−1”. This sequence designation is unique and unchaging. For example, in the Uniprot record P23093 the the sequence of the protein antigen relevant for the present disclosure is designated in the Uniprot record as “P23093-1”.

In some embodiments the pathogen-associated disorder is a prion disorder. For example, in some embodiments the pathogen-associated disorder is selected from the group consisting of Creutzfeldt-Jakob disease (CJD) including iatrogenic, variant, familial, and sporadic sub-types, fatal familial insomnia (FFI), Gertsmann-Straussler-Scheinker syndrome (GSS), kuru, and variably protease-sensitive prionopathy (VPSPr).

In some embodiments the PAA is derived from a prion protein. For example, in some embodiments the PAA is selected from the group consisting of PrP^{C}, PrP^{res}, and PrP^{Sc}. In some embodiments, PrP^{C} as referred to herein corresponds to Unipot entry ID P04156; the protein sequence is identified by the Uniprot accession number P04156-1.

In some embodiments the pathogen-associated disorder is a protein aggregate disorder. For example, in some embodiments the pathogen-associated disorder is selected from the group consisting of Alzheimer's disease, Cerebral p-amyloid angiopathy, Retinal ganglion cell degeneration in glaucoma, Parkinson's disease and other synucleinopathies, Tauopathies, Frontotemporal lobar degeneration, FTLD-FUS, Amyotrophic lateral sclerosis (ALS), Huntington's disease, Familial British dementia, Familial Danish dementia, Hereditary cerebral hemorrhage with amyloidosis, CADASIL, Alexander disease, Seipinopathies, Familial amyloidotic neuropathy, Senile systemic amyloidosis, Serpinopathies, AL amyloidosis, AH amyloidosis, AA amyloidosis, Type II diabetes, Aortic medial amyloidosis, ApoAl amyloidosis, ApoAll amyloidosis, ApoAIV amyloidosis, Familial amyloidosis of the Finnish type (FAF), Lysozyme amyloidosis, Fibrinogen amyloidosis, Dialysis amyloidosis, Inclusion body myositis/myopathy, Cat Retinitis pigmentosa with rhodopsin mutations, Medullary thyroid carcinoma, Cardiac atrial amyloidosis, Pituitary prolactinoma, Hereditary lattice corneal dystrophy, Cutaneous lichen amyloidosis, Mallory bodies, Multiple System Atrophy, Corneal lactoferrin amyloidosis, Pulmonary alveolar proteinosis, Odontogenic (Pindborg) tumor amyloid, Seminal vesicle amyloid, Apolipoprotein C2 amyloidosis, Apolipoprotein C3 amyloidosis, Lect2 amyloidosis, Insulin amyloidosis, Galectin-7 amyloidosis (primary localized cutaneous amyloidosis), Corneodesmosin amyloidosis, Enfuvirtide amyloidosis, Cystic fibrosis, and Sickle cell disease.

In some embodiments the PAA is derived from an aggregate protein. For example, in some embodiments the PAA is selected from the group consisting of Amyloid β peptide (Ap; uniprot ref. P05067), Tau (P10636), a-Synuclein (P37840), Fused in sarcoma (FUS) protein (P35637), Superoxide dismutase (P00441), TDP-43 (Q13148), C90RF72 (Q96LT7), ubiquilin-2 (UBQLN2) (Q9UHD9), Huntingtin (P42858), ABri (Q9Y287), Cystatin C (P01034), Notch3 (Q9UM47), Glial fibrillary acidic protein (GFAP; P14136), Seipin (Q96G97), Transthyretin (P02766), Serpins (P01009 & others), immunoglobulin light chains (P01834 & others), Immunoglobulin heavy chains (P01857 & others), Amyloid A protein (PODJI8), Islet amyloid polypeptide (amylin; P10997), Medin (lactadherin; Q08431), Apolipoprotein A1 (P02647), Apolipoprotein AII (P02652), Apolipoprotein AIV (P06727), Gelsolin (P06396), Lysozyme (P61626), Fibrinogen (P02671, P02675), Beta-2 microglobulin (P61769), Crystallins (P02489 & others), Rhodopsin (P08100), Calcitonin (P01258), Atrial natriuretic factor (P01160), Prolactin (P01236), Keratoepithelin (Q15582), Keratins (Q14533 & others), Keratin intermediate filament proteins (P35908 & others), Lactoferrin (P02788), Surfactant protein C (SP-C) (P11686), Odontogenic ameloblast-associated protein (A1E959), Semenogelin I (P04279), Apolipoprotein C2 (ApoC2) (P02655), Apolipoprotein C3 (ApoC3) (P02656), Leukocyte chemotactic factor-2 (Lect2) (014960), Insulin (P01308), Galectin-7 (Gal7) (P47929), Corneodesmosin (Q15517), CFTR protein (P13569), and Hemoglobin (P69905 & P68871). In each of the above Uniprot records, the sequence of the protein antigen relevant for the present disclosure is version 1, as designated in the Uniprot record by the record number followed by “−1”. This sequence designation is unique and unchaging. For example, in the Uniprot record P68871 the the sequence of the protein antigen relevant for the present disclosure is designated in the Uniprot record as “P68871-1”.

Subject Selection

In certain aspects, the subjects are selected as suitable for treatment with the treatments before the treatments are administered. In some aspects the treatment methods described herein include the step of selecting suitable subjects. In some aspects the treatment methods described herein treat subjects that have been previously selected as suitable for treatment.

As used herein, subjects who are considered suitable for treatment are those subjects who are expected to benefit from, or respond to, the treatment. Subjects may have, be suspected of having, have been diagnosed with, or be at risk of, a disorder characterized by a disorder-associated antigen (DAA) as described herein.

In some aspects the treated subject has been selected for treatment on the basis that the subject has, is suspected of having, has been diagnosed with, or is at risk of, a disorder characterized by a disorder-associated antigen (DAA).

In some aspects the subject is: (1) selected for treatment on the basis that the subject has, is suspected of having, has been diagnosed with, or is at risk of, a disorder characterized by a disorder-associated antigen (DAA); then (2) treated with a CD25-ADC as described herein.

In particular, the disorder characterized by a disorder-associated antigen (DAA) may be solid tumour as described herein.

In some aspects, subjects are selected on the basis of the amount or pattern of expression of CD25. In some aspects, the selection is based on expression of CD25 at the cell surface in a tissue or structure of interest. So, in some cases, subjects are selected on the basis they have, or are suspected of having, are at risk of having, or have received a diagnosis of a proliferative disease characterized by the presence of a neoplasm comprising or associated with cells having surface expression of CD25. The neoplasm may be composed of cells having surface expression of CD25.

In some aspects, subjects are selected on the basis they have a neoplasm comprising both CD25+ve and CD25-ve cells. The neoplasm may be composed of CD25-ve neoplastic cells, optionally wherein the CD25-ve neoplastic cells are associated with CD25+ve non-neoplastic cells such as CD25+ve Tregs. The neoplasm or neoplastic cells may be all or part of a solid tumour. The solid tumour may be partially or wholly CD25-ve, and may be infiltrated with CD25+ve cells, such as CD25+ve Tregs. In preferred aspects, the solid tumour is associated with high-levels of CD25+ve infiltrating cells, such as Treg cells. In some aspects, the solid tumour is associated with low-levels of CD25+ve infiltrating cells, such as Treg cells. In some aspects, the solid tumour is not associated with CD25+ve infiltrating cells, such as Treg cells; for example, the levels of CD25+ve cells may be below the detection limit.

In some cases, expression of CD25 in a particular tissue of interest is determined. For example, in a sample of tumor tissue. In some cases, systemic expression of CD25 is determined. For example, in a sample of circulating fluid such as blood, plasma, serum or lymph.

In some aspects, the subject is selected as suitable for treatment due to the presence of CD25 expression in a sample. In those cases, subjects without CD25 expression may be considered not suitable for treatment.

In other aspects, the level of CD25 expression is used to select a subject as suitable for treatment. Where the level of expression of the target is above a threshold level, the subject is determined to be suitable for treatment.

In some aspects, an subject is indicated as suitable for treatment if cells obtained from the tumour react with antibodies against CD25 as determined by IHC.

In some aspects, a subject is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express CD25. In some aspects disclosed herein, a subject is determined to be suitable for treatment if at least at least 5% of the cells in the sample express CD25.

Treatment of Established Tumours & Reduction of Metastatic Tumours

The methods described and exemplified herein have been shown to be effective at treating established tumous in naïve subjects as well as the reduction or prevention of metastatic tumours in previously treated subjects.

Accordingly, in some aspects a subject is selected for treatment if they have, are suspected of having, have been diagnosed with, or are at risk of, an established tumour, such as an established solid tumour. An ‘establised tumour’ as described herein may be, for example, a tumour diagnosed or identified in a naïve subject.

In some cases a naïve subject is a subject that has not yet been treated to reduce the immune-suppressive activity of an immune regulatory cell population, as defined herein; for example; treated with an anti-CD25 antibody or a CD25-ADC. In some cases a naïve subject is a subject that has not yet been treated with ADCx25, as defined herein.

In some cases, an ‘establised tumour’ as described herein may be a relapsed or resistant tumour. For example, a relapsed tumour may a new or growing tumour identified or diagnosed in a subject following a period of remission (partial or complete). The tumour may be metastatic or in the same site as the primary tumour.

In some aspects a subject is selected for treatment if they have, are suspected of having, have been diagnosed with, or are at risk of, a circulating tumour or circulating tumour cells (CTC; Gupta et al. 2006, Cell. 127 (4): 679-95; Rack et al., 2014. Journal of the National Cancer Institute. 106 (5)). The CTCs may be, or comprise, metastatic cells (i.e. CTCs capable of establishing metastatic tumours in a subject).

Subjects suspected of having, have been diagnosed with, or are at risk of, a circulating tumour or circulating tumour cells may include;
(1) subjects who have, are suspected of having, have been diagnosed with a primary tumour with metastatic characteristics, such as high-metastatic prognosis or elevated expression of one or more biomarkers of metastatic cancer (Dawood, S., Expert Rev Mol Diagn. 2010 July; 10(5):581-90);
(2) subjects who have, are suspected of having, have been diagnosed with one or more metastatic tumours;
(3) pre-operative or post-operative subjects, wherein the operation is to remove part or all of a solid tumour. Typically, selected pre-operative or post-operative subjects start their treatment not more than 4 weeks from the operation date, such as not more than 2 weeks, or not more than 1 week.

In some cases, a ‘metastatic tumour’ as described herein may be a tumour not located in the same site as the primary tumour.

Adoptive Cell Transfer

As described herein, the present authors observed that in 2 subjects with AML who had previously received bone marrow stem cell transfers, administration of ADCx25 led to complete remission of the AML.

In some aspects the subject is selected for treatment on the basis they have received an adoptive cell transfer.

In some embodiments the subject is only selected if they have, are suspected of having, have been diagnosed with, or are at risk of, a disorder characterized by a disorder-associated antigen (DAA); in some embodiments the disorder is AML.

The cells transferred during the adoptive cell transfer may be autologous or allogenic cells. The transferred cells may be stem cells, such as stem cells derived from bone marrow. The transferred cells may be immune cells

The adoptive cell transfer may be a bone marrow transplant.

In some cases the subject is only selected for treatment with a CD25-ADC if they received the adoptive cell transfer at least 3 months, at least 6 months, at least 9 months, at least 12 months, or at least 18 months before the administration of the CD25-ADC. In some cases, the subject is only selected for treatment with a CD25-ADC if they received the adoptive cell transfer at least 24 months before the administration of the CD25-ADC

In some cases the ADC25-ADC is administered to selected subjects in a QW dosage regime. In some cases the dose of CD25-ADC administered is 10, 20, 25, 27.5, 30, 32.5, 35, 37.5, 40, 42.5, 45, 47.5, 50, 55, 60, 65, 70, 75, or 80 μg/kg. For example 30 or 37.5 μg/kg of CD25 ADC may be administered in a QW dosage regime.

Samples

The sample may comprise or may be derived from: a quantity of blood; a quantity of serum derived from the subject's blood which may comprise the fluid portion of the blood obtained after removal of the fibrin clot and blood cells; a quantity of pancreatic juice; a tissue sample or biopsy, in particular from a solid tumour; or cells isolated from said subject.

A sample may be taken from any tissue or bodily fluid. In certain aspects, the sample may include or may be derived from a tissue sample, biopsy, resection or isolated cells from said subject.

In certain aspects, the sample is a tissue sample. The sample may be a sample of tumor tissue, such as cancerous tumor tissue. The sample may have been obtained by a tumor biopsy. In some aspects, the sample is a lymphoid tissue sample, such as a lymphoid lesion sample or lymph node biopsy. In some cases, the sample is a skin biopsy.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a subject's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of CD25 polypeptide or nucleic acid in the blood, in white blood cells, peripheral blood mononuclear cells, granulocytes and/or red blood cells.

The sample may be fresh or archival. For example, archival tissue may be from the first diagnosis of a subject, or a biopsy at a relapse. In certain aspects, the sample is a fresh biopsy.

Subject Status

The subject may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject may be any of its forms of development, for example, a foetus. In one preferred embodiment, the subject is a human. The terms “subject”, “patient” and “individual” are used interchangeably herein.

In some aspects the subject has, is suspected of having, has been diagnosed with, or is at risk of, a disorder characterized by a disorder-associated antigen (DAA) as described herein.

In particular, the disorder characterized by a disorder-associated antigen (DAA) may be solid tumour as described herein.

Adoptive Cell Transfer

In some aspects the subject has received an adoptive cell transfer as defined herein.

Tumour Status

In some aspects the subject has an established tumour as defined herein. In some aspects the subject is suspected of having, has been diagnosed with, or is at risk of, a circulating tumour or circulating tumour cells. In some aspects the subject has a metastatic tumour as defined herein.

Controls

In some aspects, target expression in the individual is compared to target expression in a control. Controls are useful to support the validity of staining, and to identify experimental artefacts.

In some cases, the control may be a reference sample or reference dataset. The reference may be a sample that has been previously obtained from a individual with a known degree of suitability. The reference may be a dataset obtained from analyzing a reference sample.

Controls may be positive controls in which the target molecule is known to be present, or expressed at high level, or negative controls in which the target molecule is known to be absent or expressed at low level.

Controls may be samples of tissue that are from individuals who are known to benefit from the treatment. The tissue may be of the same type as the sample being tested. For example, a sample of tumor tissue from a individual may be compared to a control sample of tumor tissue from a individual who is known to be suitable for the treatment, such as a individual who has previously responded to the treatment.

In some cases the control may be a sample obtained from the same individual as the test sample, but from a tissue known to be healthy. Thus, a sample of cancerous tissue from a individual may be compared to a non-cancerous tissue sample.

In some cases, the control is a cell culture sample.

In some cases, a test sample is analyzed prior to incubation with an antibody to determine the level of background staining inherent to that sample.

In some cases an isotype control is used. Isotype controls use an antibody of the same class as the target specific antibody, but are not immunoreactive with the sample. Such controls are useful for distinguishing non-specific interactions of the target specific antibody.

The methods may include hematopathologist interpretation of morphology and immunohistochemistry, to ensure accurate interpretation of test results. The method may involve confirmation that the pattern of expression correlates with the expected pattern. For example, where the amount of CD25 expression is analyzed, the method may involve confirmation that in the test sample the expression is observed as membrane staining, with a cytoplasmic component. The method may involve confirmation that the ratio of target signal to noise is above a threshold level, thereby allowing clear discrimination between specific and non-specific background signals.

Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Disclosed herein are methods of therapy. Also provided is a method of treatment, comprising administering to a subject in need of treatment a therapeutically-effective amount of an ADC. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The ADC may comprise an anti-CD25 antibody. The anti-CD25 antibody may be HuMax-TAC™. The ADC may comprise a drug which is a PBD dimer. The ADC may be a anti-CD25-ADC, and in particular, ADCX25/ADCT-301/Camidanlumab Tesirine. The ADC may be an ADC disclosed in WO2014/057119.

The treatment may involve administration of the ADC alone or in further combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: Lenalidomide (REVLIMID®, Celgene), Vorinostat (ZOLINZA®, Merck), Panobinostat (FARYDAK®, Novartis), Mocetinostat (MGCD0103), Everolimus (ZORTRESS®, CERTICAN®, Novartis), Bendamustine (TREAKISYM®, RIBOMUSTIN®, LEVACT®, TREANDA®, Mundipharma International), erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (P13K inhibitor, Semafore Pharmaceuticals), BEZ-235 (P13K inhibitor, Novartis), XL-147 (P13K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gammall, calicheamicin omegall (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above. Combinations of agents may be used, such as CHP (doxorubicin, prednisone, cyclophosphamide), or CHOP (doxorubicin, prednisone, cyclophopsphamide, vincristine).

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rlL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETA™, OMNITARG™, 2C4, Genentech), trastuzumab (HERCEPTIN®, Genentech), tositumomab (Bexxar, Corixia), MDX-060 (Medarex) and the antibody drug conjugate, gemtuzumab ozogamicin (MYLOTARG®, Wyeth).

Humanized monoclonal antibodies with therapeutic potential as chemotherapeutic agents in combination with the conjugates of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Compositions according to the present disclosure, including vaccine compositions, are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art.

Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Dosage Regimes

Dosing Summary

During assessment of CD25-ADC efficacy, present authors made the surprising observation that the tested CD25-ADC had significant in vivo anti-tumour activity against solid tumours, including tumours composed of CD25-ve neoplastic cells. The present authors made the further unexpected observation of synergistic anti-tumour activity between the tested CD25-ADC and PD-1 antagonists.

The present authors have therefore developed a treatment regime for the administration of a CD25-ADC, both as a single agent and in combination with PD-1 antagonists. The developed treatment regimes allow for increased clinical benefit and reduced incidence of treatment related adverse events. In particular, the present authors have identified a series of dose levels and intervals that allow for improved clinical utility, including in patient groups previously not suspected of being succeptible to treatment with CD25-ADCs.

Accordingly, the subject-matter of the present disclosure concerns the use of CD25-ADCs for treating disease, for example, proliferative diseases such as solid tumours (including CD25-ve solid tumours). These dosage regimes described herein are expected to be associated with a range of clinical benefits, including improved efficacy, reduced toxicity and side-effects, and the expansion of the population eligible to be treated to include subjects intolerant of the greater side effects of known dosage regimes and those previously not known to be susceptible to treatment with CD25-ADC.

In a first dosing aspect the disclosure provides a method of treating a proliferative disease in a subject, said method comprising administering to a subject an effective amount of a CD25-ADC in a Q3W dosage regime.

Q3W is used herein with its normal meaning of every three weeks. Accordingly, in a Q3W dosage regime a single dose of CD25-ADC is administered on day 1 of a 21-day treatment cycle.

The dose of CD25-ADC may be up to 300 ug/kg, such as 20 to 300 ug/kg. The dose may be about 20 ug/kg, about 30 ug/kg, about 45 ug/kg, about 60 ug/kg, about 80 ug/kg, about 125 ug/kg, about 150 ug/kg, about 200 ug/kg, about 250 ug/kg, or about 300 ug/kg.

The CD25-ADC is preferably ADCx25/ADCT-301/Camidanlumab Tesirine as described herein.

In a second dosing aspect, the method of the first aspect further comprises administering to the subject an effective amount of a PD-1 antagonist in combination with the CD25-ADC.

The PD1 antagonist may be pembrolizumab, nivolumab, MED10680, PDR001 (spartalizumab), Camrelizumab, AUNP12, Pidilizumab Cemiplimab (REGN-2810), AMP-224, BGB-A317 (Tisleizumab), or BGB-108. Preferably the PD1 antagonist is pembrolizumab.

The PD1 antagonist may be administered to the subject before, concurrently with, or after the CD25-ADC. Preferably, the CD25-ADC and PD1 antagonist are administered concurrently; that is the CD25-ADC and the PD1 antagonist are administered as part of the same treatment cycle.

The PD1 antagonist may be administered in a Q3W dosage regime. The dose of the PD1 antagonist may be 200 mg. Typically, the dose of the PD1 antagonist is 200 mg per treatment cycle for an adult subject. In some cases a reduced dose of PD1 antagonist may be administered; for example, a dose of 2 mg/kg (up to 200 mg) per treatment cycle. The reduced dose may be administered to specific patient groups, for example children.

When administered in combination with PD1 antagonist, the CD25-ADC is preferably administered in a dosage regime consisting of two Q3W treatment cycles. Preferably, the dose of CD25-ADC administered in each of the two treatment cycles is the same. Alternatively, the second dose may be a reduced dose.

In cases where the subject achieves CR following initial treatment with the CD25-ADC and PD1 antagonist combination, typically no further CD25-ADC is administered to the subject.

In these cases, PD1 antagonist administration may continue for up to 1 year after the completion of CD25-ADC treatment.

In cases where the subject achieves SD or PR following initial treatment with the CD25-ADC and PD1 antagonist combination, further CD25-ADC may be administered to the subject. In these cases, PD1 antagonist administration may continue after the initial treatment with the CD25-ADC and PD1 antagonist combination. If the subject has not achieved CR within 3 months after the completion of initial CD25-ADC treatment, further CD25-ADC may be administered to the subject.

The further CD25-ADC may be administered in a dosage regime consisting of two Q3W treatment cycles. Preferably the dose administered in each of the two treatment cycles is the same. Alternatively, the second dose may be a reduced dose. Typically the further CD25-ADC is administered in combination with PD1 antagonist treatment.

The subject may be human.

The subject may have, be suspected of having, or have been diagnosed with a proliferative disease.

The proliferative disease may be (classical) Hodgkin lymphoma, with mixed cellularity type (Hodgkin-/Reed-Sternbert-Cells: CD25+/−).

Classical Hodgkins lymphoma includes the subtypes nodular sclerosing, lymphocyte predominant, lymphocyte depleted and mixed cellularity. The Hodgkins lymphoma subtype may not be defined. In certain aspects, the disorder is Hodgkins lymphoma of the nodular sclerosing or mixed cellularity subtype.

The proliferative disease may be a solid tumour, or characterized by the presence of a neoplasm or neoplastic cells that are all or part of a solid tumour. The neoplasm or neoplastic cells may be CD25-ve.

“Solid tumor” herein will be understood to include solid haematological cancers such as lymphomas (Hodgkin's lymphoma or non-Hodgkin's lymphoma) which are discussed in more detail herein.

Solid tumors may be neoplasms, including non-haematological cancers, comprising or composed of CD25+ve neoplastic cells. Solid tumors may be neoplasms, including non-haematological cancers, infiltrated with CD25+ve cells, such as CD25+ve T-cells; such solid tumours may lack expression of CD25 (that is, comprise or be composed of CD25-ve neoplastic cells).

For example, the solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The proliferative disease may be relapsed or refractory.

In a third dosing aspect, the present disclosure provides a method of reducing the toxicity and/or side effects associated with administration of a CD25-ADC or CD25-ADC/PD-1 antagonist combination to a subject, the method comprising administering the CD25-ADC or CD25-ADC/PD-1 antagonist combination in a dosage regime as defined herein.

In a fourth dosing aspect, the present disclosure provides a method of increasing the treatment efficacy associated with administration of a CD25-ADC or CD25-ADC/PD-1 antagonist combination to a subject, the method comprising administering the CD25-ADC or CD25-ADC/PD-1 antagonist combination in a dosage regime as defined herein.

In a fifth dosing aspect, the present disclosure provides a method of selecting a subject for treatment by dosage regime as described herein, which selection method comprises selecting for treatment subjects that have either:
(1) Hodgkins lymphoma;
(2) a solid tumour comprising or consisting of CD25+ve cells; or
(3) a solid tumour comprising or composed of CD25−ve neoplastic cells which has high-levels of CD25+ve infiltrating cells, such as CD25+ve T-cells.

In a sixth dosing aspect the present disclosure provides a packaged pharmaceutical product comprising a CD25-ADC as described herein in combination with a label or insert advising that the CD25-ADC should be administered in a dosage regime as described herein.

The disclosure also provides a kit comprising:
a first medicament comprising a CD25-ADC; and
a package insert or label comprising instructions for administration of the CD25-ADC in dosage regime as described herein;
optionally wherein the kit further comprises a PD-1 antagonist.

In a seventh dosing aspect the present disclosure provides a CD25-ADC as defined herein, optionally in combination with a PD-1 antagonist, for use in a method of treatment as described herein.

In an eighth dosing aspect the present disclosure provides the use of a CD25-ADC as defined herein, optionally in combination with a PD-1 antagonist, in the preparation of a medicament for use in a method of treatment as described herein.

Dosing Detailed Disclosure

As described in more detail below, the present authors have developed a treatment regime for the administration of a CD25-ADC, both as a single agent and in combination with PD-1 antagonists.

The developed treatment regimes allow for increased clinical benefit and reduced incidence of treatment related adverse events. In particular, the present authors have identified a series of dose levels and intervals that allow for improved clinical utility, including in patient groups previously not suspected of being succeptible to treatment with CD25-ADCs.

In a first aspect the disclosure provides a method of treating a proliferative disease in a subject, said method comprising administering to a subject an effective amount of a CD25-ADC in a Q3W dosage regime.

CD25-ADC Dosing

A dosage regime may consist of 2, 3, 4, 5, 6, 7, 8, 9, 10, 11, 12, 13, 14, 15, 16, 17, 18, 19, 20, 21, 22, 23, 24, 25, 26, 27, 28, 29 or 30 treatment cycles. In some cases the dosage regime is ended once the subject attains CR. In some cases the dosage regime is ended when the subject experiences a DLT. In some cases the dosage regime is considered as ended if a dose delay exceeding the length of the preceding treatment cycle is required.

In some cases the CD25-ADC dose is about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 μg/kg. In some cases the CD25-ADC dose is at least 30 μg/kg. In some cases the starting dose is at least 150 μg/kg, such as at least 300 μg/kg.

In some cases the CD25 dose is about 10, 20, 30, 40, 50, 60, 70, 80, 90, 100, 120, 150, 200, 250, 300, 350, 400, 450, 500, 550, or 600 μg/kg. In some cases the starting dose is 1 to 10 μg/kg, 11 to 20 μg/kg, 21 to 30 μg/kg, 31 to 40 μg/kg, 41 to 50 μg/kg, 51 to 60 μg/kg, 61 to 70 μg/kg, 71 to 80 μg/kg, 81 to 90 μg/kg, 91 to 100 μg/kg, 101 to 120 μg/kg, 121 to 140 μg/kg, 141 to 160 μg/kg, 161 to 180 μg/kg, 181 to 200 μg/kg, 201 to 220 μg/kg, 221 to 240 μg/kg, 241 to 260 μg/kg, 261 to 280 μg/kg, 281 to 300 μg/kg, 301 to 320 μg/kg, 321 to 340 μg/kg, 341 to 360 μg/kg, 361 to 380 μg/kg, 381 to 400 μg/kg, 401 to 420 μg/kg, 421 to 440 μg/kg, 441 to 460 μg/kg, 461 to 480 μg/kg, 481 to 500 μg/kg, 501 to 520 μg/kg, 521 to 540 μg/kg, 541 to 560 μg/kg, 561 to 580 μg/kg, or 581 to 600 μg/kg.

Preferably the dose of CD25-ADC is about 20 ug/kg, about 30 ug/kg, about 45 ug/kg, about 60 ug/kg, about 80 ug/kg, about 125 ug/kg, about 150 ug/kg, about 200 ug/kg, about 250 ug/kg, or about 300 ug/kg.

The CD25-ADC may be administered in a Q1W, Q2W, Q3W, Q4W, QSW, or Q6W dosage regime. Preferably the CD25-ADC is administered in a Q3W dosage regime.

Combinations with PD-1 Antagonists and PD-1 Anagonist Dosing

The CD25-ADC may be administered in combination with a PD-1 antagonist.

The PD1 antagonist may be pembrolizumab, nivolumab, MED10680, PDR001 (spartalizumab), Camrelizumab, AUNP12, Pidilizumab Cemiplimab (REGN-2810), AMP-224, BGB-A317 (Tisleizumab), or BGB-108. Preferably the PD1 antagonist is pembrolizumab.

The PD1 antagonist may be administered to the subject before, concurrently with, or after the CD25-ADC. Preferably, the CD25-ADC and PD1 antagonist are administered concurrently; that is the CD25-ADC and the PD1 antagonist are administered as part of the same treatment cycle.

In some cases the CD25-ADC and PD1 antagonist are administered on the same day of the treatment cycle, for example on day 1 of the treatment cycle. In some cases the CD25-ADC and PD1 antagonist are not administered on the same day of the treatment cycle; for example, in some cases the CD25-ADC is administered on day 1 of the treatment cycle and PD1 antagonist is administered on day 8 of the treatment cycle.

The PD1 antagonist may be administered in a Q1W, Q2W, Q3W, Q4W, Q5W, or Q6W dosage regime. Preferably the PD1 antagonist is administered in a Q3W dosage regime.

In some cases the PD1 antagonist dose is about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 mg per treatment cycle. Preferably the dose of the PD1 antagonist is 200 mg pertreatment cycle.

Typically, the dose of the PD1 antagonist is 200 mg per treatment cycle for an adult subject. In some cases a reduced dose of PD1 antagonist may be administered; for example, a dose of 2 mg/kg (up to 200 mg) per treatment cycle. The reduced dose may be administered to specific patient groups, for example children.

When administered in combination with PD1 antagonist, the CD25-ADC is preferably administered in a dosage regime consisting of two Q3W treatment cycles. Preferably, the dose of CD25-ADC administered in each of the two treatment cycles is the same. Alternatively, the second dose may be a reduced dose.

In cases where the subject achieves CR following initial treatment with the CD25-ADC and PD1 antagonist combination, typically no further CD25-ADC is administered to the subject. In these cases, PD1 antagonist administration may continue for up to 1 year after the completion of CD25-ADC treatment.

In cases where the subject achieves SD or PR following initial treatment with the CD25-ADC and PD1 antagonist combination, further CD25-ADC may be administered to the subject. In these cases, PD1 antagonist administration may continue after the initial treatment with the CD25-ADC and PD1 antagonist combination. If the subject has not achieved CR within 3 months after the completion of initial CD25-ADC treatment, further CD25-ADC may be administered to the subject.

The further CD25-ADC may be administered in a dosage regime consisting of two Q3W treatment cycles. Preferably the dose administered in each of the two treatment cycles is the same. Alternatively, the second dose may be a reduced dose. Typically the further CD25-ADC is administered in combination with PD1 antagonist treatment.

Disorders Treated with Disclosed Dosing Regimes

In one aspect, the present disclosure provides a method of therapy comprising administering an ADC which binds CD25 for use in therapy, wherein the method comprises selecting a subject based on expression of CD25.

In one aspect, the present disclosure provides a packaged ADC for use in therapy, wherein the packaged ADC is supplied with a label that specifies that the therapy is suitable for use with a subject determined to be suitable for such use. The label may specify that the therapy is suitable for use in a subject has expression of CD25, that is, is CD25+. The label may specify that the ADC is administered in a dosage regime as described herein. The label may specify that the subject has a particular type of cancer, such as Hodgkin Lymphoma (NHL), a CD25+ve solid tumour, or a solid tumour having high levels of CD25+ve infiltrating cells.

The proliferative disease treated by the methods disclosed herein may be CD25+ve. However as explained herein, in the practice of the disclosure, in at least some of the cells in the target location (typically a neoplasm) the antigen may be absent, or present on the cell surface at an insignificant level. For example in the target neoplasm only e.g. less than 80, 70, 60, 50, 30, 20%, 10% or 5% of the cells may be CD25 positive. Thus, the proliferative disease may be characterised by the presence of a neoplasm comprising both CD25+ve and CD25-ve cells.

A proliferative disease of particular interest is a solid tumour, including an advanced solid tumour.

In some cases, for example in some solid tumours, the target neoplasm may be completely or effectively CD25-ve. That is, in some cases the target neoplasm contains no (i.e. 0%) CD25+ve neoplastic cells. In these cases, the neoplasm typically has high levels of CD25+ve infiltrating cells, such as CD25+ve T-cells. That is, the proliferative disease may be characterised by the presence of a neoplasm composed of CD25-ve neoplastic cells, optionally wherein the CD25-ve neoplastic cells are associated with CD25+ve non-neoplastic cells such as CD25+ve T-cells.

The solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 January; 27(1):109-118).

Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

As noted above, in some cases the CD25+ve cell is a tumour infiltrating cell. In some cases the neoplasm or neoplastic cells are, or are present in, a hematological cancer. In some cases the neoplasm or neoplastic cells are, or are present in, a solid tumor. “Solid tumor” herein will be understood to include solid hematological cancers such as lymphomas (Hodgkin's lymphoma or non-Hodgkin's lymphoma) which are discussed in more detail below.

Another proliferative disorders of particular interest is (classical) Hodgkin lymphomas, with mixed cellularity type (Hodgkin-/Reed-Sternbert-Cells: CD25+/−). Classical Hodgkins lymphoma includes the subtypes nodular sclerosing, lymphocyte predominant, lymphocyte depleted and mixed cellularity. The Hodgkins lymphoma subtype may not be defined. In certain aspects, the subjects have Hodgkins lymphoma of the nodular sclerosing and mixed cellularity subtypes.

In some cases the neoplasm or neoplastic cells are malignant. In come cases the neoplasm or neoplastic cells are metastatic.

The disease may be resistant, relapsed or refractory. As used herein, relapsed disease constitutes conditions in which a previously treated tumor which became undetectable by conventional imaging technology again becomes detectable; refractory disease a condition in which the cancer—despite anti-tumor therapy—continues to grow.

Reduced Toxicity and Improved Efficacy of Discloses Dosing Regimes

The present disclosure provides a method of reducing the toxicity and/or side effects associated with administration of a CD25-ADC or CD25-ADC/PD-1 antagonist combination to a subject, the method comprising administering the CD25-ADC or CD25-ADC/PD-1 antagonist combination in a dosage regime as defined herein.

In some cases the reduction in toxicity is measured relative to the treatment regime administered to the subject before commencing treatment with the CD25-ADC as decribed herein. The treatment regime administered to the subject before commencing treatment with the CD25-ADC as described herein may be treatment with a CD25-ADC other than the CD25-ADCs described herein and/or in a dosage regime other than those described herein.

In some cases the level of toxicity is measured as the incidence of Treatment Emergent Adverse Events (TEAE) occurring after one treatment cycle of CD25-ADC. A treatment-emergent AE (TEAE) is defined as any event not present before exposure to the CD25-ADC or any event already present that worsens in either intensity or frequency after exposure to the CD25-ADC. The incidence of AE with the CD25-ADC may be no more that 95%, such as no more than 90%, no more than 80%, no more than 70%, no more than 60%, no more than 50%, no more than 40%, no more than 30%, no more than 20%, no more than 10%, or no more than 5% of the incidence of AE in the treatment regime administered to the subject before commencing treatment with the CD25-ADC as described herein. Adverse events will be graded according to CTCAE Version 4.0 (v4.03, published Jun. 14, 2010; NIH Publication No. 09-5410).

For example, if the treatment regime administered to the subject before commencing treatment with the CD25-ADC as described herein when administered to 100 subjects leads to 10 AEs and the CD25-ADC regime leads to 5 AEs, the incidence of AEs with the CD25-ADC regime is 50% of the incidence of AE in the preceding regim

The above adverse events will be graded according to CTCAE Version 4.0 (v4.03, published Jun. 14, 2010; NIH Publication No. 09-5410).

The present disclosure also provides a method of increasing the treatment efficacy associated with administration of a CD25-ADC or CD25-ADC/PD-1 antagonist combination to a subject, the method comprising administering the CD25-ADC or CD25-ADC/PD-1 antagonist combination in a dosage regime as defined herein.

In some cases the increase in efficacy is measured relative to the treatment regime administered to the subject before commencing treatment with the CD25-ADC as described herein. The treatment regime administered to the subject before commencing treatment with the CD25-ADC as described herein may be treatment with a CD25-ADC other than the CD25-ADCs described herein and/or in a dosage regime other than those described herein.

In some cases the level of efficacy is measured as the proportion of subjects achieving at least a partial response [PR] after one treatment cycle of a dosage regime as described herein (i.e the proportion of subjects achieving either a partial response [PR], or a complete response [CR]). The proportion of subjects achieving at least PR with the dosage regime as described herein may be at least 110%, such as at least 120%, at least 130%, at least 140%, at least 150%, at least 160%, at least 170%, at least 180%, at least 190%, or at least 200%, of the proportion of subjects achieving at least a partial response [PR] with the treatment regime administered to the subject before commencing treatment with the CD25-ADC as decribed herein.

For Hodgkins lymphoma, assessment of response to treatment with ADC may be based on bone marrow samples (aspirate or biopsy if aspirate unattainable) taken toward the end of each treatment cycle. For example, on day 19±3 days in a 21-day treatment cycle. The subject's response to ADC may be categorised as CR, PR, SD, or PD according to the 2014 Lugano Classification Criteria (using the New “Cheson” Criteria), in which:
Complete response (CR) is defined as achieving each of the following:
   Nodal Disease<1.5 cm in LDi
   Extranodal Disease: Absent
   Spleen: regress to normal
   No new lesions
   Bone marrow: Normal by morphology; if indeterminate, IHC negative
Partial response (PR) is defined as achieving each of the following:
   Nodal Disease>=50% decrease from baseline in SPD of all target lesions
   No increase in non-target
   Spleen: >50% decrease from baseline in enlarged portion of spleen (value>13 cm)
   No new lesions
Stable Disease (SD) is defined as achieving each of the following:
   Nodal Disease<50% decrease from baseline in SPD of all target lesions
   No criteria for nodal PD are met
   No progression in non-target
   No progression in spleen enlargment
   No new lesions

Nodal PD criteria:
An individual node/lesion must be abnormal with:
   LDi>1.5 cm AND
   Increase by >=50% from PPD nadir AND
   An increase in LDi or SDi from nadir
   λ2 0.5 cm for lesions≤2 cm
   ≥1.0 cm for lesions>2 cm

For solid tumours, assessment of the criteria for objective response level (PR or CR) is preferably according to the criteria set out in RECIST Version 1.1 (see http://www.irrecist.com/recist/; Schwartz, Lawrence H. et al., European journal of cancer 62 (2016): 132-137. PMC. Web. 3 May 2018).

Selection of Patients for Treatment with Disclosed Dosage Regimes

In certain cases, the subjects are selected as suitable for treatment with the dosage regimes disclosed herein before the treatment is administered.

As used herein, subjects who are considered suitable for treatment are those subjects who are expected to benefit from, or respond to, the treatment. Subjects may have, or be suspected of having, or be at risk of having cancer. Subjects may have received a diagnosis of cancer. In particular, subjects may have, or be suspected of having, or be at risk of having, Hodgkin lymphoma, a solid tumour comprising CD25+ve neoplastic cells, or a solid tumour comprising high levels of CD25+ve cells associated with the target neoplastic cells (for example, high levels of infiltrating CD25+ve T-cells). In some cases, subjects may have, or be suspected of having, or be at risk of having, a solid CD25-ve cancer that has tumour associated infiltrating T-cells that express CD25.

In some cases, subjects are selected on the basis of the amount or pattern of expression of CD25. In some cases, the selection is based on expression of CD25 at the cell surface.

In some cases, expression of CD25 in a particular tissue of interest is determined. For example, in a sample of lymphoid tissue or tumor tissue. In some cases, systemic expression of CD25 is determined. For example, in a sample of circulating fluid such as blood, plasma, serum or lymph.

In some cases, the subject is selected as suitable for treatment due to the presence of CD25 expression in a sample. In those cases, subjects without CD25 expression may be considered not suitable for treatment.

In other cases, the level of CD25 expression is used to select a subject as suitable for treatment. Where the level of expression of CD25 is above a threshold level, the subject is determined to be suitable for treatment.

In some cases, the presence of CD25+ in cells in the sample indicates that the subject is suitable for treatment with a combination comprising an ADC. In other cases, the amount of CD25 expression must be above a threshold level to indicate that the subject is suitable for treatment. In some cases, the observation that CD25 localisation is altered in the sample as compared to a control indicates that the subject is suitable for treatment.

In some cases, subjects are selected on the basis they have a neoplasm comprising both CD25+ve and CD25-ve cells. The neoplasm may be composed of CD25-ve neoplastic cells, optionally wherein the CD25-ve neoplastic cells are associated with CD25+ve non-neoplastic cells such as CD25+ve T-cells. The neoplasm or neoplastic cells may be all or part of a solid tumour. The solid tumour may be partially or wholly CD25-ve, and may be infiltrated with CD25+ve cells, such as CD25+ve T-cells.

In some cases, a subject is indicated as suitable for treatment if cells obtained from lymph node or extra nodal sites react with antibodies against CD25 as determined by IHC.

In some cases, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express CD25. In some cases disclosed herein, a patient is determined to be suitable for treatment if at least at least 5% of the cells in the sample express CD25.

In some cases, a subject undergoes a neurological examination prioir to treatment with the ADC. Preferably the neurological examination includes tests of strength, sensation, and deep-tendon reflexes.

In some cases, a subject is determined to be not suitable for treatment with the ADC if they have, or have recently had, a neurologic disorder. Examples of such disorders include poliomyelitis and multiple sclerosis Generally, neurological disorders that are explained by the subject's previous medical history and known not to be related to, or a risk factor for, to treatment with ADC do not render a subject unsuitable for treatment with the ADC. An example of such a disorder is a left-sided weakness known to be a result of a previous cerebral vascular accident, such as a stroke.

The neurologic disorder, as discussed herein, may be polyradiculopathy (including acute inflammatory demyelinating polyradiculoneuropathy (AIDP)), Guillain-Barre syndrome (GBS), myasthenia gravis, or neurologic disorder that is linked to or is an early indicator of polyradiculitis, GBS, or myasthenia gravis (e.g. ascending (bilateral) sensory loss and/or motor weakness).

In some cases, a subject undergoes a neurological examination after administration of the ADC. In some cases the results of the neurological examination of a subject after administration of the ADC are compared to the results from before administration of the ADC in order to assess any change in the tested neurological parameters. In some cases, treatment with the ADC is reduced, suspended, or permenantly discontinued if the subject experiences a neurologic toxicity.

The neurologic toxicity, as discussed herein, may be polyradiculopathy (including acute inflammatory demyelinating polyradiculoneuropathy (AIDP)), Guillain-Barre syndrome (GBS), myasthenia gravis, or neurologic disorder that is linked to or is an early indicator of polyradiculitis, GBS, or myasthenia gravis (e.g. ascending (bilateral) sensory loss and/or motor weakness).

In some cases, a subject undergoes a neurological examination after each administration of the ADC. In some cases the results of the neurological examination of a subject after each administration of the ADC are compared to the results from before the most recent administration of the ADC in order to assess any change in the tested neurological parameters. In some cases the results of the neurological examination of a subject after each administration of the ADC are compared to the results from before the first administration of the ADC in order to assess any change in the tested neurological parameters.

In some cases, a subject undergoes a neurological examination if they experience a neurologic toxicity following administration of the ADC.

In some cases, treatment with the ADC is reduced, suspended, or permenantly discontinued if the subject has a neurological disorder or experiences a neurologic toxicity. For example, if a subject experiences≥grade 1 neurologic toxicity, such as a grade 1 neurologic toxicity that is linked to or is an early indicator of polyradiculitis (e.g. ascending (bilateral) sensory loss and/or motor weakness) treatment with the ADC may be reduced or suspended. In some case, if the subject experiences a ≥grade 2 neurologic toxicity (e.g. grade 2 polyradiculitis or GBS), treatment with the ADC may be permenantly discontinued.

Adverse events will be graded according to CTCAE Version 4.0 (v4.03, published Jun. 14, 2010; NIH Publication No. 09-5410).

In some cases, treatment with the ADC is reduced by reducing the dose of ADC that is administered to the subject in each subsequent treatment cycle. In some cases, treatment with the ADC is reduced by increasing the length of each subsequent treatment cycle for example, from a 3 week cycle to a 6 week cycle). In some cases, treatment with the ADC is reduced by reducing the dose of ADC that is administered to the subject in each subsequent treatments cycle and increasing the length of each subsequent treatment.

In some cases, treatment with the ADC is suspended by stopping treatment with the ADC until the toxicity is resolved. In some cases, treatment with the ADC is resumed after resolution of the toxicity to baseline. The subject may be monitored weekly until the neurologic toxicity is resolved. In some cases the treatment is suspended for up to 3 weeks (21 days).

For example, in some cases a subject undergoes a neurological examination if they experience≥grade 1 neurologic toxicity, such as a grade 1 neurologic toxicity that is linked to or is an early indicator of polyradiculitis (e.g. ascending (bilateral) sensory loss and/or motor weakness). In some cases, if a subject experiences a ≥grade 1 neurologic toxicity (e.g. grade 1 polyradiculitis or GBS), treatment with the ADC is resumed after resolution of the toxicity to baseline. The subject may be monitored weekly until the neurologic toxicity is resolved.

In some cases, if a subject experiences a ≥grade 2 neurologic toxicity (e.g. grade 2 polyradiculitis or GBS), treatment with the ADC is permenantly discontinued.

In some cases, a subject is determined to be not suitable for treatment with the ADC if they have, have recently had, or hisotically had, an infection caused by a pathogen that may be associated with neurologic and/or immune-related disease. Examples of such pathogens include HSV1, HSV2, VZV, EBV, CMV, measles, Influenza A, Zika virus, Chikungunya virus, Mycoplasma pneumonia, Campylobacter jejuni, or enterovirus D68.

In some cases, treatment with the ADC is reduced, suspended, or permenantly discontinued if the subject experiences has, or aquires, an infection caused by a pathogen that may be associated with neurologic and/or immune-related disease. Examples of such pathogens include HSV1, HSV2, VZV, EBV, CMV, measles, Influenza A, Zika virus, Chikungunya virus, Mycoplasma pneumonia, Campylobacter jejuni, or enterovirus D68. In some cases, treatment with the ADC is suspended until at least 4 weeks after symptoms of the infection are resolved.

Examples of immune-relatyed diseases include rheumatoid arthritis, systemic progressive sclerosis [scleroderma], systemic lupus erythematosus, Sj6gren's syndrome, autoimmune vasculitis [e.g., Wegener's granulomatosis].

In some cases, treatment with the ADC is reduced, suspended, or permenantly discontinued if the subject experiences any ≥grade 1 autoimmune toxicities (e.g. endocrinopathies,).

Dosinq Subject Status

The subject may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject may be any of its forms of development, for example, a foetus. In one preferred embodiment, the subject is a human. The terms “subject”, “patient” and “individual” are used interchangeably herein.

In some cases disclosed herein, a subject has, or is suspected as having, or has been identified as being at risk of, cancer. In some cases disclosed herein, the subject has already received a diagnosis of cancer. The subject may have received a diagnosis of (classical) Hodgkins lymphoma (including nodular sclerosing, lymphocyte predominant, lymphocyte, or mixed cellularity type, or where the type is unspecified) and/or a solid tumour.

In some cases disclosed herein, a subject has, or is suspected as having, has been identified as being at risk of, or has received a diagnosis of a solid cancer containing CD25+ expressing infiltrating T-cells.

In some cases disclosed herein, a subject has, or is suspected as having, has been identified as being at risk of, or has received a diagnosis of, a proliferative disease characterised by the presence of a neoplasm comprising both CD25+ve and CD25-ve cells. The neoplasm may be composed of CD25-ve neoplastic cells, optionally wherein the CD25-ve neoplastic cells are associated with CD25+ve non-neoplastic cells such as CD25+ve T-cells. The neoplasm or neoplastic cells may be all or part of a solid tumour. The solid tumor may be a neoplasm, including a non-haematological cancer, comprising or composed of CD25+ve neoplastic cells. The solid tumor may be a neoplasm, including a non-haematological cancer, infiltrated with CD25+ve cells, such as CD25+ve T-cells; such solid tumours may lack expression of CD25 (that is, comprise or be composed of CD25-ve neoplastic cells).

In some cases disclosed herein, a subject has, or is suspected as having, has been identified as being at risk of, or has received a diagnosis of a solid tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 January; 27(1):109-118). The some or all of the neoplastic cells in the tumour may be CD25-ve. The solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The Subject may be undergoing, or have undergone, a therapeutic treatment for that cancer. The subject may, or may not, have previously received ADCX25. In some cases the cancer is lymphoma, including Hodgkins or non-Hodgkins lymphoma.

Some Dosing Embodiments

The disclosure provides a method of treating a proliferative disease in a subject, said method administering to a subject an effective amount of a CD25-ADC in a Q3W dosage regime.

The dose of CD25-ADC may be about 20 ug/kg, about 30 ug/kg, about 45 ug/kg, about 60 ug/kg, about 80 ug/kg, about 100 ug/kg, about 125 ug/kg, about 150 ug/kg, about 200 ug/kg, about 250 ug/kg, or about 300 ug/kg.

The CD25-ADC is preferably ADCx25/ADCT-301/Camidanlumab Tesirine as described herein.

The disclosure further provides a method of treating a proliferative disease in a subject, said method administering to a subject an effective amount of a CD25-ADC in a Q3W dosage regime, an effective amount of a PD-1 antagonist is administered in combination with the CD25-ADC.

Preferably, the CD25-ADC and PD1 antagonist are administered concurrently; that is the CD25-ADC and the PD1 antagonist are administered as part of the same treatment cycle.

Preferably the PD1 antagonist is administered n a Q3W dosage regime. Preferably the dose administered is 200 mg per treatment cycle.

Preferably the PD1 antagonist is pembrolizumab.

When administered in combination with PD1 antagonist, the CD25-ADC is preferably administered in a dosage regime consisting of two, identical Q3W treatment cycles.

In cases where the subject achieves CR following initial treatment with the CD25-ADC and PD1 antagonist combination, preferably no further CD25-ADC is administered to the subject.

In cases where the subject achieves SD or PR following initial treatment with the CD25-ADC and PD1 antagonist combination, preferably PD1 antagonist administration continues after the initial treatment with the CD25-ADC and PD1 antagonist combination.

If the subject has not achieved CR within 3 months after the completion of initial CD25-ADC treatment, further CD25-ADC is preferably administered to the subject. The further CD25-ADC is preferably administered in a dosage regime consisting of two, equal Q3W treatment cycles. Preferably the further CD25-ADC is administered in combination with PD1 antagonist treatment.

The subject may be human.

The subject may have, be suspected of having, or have been diagnosed with a proliferative disease.

The proliferative disease may be (classical) Hodgkin lymphoma, with mixed cellularity type (Hodgkin-/Reed-Sternbert-Cells: CD25+/−).

Classical Hodgkins lymphoma includes the subtypes nodular sclerosing, lymphocyte predominant, lymphocyte depleted and mixed cellularity. The Hodgkins lymphoma subtype may not be defined. In certain aspects, the disorder is Hodgkins lymphoma of the nodular sclerosing or mixed cellularity subtype.

The proliferative disease may be a solid tumour, or characterized by the presence of a neoplasm or neoplastic cells that are all or part of a solid tumour. The neoplasm or neoplastic cells may be CD25-ve.

“Solid tumor” herein will be understood to include solid haematological cancers such as lymphomas (Hodgkin's lymphoma or non-Hodgkin's lymphoma) which are discussed in more detail herein.

Solid tumors may be neoplasms, including non-haematological cancers, comprising or composed of CD25+ve neoplastic cells. Solid tumors may be neoplasms, including non-haematological cancers, infiltrated with CD25+ve cells, such as CD25+ve T-cells; such solid tumours may lack expression of CD25 (that is, comprise or be composed of CD25-ve neoplastic cells).

For example, the solid tumour may be a tumour with high levels of infiltrating T-cells, such as infiltrating regulatory T-cells (Treg; Menetrier-Caux, C., et al., Targ Oncol (2012) 7:15-28; Arce Vargas et al., 2017, Immunity 46, 1-10; Tanaka, A., et al., Cell Res. 2017 January; 27(1):109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

The proliferative disease may be relapsed or refractory.

In a third aspect, the present disclosure provides a method of reducing the toxicity and/or side effects associated with administration of a CD25-ADC or CD25-ADC/PD-1 antagonist combination to a subject, the method comprising administering the CD25-ADC or CD25-ADC/PD-1 antagonist combination in a dosage regime as defined herein.

In a fourth aspect, the present disclosure provides a method of increasing the treatment efficacy associated with administration of a CD25-ADC or CD25-ADC/PD-1 antagonist combination to a subject, the method comprising administering the CD25-ADC or CD25-ADC/PD-1 antagonist combination in a dosage regime as defined herein.

In a fifth aspect, the present disclosure provides a method of selecting a subject for treatment by dosage regime as described herein, which selection method comprises selecting for treatment subjects that have either:
(1) Hodgkins lymphoma;
(2) a solid tumour comprising or consisting of CD25+ve cells; or
(3) a solid tumour comprising or composed of CD25-ve neoplastic cells which has high-levels of CD25+ve infiltrating cells, such as CD25+ve T-cells.

In a sixth aspect the present disclosure provides a packaged pharmaceutical product comprising a CD25-ADC as described herein in combination with a label or insert advising that the CD25-ADC should be administered in a dosage regime as described herein.

The disclosure also provides a kit comprising:
a first medicament comprising a CD25-ADC; and
a package insert or label comprising instructions for administration of the CD25-ADC in dosage regime as described herein;
optionally wherein the kit further comprises a PD-1 antagonist.

In a seventh aspect the present disclosure provides a CD25-ADC as defined herein, optionally in combination with a PD-1 antagonist, for use in a method of treatment as described herein.

In an eighth aspect the present disclosure provides the use of a CD25-ADC as defined herein, optionally in combination with a PD-1 antagonist, in the preparation of a medicament for use in a method of treatment as described herein.

ministering to a subject a CD25-ADC, wherein the CD25-ADC is administered to the subject in a tapered and/or elongated dosage regimes.

In some cases the dosage regime comprises dosing about 120 μg/kg every 3 weeks for 2 cycles, then continuing treatment with the third and subsequent cycles at a reduced dose of about 60 μg/kg every 6 weeks, beginning 6 weeks after cycle 2 administration. Preferably only subjects who have attained at least SD after the second cycle will continue with the reduced dose and increased cycle length.

In some cases the dosage regime comprises dosing about 150 μg/kg every 3 weeks for 2 cycles, then continuing treatment with the third and subsequent cycles at a reduced dose of about 60 μg/kg every 6 weeks, beginning 6 weeks after cycle 2 administration. Preferably only subjects who have attained at least SD after the second cycle will continue with the reduced dose and increased cycle length.

In some cases the dosage regime comprises dosing about 200 μg/kg every 6 weeks for 2 cycles, then continuing treatment with the third and subsequent cycles at a reduced dose of about 60 μg/kg every 6 weeks, beginning 6 weeks after cycle 2 administration. Preferably only subjects who have attained at least SD after the second cycle will continue with the reduced dose.

In some cases the dosage regime comprises dosing about 200 μg/kg every 6 weeks for 1 cycle, then continuing treatment with the second and subsequent cycles at a reduced dose of about 60 μg/kg every 6 weeks, beginning 6 weeks after cycle 1 administration. Preferably only subjects who have attained at least SD after the first cycle will continue with the reduced dose.

In some cases the dosage regime comprises dosing about 45 μg/kg every 3 weeks for up to 4 treatment cycles, then continuing treatment every 3 weeks at a reduced dose of about 30 μg/kg or about 20 μg/kg (such as 20 to 30 μg/kg). In some cases, the starting dose of 45 μg/kg is administered for only 1 treatment cycle before the dose is reduced. In some cases, the starting dose of 45 μg/kg is administered for only 2 treatment cycles before the dose is reduced. In some cases, the starting dose of 45 μg/kg is administered for only 3 treatment cycles before the dose is reduced. In some cases, the starting dose of 45 μg/kg is administered for 4 treatment cycles before the dose is reduced.

Preferably the CD25-ADC is administered as single dose on Day 1 of each cycle, unless otherwise specified.

Preferably the CD25-ADC is ADCx25/ADCT-301/Camidanlumab Tesirine as described herein.

Preferably the proliferative disease is lymphoma, such as Non Hodgkin Lymphoma. The disease may be relapsed or refractory.

Administration of ADCx25 in combination with SGN-CD33A for the treatment of AML is envisaged. Administration of ADCx25 in combination with inotuzumab ozogamicin for the treatment of ALL is envisaged.

Preferably the subject is human.

Preferably the CD19-ADC is administered in combination with dexamethasone, as described herein.

Statements of Dosing Disclosure

1d. A method of treating a proliferative disease in a subject, said method comprising administering to a subject a CD25-ADC,
wherein the CD25-ADC comprises a conjugate of formula L—(D^{L})ₚ, where D^{L} is of formula I or II:

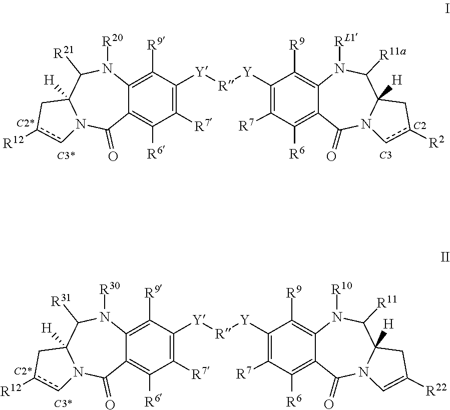

wherein:

L is an antibody (Ab) which is an antibody that binds to CD25;
when there is a double bond present between C2′ and C3′, R¹² is selected from the group consisting of:
   (ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
   (ib) C₁₋₅ saturated aliphatic alkyl;
   (ic) C₃₋₆ saturated cycloalkyl;
   (id)

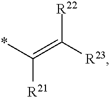

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;
(ie)

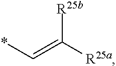

wherein one of R^{25a} and R^{25b} is H and the other is selected from: phenyl, which phenyl is optionally substituted by a group selected from halo, methyl, methoxy; pyridyl; and thiopheny rrN1
(if)

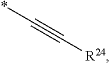

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,

### R¹² is

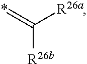

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;

### [Formula I]

R^{L1′} is a linker for connection to the antibody (Ab);
R^{11a} is selected from OH, OR^{A}, where R^{A} is C₁₋₄ alkyl, and SOzM, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id)

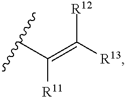

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;
(ie)

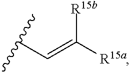

wherein one of R^{15a} and R^{15b} is H and the other is selected from: phenyl, which phenyl is optionally substituted by a group selected from halo, methyl, methoxy; pyridyl; and thiophenyl; and
(if)

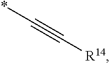

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,

### R² is

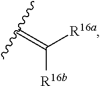

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

### [Formula II]

R²² is of formula Illa, formula Illb or formula Illc:
(a)

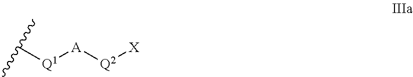

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;
(b)

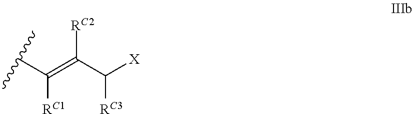

where;
R^{C1}, R^{C2} and R^{C3} are independently selected from H and unsubstituted C₁₋₂ alkyl;
(c)

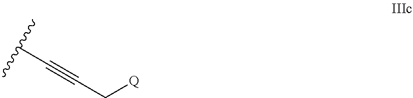

where Q is selected from O—R^{L2′}, S—R^{L2′} and NR^{N}—R^{L2′}, and RN is selected from H, methyl and ethyl
X is selected from the group comprising: O—R^{L2′}, S—R^{C}, CO₂—R^{L2′}, O—R^{L2′}, NH—C(═O)—R^{L2′}, NHNH—R^{L2′}, CONHNH—R^{L2′},

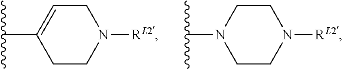

NR^{N}R^{L2′}, wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SOzM.
2d. The method according to statement 1d, wherein the CD25-ADC is administered to the subject Q1W, Q2W, Q3W, Q4W, Q5W, or Q6W dosage regime.
3d. The method according to statement 2d, wherein the CD25-ADC is administered to the subject Q3W dosage regime.
4d. The method according to any one of statements 1d to 3d wherein the CD25-ADC has the chemical structure:

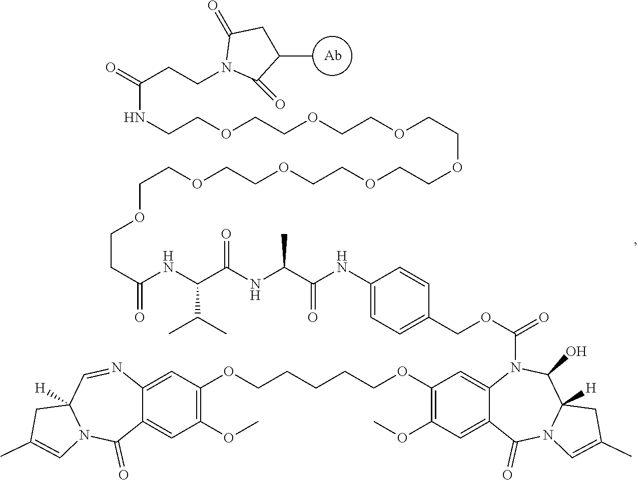

where the Ab is a CD25 antibody, and the DAR is between 1 and 8.
5d. The method according to any one of statements 1d to 4d wherein Ab comprises:

a VH domain comprising a VH CDR1 with the amino acid sequence of SEQ ID NO.3, a VH CDR2 with the amino acid sequence of SEQ ID NO.4, and a VH CDR3 with the amino acid sequence of SEQ ID NO.5; and, optionally,

a VL domain comprising a VL CDR1 with the amino acid sequence of SEQ ID NO.6, a VL CDR2 with the amino acid sequence of SEQ ID NO.7, and a VL CDR3 with the amino acid sequence of SEQ ID NO.8.

6d. The method according to any one of statements 1d to 5d wherein Ab comprises a VH domain having the sequence of SEQ ID NO. 1 and a VL domain having the sequence of SEQ ID NO. 2.
7d. The method according to any one of statements 1d to 6d wherein the CD25-ADC is ADCx25, ADCT-301, or Camidanlumab Tesirine.
8d. The method according to any preceding statement wherein the dose of CD25-ADC is at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 μg/kg.
9d. The method according to any preceding statement wherein the CD25-ADC dose is about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, or 600 μg/kg.
10d. The method according to any preceding statement wherein the CD25-ADC dose is 1 to 10 μg/kg, 11 to 20 μg/kg, 21 to 30 μg/kg, 31 to 40 μg/kg, 41 to 50 μg/kg, 51 to 60 μg/kg, 61 to 70 μg/kg, 71 to 80 μg/kg, 81 to 90 μg/kg, 91 to 100 μg/kg, 101 to 120 μg/kg, 121 to 140 μg/kg, 141 to 160 μg/kg, 161 to 180 μg/kg, 181 to 200 μg/kg, 201 to 220 μg/kg, 221 to 240 μg/kg, 241 to 260 μg/kg, 261 to 280 μg/kg, 281 to 300 μg/kg, 301 to 320 μg/kg, 321 to 340 μg/kg, 341 to 360 μg/kg, 361 to 380 μg/kg, 381 to 400 μg/kg, 401 to 420 μg/kg, 421 to 440 μg/kg, 441 to 460 μg/kg, 461 to 480 μg/kg, 481 to 500 μg/kg, 501 to 520 μg/kg, 521 to 540 μg/kg, 541 to 560 μg/kg, 561 to 580 μg/kg, or 581 to 600 μg/kg.
11d. The method according to any preceding statement wherein the CD25-ADC dose is about 20 μg/kg.
12d. The method according to any preceding statement wherein the CD25-ADC dose is about 30 μg/kg.
13d. The method according to any preceding statement wherein the CD25-ADC dose is about 45 μg/kg.
14d. The method according to any preceding statement wherein the CD25-ADC dose is about 60 μg/kg.
15d. The method according to any preceding statement wherein the CD25-ADC dose is about 80 μg/kg.
16d. The method according to any preceding statement wherein the CD25-ADC dose is about 125 μg/kg.
17d. The method according to any preceding statement wherein the CD25-ADC dose is about 150 μg/kg.
18d. The method according to any preceding statement wherein the CD25-ADC dose is about 200 μg/kg.
19d. The method according to any preceding statement wherein the CD25-ADC dose is about 250 μg/kg.
20d. The method according to any preceding statement wherein the CD25-ADC dose is about 300 μg/kg.
21d. The method according to any preceding statement further comprising administering a PD1 antagonist in combination with the CD25-ADC.
22d. The method according to statement 21d wherein the PD1 antagonist is pembrolizumab, nivolumab, MED10680, PDR001 (spartalizumab), Camrelizumab, AUNP12, Pidilizumab Cemiplimab (REGN-2810), AMP-224, BGB-A317 (Tisleizumab), or BGB-108.
23d. The method according to statement 21d wherein the PD1 antagonist is pembrolizumab.
24d. The method according to any one of statements 21d to 23d, wherein the PD1 antagonist is administered concurrently with the CD25-ADC.
25d. The method according to statement 24d wherein the PD1 antagonist is administered on the same day as the CD25-ADC.
26d. The method according to any one of statements 21d to 25d, wherein the PD1 antagonist is administered in a Q3W dosage regime.
27d. The method according to any one of statements 21d to 26d, wherein the dose of PD1 antagonist is 200 mg per treatment cycle.
28d. The method according to any one of statements 21d to 27d, wherein the CD25-ADC is administered to the subject for two, 3-week treatment cycles.
29d. The method according to statement 28d, wherein the dose of CD25-ADC administered in each of the two, 3-week treatment cycles is the same.
30d. The method according to any one of statements 21d to 29d, wherein the administration of PD1 antagonist continues after the completion of CD25-ADC treatment.
31d. The method according to any one of statements 28d to 30d, wherein the CD25-ADC is administered to the subject for a further two, 3-week treatment cycles.
32d. The method according to statement 31d, wherein the further two CD25-ADC treatment cycles are administered If the subject has not achieved CR within 3 months after the completion of initial two, 3-week treatment cycles.
33d. The method according to any preceding statement wherein the subject has been diagnosed as having the proliferative disease prior to the start of treatment with the CD25-ADC.
34d. The method according to any preceding statement, wherein the proliferative disease is Hodgkin Lymphoma.
35d. The method according to any preceding statement, wherein the proliferative disease is a solid tumour.
36d. The method according to statement 35d, wherein solid tumour is pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, or head and neck cancer.
37d. The method according to any preceding statement wherein the proliferative disease is characterised by the presence of a neoplasm comprising CD25+ve neoplastic cells
38d. The method according to statement 3d7, wherein the neoplasm also comprises CD25-ve neoplastic cells.
39d. The method according to any one of statements 1 to 36d, wherein the proliferative disease is characterised by the presence of a neoplasm composed of CD25-ve neoplastic cells.
40d. The method according to any one of statements 33d to 35d, wherein the CD25+ve and/or the CD25-ve neoplastic cells are associated with CD25+ve non-neoplastic cells.
41d. The method according to statement 40d, wherein the CD25+ve non-neoplastic cells are CD25+ve T-cells, such as CD25+ve regulatory T-cells.
42d. The method according to any preceding statement wherein the method comprises the step of selecting a subject for treatment based on expression of CD25 on neoplastic cells or cells associated with neoplastic cells.
43d. The method according to statement 42d, wherein a subject is selected if at least 5% of neoplasm cells express CD25.
44d. The method according to any preceding statement wherein the proliferative disease is resistant, relapsed or refractory.
45d. The method according to any preceding statement wherein the subject is human.
46d. The method according to any preceding statement wherein the CD25-ADC is administered intravenously.
47d. The method according to any preceding statement, wherein the CD25-ADC is administered in combination with a steroid.
48d. The method according to statement 47d, wherein a first dose of steroid is administered on the same day as the ADC.
49d. The method according to statement 48d, wherein the first dose of steroid is administered at least 2 hours before the ADC.
50d. The method according to either one of statements 47d or 48d, wherein a second dose of steroid is administered the day after the ADC.
51d. The method according to statement 47d, wherein a first dose of steroid is administered the day before the ADC.
52d. The method according to statement 51d, wherein a second dose of steroid is administered on the same day as the ADC.
53d. The method according to statement 52d, wherein the second dose of steroid is administered at least 2 hours before the ADC.
54d. The method according to either one of statements 52d or 53d, wherein a third dose of steroid is administered the day after the ADC.
55d. The method according to any one of statements 47d to 54d, wherein the steroid or steroid doses are administered only in conjunction with the first administration of ADC in each treatment cycle.
56d. The method according to any one of statements 47d to 55d, wherein the steroid is administered orally.
57d. The method according to any one of statements 47d to 56d, wherein each dose of steroid is 8 mg.
58d. The method according to any one of statements 47d to 57d, wherein each dose of steroid is 16 mg.
59d. The method according to any one of statements 47d to 58d, wherein each dose of steroid is administered as two equal, partial doses.
60d. The method according to any one of statements 47d to 59d, wherein each partial dose is 4 mg.
61d. The method according to any one of statements 47d to 60d, wherein each partial dose is 8 mg.
62d. The method according to any one of statements 45d to 50d, wherein the steroid is dexamethasone.
63d. The method according to statement 47d, wherein 4 mg or 8 mg dexamethasone is administered orally twice daily: (i) the day before ADC administration on week 1, day 1 of the treatment cycle, (ii) the day of ADC administration on week 1, day 1 of the treatment cycle, and (iii) the day after ADC administration on week 1, day 1 of the treatment cycle.
64d. The method according to statement 47d, wherein 4 mg or 8 mg dexamethasone is administered orally twice daily: (i) the day of ADC administration on week 1, day 1 of the treatment cycle, and (ii) the day after ADC administration on week 1, day 1 of the treatment cycle.
65d. The method according to either one of statements 63d and 64d, wherein the dexamethasone administered on the same day as the ADC is administered at least two hours before the ADC.
66d. The method according to any one of statements 63d to 65d, wherein the dexamethasone is administered only in conjunction with the first administration of ADC in each treatment cycle.
67d. The method according to any preceding statement, wherein the subject undergoes a neurological examination prior to treatment with the ADC.
68d. The method according to any preceding statement, wherein the subject undergoes a neurological examination after administration of the ADC.
69d. The method according to any preceding statement, wherein the subject undergoes a neurological examination after each administration of the ADC.
70d. The method according to any preceding statement, wherein the subject undergoes a neurological examination if they experience a neurologic toxicity following administration of the ADC.
71d. The method according to any one of statements 67d to 70d, wherein the neurological examination includes tests of strength, sensation, and/or deep-tendon reflexes.
72d. The method according to any preceding statement, wherein treatment with the ADC is reduced, suspended, or permenantly discontinued if the subject has a neurological disorder or experiences a neurologic toxicity.
73d. The method according to any preceding statement, wherein treatment with the ADC is reduced or suspended if the subject experiences a grade 1 neurologic toxicity.
74d. The method according to any preceding statement, wherein treatment with the ADC is permenantly discontinued if the subject experiences a grade 2 neurologic toxicity.
75d. The method according to any one of statements 72d to 74d, wherein treatment with the ADC is reduced by reducing the dose of ADC that is administered to the subject in each subsequent treatment cycle, and/or by increasing the length of each subsequent treatment cycle.
76d. A method of selecting a subject for treatment by a method according to any one of statements 1d to 75d, which method comprises determining if the subject has, or recently had, a neurologic disorder, wherein the subject is determined to be not suitable for treatment with the ADC if they have, or have recently had, a neurologic disorder.
77d. A method of selecting a subject for treatment by a method according to any one of statements 1d to 75d, which method comprises determining if the subject has, or recently had, an infection caused by a pathogen that may be associated with neurologic and/or immune-related disease; wherein the subject is determined to be not suitable for treatment with the ADC if they have, or have recently had, such an infection and/or immune-related disease.
78d. The method according to any one of statements 70d to 77d, wherein the neurologic disorder or neurological toxicity is polyradiculopathy, acute inflammatory demyelinating (AIDP), Guillain-Barre syndrome (GBS), myasthenia gravis, or a neurologic disorder that is linked to or is an early indicator of polyradiculitis, GBS, or myasthenia gravis, such as ascending sensory loss and/or motor weakness.
79d. The method according to any one of statements 70d to 77d, wherein the neurologic disorder or neurological toxicity is Guillain-Barre syndrome (GBS).
80d. A method of reducing the toxicity and/or side effects associated with administration of a CD25-ADC to a subject, the method comprising administering the CD25-ADC according to the method of any preceding statement.
81d. A method of increasing the treatment efficacy associated with administration of a CD25-ADC to a subject, the method comprising administering the CD25-ADC according to the method of any preceding statement.
82d. A method of selecting a subject for treatment by a method according to any one of statements 1d to 79d, which method comprises selecting for treatment subjects that express CD25 in a tissue of interest.
83d. The method according to statement 82d wherein subjects are selected if at least 5% of cells in a sample of the tissue of interest express CD25.
84d. The method according to either one of statements 82d or 83d wherein the tissue of interest is lymphoid tissue or tumour tissue.
85d. A packaged pharmaceutical product comprising a CD25-ADC as defined in any one of statements 1d to 7d, in combination with a label or insert advising that the CD25-ADC should be administered according to the method of any one of statements 1d to 79d.
86d. A kit comprising:

a first medicament comprising a CD25-ADC as defined in any one of statements 1d to 7d;

a second medicament as defined in either one of statements 22d or 23d; and, optionally,

a package insert or label comprising instructions for administration of the CD25-ADC according to the method of any one of statements 1d to 79d.

87d. A CD25-ADC as defined in any one of statements 1d to 7d for use in a method of any one of statements 1d to 79d.
88d. A pharmaceutical composition comprising a CD25-ADC as defined in any one of statements 1d to 7d, optionally in combination with a pharmaceutically acceptable excipient, for use in a method of any one of statements 1d to 79d.
89d. Use of a CD25-ADC as defined in any one of statements 1d to 7d in the preparation of a medicament for use in a method of any one of statements 1d to 79d.

It will be appreciated by one of skill in the art that appropriate dosages of the ADC and compositions comprising this active element, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of ADC is determined by the expression of CD25 observed in a sample obtained from the subject. Thus, the level or localisation of expression of CD25 in the sample may be indicative that a higher or lower dose of ADC is required. For example, a high expression level of CD25n may indicate that a higher dose of ADC would be suitable.

In some cases, a high expression level of CD25 may indicate the need for administration of another agent in addition to the ADC. For example, administration of the ADC in conjunction with a chemotherapeutic agent. A high expression level of CD25 may indicate a more aggressive therapy.

In certain aspects, the dosage level is determined by the expression of CD25 on neoplastic cells in a sample obtained from the subject. For example, when the target neoplasm is composed of, or comprises, neoplastic cells expressing CD25 In certain aspects, the dosage level is determined by the expression of CD25 on cells associated with the target neoplasm. For example, the target neoplasm may be a solid tumour composed of, or comprising, neoplastic cells that express CD25. For example, the target neoplasm may be a solid tumour composed of, or comprising, neoplastic cells that do not express CD25. The cells expressing CD25 may be non-neoplastic cells infiltrating the solid tumour, such as infiltrating lymphocytes (eg. Treg cells).

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of each active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, each active compound is administered to a human subject according to the following dosage regime: about 100 mg, 3 times daily.

In one embodiment, each active compound is administered to a human subject according to the following dosage regime: about 150 mg, 2 times daily.

In one embodiment, each active compound is administered to a human subject according to the following dosage regime: about 200 mg, 2 times daily.

However in one embodiment, each conjugate compound is administered to a human subject according to the following dosage regime: about 50 or about 75 mg, 3 or 4 times daily.

In one embodiment, each conjugate compound is administered to a human subject according to the following dosage regime: about 100 or about 125 mg, 2 times daily.

For the ADC the dosage amounts described above may apply to the conjugate (including the PBD moiety and the linker to the antibody) or to the effective amount of PBD compound provided, for example the amount of compound that is releasable after cleavage of the linker.

The CD25-ADC comprises an anti-CD25 antibody. The anti-CD25 antibody may be HuMax-TAC™. The ADC may comprise a drug which is a PBD dimer. The ADC is preferably ADCX25/ADCT-301/Camidanlumab Tesirine. The ADC may be an ADC disclosed in WO2014/057119.

Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind a first target protein (Miller et al (2003) Jour. of Immunology 170:4854-4861). Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1 ml, G1m2, G1 m3, non-G1 m1 [that, is any allotype other than G1 m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include Clq binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called a, 6, e, y, and p, respectively. The subunit structures and three-dimensional configurations of different classes of immunoglobulins are well known.

Anti-CD25 antibodies are known in the art and are useful in the methods disclosed herein. These include antibodies 4C9 (obtainable from Ventana Medical Systems, Inc.). Other suitable antibodies include antibody AB12 described in WO 2004/045512 (Genmab A/S), IL2R.1 (obtainable from Life Technologies, catalogue number MA5-12680) and RFT5 (described in U.S. Pat. No. 6,383,487). Other suitable antibodies include B489 (143-13) (obtainable from Life Technologies, catalogue number MA1-91221), SP176 (obtainable from Novus, catalogue number NBP2-21755), 1B5D12 (obtainable from Novus, catalogue number NBP2-37349), 2R12 (obtainable from Novus, catalogue number NBP2-21755), or BC96 (obtainable from BioLegend, catalogue number V T-072) and M-A251 (obtainable from BioLegend, catalogue number IV A053). Other suitable anti-CD25 antibodies are daclizumab (Zenapax™) and basiliximab (Simulect™), both of which have been approved for clinical use.

### BRIEF DESCRIPTION OF THE FIGURES

Embodiments and experiments illustrating the principles of the disclosure will now be discussed with reference to the accompanying figures in which:

FIG. 1. Sequences

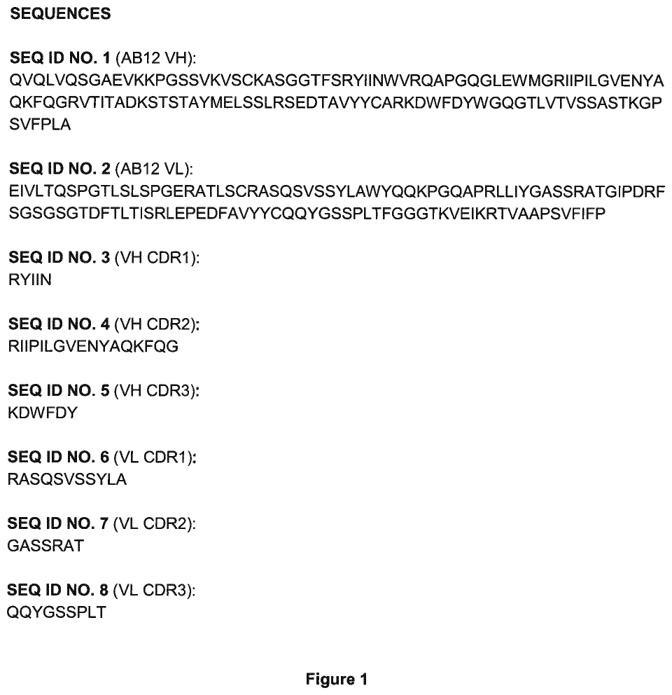

FIG. 2. In vivo MC38 tumour volume following mono-treatment with surrogate ADCx25, anti-PD1 treatment, or control ADC (as per Example 1)

FIG. 3. In vivo MC38 tumour volume showing synergy between low-dose surrogate ADCx25 and anti-PD1 treatment (as per Example 1)

FIG. 4. Re-challenge of tumour-free survivors from MC38 efficacy study (as per Example 3)

FIG. 5. In vivo CT26 tumour volume following mono-treatment with surrogate ADCx25, anti-PD1 treatment, or control ADC (as per Example 4)

FIG. 6. In vivo CT26 tumour volume showing synergy between low-dose surrogate ADCx25 and anti-PD1 treatment (as per Example 4)

FIG. 7. Re-challenge of tumour-free survivors from CT26 efficacy study (as per Example 5)

FIG. 8. ADCx25 anti-tumour activity is dependent on CD8+ T-cells

FIG. 9. ADCx25 plus PD-1 anti-tumour activity is dependent on CD8+ T-cells

FIG. 10. Spleen T-cell immuno-profiling after SurADCx25 dosing in healthy immuno-competent mice

FIG. 11. Lymph node T-cell immuno-profiling after SurADCx25 dosing in healthy immuno-competent mice

FIG. 12. Blood T-cell immuno-profiling after SurADCx25 dosing in healthy immuno-competent mice

FIG. 13. Thymus T-cell immuno-profiling after SurADCx25 dosing in healthy immuno-competent mice

FIG. 14. Tumour T-cell immuno-profiling after SurADCx25 dosing in CT26 tumor-bearing immuno-competent mice

FIG. 15. Spleen T-cell immuno-profiling after SurADCx25 dosing in CT26 tumor-bearing immuno-competent mice

FIG. 16. Blood T-cell immuno-profiling after SurADCx25 dosing in CT26 tumor-bearing immuno-competent mice

### STATEMENTS OF INVENTION

1. A method of inducing or enhancing an immune response in a subject, the method comprising the step of administering a CD25-ADC to the subject.

2. The method of statement 1, wherein the immune response is a disorder-associated antigen (DAA) specific immune response.

3. The method of either one of statements 1 and 2, wherein the DAA-specific immune response is a CD8+ T cell response, a CD4+ T cell response, or an antibody response.

4. The method of any one of statements 1 to 3, wherein the DAA-specific immune response is a CD8+ T cell response.

5. The method of either one of statements 1 and 2, wherein the DAA-specific immune response is a memory cell response.

6. A method of treating or preventing a disorder a subject, wherein the disorder is characterized by a disorder-associated antigen (DAA), the method comprising administering a CD25-ADC to the subject.

7. A method of treating or preventing a disorder a subject, the method comprising administering a CD25-ADC to the subject.

8. The method of any one of statements 1 to 7, wherein the CD25-ADC is administered in combination with a DAA.

9. The method of any one of statements 2 to 8, wherein the DAA is a:
(a) protein, polypeptide, peptide, peptide mimetic;
(b) a nucleic acid encoding a protein, polypeptide, peptide, peptide mimetic
(c) a sugar or oligiosaccharide;
(d) a lipid, phospholipid, liposaccharide, or lipoprotein;
   optionally wherein the DAA is a cell-surface antigen.

10. The method of either one of statements 8 and 9, wherein the DAA is administered as part of a vaccine composition, optionally wherein the CD25-ADC is also part of the same vaccine composition

11. The method of either one of statements 9 and 10, wherein the vaccine composition comprises an adjuvant and, optionally a pharmaceutically acceptable excipient.

12. The method of any one of statements 9 to 11, wherein the CD25-ADC is administered before the DAA or vaccine composition.

13. The method of any one of statements 9 to 12, wherein the immune-suppressive activity of a population of regulatory immune cells in the subject is reduced by at least 90% before the DAA or vaccine composition is administered.

14. The method of any one of statements 8 to 13, wherein the size of a population of regulatory immune cells in the subject is reduced by at least 90% before the DAA or vaccine composition is administered.

15. The method of statement 14, wherein the regulatory immune cells are Treg cells.

16. The method of any one of statements 8 to 11, wherein the CD25-ADC is administered concomitantly with the DAA or vaccine composition.

17. The method of any one of statements 2 to 16, wherein, the dose of CD25-ADC administered is about 20 μg/kg to 80 μg/kg, such as about 20, 25, 30, 35, 40, 45, 50, 55, 60, 65, 70, 75, or 80 μg/kg.

18. The method of any one of statements 2 to 17, wherein the DAA is a tumour-associated antigen (TAA);
optionally wherein the TAA is selected from the group consisting of BMPR1B, E16, STEAP1, 0772P, MPF, Napi3b, Sema 5b, PSCA hlg, ETB, MSG783, STEAP2, TrpM4, CRIPTO, CD21, CD79b, FcRH2, HER2, NCA, MDP, IL20R-alpha, Brevican, EphB2R, ASLG659, PSCA, GEDA, BAFF-R, CD22, CD79a, CXCR5, HLA-DOB, P2X5, CD72, LY64, FcRH1, IRTA2, TENB2, PSMA, SST, ITGAV, ITGB6, CEACAM5, MET, MUC1, CA9, EGFRvIII, CD33, CD19, IL2RA, AXL, CD30, BCMA, CT Ags, CD174, CLEC14A, GRP78-HSPA5, CD70, Stem Cell specific antigens, ASG-5, ENPP3, PRR4, GCC-GUCY2C, Liv-1-SLC39A6, 5T4, CD56-NCMA1, CanAg, FOLR1, GPNMB, TIM-1-HAVCR1, RG-1, B7-H4-VTCN1, PTK7, CD37, CD138, CD74, Claudins, EGFR, Her3, RON-MST1R, EPHA2, CD20-MS4A1, Tenascin C-TNC, FAP, DKK-1, CD52, CS1-SLAMF7, Endoglin, Annexin A1, V-CAM (CD106), DLK-1, and KAAG1.

19. The method of any one of statements 2 to 17, wherein the DAA is a pathogen-associated antigen (PAA).

20. The method of statement 19, wherein the PAA is derived from a pathogen selected from the group consisting of a virus, bacterium, fungus, protozoa, parasite, prion protein, or protein aggregate;
optionally wherein the pathogen is selected from:
Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirusalitis, GB virus C/Hepatitis G virus Pegivirus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus O, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus;
Acetobacter aurantius, Acinetobacter baumannii, Actinomyces israelii, Agrobacterium radiobacter, Agrobacterium tumefaciens, Anaplasma sp. inc. Anaplasma phagocytophilum, Azorhizobium caulinodans, Azotobacter vinelandii, Bacillus, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus Thuringiensis, Bacteroides sp. inc Bacteroides fragilis, Bacteroides gingivalis, Bacteroides melaninogenicus, Prevotella melaninogenica, Bartonella sp. inc. Bartonella henselae, Bartonella Quintana, Bordetella sp. inc. Bordetella bronchiseptica, Bordetella pertussis, Borrelia burgdorferi, Brucella sp. inc. Brucella abortus, Brucella melitensis, Brucella suis, Burkholderia sp. inc. Burkholderia mallei, Burkholderia pseudomallei, Burkholderia cepacia, Calymmatobacterium granulomatis, Campylobacter sp. inc. Campylobacter coli, Campylobacter fetus, Campylobacter jejuni, Campylobacter pylori, Chlamydia sp. inc. Chlamydia trachomatis, Chlamydophila sp. inc. Chlamydophila pneumoniae, Chlamydophila psittaci, Clostridium sp. inc. Clostridium botulinum, Clostridium difficile, Clostridium perfringens, Clostridium tetani, Corynebacterium sp. inc. Corynebacterium diphtheriae, Corynebacterium fusiforme, Coxiella burnetii, Ehrlichia chaffeensis, Enterobacter cloacae, Enterococcus sp. inc. Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus sp. inc. Haemophilus ducreyi, Haemophilus influenzae, Haemophilus parainfluenzae, Haemophilus pertussis, Haemophilus vaginalis, Helicobacter pylon, Klebsiella pneumoniae, Lactobacillus sp. inc. Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium sp. inc. Mycobacterium avium, Mycobacterium bovis, Mycobacterium diphtheria, Mycobacterium intracellulare, Mycobacterium leprae, Mycobacterium lepraemurium, Mycobacterium phlei, Mycobacterium smegmatis, Mycobacterium tuberculosis, Mycoplasma sp. inc. Mycoplasma fermentans, Mycoplasma genitalium, Mycoplasma hominis, Mycoplasma penetrans, Mycoplasma pneumoniae, Neisseria sp. inc. Neisseria gonorrhoeae, Neisseria meningitides, Pasteurella sp. inc. Pasteurella multocida, Pasteurella tularensis, Peptostreptococcus, Porphyromonas gingivalis, Prevotella melaninogenica, Pseudomonas aeruginosa, Rhizobium radiobacter, Rickettsia sp. inc. Rickettsia prowazekii, Rickettsia psittaci, Rickettsia quintana, Rickettsia rickettsia, Rickettsia trachomae, Rochalimaea sp. inc. Rochalimaea henselae, Rochalimaea quintana, Rothia dentocariosa, Salmonella sp. inc. Salmonella enteritidis, Salmonella typhi, Salmonella typhimurium, Serratia marcescens, Shigella dysenteriae, Spirillum volutans, Staphylococcus sp. inc. Staphylococcus aureus, Staphylococcus epidermidis, Stenotrophomonas maltophilia, Streptococcus sp. inc. Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema sp. inc. Treponema pallidum, Treponema denticola, Vibrio sp. inc. Vibrio cholerae, Vibrio comma, Vibrio parahaemolyticus, Vibrio vulnificus, Wolbachia, Yersinia sp. inc. Yersinia enterocolitica, Yersinia pestis, and Yersinia pseudotuberculosis;
Aspergillus sp., Candida sp. including C. albicans and C. auris, Coccidioides sp., Cryptococcus gattii, Pneumocystis jirovecii, Sporothrix sp., Blastomyces sp., Cryptococcus neoformans, Histoplasma sp., Talaromyces sp. including T.marneffei, Anncaliia algerae, A. connori, A. vesicularum, Encephalitozoon cuniculi, E. hellem, E. intestinalis, Enterocytozoon bieneusi, Microsporidium ceylonensis, M. africanum, Nosema ocularum, Pleistophora sp., Trachipleistophora hominis, T. anthropophthera, Vittaforma corneae, and Tubulinosema acridophagus;
Acanthamoeba, Trypanosoma sp. including T. brucei, T. cruzi, and T. evansi, Plasmodium sp. including P. falciparum, P. vivax, P. ovale, P. malariae, P. knowlesi, P. relictum, P. anasum, and P. gallinaceum, Hemoproteus sp., Babesia sp., Besnoitia sp., Blastocystis sp., Cryptosporidium sp. including C. parvum and C. hominis, Cyclospora cayetanensis, Dientamoeba fragilis, Giardia so. including G. lamblia and G. intestinalis, Histomonas meleagridis, Sappinia sp. including S. diploidea and S.pedata, Toxoplasma gondii, and Trichomonas vaginalis;
Echinococcus sp. including E. granulosus and E. multilocularis, Entamoeba histolytica, Ancylostoma sp. including A. brazilense, A. caninum, and A. ceylanicum, Uncinaria stenocephala, Angiostrongylus sp. including Angiostrongylus cantonensis, Ascaris sp. including A. lumbricoides and A. suum, Balantidium coli, Balamuthia mandrillaris, Baylisascaris procyonis, Schistosoma sp. including S. mansoni, S. haematobium, and S. japonicum, Capillaria hepatica, Capillaria philippinensis, Clonorchis sp., Taenia sp. including T. solium, T. saginata, and T. asiatica, Diphyllobothrium sp. including Diphyllobothrium latum, Dipylidium sp., Dracunculus medinensis, Enterobius vermicularis, Fasciola sp. including Fasciola hepatica, Fasciolopsis buski, Gnathostoma sp., Heterophyes heterophyes, Naegleria fowleri, Onchocerca volvulus, Paragonimus sp., Sarcocystis sp., Strongyloides sp., Toxocara canis, Toxocara cati, and Trichinella sp.; and PrPI, PrPres, and PrPSc

21. The method of statement 19, wherein the PAA is selected from the group consisting of:

Q69549, Q07852, Q2HR82, Q7T928, P0C742, AOAOPOEFP7, Q80908, Q98839, Q9QJ49, P06427, P68564, Q5GFC6, Q3KSQ7, AOAOPOERXO, PODJZ1, Q98223, P03193, P04016, P04364, ElU5P1, P14353, AOAOPOELC3, Q9PX75, Q7T5D1, F5HG51, P17384, Q9QR71, E1U5N7, P29170, AOAON716U3, P03285, Q98226, P89471, P27225, Q3KSP4, Q5VHB0, P03493, B5SNS7, P20879, Q98200, P52540, Q07857, Q3KSP5, Q5GFC1, P0C723, B5SNR2, P09295, Q2KS45, F5HINO, P21735, P20540, Q76DN6, P21043, B5SNS8, P03286, Q2KS32, Q9WF19, P22422, P35986, Q8BEL8, P33861, B5SNRO, P09731, Q9PWVO, P33828, P24836, Q1HVC3, Q76RB7, P0C733, B5SNS9, F5HDD3, Q2KS76, Q0R5R3, P26555, Q8AZJ5, Q76RD5, Q3KSS5, B5SNS4, P09730, Q9PX47, P03086, P50782, P0C726, A9J188, Q1HVD3, B5SNT9, Q7M6G6, 11V199, P21097, P36825, P29996, A9J194, P0C722, B5SNS6, P09721, Q2TQY6, Q89187, P17387, P33803, Q77DT3, Q1HVF8, A9JOQ5, P09720, Q3S8C7, P20988, F5HAY6, P33042, Q6J3L8, Q1HVG1, A9JOW5, P09716, Q6QCM3, P20992, P03105, P03263, Q8QMU7, P68599, A9J1E2, F5H9N9, Q6QCP6, P68614, P36741, Q1HVD1, Q81971, P21041, A9J1J8, P21045, Q77JF8, P33843, Q9JH45, P03177, Q8QMX2, P24937, A9J1G6, P21099, Q6QCK2, P21057, P50798, P20843, 057247, Q81871, A9J1E9, P21100, Q6QCQ0, P33851, P03101, P33041, Q8QN10, P03277, A9JOX4, Q80923, Q6QCV8, P68618, P50822, P89446, C3V9V5, P03276, A9J141, P36723, Q6QCP2, P21065, P50800, Q3KSQ2, Q75Q70, P0C738, A9JOZ6, P26547, Q6QCM2, Q76QZ9, Q07861, P09250, Q91W62, P21036, A9JOS4, P17382, Q6QCWO, P21074, P50788, P21028, B9A5A8, P33859, A9JOV2, P36725, Q6QCL6, P68597, P36736, P18095, B9A5C6, P03284, A9JOW7, P06927, Q98222, P33835, Q80912, Q73367, B9A5D6, 012792, A9JOR2, P03118, Q806A8, P33836, P68320, P04591, Q3KSQ4, Q9DUC4, A9JON9, P04015, Q98261, P33837, P22425, P20874, Q4TUF4, P36716, A9J170, P27221, Q98216, PODJY9, Q98157, P08666, Q5VHB9, P21089, A9J138, P06463, Q98317, POCK28, P35256, P28864, Q5GFC8, Q9PX71, A9JOX6, P0DOD0, Q805Y6, Q6TUQ9, Q05128, P10189, Q8BEL5, Q9PX43, A9J107, Q80948, Q7T5D7, P33840, F5HC97, P52450, Q5VHC3, P21000, A9J1M0, Q45UF8, Q98315, P33842, Q9QJ40, P20978, Q5VHC6, P21001, A9J1K3, Q73369, Q98319, POCK20, Q9ENL1, F5HIG1, Q76RC6, Q9QJ58, A9J1R0, 070901, Q98275, P33847, Q89865, P10192, B9A5C1, P21115, A9J1T7, P60170, Q98311, P60672, Q77375, P89431, B9A5E0, P03224, A9J152, Q01351, Q76SZ3, P68616, Q9WT46, P52375, B9A5D2, P68345, A9J1U3, P52516, Q98210, P33854, P60505, P52451, B9A5A7, P03199, A9J101, P24435, Q98256, Q89183, Q9WT54, P04850, B9A5BO, Q3KST3, A9J1L5, Q9QJ28, Q98188, P21060, F5HCP3, Q6WB98, B9A5D9, P03197, A9JOU9, Q01352, Q76SZ4, P33832, P16773, 036634, B9A5D8, Q3KST5, A9J127, P52444, Q806B9, P21061, Q6SW10, P26629, Q3KSQ9, P30117, A9J1J4, P52445, Q7T5E1, P33855, F5HGQ8, Q77372, B9A5C5, Q3KSU2, A9J1K6, P20511, Q98294, P21069, F5H9Z4, P10211, B9A5B7, Q1HVH3, A9J136, P20523, Q805Y9, P21070, F5HAM0, P21022, Q5GFC2, P0C737, A9JOW9, P21085, Q806B8, P21066, P16741, P32991, E1U5P5, P03210, A9JOR8, P21119, Q89900, P21063, A8W995, P03347, Q5GFA9, P03206, A9J1K5, P21121, 11V175, P20990, P68620, P04592, E1U5P9, P21044, A9J114, P20542, 11V198, P33838, Q9QJ45, Q8JSZ3, Q8BEL7, P21104, A9J1E5, P20559, Q2KS4I, POCK27, Q69569, PODOEO, B9A5A6, P33797, A9JOP1, P68491, Q6JGG8, P20991, Q6SW14, P68762, B9A5B8, P12537, A9JOV4, P20551, Q2KS68, Q89489, P89436, P11235, K7ZLNO, P03274, A9J161, P20510, Q82009, P20535, P52463, P21030, Q3KSRO, P0C717, A9J1L4, P68485, Q4JEP8, P33816, Q9QJ33, P36320, Q6TV17, P30119, A9JOR6, P20512, Q82005, P33848, P52464, P0C763, B9A5B2, P0C734, A9J112, P68487, Q6QC16, P33850, F5HF47, Q69091, F8S4P1, P34016, A9JOT9, P20513, Q77JF7, P33852, P16821, P04488, C9EA16, 057211, A9J103, Q9DUC3, Q6QCL5, P33853, F5H8Q3, P89475, Q5VHA2, P33065, A9J148, Q9WGZ1, Q6QCL3, P33876, P16756, P68324, E1U5N3, P36713, A9J110, P68475, Q6QCT9, P21071, P16766, F5HAK9, Q6QCL1, 070739, A9J1V5, P68477, Q6QCQ2, P21067, Q6SW63, Q9QOU6, Q67721, Q9QU30, A9JOXO, P20547, Q6QCJ5, P33856, F5HAE6, P16827, Q6QCT4, P03207, A9J123, P20548, Q6X674, P21068, P52548, P52376, Q6QCQ1, Q3KSS8, A9JOU1, P20553, Q6QCP5, Q89186, Q06092, Q2HR92, Q6QCJ9, P21106, A9JOU3, P68626, Q6QCM5, P68595, P16798, P09300, Q3KSU9, P21090, B8XTP8, P20526, Q775H9, P33794, P08560, P90489, Q3KSVO, P21039, A9J1G1, P21120, Q6QCN1, P68594, F5HFB4, P03179, Q3KSU6, P34013, A9J1J5, P20527, Q6QCU3, P20534, Q6SW55, Q3KSV4, Q9QCF1, P34014, A9J1L3, P20530, Q6QCN2, P33839, P16813, Q1HVJO, Q98198, P33860, A9J114, P21123, Q6QCN5, P20994, P09288, Q6WB99, Q98255, P21042, A9J134, P20552, Q98213, Q07032, P16731, PODJX6, Q98274, Q9WSV7, A9JOQ7, F5HET4, Q98215, P21114, Q9WT06, Q3KSR5, Q7T5C1, P04133, A9J129, Q69548, Q98280, P68632, P52472, F5HF49, Q805Z9, Q9PZTO, A9JOW3, Q9QJ50, Q98197, P21096, P16762, P24935, Q98236, P12538, A9JOT7, F5H8R0, Q98259, P33849, P16787, P52448, Q98282, P21040, A9JOX5, Q69550, Q98248, P20993, Q6SWA4, Q2HR95, 040975, P20508, A9J1N4, P90463, Q98273, P68592, Q6SW89, Q1HVE7, Q98244, P03279, A9J1M1, Q9QJ46, Q98193, P33827, P16786, P16789, Q806C1, P36712, A9J1NO, Q69558, Q98283, P21058, Q6SW82, P04294, Q98278, P35988, A9JOX2, Q9QJ43, Q98307, P21064, P16775, P59632, Q7T5D6, P03281, A9J1R4, Q9QJ41, Q98196, P33844, P17146, P16728, Q806C0, P24447, A9J1F0, Q69559, Q7T5C5, P20985, Q6SWC3, F5HGN8, Q98271, P52346, A9J117, P52529, Q7T5C4, P33833, P16833, P09291, Q98302, P09253, A9J1T0, P03293, Q7T5E9, P20987, P16721, F5HE74, Q8V727, P03195, A9J1V3, Q7TLC7, Q7T5D4, PODJZ0, P10186, Q69551, Q7T400, Q3KST7, A9J1J3, PODKB2, Q805Z8, P33841, Q20MD0, Q89882, Q98175, P89469, A9JOU5, Q9ENK7, Q7T5E8, P20989, Q20MD6, F5HHS3, Q806A4, Q2HRC6, A9J1R6, A5HBD5, Q89478, Q98300, Q9WF16, P16845, Q806C2, P21050, A9JOY6, P35258, Q9YIE0, P20995, Q9WF14, P16849, Q98272, P68449, A9J1L0, Q9ENL4, Q6RK96, Q89485, Q9WF20, P17148, Q98301, P33822, A9J1F7, Q91N14, Q9PWU3, P20996, Q98172, P16822, Q98237, P21081, A9JOQ2, Q89843, Q5EY84, P20983, Q98281, P52545, Q98310, P21080, A9J132, P20882, Q4JEP6, P33858, Q98203, Q2HRB2, Q98227, P33862, A9J1E3, POC1C6, Q9PX68, P33857, Q98293, Q69554, Q7T5E6, P14351, A9J172, P03290, Q2KS40, P09279, Q98322, Q9WT45, Q7T401, P14075, A9J150, P03294, Q2KS44, P14348, Q98297, P24437, Q98284, QOR5Q9, A9J1N6, P27557, Q6RK77, Q76QZ8, Q98312, P16801, Q806C4, P03383, A9J1C9, P03107, 11V197, P20531, Q87644, Q6SW48, Q805Z1, P04581, A9J1M3, Q05138, Q5EY48, P20842, Q98204, F5HFG3, Q89811, Q77377, A9J110, P03099, Q2KS77, Q5MQDO, Q98219, P16814, Q1WDN6, P20872, A9J1F2, P36753, Q2KS62, P03083, Q98328, P16797, Q806B4, P33064, A9J1 16, P36755, Q6RK85, A5HBD7, Q98206, F5HFJ8, Q98289, P03255, A9J112, P06794, Q9PX39, P20880, Q98205, P24439, Q85288, P03259, A9J1P3, P06418, 11V161, Q73370, Q6TVS6, P16781, Q7T5D2, P03243, A9J157, P32990, Q77SK4, P04611, Q6TVL3, P16847, Q806A5, P04491, A9JOZ4, P36735, Q9WF18, P04608, Q6TVMO, POCK56, Q98270, P36705, A9JOX9, P33814, Q7ZBU5, P14079, Q6TVT7, P16719, Q98309, PODJX0, A9J1D8, P25486, Q76630, F5HF23, Q6TVJ4, Q6SW79, Q7T5C2, Q3KSU3, A9J1D1, Q02515, 11V8P0, P04487, Q6TVJ8, P28282, Q98212, P03211, A9J0P3, P27558, Q6TUQ1, F7V996, Q6TVT5, P16816, Q98316, Q3KSS4, A9JOS7, Q80918, Q6TUQ0, P68608, Q6TVK9, P16746, Q98269, Q69022, Q80945, P06918, Q6TUP1, P33805, Q6TVL6, Q6SWC6, A7KCN3, Q1HVG4, Q89184, Q80946, Q6TUS9, P15423, Q6QCV5, P16776, Q98286, P68634, Q07045, Q80953, Q6TUY4, P24940, Q6TVN7, P16737, Q8V721, P06498, 013311, Q5MQC7, DOZ5U4, P21077, Q6TVKO, P16828, Q805Y1, P36707, Q20MC9, Q6Q1R9, DOZ5T3, P21062, Q6TVS8, P16829, Q7T5C8, P68978, Q83977, P35260, DOZ5S5, P24933, Q6TVK5, P16738, Q9YIWO, P36709, Q20MD1, Q91N13, DOZ5T4, P68340, Q6TVJO, P16770, Q98202, P21047, Q20MC7, P69481, DOZ5U8, PODOE5, Q6TVR8, P28275, Q98321, P33819, Q83976, P52439, Q9PWU2, P36714, Q6TVU9, P32988, Q805Z3, P06497, Q9WF13, P52440, DOZ5U9, P22112, Q6TVU1, P04413, Q7T5C6, P03242, G8H3V2, Q9YJJ8, DOZ5U3, P21132, Q6TVK6, P09722, Q806A6, P03191, Q80924, P05956, DOZSU1, P20841, Q6TV18, Q6SW00, Q98279, POC6Z1, D6NGF9, P69726, DOZ5R9, P33831, Q6TVM5, F5HDK1, Q805Y0, F5HGH5, 11V8N7, P05954, Q80929, Q6Q1S2, Q6TVK4, P69332, Q98186, Q82040, 11V8N8;
P13423, Q9S5G6, Q7CCJ3, Q9L6R5, Q00473, C6DHE3, P9WIQ6, P37669, P42382, P31494, Q9AIX9, P9WL65, B1JS09, P9WLS9, Q83F12, Q2FDM1, P39180, P58237, P21979, Q9L6Q8, Q9HUF7, POAG01, L8FKX3, B1LLW8, P26950, P45680, Q9AJ64, 053379, Q56978, QOSYY3, P62605, Q2FUX3, P9WGU1, P72139, Q07297, Q57HS2, P26403, B4TNU6, P0A569, Q1R4E1, QOPAS1, C3PCX2, Q9AJ75, POAB35, P9WG11, P27833, P11089, Q6GDG4, P9WQP1, P9WJA3, Q7A3D7, B4SZ40, P26406, A8G841, PODOA7, B5QVI4, P18194, Q73DZ5, BORVK4, POAB37, P9WGU0, B7MH57, Q06968, Q6G636, P9WIR7, Q9ZGM0, Q6GDN3, POAAA8, P37746, QOSYY4, P9WJD7, B5XYX5, P0C109, Q63GDO, 067998, P33792, QOP9X8, Q1CNJ1, Q06970, Q31SH3, P9WMK1, P60665, Q6G6A8, B7NTE8, Q07024, P9WLS5, P9WNK4, P27828, POA3P1, Q7A6D2, Q04974, Q04976, A1VZQ4, A4TRB6, Q06982, Q3YTH5, P9WPE7, Q4A122, Q79ZY3, A8A6P9, Q48476, P19478, 032629, B5BIU1, POA3P2, A8ZOC1, 066256, 031822, Q6F7F9, B5BIU3, Q06983, P18012, P9WK61, Q05852, Q5HDQ9, A9R8J0, Q06951, P18164, P44935, Q7MYM6, E4QEX4, B9DQ98, P16665, Q8FVC3, Q57254, Q66G07, P02975, Q6G6A5, P9WQN9, Q5HD54, Q8NV83, B1LLW7, Q50864, Q92BW7, Q83AF7, B4F1W8, POCOJ7, Q5HQE7, P37917, Q83BT6, P9WMK0, POAC79, E3PPC4, P18014, J714B7, Q6GDU6, Q53587, Q04866, P52642, Q48754, P14727, B5QVI6, Q9LBG3, P31495, Q83F59, P9WN15, P50927, POAC78, POCK93, B2TT54, P9WQP3, Q5HLD1, P31784, Q6CZF1, Q8Z514, P9WM17, 030620, B7UNB2, P11657, PODJH1, P07643, Q6U113, P50928, A7FD62, P11312, Q03947, POC2T2, Q8CR67, Q5HCY4, 033789, P37748, Q83AX3, P68641, A1AHW3, Q9KKA3, Q6G2B2, P29721, Q9RNC7, Q84168, B7M5E2, P04740, Q54150, P9WQB1, P77682, P16952, POAG02, P0A236, Q83A32, P21762, Q1CNJ0, Q2YLR6, Q9S3N1, P19649, F7YJG6, Q1RGK9, C4ZZ62, P02973, P9W155, P9WPE5, P43413, Q7BQ98, A9MXG6, Q56083, Q2YIU6, P26276, Q8FBP8, P05430, Q81IA1, P29720, Q9R2W4, P29697, B1LLW6, P33781, A7X6T9, P9WNK5, Q9S5G3, P46861, B5BIU2, Q00329, Q579D9, POCOB3, P27835, POCL66, P54166, P29724, Q04972, POA3N4, A8A6Q0, Q06971, Q7A377, P9WK45, Q06963, Q83D73, Q8Z396, POA1P4, Q9ZDHO, P27951, 033953, 031178, A6U0C2, P38369, Q45207, Q9F9F2, Q1CNI9, Q06974, Q8VSC3, Q06277, Q7DBF3, Q9HTB6, A7FD61, P27243, P43112, P45430, B614D1, Q8KQL6, Q2F160, P37761, G3XD61, Q52764, BlIWA5, P06179, P18011, B5XK69, Q8DR60, P55293, P43109, P37742, Q45214, Q661N6, B7LU62, P9WIG7, A5IRJ3, P55294, P37749, Q9KH57, Q9L6R7, POA2K7, Q03946, P9WMU1, Q5LGZ8, P37745, P43110, P37755, P0A687, Q9X6B1, P95730, P96989, Q8CPR3, P26391, P37750, Q04102, B614D2, POAEP6, Q2FDT8, Q53020, Q9S5G4, P37763, Q9RND2, P26405, P9WIG4, Q63K34, P37792, Q83EK8, AOR9F0, BORVLO, P71241, POA5B8, B5FN86, P25733, Q2FV52, P29723, P9WG64, Q46769, P21876, P26404, P9WIG5, P26949, B5EZ53, PODJ01, B7H9Q4, POC7J0, P26388, P50929, Q8ZAE1, Q3KMQ9, Q2YWD9, P9WJD9, P9WGM7, P61888, P29228, Q48478, P9WMU0, Q02192, POAG00, P18195, Q651A4, P37780, Q9XC60, P50930, B7LU61, POCOZ7, Q5HCY1, P02974, Q9L5X1, P31782, Q45215, Q05347, Q03084, Q63K37, B5FN87, PODH58, Q8NXC3, Q51832, Q8KIU8, P16624, A6TGI3, Q5ZXP3, Q6GDN1, Q49575, Q9L5X0, P9WGE7, Q45212, P37781, G3XD23, Q9X6B2, B5QV15, P15921, Q81YW9, P26392, PODMP6, P50931, COQ3A6, P16625, P60157, Q8FDQ2, PODF61, P23504, P11000, P0A235, B2TU00, P26948, C3LHC1, POCL67, C1EWE6, P37759, P67066, POA3N5, Q8Z395, P48219, Q5HL49, Q53047, POA4G3, Q53781, POA5N3, P37784, Q8Z389, Q63K35, B7JNE4, B7J1T8, A8FED1, Q6E7F4, Q9L6R4, Q7PA29, B5L3X1, P48214, Q8CMZ9, P33406, P31304, Q2G2J2, P37751, P26395, Q8ZAE4, QOSNDO, G3XD94, Q9HUG6, P22940, Q9S642, P67067, P22882, B5L3F2, G4WJD4, Q4AOG5, POCOJ8, P42363, Q6G723, P36667, Q05342, Q1CBP2, Q45010, P31496, Q83BSO, P37916, P37777, Q3YVHO, 052972, A8ACU6, P24301, A71Y64, PODJ00, Q9L5W9, Q2G2U9, Q4KXC9, P26471, A8ACU5, Q9ZLT1, P37918, P80369, 032270, P26394, A9R819, AOQU51, B7MH58, P12834, Q2YZ63, P26493, PODF60, A6QES8, P43111, Q48479, A7ZTZ6, P55980, Q02938, P20148, P17915, Q2SYI1, Q8XAS7, P9WQP0, B7L952, P69050, POA3L9, Q81QT1, P9WIN9, P13367, Q8KQC3, Q50863, B1JPZ9, A1VYV6, Q04973, P85410, Q2MGH6, Q7VJ79, P37457, 006052, B1IWA6, P24428, B1MHR6, Q9ABRO, Q07408, P80582, P29230, Q56902, B7MR14, P26926, Q04975, P13415, Q8A2Z5, P57041, B7L953, Q05861, P35272, P33548, J9W7B2, Q8P120, POA5Q3, P31781, Q49536, Q56903, A8ACU7, P94851, Q4L524, 006653, B1V8K7, E6MXWO, Q8XAQ3, P46729, B7NF99, POA2U4, AOAOH3MGR5, Q9AHT6, P9WNF5, P37779, Q49537, Q2FE11, B7M5E0, Q83ES6, B7HU46, P9W141, 030405, Q52657, Q8FBP9, P46730, Q1R4E2, Q326Z6, AOAOH3M3S8, Q99ZN9, P42364, P61887, Q49538, Q2FVC1, A7MQI6, P9WNB1, A9VSQ8, P9WHZ3, Q9A9H3, Q08137, P56258, P46731, B2VG51, Q03945, P9WK44, PODC90, Q9KIJ3, P55254, 085341, Q9ZB73, B7UNB3, P9WNQ7, Q2YWW6, Q6GDU9, Q826C5, P9WP33, B1XAG6, P46732, Q7MYM5, P65645, 007750, Q5XC63, Q9CFZ5, P26393, PODMP7, Q79FB3, A8A6P8, P9WNR7, Q6G167, Q6G613, Q06952, P9WQB0, A9R8J1, P12809, A9MJ16, Q9LAB5, Q49803, Q49588, POA4G4, BORVK9, P26389, Q83AH2, B5YY43, P9WNK3, Q49WE6, Q8NUV4, Q49VP4, Q00488, B5EZ52, P58248, Q57HS3, Q8X8T3, A5U990, P9WGT7, P31305, P44914, Q5JBG6, Q9KW51, B1IWA7, PODOA6, B71W03, P18159, Q2FE05, P13719, A9MJ17, A1KJU9, B4TB26, Q51473, P9WK60, P46024, POA4V7, P37760, Q58YV9, Q99RE2, B2K060, P0A567, A7GKYO, Q2YW66, Q99RD4, P0C934, B5RFR1, P21160, B4SZ39, P72138, P24094, P29725, Q49771, POC8S3, Q9HZ76, Q7A3K7, A1AHW4, P54925, A7XOP5, Q5HD61, Q6G6H5, P19993, Q8XAR8, Q50397, Q5PKK8, Q9ZGM1, Q9ZJ31, 031357, P9WIN6, P37778, A8G840, Q8CN38, Q6CZF0, P9WP32, Q2FZP7, P41484, Q3S2Y1, P24093, Q5PKK9, POA4V5, BORVK6, Q83ELO, Q72U69, P0C223, P9WIN7, P37744, Q31UJ1, Q4L9R5, Q8FBP7, P9WNZ7, A6QFM7, P9WIM6, P77293, P43313, B5EZ54, POC2T1, Q329X2, Q7A3J9, T2GP47, Q45011, Q5XDI6, P55253, Q329X1, Q9ZCX6, Q7MYM4, POC1D7, Q99V75, P9WIM7, Q9JRN5, 068770, B5RFQ9, Q2YKY9, Q83IX6, Q9CCP6, P9WK74, Q83CY8, Q8P280, POC7J4, Q83PH8, Q7DBF7, B1JPF1, P0A647, Q6GARO, Q50703, Q51366, P9WNB3, B4TB27, P19361, P29229, A5U2B3, POA5J1, POA3U8, POA4G2, P55257, A9MXG7, Q79FU3, B4TB25, P40136, Q3S2Y2, Q6FFS6, Q56598, Q93GW2, B7MR15, P14062, P21875, Q01411, AOAOH3M9Z0, P17953, P9WNF3, P27830, B7UNB1, Q5HLD2, C6DHE2, Q9ZKJ5, Q4L8Y7, P9WIB5, Q56623, P19421, C4ZZ61, P9WQP2, Q45206, 085342, P31502, P25393, P0A669, 066251, C0Q3A8, Q49WH7, B4TNU5, P9WPS9, Q9ZH99, Q9A1S2, Q57301, P35635, B1JPZ8, Q06947, Q45209, P37753, P9WK65, P9WPE4, 052957, Q52938, A7ZTZ8, P9WIB4, Q8ZAE3, Q06972, A9WNAO, Q56127, Q06953, 088005, P37458, P31951, Q8FBQ3, P62586, AOQWU8, P15599, Q05868, P18481, B2TTZ8, Q79FW5, POAAA7, Q06981, Q48485, PODMDO, P37747, G4WJD3, Q1CBP1, A5U3Q3, A1JI79, P42216, P9WK75, 034194, P9WQN6, Q9JRN7, Q31UI9, P9WJQ1, A7MQI7, P15488, Q48481, P0DC91, P37785, P19422, B2K059, Q05862, A7FD60, P42217, POAOVO, P25734, P9WQN7, 083346, Q329X3, P9WJY7, A7ZTZ7, P11933, Q5L7M8, Q8DPY9, P68667, P60533, Q8ZAF1, 052956, Q8ZAF0, P0C7U7, P46733, POA5P3, P0A619, Q83DJ3, A1AHW2, POA5P9, Q8XAQ5, P04953, Q56872, Q2YKV1, Q2G1T6, 034191, Q6G2B4, P9WQN8, B7L8D5, A9R9H4, P9WK55, POA5N9, P9WG65, Q5HLV2, QOSYY5, P9W101, B5YY42, P19528, Q7BJX9, Q53107, Q2YW63, D3QY10, Q6G2B3, A1KQD8, B3FN88, A4TSQ1, P42218, P15598, P08180, Q4UNEO, Q1CBP0, P9WHW9, B5RFR0, Q00045, Q89ZXO, Q00474, Q8NUU9, P26879, Q9R3F2, Q83CP9, Q8Z397, Q9X6BO, Q7BHI8, P23033, Q03155, Q9AJ83, B614D0, POA5Q7, B2TTZ9, Q00046, A4Q8F7, P61711, Q45208, POA521, Q6FYW8, P94217, A8G842, P69957, P18010, P26194, 051401, Q52658, Q8ZAF2, Q81U45, Q31UJ0, P57039, Q8X7P7, P26397, Q47592, P9WPE6, P46815, P38016, B1XAG4, P69958, POCAX7, 032606, P58827, Q9AJ37, B7LU63, 033075, Q3YVG9, Q83E37, 031314, P26398, Q2SYH7, Q05433, AOR006, Q83E09, Q83IX5, P21207, P9WIP0, P9WIR0, P80069, Q9AJ79, QOTAS9, P9WIM8, P26880, P02970, 030511, P14168, G3XD01, P24251, B4TNU7, Q8VQ99, C6DHE4, P23995, P9WIP1, P9WIR1, P46842, Q9AJ77, Q8Z386, P9WIM9, P26881, P14190, C5WO22, P26402, P27829, POABW7, B7NTE9, P31631, Q66G05, P69960, POA5Q5, P9WIR3, P9WIR6, Q9AJ80, A9MJ15, P0C873, Q7ARC3, P14191, P18009, Q99191, P27832, P9WPH9, C4ZZ60, Q7A423, P27836, P15917, B2RHG2, P25732, Q48919, Q9AJ82, B5FN88, P68588, P37919, Q6E7F2, D2AJUO, P26400, B2K058, P9WJD8, B7MH59, Q6GED5, B7NFAO, P42502, B2RHG4, P9WIR2, Q9F9L1, Q9AJ63, Q5PKK7, P69966, P9WIZ7, P21982, A6QK59, Q50862, Q8XAQ8, P9WNK7, P45341, Q2FV55, POAC80, P23994, P9WIN8, P80200, Q9ZJD1, Q92JP8, Q8DVU8, POA5Y3, Q68X15, P52616, P99160, Q03584, B5YY41, POA565, QOTAT1, Q9KJT6, Q077R2, P42213, Q83A83, P55746, P56876, Q4UN15, Q1RJI4, P9WGT6, Q8YD01, P13253, P60158, Q83DP8, B7M5E1, P9WNK6, Q3YVG8, Q97PA9, Q57HS4, Q47334, Q03490, P16626, P37915, Q9ZD49, Q92ID4, Q81TU1, P9WKW3, P11764, Q4L980, Q56128, B1XAG5, Q6E7F1, B7NTE7, Q83AY0, A4WG18, Q8X8T4, P46841, P60532, Q04971, Q9AJ81, Q4UM04, P69965, P44067, 051941, Q5HCQ9, P26401, Q0TAT0, P9WIQ7, B7N288, P36429, B4SZ38, Q9S5G5, Q7MXKO, P07889, B9J2U2, Q92JF7, P13422, P31522, Q83CZ8, Q06969, Q7A2K6, Q48475, A4TRB5, A0A0H2VDN9, B7NF98, Q53782, A9MXG5, Q01410, Q48899, P09239, Q6HNU4, Q3L8P3, Q83DN9, P68589, P34001, Q06973, Q79ZW0, Q03583, Q66G06, P9WNK2, Q1R4E3, Q7A2K8, Q6CZF2, Q9ZN40, 085343, P42384, Q5HH69, POCl U6, P0AB36, P31527, Q48456, P05431, P18013, P26396, Q05032, P51836, Q9CNG8, Q99RX4, Q8Z388, P65301, A0Q4N6, Q00330, P76372, POA5P5, A4TRB4, P0A519, and P37741;
P87020, P43588, Q9UQY2, A6ZTR3, C5P230, Q1E3R8, B3STN5, Q4WMJ7, Q03392, Q08723, 013834, 094275, P20967, B2ZRS9, Q9XZV1, E9RCR4, P15873, Q9P7S2, P53549, E9CX44, 074531, P53119, Q95WA3, Q4WMJ1, POCB51, Q12377, P33298, P46589, 074783, P40985, Q95WA4, Q4WMJ9, Q59L12, P38204, 042931, Q1E8D2, Q09855, P24814, P00016, Q4WMJ8, G1UB63, Q03280, P41836, 074623, P87060, A4GYZO, P39940, Q4WMJ0, Q5AOX8, Q9UT05, 014250, 060022, Q5AB48, E9R9Y3, POCH07, Q9P3U4, Q59XX2, POCX83, 059770, Q9USX1, Q5F2J0, P46984, POCH09, P38199, P50142, 074440, Q10329, Q00746, 060182, Q4WMJ5, P38764, Q9USS7, Q00022, Q9US13, Q9UT97, P06104, Q5ABZ2, Q92211, P40016, Q1K9C4, Q03834, Q04062, P38624, 000103, POC7N7, Q04638, P38766, 060106, P25847, P87048, P25451, 000102, 074111, Q9US46, P35178, P38109, P25336, POCH06, P23724, P21734, Q8X082, Q5ALO3, P05759, Q10435, P32454, POCH08, Q12417, P52492, Q3E833, P40034, Q06103, P38202, P36049, QOC9L7, P21242, P15731, 014170, P36132, P53953, 014099, P53874, QOC9L6, Q9USQ9, POCG72, P25375, P38203, P38200, P34247, 042721, Q05583, P50086, Q08562, 094579, 074349, P52286, P38201, POCW95, P85437, P40303, Q09765, Q09682, Q04781, Q9Y709, 094264, Q12018, Q03071, 043063, P23566, 094393, P35728, P40482, Q10311, P38205, F2Z266, P25043, P38820, P40555, C5P3X6, P32565, 074549, 013959, Q5AMT2, Q01939, POCG63, Q92462, P53152, Q12250, 013685, P38630, 043069, P40302, 042646, Q09841, P29469, P38886, P22515, P38629, P38862, P30657, Q02159, P30656, P39014, P41878, P46595, Q08273, P53323, 014126, 094609, P33297, P33310, P50524, P33296, P23639, Q01532, P36612, P52490, P40327, P33311, P32496, Q99344, P32379, P07267, P23638, Q9P7R4, P33299, Q2UNX8, P20606, P52491, 074445, P00729, P18239, P28263, 094517, Q8TGEO, 094672, 074810, 074894, 013807, P37898, 013731, P21243, P36113, 074873, P15732, Q9UTG2, QOC9L4, 074762, P19812, Q09720, POCW94, Q9UUl5, Q07963, 014326, QOC9L5, 042897, P33202, P22141, O74983, P15303, P54860, 075004, 013790, Q10335, 060152, P30655, Q8TG42, POC8R3, Q5AJCO, Q03705, P15646, P40825, Q9C1X4, Q9Y7T8, Q9HFP8, Q01475, Q9UTN8, O94444, and Q9Y818;
A0A074T1L1, A0A0S4KF97, A0A0S4JQZ4, A0A0S4JAS1, A0A0S4J2H8, A0A0S4J8R3, A0A0S4IP92, A0A0S4IVI6, A0A074SMM4, A0A0S4JU95, A0A0S4JUU2, A0A0S4J2G0, A0A0S4JKP3, A0A0S4JNU2, A0A0S4J4S8, A0A0S4J1D6, A0A0S4JJ69, A0A0S4JQW3, A0A0S4KGR9, A0A0S4JOSO, A0A0S4IJQ6, A0A0S4J021, A0A0S4JLK8, A0A0S4JET1, A0A0S4IM54, A0A0S4JTM6, A0A0S4JEP5, A0A0S4IXR8, A0A0S4J1A6, A0A0S4IT85, A0A0S4JB95, A0A0S4IP11, A0A0S4JG58, A0A0S4J014, A0A0S4JAG3, A0A0S4JBV9, A0A0S4IMDO, A0A0S4KGC5, A0A0S4IKB9, A0A0S4JMF9, A0A0S4JJX3, A0A0S4IV8, A0A074TOF7, A0A0S4KMS7, A0A0S4IQ67, A0A0S4JAF7, A0A0S4J3C2, A0A0S4J897, A0A0S4J998, A0A0S4JAD5, A0A074SUM1, A0A0S4ITE7, A0A0S4IJN2, A0A0S4KHF9, A0A0S4J6W7, A0A0S4J872, A0A0S4JS05, A0A0S4JQZO, A0A074TVK6, B6DTN7, A0A0S4KLY4, A0A0S4JS11, A0A0S4J353, A0A0S4IXE4, A0A0S4J7G3, A0A0S4J4W4, A0A074TOB9, A0A0S4IXG7, A0A0S4IVU4, A0A0S4JB29, A0A0S4IIQ2, A0A0S4JB24, A0A0S4J5S1, A0A0S4IZD9, A0A074T0J3, A0A0S4JL29, A0A0S4IR72, A0A0S4JB58, A0A0S4JB16, A0A0S4IL58, A0A074STGO, A0A0S4JJQ5, A0A074T2W9, A0A0S4JBM4, A0A0S4IXS4, A0A0S4JXN2, A0A0S4ILC9, A0A0S4IW93, A0A0S4JIN3, A0A0S4KK21, A0A074TOJ7, A0A0S4JD35, A0A0S4IS00, A0A0S4JBS2, A0A0S4J5K3, A0A0S4J654, A0A0S4IJSO, A0A0S4J299, A0A074TCC9, A0A0S4J4B4, A0A0S4JON5, A0A0S4IRK3, A0A0S4JNX5, A0A0S4J9S4, A0A0S4JUB6, A0A0S4JDR2, A0A074T113, A0A0S4J954, A0A0S4KLL4, A0A0S4KEG2, A0A0S4J8T9, A0A0S4JJVO, A0A0S4JF82, A0A0S4J7H1, A0A074TWU5, A0A0S4JF41, A0A0S4JFAO, A0A0S4JS89, A0A0S4JE28, A0A0S4KFY6, A0A0S4J5C1, A0A0S4J603, A0A074T241, A0A0S4IL47, A0A0S4J985, A0A0S4IT79, A0A0S4JHS5, A0A0S4JCG7, A0A0S4KQ64, A0A0S4JQ27, A0A074SL18, A0A0S4J840, A0A0S4JIC5, A0A0S4KEN7, A0A0S4IPW7, A0A0S4IR61, A0A0S4IS67, A0A0S4IMD6, A0A074SV31, A0A0S4JEJ4, A0A0S4KIF1, A0A0S4JD68, A0A0S4JLCO, A0A0S4JTS4, A0A0S4JNW2, A0A0S4JRC8, A0A074SKW9, A0A0S4IZX6, A0A0S4JHEO, A0A0S4JT29, A0A0S4JSD2, A0A0S4J100, A0A0S4J3U1, A0A0S4IMY9, A0A0S4JMU5, A0A0S4J9N3, A0A0S4IVH1, A0A0S4JQW5, A0A0S4IS12, A0A0S4JAQ6, A0A0S4JC49, A0A0S4JDY8, A0A0S4JN05, A0A0S4ILK6, A0A0S4JHG5, A0A0S4KGV4, A0A0S4J515, A0A0S4IQA7, A0A0S4IV27, A0A0S4JAM9, A0A0S4JJG5, A0A0S4JQB6, A0A0S4JLH4, A0A0S4IVN7, A0A0S4JEZ5, A0A0S4IM08, A0A0S4IH88, A0A0S4JLB2, A0A0S4JEE7, A0A0S4IVX6, A0A0S4IU55, A0A0S4IN83, A0A0S4IZ03, A0A0S4IVV6, A0A0S4KEL3, A0A0S4ISG8, A0A0S4JV10, A0A0S4IIK7, A0A0S4JC54, A0A0S4IKB3, A0A0S4J7S2, A0A0S4JD74, A0A0S4JQ06, A0A0S4J746, A0A0S4JA77, A0A0S4JF29, A0A0S4JAE3, A0A0S4JFY5, A0A0S4JMQ7, A0A0S4J7Q5, A0A0S4J1U3, A0A0S4KK93, A0A0S4IHU9, A0A0S4J2L4, A0A0S4ITD5, A0A0S4JTQ7, A0A0S4JP32, A0A0S4IWS5, A0A0S4JB17, A0A0S4ILT2, A0A0S4JLL8, A0A0S4JDS1, A0A0S4JBL4, A0A0S4JU85, A0A0S4JRD7, A0A0S4J2K2, A0A0S4IU91, A0A0S4IXT3, A0A0S4KJT6, A0A0S4JFC9, A0A0S4JNK9, A0A0S4JV99, A0A0S4JVN7, A0A0S4J113, A0A0S4JAQ3, A0A0S4IXN5, A0A0S4ILX2, A0A0S4IQE4, A0A0S4KF94, A0A0S4JEZ2, A0A0S4IK14, A0A0S4KEC2, A0A0S4JE78, A0A0S4IND6, A0A0S4J3T7, A0A0S4JTK3, A0A0S4JJFO, A0A0S4IMBO, A0A0S4JL12, A0A0S4IQP3, A0A0S4JV52, A0A0S4ITN5, A0A0S4J2M0, A0A0S4IX99, A0A0S4J6B1, A0A0S4JOU7, A0A0S4IMZO, A0A0S4J7H0, A0A0S4JF05, A0A0S4JGN7, A0A0S4KK37, A0A0S4JDTO, A0A0S4JS26, A0A0S4JD14, A0A0S4ITR1, A0A0S4JOH7, A0A0S4J260, A0A0S4JWD6, A0A0S4J8Q8, A0A0S4JE04, A0A0S4JNM8, A0A0S4J7L5, A0A0S4JP43, A0A0S4J3A2, A0A0S4IXY3, A0A0S4J419, A0A0S4JOZO, A0A0S4IY44, A0A0S4JRN5, A0A0S4KHD7, A0A0S4JOFO, A0A0S4J4L7, A0A0S4J539, A0A0S4J3Q1, A0A0S4J542, A0A0S4IL46, A0A0S4KL26, A0A0S4JEG7, A0A0S4JVS2, A0A0S4KP55, A0A0S4J0G9, A0A0S4JJ63, A0A0S4IP14, A0A0S4J8P6, A0A0S4J2R4, A0A0S4J761, A0A0S4IJN8, A0A0S4JSG5, A0A0S4IP99, A0A0S4JHM1, A0A0S4IN27, A0A0S4KEC0, A0A0S4JSC5, A0A0S4J4G2, A0A0S4JAN4, A0A0S4JEP2, A0A0S4J9M5, A0A0S4ISU4, A0A0S4J604, A0A0S4ILR7, A0A0S4JGL5, A0A0S4JOKO, A0A0S4JSB8, A0A0S4JLZO, A0A0S4JOQ7, A0A0S4IVU5, A0A0S4J1U5, A0A0S4IQP4, A0A0S4IT62, A0A0S4JUR5, A0A0S4IU73, A0A0S4J6BO, A0A0S4JCX5, A0A0S4JCT9, A0A0S4J2U8, A0A0S4KJA7, A0A0S4J8U4, A0A0S4JS12, A0A0S4JWB3, A0A0S4ISN8, A0A0S4JDX7, A0A0S4J592, A0A0S4IU40, A0A0S4IJ71, A0A0S4J3B3, A0A0S4JCC6, A0A0S4JAU0, A0A0S4J360, A0A0S4JG36, A0A0S4JNL2, A0A0S4IWB9, A0A0S4J805, A0A0S4JMGO, A0A0S4JG17, A0A0S4J206, A0A0S4J4C3, A0A0S4ILW5, A0A0S4KIB1, A0A0S4JZB5, A0A0S4IR85, A0A0S4IVQ8, A0A0S4JSH3, A0A0S4KIR5, A0A0S4JK76, A0A0S4IZC7, A0A0S4JSA1, A0A0S4JJG7, A0A0S4IMS3, A0A0S4IY39, A0A0S4IWU0, A0A0S4J4P0, A0A0S4JEG9, A0A0S4IW61, A0A0S4ITN3, A0A0S4J2P7, A0A0S4IS4I, A0A0S4KMV2, A0A0S4J278, A0A0S4JVZ3, A0A0S4JU20, A0A0S4JPM5, A0A0S4IRQ2, A0A0S4JTY6, A0A0S4J814, A0A0S4J2V2, A0A0S4IVC7, A0A0S4IZW2, A0A0S4JH12, A0A0S4JU35, A0A0S4J6M1, A0A0S4JMU7, A0A0S4JLM8, A0A0S4JMV1, A0A0S4JEK1, A0A0S4J5X0, A0A0S4JL76, A0A0S4JJI3, A0A0S4JPN6, A0A0S4J254, A0A0S4IQ0, A0A0S4KK75, A0A0S4JEXO, A0A0S4JEH8, A0A0S4IV96, A0A0S4JFV3, A0A0S4IZX5, A0A0S4KIT5, A0A0S4JAW7, A0A0S4IIS0, A0A0S4J5A0, A0A0S4KHE4, and A0A0S4IUK8;
B6KAMO, P90661, Q81E47, P14593, P18269, Q4U9M9, P23253, P13826, P50498, P04934, P13828, P13814, P50490, P19597, P02897, P02890, P09792, Q02752, P18270, P06914, P15714, P08569, P68874, P06016, P08515, Q8MZJ8, P15964, P08676, P22622, P08677, Q25540, P02892, P87020, P42665, P18271, P13815, P20287, 096175, P35661, P08418, P29030, P62884, P67877, Q07828, P50491, C6KTB7, P04926, P02894, Q9TY95, Q5F2J0, Q03400, P08675, Q95WA4, Q9F9F2, P35666, Q9AJ37, Q81E95, P09841, Q95WA3, P04922, P50492, P08672, Q05870, P05691, Q25619, P26332, Q03994, P08673, P19260, P06015, P31008, P17503, P16445, P02893, P67878, P15744, Q9XZV1, P08307, P21849, P32072, P69192, P22545, Q815D2, P08674, P14223, Q4UNEO, P61074, Q4FX73, P22621, P26694, P54190, Q25306, P13399, Q01443, P02898, Q4UM04, P81860, Q81KW2, P26624, P06915, P50489, Q4UN15, Q7KQM2, P02891, B6KV60, Q03110, P21303, P62883, B3STN5, and P23093;
P04156; and

Amyloid β peptide (AR; P05067), Tau (P10636), α-Synuclein (P37840), Fused in sarcoma (FUS) protein (P35637), Superoxide dismutase (P00441), TDP-43 (Q13148), C90RF72 (Q96LT7), ubiquilin-2 (UBQLN2) (Q9UHD9), Huntingtin (P42858), ABri (Q9Y287), Cystatin C (P01034), Notch3 (Q9UM47), Glial fibrillary acidic protein (GFAP; P14136), Seipin (Q96G97), Transthyretin (P02766), Serpins (P01009 & others), immunoglobulin light chains (P01834 & others), Immunoglobulin heavy chains (P01857 & others), Amyloid A protein (PODJI8), Islet amyloid polypeptide (amylin; P10997), Medin (lactadherin; Q08431), Apolipoprotein AI (P02647), Apolipoprotein AII (P02652), Apolipoprotein AIV (P06727), Gelsolin (P06396), Lysozyme (P61626), Fibrinogen (P02671, P02675), Beta-2 microglobulin (P61769), Crystallins (P02489 & others), Rhodopsin (P08100), Calcitonin (P01258), Atrial natriuretic factor (P01160), Prolactin (P01236), Keratoepithelin (Q15582), Keratins (Q14533 & others), Keratin intermediate filament proteins (P35908 & others), Lactoferrin (P02788), Surfactant protein C (SP-C) (P11686), Odontogenic ameloblast-associated protein (A1E959), Semenogelin I (P04279), Apolipoprotein C2 (ApoC2) (P02655), Apolipoprotein C3 (ApoC3) (P02656), Leukocyte chemotactic factor-2 (Lect2) (014960), Insulin (P01308), Galectin-7 (Gal7) (P47929), Corneodesmosin (Q15517), and CFTR protein (P13569).

22. The method of any one of statements 1 to 21, wherein the subject has, is suspected of having, has been diagnosed with, or is at risk of, a disorder characterized by a disorder-associated antigen (DAA).

23. The method of any one of statements 1 to 22, wherein the subject has been selected for treatment on the basis that the subject has, is suspected of having, has been diagnosed with, or is at risk of, a disorder characterized by a disorder-associated antigen (DAA).

24. The method of any one of statements 1 to 23, comprising the steps of:
(i) selecting the subject for treatment on the basis that the subject has, is suspected of having, has been diagnosed with, or is at risk of, a disorder characterized by a disorder-associated antigen (DAA);
(ii) administering the CD25-ADC to the subject.

25. The method of any one of statements 6 to 24, wherein the disorder is a pathogen-associated disorder, or a proliferative disorder such as cancer.

26. The method of statement 25, wherein the proliferative disorder or cancer is a solid tumour.

27. The method of statement 26, wherein the solid tumour is an established tumour.

28. The method of statement 27, wherein the establised tumour is a tumour diagnosed or identified in a naïve subject.

29. The method of statement 27, wherein the establised tumour is a relapsed tumour.

30. The method of statement 26, wherein the solid tumour is a metastatic tumour.

31. The method of statement 26, wherein the solid tumour comprises or consists of CD25-ve neoplastic cells.

32. The method of any one of statements 25 to 31, wherein the solid tumour is associated with CD25+ve infiltrating cells;
optionally wherein the solid tumour is associated with high levels of CD25+ve infiltrating cells.

33. The method of statement 32, wherein the solid tumour is selected from the group consisting of pancreatic cancer, breast cancer (including triple negative breast cancer), colorectal cancer, gastric and oesophageal cancer, melanoma, non-small cell lung cancer, ovarian cancer, hepatocellular carcinoma, renal cell carcinoma, bladder, and head and neck cancer.

34. The method of statement 31, wherein the solid tumour is associated with low levels of CD25+ve infiltrating cells.

35. The method of any one of statements 25 to 31, wherein the solid tumour is not associated with CD25+ve infiltrating cells.

36. The method of statement 25, wherein the proliferative disorder or cancer is lymphoma or leukaemia.

37. The method of statement 36, wherein the proliferative disorder or cancer is selected from:
Hodgkin's Lymphoma;
non-Hodgkin's, including diffuse large B-cell lymphoma (DLBCL), follicular lymphoma, (FL), Mantle Cell lymphoma (MCL), chronic lymphatic lymphoma (CLL) Marginal Zone B-cell lymphoma (MZBL); and
leukemias, including Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph-ALL).

38. The method of any one of statements 25, 36, or 37, wherein the proliferative disorder or cancer is associated with elevated levels of regulatory immune cells, such as Treg cells.

39. The method of statements 38, wherein the proliferative disorder or cancer is chronic lymphatic lymphoma (CLL), T-cell Acute Lymphoblastic Leukaemia (T-ALL), and B-cell non-Hodgkin's Lymphoma.

40. The method any one of statements 36 to 39, wherein the proliferative disorder or cancer is AML.

41. The method of statement 25, wherein the proliferative disorder or cancer is a circulating tumour or circulating tumour cells.

42. The method of statement 41, wherein the circulating tumour or circulating tumour cells are, or comprise, metastatic cells.

43. The method of either one of statements 41 or 42, wherein the subjects are:
a) subjects who have, are suspected of having, have been diagnosed with a primary tumour with metastatic characteristics, such as high-metastatic prognosis or elevated expression of one or more biomarkers of metastatic cancer;
b) subjects who have, are suspected of having, have been diagnosed with one or more metastatic tumours;
c) pre-operative or post-operative subjects, wherein the operation is to remove part or all of a solid tumour. Typically, selected pre-operative or post-operative subjects start their treatment not more than 4 weeks from the operation date, such as not more than 2 weeks, or not more than 1 week.

44. The method of statement 25, wherein the pathogen-associated disorder is viral, bacterial, fungal, protozoan, parasitical, prion, or protein aggregate.

45. The method of either one of statement 25 or 44, wherein the pathogen-associated disorder is selected from:
Adenovirus infection, HIV/AIDS, Alphavirus Encephalitis, Arenavirus, Argentine hemorrhagic fever, Arthropod-borne viral encephalitis, Avian Influenza, Bolivian Hemorrhagic Fever, Borna disease, Chickenpox, Chikungunya, Coxsackievirus Infection, Crimean-Congo Hemorrhagic Fever, Cytomegalovirus infection, Dengue fever, Eastern equine encephalitis, Ebola, Echovirus Infection, Epstein-Barr Virus infection, Epstein-Barr Virus Related Tumours, Fifth Disease, Filovirus, Flavivirus, German measles, Hand, foot & mouth disease, Hemorrhagic fever with renal syndrome, Herpes Virus (Herpesviridae) infection, Herpes Simplex Virus Infection, Herpes Zoster Virus, Human Papilloma Virus Associated Epidermal Lesions, Human Papilloma Virus related Cervical Cancer, Mononucleosis, Influenza, Japanese Encephalitis, Kaposi Sarcoma, Korean Hemorrhagic Fever, Kyasanur Forest Disease, Lassa Fever, Lymphocytic choriomeningitis, Marburg Virus Disease, Measles, Molluscum Contagiosum, Mumps, Murray Valley encephalitis, Norwalk Virus related Diarrhea, Omsk hemorrhagic fever, Orthomyxoviruses, Parainfluenza Virus Infection, Paramyxovirus, Parvovirus B19 Infection, Picornavirus, Poxviruses, Rabies, Respiratory syncytial virus infection, Rift Valley Fever, Rotavirus diarrhea, Rubella, Rubeola, Smallpox, St. Louis Encephalitis, Tick-borne Encephalitis, Varicella, Variola, Venezuelan equine encephalitis, Viral hemorrhagic fevers, Western equine encephalitis, West Nile Virus disease, Yellow Fever, and Zika;
Actinomycosis, Acute prostatitis, Anaerobic infection, Bacillary peliosis, Bacteremia, Bacterial pneumonia, Bacteroides ureolyticus, Baggio-Yoshinari syndrome, Barcoo fever, Bartonellosis, Biliary fever, Botryomycosis, Bovine campylobacteriosis, Brazilian purpuric fever, Brazilian Purpuric Fever, Brodie abscess, Burkholderia cepacia complex, Buruli ulcer, Campylobacteriosis, Capnocytophaga canimorsus, Cariogram, Carrion's disease, Chlamydia suis, Cholera, bacterial prostatitis, osteomyelitis, endodontic lesions, bovine pleuropneumonia, dermatitis, Diphtheria, Diphtheritic stomatitis, Epidural abscess, Epiglottitis, Erysipelas, Legionaires' disease, Far East scarlet-like fever, Fitz-Hugh-Curtis syndrome, Foot rot, Gardnerella vaginalis, Garre's sclerosing osteomyelitis, Granuloma inguinale, Haemophilus meningitis, monocytotropic ehrlichiosis, Lemierre's syndrome, Leprosy, Listeriosis, Lyme disease, Meningococcal disease, Mycobacterium avium-intracellulare infection, Necrotizing fasciitis, Nocardiosis, Noma, Omphalitis, Orbital cellulitis, Periodontal abscess, Periorbital cellulitis, Peritonsillar abscess, Pott disease, Proctitis, Pseudomonas infection, Psittacosis, Pyaemia, Pyomyositis, Q fever, Retropharyngeal abscess, Salmonellosis, Serratia infection, Shigellosis, Southern tick-associated rash illness, Staphylococcal scalded skin syndrome, brucellosis, Syphilis, Tetanus, Toxic shock syndrome, Trench fever, Tropical ulcer, Tubo-ovarian abscess, Ureaplasma urealyticum infection, Tuberculosis, Vertebral osteomyelitis, Waterhouse-Friderichsen syndrome, Whooping cough, Xanthogranulomatous osteomyelitis, and Yersiniosis;
Aspergillosis, Candidiasis, Coccidioidomycosis, Cryptococcus gattil infection, onychomycosis, microsporidiosis, mucormycosis, pneumocystis pneumonia, sporotrichosis, blastomycosis, Candida auris infection, Cryptococcus neoformans infection, fungal eye infection including keratitis and endophthalmitis, Histoplasmosis, mycetoma, dermatophytosis inc. tinea pedis tinea corporis, and talaromycosis;
Acanthamoeba keratitis, African trypanosomiasis, Avian malaria, Babesiosis, Besnoitiosis, Blastocystosis, Chagas disease, Cryptosporidiosis, Cyclosporiasis, Dientamoebiasis, Giardiasis, Histomoniasis, Malaria, Premunity, Sappinia amoebic encephalitis, Surra, Toxoplasmosis, Trichomoniasis, and Trypanosomiasis;
Echinococcosis, Amebiasis, Ancylostomiasis, Angiostrongyliasis, Anisakiasis, Ascariasis, Balantidiasis, Granulomatous Amebic Encephalitis, Baylisascariasis, Schistosomiasis, Capillariasis, Clonorchiasis, Cysticercosis, Diphyllobothriasis, Filariasis, Enterobiasis, Fascioliasis, Fasciolopsiasis, gnathostomiasis, Heterophyiasis, Hymenolepiasis, Leishmaniasis, Opisthorchiasis, Loiasis, Onchocerciasis, Paragonimiasis, Sarcocystosis, Taeniasis, Toxocariasis, Trichinellosis, and Trichuriasis;
Creutzfeldt-Jakob disease (CJD) including iatrogenic, variant, familial, and sporadic sub-types, fatal familial insomnia (FFI), Gertsmann-Straussler-Scheinker syndrome (GSS), kuru, and variably protease-sensitive prionopathy (VPSPr);
Alzheimer's disease, Cerebral p-amyloid angiopathy, Retinal ganglion cell degeneration in glaucoma, Parkinson's disease and other synucleinopathies, Tauopathies, Frontotemporal lobar degeneration, FTLD-FUS, Amyotrophic lateral sclerosis (ALS), Huntington's disease, Familial British dementia, Familial Danish dementia, Hereditary cerebral hemorrhage with amyloidosis, CADASIL, Alexander disease, Seipinopathies, Familial amyloidotic neuropathy, Senile systemic amyloidosis, Serpinopathies, AL amyloidosis, AH amyloidosis, AA amyloidosis, Type II diabetes, Aortic medial amyloidosis, ApoAl amyloidosis, ApoAll amyloidosis, ApoAIV amyloidosis, Familial amyloidosis of the Finnish type (FAF), Lysozyme amyloidosis, Fibrinogen amyloidosis, Dialysis amyloidosis, Inclusion body myositis/myopathy, Cat Retinitis pigmentosa with rhodopsin mutations, Medullary thyroid carcinoma, Cardiac atrial amyloidosis, Pituitary prolactinoma, Hereditary lattice corneal dystrophy, Cutaneous lichen amyloidosis, Mallory bodies, Multiple System Atrophy, Corneal lactoferrin amyloidosis, Pulmonary alveolar proteinosis, Odontogenic (Pindborg) tumor amyloid, Seminal vesicle amyloid, Apolipoprotein C2 amyloidosis, Apolipoprotein C3 amyloidosis, Lect2 amyloidosis, Insulin amyloidosis, Galectin-7 amyloidosis (primary localized cutaneous amyloidosis), Corneodesmosin amyloidosis, Enfuvirtide amyloidosis, Cystic fibrosis, and Sickle cell disease.

46. The method of any one of statements 1 to 45, wherein the subject has received an adoptive cell transfer.

47. The method of any one of statements 1 to 46, wherein the subject has been selected for treatment on the basis that the subject has received an adoptive cell transfer.

48. The method of any one of statements 1 to 47, comprising the steps of:
(i) selecting the subject for treatment on the basis that the subject has received an adoptive cell transfer;
(ii) administering the CD25-ADC to the subject.

49. The method of any one of statements 46 to 48, wherein the adoptive cell transfer is a bone marrow transplant.

50. The method of any one of statements 46 to 49, wherein the adoptive cell transfer is an autologous cell transfer.

51. The method of any one of statements 46 to 49, wherein the adoptive cell transfer is an allogenic cell transfer.

52. The method of any one of statements 46 to 51, wherein the adoptive cell transfer is a stem cell transfer.

53. The method of any one of statements 46 to 52, wherein the adoptive cell transfer is an immune cell transfer.

54. The method of any one of statements 46 to 53, wherein the subject received the adoptive cell transfer at least 3 months prior to the administration of the CD25-ADC, such as at least 6 months, at least 12 months, at least 18 months, or at least 24 months prior to the administration of the CD25-ADC.

55. The method of any one of statements 46 to 54, wherein the CD25-ADC is administered in a QW dosage regime.

56. The method of any one of statements 46 to 55, wherein the dose of CD25-ADC administered is 10, 20, 25, 27.5, 30, 32.5, 35, 37.5, 40, 42.5, 45, 47.5, 50, 55, 60, 65, 70, 75, or 80 μg/kg.

57. The method of any one of statements 1 to 7 or 22 to 56, wherein the CD25-ADC is administered in combination with a cell therapy.

58. The method of statement 57, wherein the CD25-ADC is administered before the cell therapy;
ptionally wherein the CD25-ADC is administered 1 day, 2 days, 3 days, 4 days, 5 days, 6 days, 7 days, 8 days, 9 days, 10 days, 11 days, 12 days, 13 days, or 14 days before the cell therapy.

59. The method of either one of statements 57 or 58, wherein the immune-suppressive activity of a population of regulatory immune cells in the subject is reduced by at least 90% before the cell therapy is administered.

60. The method of any one of statements 57 to 59, wherein the size of a population of regulatory immune cells in the subject is reduced by at least 90% before the cell therapy is administered.

61. The method of statement 60, wherein the regulatory immune cells are Treg cells.

62. The method of statement 57, wherein the CD25-ADC is administered concomitantly with the cell therapy.

63. The method of any one of statements 57 to 62, wherein the cell therapy comprises the administration of autologous cells.

64. The method of any one of statements 57 to 62, wherein the cell therapy comprises the administration of allogenic cells.

65. The method of any one of statements 57 to 64, wherein the cell therapy comprises the administration of stem cells.

66. The method of any one of statements 57 to 65, wherein the cell therapy comprises the administration of immune cells.

67. The method of statement 66, wherein the immune cells are T-cells, Natural Killer (NK) cells, Natural Killer T-cell (NKT), Lymphokine-activated Killer (LAK) cells, or macrophages.

68. The method of either one of statements 63 or 67, wherein the immune cells express a chimeric antigen receptor (CAR).

69. The method of any one of statements 57 to 68, wherein the cell therapy comprises the administration of CAR T-cells.

70. The method of statement 69, wherein the CAR T-cell is a 1^{st} generation CAR T-cell, a 2^{nd} generation CAR T-cell, a 3^{rd} generation CAR T-cell, a 4^{th} generation CAR T-cell, a TRUCK, a smart CAR, or an iCAR.

71. A method of treating a disorder a subject, wherein the disorder is characterized by a disorder-associated antigen (DAA), the method comprising administering a CD25-ADC to the subject, wherein the subject:
(i) has received an adoptive cell transfer; or
(ii) has been selected for treatment on the basis they have received an adoptive cell transfer.

72. The method of statement 71 further comprising the step of selecting the subject for treatment on the basis that the subject has received an adoptive cell transfer.

73. The method of either one of statement 71 and 72, wherein the disorder is as defined in any one of statements 24 to 37.

74. The method of any one of statements 71 to 73, wherein the adoptive cell transfer is as defined in any one of statements 41 to 45.

75. The method of any one of statements 71 to 74, wherein the subject received the adoptive cell transfer at least 3 months prior to the administration of the CD25-ADC, such as at least 6 months, at least 12 months, at least 18 months, or at least 24 months prior to the administration of the CD25-ADC.

76. The method of any one of statements 71 to75, wherein the CD25-ADC is administered in a QW dosage regime.

77. The method of any one of statements 71 to 76, wherein the dose of CD25-ADC administered is 10, 20, 25, 27.5, 30, 32.5, 35, 37.5, 40, 42.5, 45, 47.5, 50, 55, 60, 65, 70, 75, or 80 μg/kg.

78. A method of enhancing the efficacy of cell therapy in a subject, the method comprising administering the cell therapy in combination with a CD25-ADC.

79. A method of treating a disorder a subject, wherein the disorder is characterized by a disorder-associated antigen (DAA), the method comprising administering a CD25-ADC to the subject in combination with cell therapy.

80. The method of statement 79, wherein the disorder is as defined in any one of statements 25 to 45.

81. The method of any one of statements 78 to 80, wherein the CD25-ADC is administered before the cell therapy.

82. The method of any one of statements 78 to 81, wherein the immune-suppressive activity of a population of regulatory immune cells in the subject is reduced by at least 90% before the cell therapy is administered.

83. The method of any one of statements 78 to 82, wherein the size of a population of regulatory immune cells in the subject is reduced by at least 90% before the DAA or vaccine composition is administered.

84. The method of statement 83, wherein the regulatory immune cells are Treg cells.

85. The method of any one of statements 78 to 80, wherein the CD25-ADC is administered concomitantly with the cell therapy.

86. The method of any one of statements 78 to 85, wherein the cell therapy is as defined in any one of statements 63 to 70.

87. A method of selecting a subject as suitable for treatment with a CD25-ADC, the method comprising the steps of:
(a) identifying a subject who:
   (i) has, is suspected of having, has been diagnosed with, or is at risk of, a disorder characterized by a disorder-associated antigen (DAA), and/or
   (ii) has received an adoptive cell transfer, and/or
   (iii) has been selected for treatment with cell therapy; and
(b) selecting the subject for treatment with the CD25-ADC.

88. The method of statement 87, wherein the disorder is as defined in any one of statements 25 to 46.

89. The method of either one of statements 87 or 88, wherein the adoptive cell transfer is as defined in any one of statements 49 to 53.

90. The method of any one of statements 87 to 89, wherein the subject received the adoptive cell transfer at least 3 months prior to the administration of the CD25-ADC, such as at least 6 months, at least 12 months, at least 18 months, or at least 24 months prior to the administration of the CD25-ADC.

91. The method of any one of statements 87 to 90, wherein the cell therapy is as defined in any one of statements 63 to 70.

92. The method of any one of statements 1 to 91, wherein the CD25-ADC is administered in combination with a checkpoint inhibitor or other immunostimulatory agent.

93. The method of statement 92, wherein the CD25-ADC may be administered before the checkpoint inhibitor or other immunostimulatory agent, simultaneous with the checkpoint inhibitor or other immunostimulatory agent, or after the checkpoint inhibitor or other immunostimulatory agent.

94. The method of either one of paragraphs 92 or 93, wherein the checkpoint inhibitor is a PD1 antagonist.

95. The method of paragraph 94, wherein the PD1 antagonist is selected from pembrolizumab, nivolumab, MED10680, PDR001 (spartalizumab), Camrelizumab, AUNP12, Pidilizumab Cemiplimab (REGN-2810), AMP-224, BGB-A317 (Tisleizumab), and BGB-108.

96. The method of either one of paragraphs 92 or 93, wherein the checkpoint inhibitor is a PD-L1 antagonist.

97. The method of paragraph 96, wherein the PD-L1 antagonist is selected from atezolizumab (Tecentriq), BMS-936559/MDX-1105, durvalumab/MED14736, and MSB0010718C (Avelumab).

98. The method of either one of paragraphs 92 or 93, wherein the checkpoint inhibitor is a GITR (Glucocorticoid-Induced TNFR-Related protein) agonist.

99. The method of paragraph 98, wherein the GITR (Glucocorticoid-Induced TNFR-Related protein) agonist is selected from MED11873, TRX518, GWN323, MK-1248, MK 4166, BMS-986156 and INCAGN1876.

100. The method of either one of paragraphs 92 or 93, wherein the checkpoint inhibitor is an OX40 agonist.

101. The method of paragraph 100, wherein the OX40 agonist is selected from MED10562, MED16383, MOXR0916, RG7888, OX40mAb24, INCAGN1949, GSK3174998, and PF-04518600.

102. The method of either one of paragraphs 92 or 93, wherein the checkpoint inhibitor is a CTLA-4 antagonist.

103. The method of paragraph 102, wherein the CTLA-4 antagonist is selected from ipilimumab and Tremelimumab.

104. The method of either one of paragraphs 92 or 93, wherein the other immunostimulatory agent is a bi-specific T-cell engager (BiTE).

105. The method of paragraph 104, wherein the BiTE specifically binds CD3.

106. The method of either one of paragraphs 104 or 105, wherein the BiTE specifically binds the DAA.

107. The method of any one of statements 1 to 106, wherein the CD25-ADC is administered in combination with radiotherapy.

108. The method of statement 107, wherein the CD25-ADC may be administered before the radiotherapy, simultaneous with the radiotherapy, or after the radiotherapy.

109. The method of either one of paragraphs 107 or 108, wherein the radiotherapy is selected from the group consisting of: external beam radiotherapy, stereotactic radiation therapy, Intensity-Modulated Radiation Therapy, particle therapy, brachytherapy, delivery of radioisotopes, intraoperative radiotherapy, Auger therapy, Volumetric modulated arc therapy, Virtual simulation, 3-dimensional conformal radiation therapy, and intensity-modulated radiation therapy.

110. The method of any one of paragraphs 107 to 109, wherein each radiotherapy dose is no greater than 18 Gy.

111. The method of any one of paragraphs 107 to 110, wherein each radiotherapy dose is no greater than 12 Gy.

112. The method of any one of paragraphs 107 to 111, wherein the total radiotherapy dose is no greater than 18 Gy.

113. The method of any one of paragraphs 107 to 112, wherein the total radiotherapy dose is no greater than 12 Gy.

114. The method of any one of statements 1 to 113, wherein the CD25-ADC is as defined herein in paragraphs 1-110 of the section entitled “CD25-ADCs”.

115. The method of any one of statements 1 to 114, wherein the CD25-ADC is ADCx25.

116. The method of any one of statements 1 to 114, wherein the CD25-ADC is ADCT-301.

117. The method of any one of statements 1 to 114, wherein the CD25-ADC is Camidanlumab Tesirine.

118. An antibody-drug conjugate compound as defined in any one of statements 1 to 112 for use in a method of any one of statements 1 to 86 or 92 to 117.

119. A composition or pharmaceutical composition comprising an antibody-drug conjugate compound as defined in any one of statements 1 to 117 for use in a method of any one of statements 1 to 86 or 92 to 117.

120. Use of an antibody-drug conjugate compound as defined in any one of statements 1 to 112 in the preparation of a medicament for use in a method of any one of statements 1 to 86 or 92 to 117.

### EXAMPLES

### Example 1

In Vivo Efficacy Study of Surrogate-ADCx25 in an Immuno-Competent Syngeneic Mouse Model Using Mouse Colon Cancer MC38 Cells.

Introduction

MC38 is a CD25-ve mouse colon cancer-derived model used pre-clinically in immunotherapy-type studies which is known to have infiltration of Treg and Teff cells.

In Arce Vargas et al., 2017, Immunity 46, 1-10, Apr. 18, 2017 (http://dx.doi.org/10.1016/i.immuni.2017.03.013) selective depletion of tumor infiltrating Treg cells in the MC38 model was shown using an Fc enhanced version of PC61, a rat antibody directed against mouse CD25 and synergy with PD1 was described. The wild-type PC61 was conjugated to the PBD dimer drug-linker SG3249 (the PBD drug-linker used in ADCx25/ADCT-301/Camidanlumab Tesirine) and designated as Surrogate-ADCx25 (or SurADCx25). The efficacy of Surrogate-ADCx25 was studied as monotherapy or in combination with anti-PD1 (Anti-PD1, clone RPM1-14, BioXcell cat#BE0146) in the MC38 syngeneic mouse model.

Study Design

Female C57BL/6 mice (C57BL/6NCrl, Charles River) were nine weeks old on Day 1 of the study and had a body weight (BW) range of 17.8 to 24.2 g. At the completion of the initial study described in this example, tumor-free survivors were transferred to a secondary rechallenge study, described in Example 3.

On the day of implant 5×10⁵ MC38 cells (0.1 mL suspension) were subcutaneously implanted into the right flank of each test animal. Tumors were monitored as their volumes approached the target range of 80-120 mm³. Fifteen days after tumor cell implantation, on Day 1 of the study, animals were sorted into ten groups (n=10/group) with individual tumor volumes of 63 to 172 mm3, and group mean tumor volumes of 103-172 mm3.

All doses were administered intraperitoneally (i.p.) on Day 1 except for anti-PD-1 which was administered once on Days 2, 5, 8. The dosing volume was 0.2 mL per 20 grams of body weight (10 mL/kg), and was scaled to the body weight of each individual animal. Tumors were measured twice per week until the study was ended on Day 59. Each animal was euthanized when its tumor attained the endpoint tumor volume of 1000 mm3 or on the final day, whichever came first.

Surrogate-ADCx25 was administered intraperitoneally (i.p.) as single dose (0.1, 0.5 and 1 mg/kg) on day 1 either alone or in combination with anti-PD1 antibody (given at standard dosing regime, i.e. 5 mg/kg at day 2, 5 and 8). As a control, Isotype Control ADC (B12-SG3249) was administered as single dose (1 mg/kg) on day 1 either alone or in combination 45 with anti-PD1 antibody (given at standard dosing regime), while anti-PD1 antibody was administered alone at standard dosing regimen.

Tumors were measured in two dimensions using calipers, and volume was calculated using the formula:

Tumor Volume (mm³)=w²×l/2, where w=width and l=length,in mm, of the tumor.

Tumor weight may be estimated with the assumption that 1 mg is equivalent to 1 mm3 of tumor volume.

Results

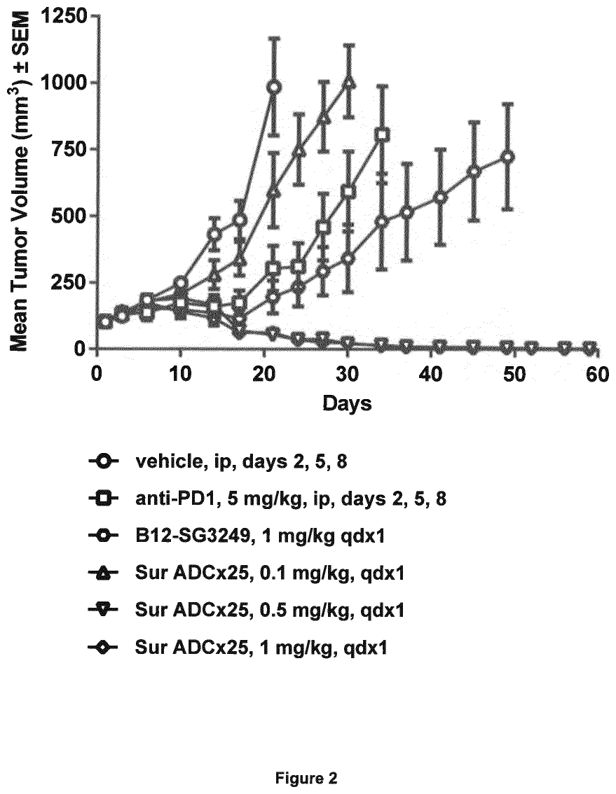

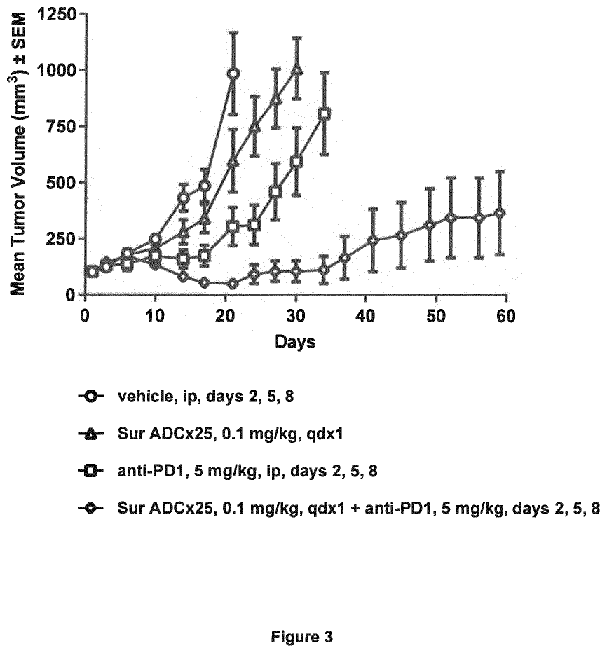

Surrogate-ADCx25 had strong and dose-dependent anti-tumor activity per se in the MC38 syngeneic model. The isotype control ADC had significant lower activity than surrogate-ADCx25 at 1 mg/kg. (FIG. 2). A strong synergy was observed when combining a low single dose of surrogate-ADCx25 with anti-PD1 antibody (FIG. 3). High efficacy of higher doses of surrogate-ADCx25 in the present model prevented assessment of synergy at higher doses.

In vivo, a single dose of sur-ADCx25 at 0.5 or 1 mg/kg induced strong and durable anti-tumor activity against established CD25-negative solid tumors with infiltrating Treg cells (MC38 syngeneic model).

| Response summary | PR | CR | TFS |
|---|---|---|---|
| Vehicle | 0 | 0 | 0 |
| Sur ADCX25, 0.1 mg/kg | 0 | 1 | 1 |
| Sur ADCX25, 0.5 mg/kg | 2 | 8 | 8 |
| Sur ADCX25, 1 mg/kg | 2 | 8 | 8 |
| Anti-PD1, 5 mg/kg | 0 | 3 | 3 |
| B12-SG3249, 1 mg/kg | 2 | 2 | 2 |
| Sur ADCX25, 0.1 mg/kg + | 1 | 6 | 6 |
| anti-PD1 | | | |
| Sur ADCX25, 0.5 mg/kg + | 1 | 9 | 9 |
| anti-PD1 | | | |
| Sur ADCX25, 1 mg/kg + anti-PD1 | 0 | 10 | 10 |
| B12-SG3249, 1 mg/kg + anti-PD1 | 2 | 5 | 5 |

Coefficient of Drug Interaction (CDI)

Sur ADCX25, 0.1 mg/kg;
anti-PD1, 5 mg/kg;
Sur ADCX25, 0.1 mg/kg + anti-PD1
Day 21 0.268 (Synergism)

Response Table Criteria (Also Applicable to Example 4 and Example 6):

Treatment may cause partial regression (PR) or complete regression (CR) of the tumor in an animal.

In a PR response, the tumor volume is 50% or less of its Day 1 volume for three consecutive measurements during the course of the study, and equal to or greater than 13.5 mm³ for one or more of these three measurements.

In a CR response, the tumor volume is less than 13.5 mm³ for three consecutive measurements during the study.

Any animal with a CR response at the end of the study was additionally classified as a tumor-free survivor (TFS).

Animals were scored only once during the study for a PR or CR event and only as CR if both PR and CR criteria were satisfied.

CDI Methodology Also Applicable to Example 4 and Example 6):

The Coefficient of Drug Interaction (CDI) (11) in were assessed for subadditive, additive, or supra-additive (synergism) properties on Day 21, the last day all evaluable animals remained on study.

The CDI was determined according to the equation below:

CDI=AB/AxB

Where,
x=mean tumor volume
AB=xAB/xC
A=xA/xC
B=xB/xC

CDI<1 is supra-additive (i.e. synergism); CDI=1 is additive; CDI>1 is sub-additive

### Example 2: Synopsis of Fractionated Dosage Protocol

Indication

Patients with relapsed or refractory cluster of differentiation 25 (CD25)-positive acute myeloid leukemia (AML) or CD25-positive acute lymphoblastic leukemia (ALL) who have failed, or are intolerant to, any established therapy known to provide clinical benefit at current state of disease. Patients with myelodysplastic syndrome who have received treatment with hypomethylating agents and subsequently present with CD25+ AML and who failed, or are ineligible for standard induction therapy, are eligible for treatment with ADCx25.

Objectives

Primary Objectives:

The primary objectives for Part 1 (dose-escalation) and Part 2 (expansion) of the study are:
Evaluate the safety and tolerability and determine the maximum tolerated dose (MTD) of ADCX25 in patients with CD25-positive relapsed or refractory AML and CD25-positive ALL (Part 1).
Determine the recommended dose of ADCX25 for Part 2.
Evaluate the safety and tolerability of ADCX25 in Part 2 at the dose level recommended in Part 1.

Secondary Objectives:

The secondary objectives for Part 1 and Part 2 of the study are:
Evaluate the clinical activity of ADCX25, based on the patient's response to treatment (complete response [CR], CR with incomplete blood count recover [CRi], partial response [PR], progressive disease [PD], no response [NR]) and determination of the overall duration of response (DOR), overall response rate (ORR), overall survival (OS), and progression-free survival (PFS).
Characterize the pharmacokinetic (PK) profile of ADCX25 (total antibody, drug-to-antibody ratio [DAR] 0), PBD-conjugated antibody (DAR 1), and free warhead SG3199.
Evaluate anti-drug antibodies (ADAs) to ADCX25 in blood before, during, and after treatment with ADCX25.

Efficacy Assessment

Assessment of response to treatment with ADCX25 will be based on bone marrow samples (aspirate or biopsy, if aspirate unattainable). The activity of ADCX25 will be evaluated based on the Investigator's evaluation of the patient's response to ADCX25 as CR, CRi, PR, PD, or NR as defined herein.

PK Assessment

The PK profile of ADCX25 (total antibody; drug-to-antibody ratio [DAR]≥0), PBD-conjugated antibody (DAR≥1), and free warhead will be assessed. Additional PK, ADA, cytokines, and serum CD25 (sCD25); blood samples will be collected at the discretion of the Investigator during any visit where toxicity is observed. A PK, ADA, cytokines, and sCD25 sample will also be collected concurrently with any other blood draw to assess safety (e.g., Unscheduled Visit), if possible. The PK profile will include determination of standard PK parameters (e.g., maximum concentration [Cmax], time to Cmax [Tmax], AUC0-last, AUC0-τ, AUC0-∞, AI, Vss, MRT, λz, t1/2, CL, and Vz.).

Safety Assessment

Safety will be assessed based on AEs, serious AEs (SAEs), treatment discontinuations due to AEs, DLTs (as defined herein) measurements of cytokines in serum, periodic 12-lead electrocardiogram (ECG) recordings, physical examinations, vital signs measurements, ECOG performance status, and hematology, biochemistry, coagulation panel, pregnancy testing (for women of child-bearing potential) and urinalysis test results. Adverse events will be graded according to CTCAE Version 4.0 (v4.03, published Jun. 14, 2010; NIH Publication No. 09-5410).

Product Dosage and Mode of Administration

ADCX25 is a sterile formulation containing PBD-conjugated HuMax®-TAC (DAR≥1), HuMax®-TAC (DAR=0), and SG3249. It is provided pre-formulated in 10-mL glass vials containing approximately 30 mg of ADCX25 per vial (deliverable volume 5.4 mL at 6 mg/mL). The appropriate quantity of ADCX25 will be diluted in 50 mL of 5% dextrose in water (D5W).

Patients will receive a 1-hour intravenous (IV) infusion of ADCX25 on Day 1 of Cycle 1. If ADCX25 is well tolerated after the first infusion, the infusion duration may be shortened to 30 minutes for subsequent cycles for that patient, at the Investigator's discretion.

The investigational product administration schedule is as follows:

Patients will be given ADCX25 (weekly [QW]) on Days 1, 8, and 15 of each 3-week (21-day) treatment cycle.

A patient will maintain the same treatment schedule throughout the duration of the trial.

Once a patient achieves CR/CRi, frequency or dose may be adjusted by the DESC based on emerging safety, efficacy, and PK profile.

The trial will be continuously monitored for emerging safety, efficacy and/or PK profile, and the DESC will determine if it is appropriate to maintain a QW schedule, revert to an every 3-week (Q3W) schedule, or test other dosing regimens.

Dose Escalation Design

Dose-escalation (Part 1) will be conducted according to a 3+3 design. The initial dose of ADCX25 will be 3 μg/kg (Dose Level 1), and the highest allowed dose will be 300 μg/kg.

The DLT observation period for dose-escalation will be 1 cycle. The first patient at each new dose level must be observed for 7 days for occurrence of AEs prior to treating the second patient at that dose level. Patients will be entered sequentially to each dose level.

For each dose level, if none of the first 3 patients at that level experiences a DLT, new patients may be entered at the next higher dose level. If 1 of 3 patients experiences a DLT, up to 3 more patients are to be treated at that same dose level. If none of the additional 3 patients at that dose level experiences a DLT, new patients may then be entered at the next higher dose level. However, if 1 or more of the additional 3 patients experience a DLT, then no further patients are to be started at that dose level and the preceding dose is identified as the MTD. The MTD; therefore, is defined as the highest dose level at which none of the first 3 treated patients, or no more than 1 of the first 6 treated patients, experiences a DLT.

No intra-patient dose-escalation is allowed.

The number of dose levels will depend on the emergent toxicity profile of ADCX25 and will be decided by the DESC; PK and PD evaluations may also inform decision making. During Part 1 (dose-escalation), the DESC may expand enrolment at doses below the current dose level as part of the dose-escalation process.

Additional patients may only be added at a lower dose level provided there is at least 1 patient who has achieved a PR or better (Section 7.1). No more than 10 patients in total can be treated at any dose level unless ≥3 of the 10 patients have achieved a PR or better.

Patients will be given ADCX25 (QW) on Days 1, 8 and 15 of each 3-week treatment cycle.

The first dose level for the weekly fractionated dosage regime/3 week treatment cycle (QW) dosing will be based on the safety and tolerability of patients who have been treated on the single dose/3-week treatment cycle schedule(Q3W). The first 3 patients will be given a cumulative dose each cycle that is comparable to (but not higher than) the highest dose tested at the Q3W dose schedule at which 3 patients completed the DLT observation period without a DLT. For example, if the highest Q3W dose tested at which 3 patients did not experience a DLT was cohort 92 μg/kg, the first cohort to receive QW dosing will receive 30 μg/kg each week for 3 weeks.

When the dose is escalated, the dose may increase by 50% if no DLTs are observed at the current level. Once a DLT is observed at a given dose level, the next dose may only increase by 25%. The dose may never increase by more than 50%, or more than an absolute value of 20 μg/kg/week, whichever is less. During Part 1, the DESC may expand enrolment at doses below the current dose level as part of the dose-escalation process. Additional patients may only be added at a lower dose level provided there is at least 1 patient who has achieved a partial response (PR or better). No more than 10 patients in total can be treated at any dose level unless ≥3 of the 10 patients have achieved a PR or better.

During Part 2 (dose expansion), patients will be monitored for safety using the same DLT criteria employed during dose-escalation. If during the treatment period, >30% of patients experience safety events that would meet the criteria that define a DLT in the dose-escalation phase of the study, enrolment in the expansion cohort(s) may be paused and the study data reviewed to determine whether additional monitoring or other action (such as alternate dose levels) should be evaluated prior to further enrolment.

A maximum of 80 patients (up to 50 patients in Part 1 and up to 30 patients in Part 2) may be enrolled at approximately 10 study sites in Part 1 and 10 study sites in Part 2.

Selected Results

It was observed that in 2 subjects with AML who had previously received bone marrow stem cell transfers, administration of ADCx25 led to complete remission of the AML.

Patient 1: (50 y/o M Relapsd AML, FAB M0, 30 Uq/Kq QW Cohort)
Date of AML diagnosis: 12 Jun. 2017
No FLT3-ITD Mutation
No Multilineage Dysplasia Syndrome
No Cytogenetic translocations
Immunophenotypic Analysis not available
10 Dec. 2013 Allogenic Transplant (matched sibling Stem Cell Transplant)
Patient achieved CRi after 5 cycles of ADCx25(˜September 2017)
Patient came off study November 2017

Patient 2: (73 y/o M; Relapsed AML FAB M4, 37.5 ug/Kq QW Cohort)
Date of AML diagnosis: September-2013
No FLT3-ITD Mutation
No Multilineage Dysplasia Syndrome
Immunophenotypic Analysis Available
CD2 (−), CD3 (−), CD4 (+), CD5 (−), CD7 (−), CD8 (−), CD9 (Not Done), CD10 (−), CD11b (−), CD11c (−), CD13 (+) and CD14 (−)
30 Jul. 2015 Allogenic Transplant

### Example 3

Re-Challenge of Tumor-Free Survivors from Example 1 MC38 Efficacy Study

The complete responders from Example 1 and 10 naïve control female C57BL/6 mice were or 17-18 weeks old on Day 1 of this study and had a BW range of 20.9 to 39.0 g.

On Day 1 of the re-challenge study, 5×105 MC38 cells (0.1 mL suspension) were subcutaneously implanted into the left flank (contralateral to the original cell implant) and tumor growth was monitored. No ADC or anti-PD-1 treatment was administered in the rechallenge study.

Animal handling and tumour measurement was as in Example 1 unless otherwise stated.

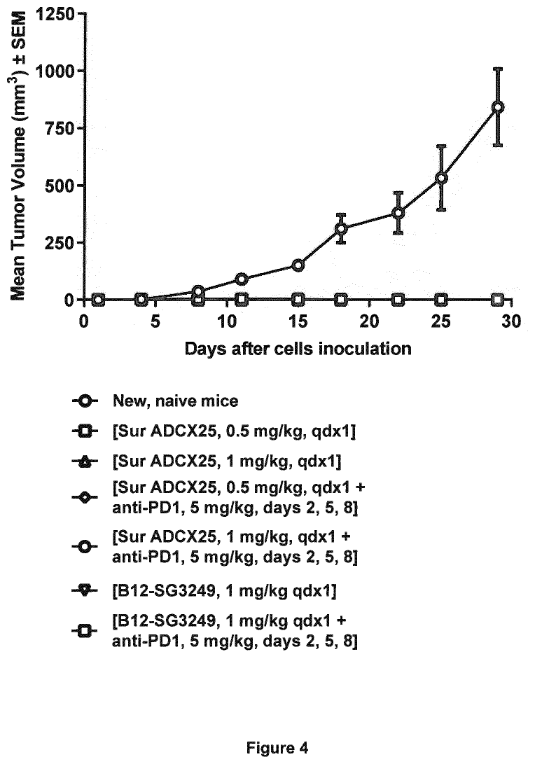

Results: Re-challenged animals did not develop new tumors indicating ADCx25 was able to induce tumor-specific protective immunity (see FIG. 4).

### Example 4

In Vivo Efficacy Study of Surrogate-ADCx25 in an Immuno-Competent Syngeneic Mouse Model Using Mouse Colon Cancer CT26 Cells

Introduction

CT26 is a CD25-ve mouse colon cancer-derived model used pre-clinically in immunotherapy-type studies which is known to have infiltration of Treg and Teff cells.

Study Design

Female BALB/c mice (BALB/cNCrl, Charles River) were nine weeks old on Day 1 of the study and had a body weight (BW) range of 17.2 to 23.3 g. At the completion of the study,, tumor-free survivors were transferred to they re-challenge study described in Example 5.

On the day of implant 3×10⁵ CT26 cells (0.1 mL suspension) were subcutaneously implanted into the right flank of each test animal. Tumors were monitored as their volumes approached the target range of 80-120 mm³. Ten days after tumor cell implantation, on Day 1 of the study, animals were sorted into ten groups (n=10/group) with individual tumor volumes of 75 to 162 mm³, and group mean tumor volumes of 110-111 mm³. All doses were administered intraperitoneally (i.p.) on Day 1 except for anti-PD-1 which was administered once on Days 2, 5, 8. The dosing volume was 0.2 mL per 20 grams of body weight (10 mL/kg), and was scaled to the body weight of each individual animal. Tumors were measured twice per week until the study was ended on Day 48. Each animal was euthanized when its tumor attained the endpoint tumor volume of 2000 mm³ or on the final day, whichever came first.

Tumors were measured in two dimensions using calipers, and volume was calculated using the formula:

Tumor Volume (mm³)=w2×l/2, where w=width and l=length,in mm,of the tumor.

Tumor weight may be estimated with the assumption that 1 mg is equivalent to 1 mm³ of tumor volume.

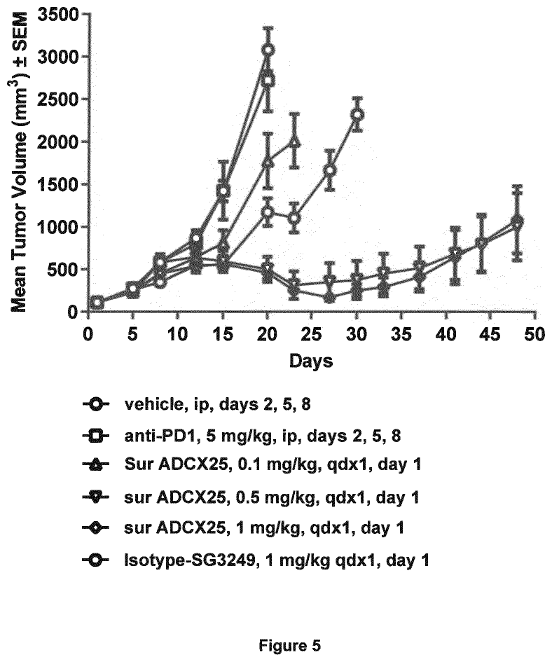

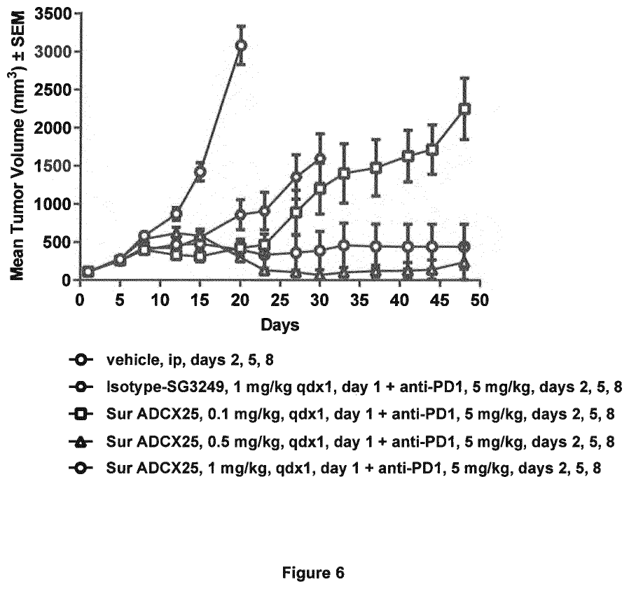

Results: In vivo, a single dose of sur-ADCx25 at 0.5 or 1 mg/kg induced strong and durable anti-tumor activity against established CD25-negative solid tumors with infiltrating Treg cells (CT26 syngeneic model); see FIG. 5 FIG. 6.

| Response summary | PR | CR | TFS |
|---|---|---|---|
| Vehicle | 0 | 0 | 0 |
| Sur ADCX25, 0.1 mg/kg | 0 | 0 | 0 |
| Sur ADCX25, 0.5 mg/kg | 1 | 2 | 2 |
| Sur ADCX25, 1 mg/kg | 1 | 3 | 3 |
| Anti-PD1, 5 mg/kg | 0 | 0 | 0 |
| Isotype-ADC, 1 mg/kg | 0 | 0 | 0 |
| Sur ADCX25, 0.1 mg/kg + | 1 | 1 | 1 |
| anti-PD1 | | | |
| Sur ADCx25, 0.5 mg/kg + anti-PD1 | 0 | 7 | 7 |
| Sur ADCX25, 1 mg/kg + anti-PD1 | 0 | 8 | 8 |
| Isotype-ADC, 1 mg/kg + anti-PD1 | 0 | 1 | 1 |

Coefficient of Drug Interaction (CDI)

Sur ADCX25, 0.1 mg/kg;
anti-PD1, 5 mg/kg;
Sur ADCX25, 0.1 mg/kg + anti-PD1
Day 20 0.285 (Synergism)

### Example 5

Re-Challenge of Tumor-Free Survivors from Example 4 CT26 Efficacy Study

The complete responders from Example 4 and 10 naïve control female BALB/c mice were or 16-17 weeks old on Day 1 of the study and had a BW range of 18.2 to 24.4 g.

On Day 1 of the re-challenge study 3×10¹ CT26 cells (0.1 mL suspension) were subcutaneously implanted into the left flank (contralateral to the cell implant of example 4) and tumor growth was monitored and measured as described in example 4.

No treatment was administered in the re-challenge study.

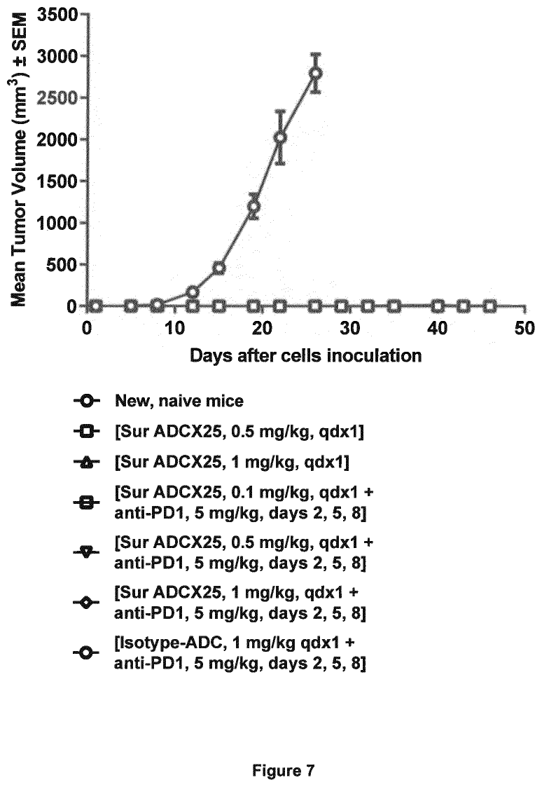

Results: Re-challenged animals did not develop new tumors indicating ADCx25 was able to induce tumor-specific protective immunity (see FIG. 7).

### Example 6

surADCx25 Anti-Tumour Activity Against CD25-Ve Tumours is Dependent on C8+ T-Cells

Study Design

Female C57BL/6 mice (C57BL/6NCrl, Charles River) were eleven weeks old on Day 1 of the study and had a body weight (BW) range of 18.6 to 28.2 g.

On the day of implant 5×10⁵ MC38 cells (0.1 mL suspension) were subcutaneously implanted into the right flank of each test animal. Tumors were monitored as their volumes approached the target range of 80-120 mm³.

Fifteen days after tumor cell implantation, on Day 0 of the study, animals were sorted into groups (n=10/group) with individual tumor volumes of 75 to 126 mm3, and with group mean tumor volumes of 86-89 mm³. All doses were administered intraperitoneally (i.p.).

Anti-CD8-2.43 murine monoclonal antibody was administered once daily on Days 0, 5, 8 and 13 in order to deplete and suppress levels of CD8+ T-cells.

SurADCx25 dose was administered on Day 1.

Anti-PD-1 was administered once daily on Days 2, 5, 8.

The dosing volume was 0.2 mL per 20 grams of body weight (10 mL/kg), and was scaled to the body weight of each individual animal.

Tumors were measured twice per week until the study was ended (day29 for anti-CD8+surADCx25 and anti-CD8+anti-PD1+surADCx25 groups; day 44 for surADCx25 alone and surADCx25+anti-CD8).

Each animal was euthanized when its tumor attained the endpoint tumor volume of 1000 mm³ or on the final day, whichever came first.

Results

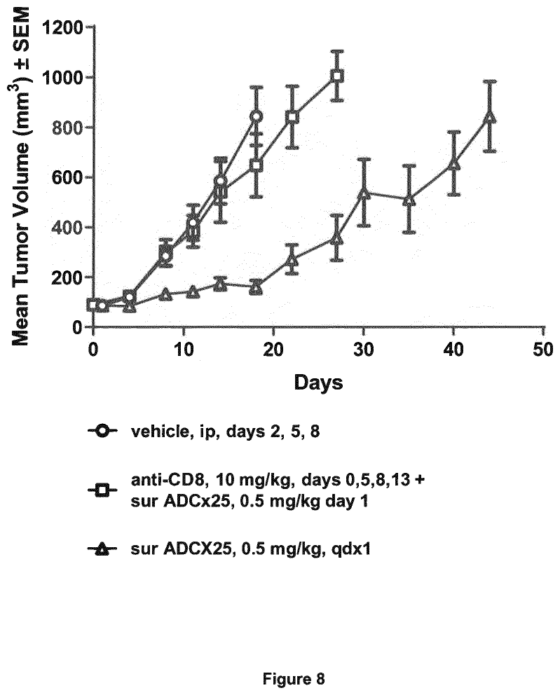

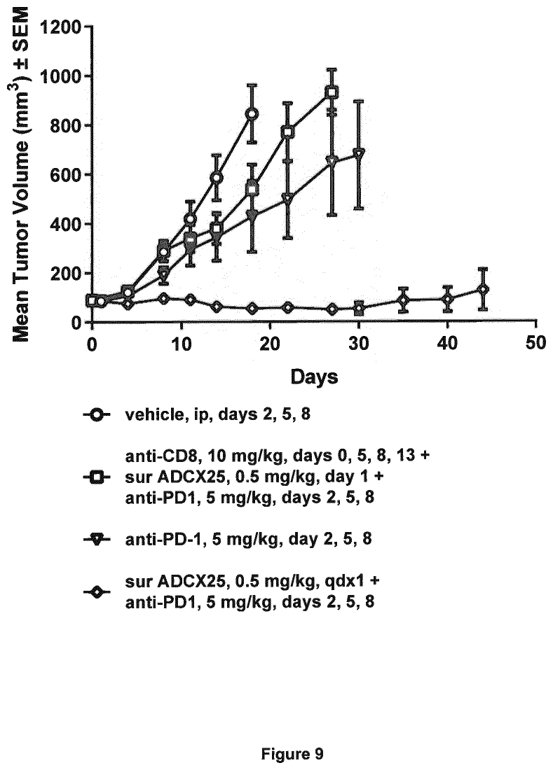

Sur-ADCx25 anti-tumor activity, either alone (FIG. 8) or combined with an anti-PD1 antibody (FIG. 9), was significantly reduced when CD8+ T-cells are depelted, indicating that surADCx25 activity is CD8+ T-cell-dependent and that overall effector T-cell responses were not negatively impacted by sur-ADCx25.

| Response summary | PR | CR | TFS |
|---|---|---|---|
| Vehicle | 0 | 0 | 0 |
| Anti-PD1 | 2 | 0 | 0 |
| Sur ADCX25 | 0 | 1 | 1 |
| Sur ADCX25 + anti-CD8 | 0 | 0 | 0 |
| Sur ADCX25 + anti-PD1 | 2 | 5 | 5 |
| Sur ADCX25 + anti-PD1 + anti-CD8 | 0 | 0 | 0 |

Coefficient of Drug Interaction (CDI)

Sur ADCX25, 0.5 mg/kg;
anti-PD1, 5 mg/kg;
Sur ADCX25, 0.5 mg/kg + anti-PD1
Day 22 0.471 (Synergism)

### Example 7

T-Cell Immuno-Profiling after SurADCx25 Dosing in Healthy Immuno-Competent Mice

Study Design

Eight to twelve week old, female C57BL/6 mice were dosed intravenously on day 1 with either surADCx25 (0.5 mg/kg) or an isotype control ADC (0.5 mg/kg). A non-dosed group acted as control (each group contained 24 mice).

Terminal samples (blood, spleen, lymph node and thymus) were obtained on days 1 (4 hrs post-dose), 7, 14 and 21 from 6 animals per group at each time-point. Additional non-terminal blood samples (mandibular bleeds) were obtained on days 4, 11 and 18 from 6 animals per group at each time-point.

Samples (tissue and blood) were processed for flow cytomtetry assessment and CD4⁺ T-cells (CD45⁺ CD3⁺ CD4⁺ CD8⁻), CD8⁺ T-cells (CD45⁺ CD3⁺ CD4⁻ CD8⁺) and T_{reg} cell (CD45⁺ CD3⁺ CD4⁺ CD25⁺ FoxP3⁺) content determined. Data represent the mean±SEM of T_{reg} cell population in the assayed tissue as a percentage of CD45⁺.

Results

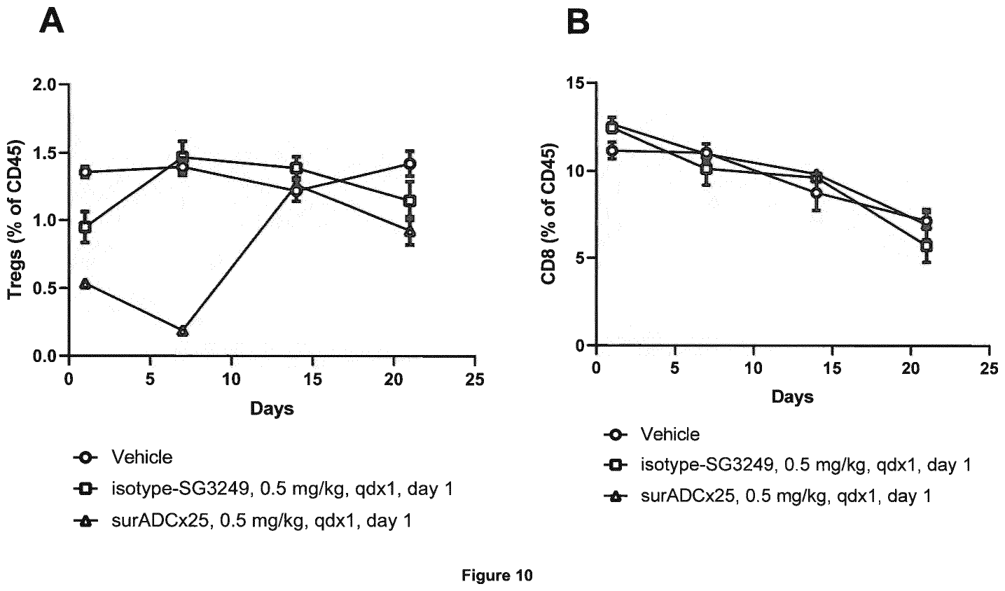

Spleen:
a clear depletion of spleen Tregs was observed at 1 day & 7 days post surADCx25 administration, with Treg levels mostly recovering by day 14 (see FIG. 10A; % shown indicates the % reduction compared to vehicle)
there was no observed impact on the level of spleen CD8+ Teff cells (see FIG. 10B)

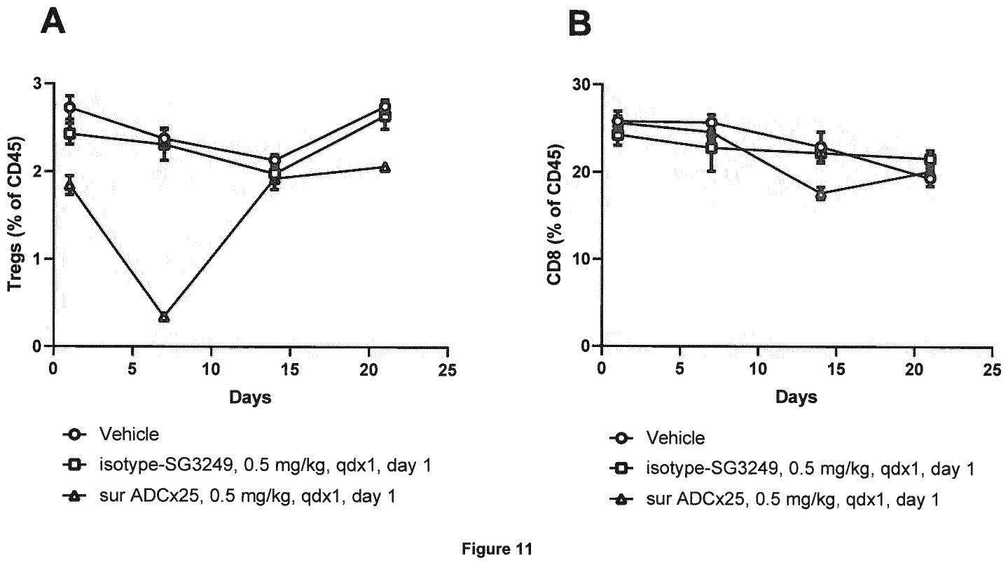

Lymph Nodes:
a clear depletion of lymph node Tregs was observed at 1 day & 7 days post surADCx25 administration, with Treg levels mostly recovering by day 14 (see FIG. 11A; % shown indicates the % reduction compared to vehicle)
there was no observed impact on the level of lymph node CD8+ Teff cells (see FIG. 11B)

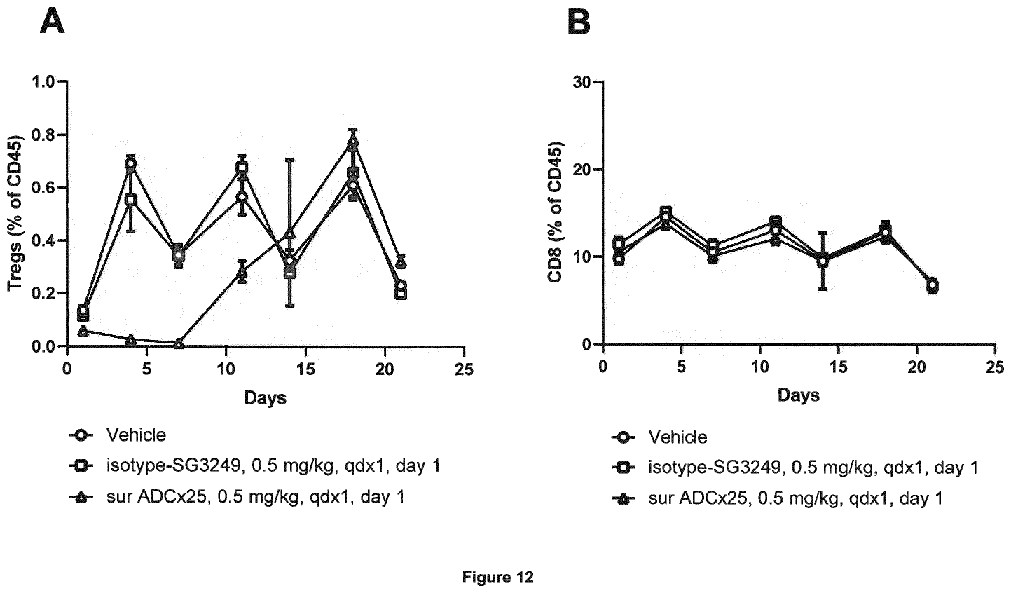

Blood:
Increased variability in vehicle and isotype-control values due to low levels of events; measured with additional non-terminal blood samples (days 4, 11 and 18).
a clear depletion of blood Tregs was observed at 1 day, 7 days, and 11 days post surADCx25 administration, with Treg levels recovering by day 14 (see FIG. 12A; % shown indicates the % reduction compared to vehicle)
there was no observed mpact on the level of blood CD8+ Teff cells (see FIG. 12B)

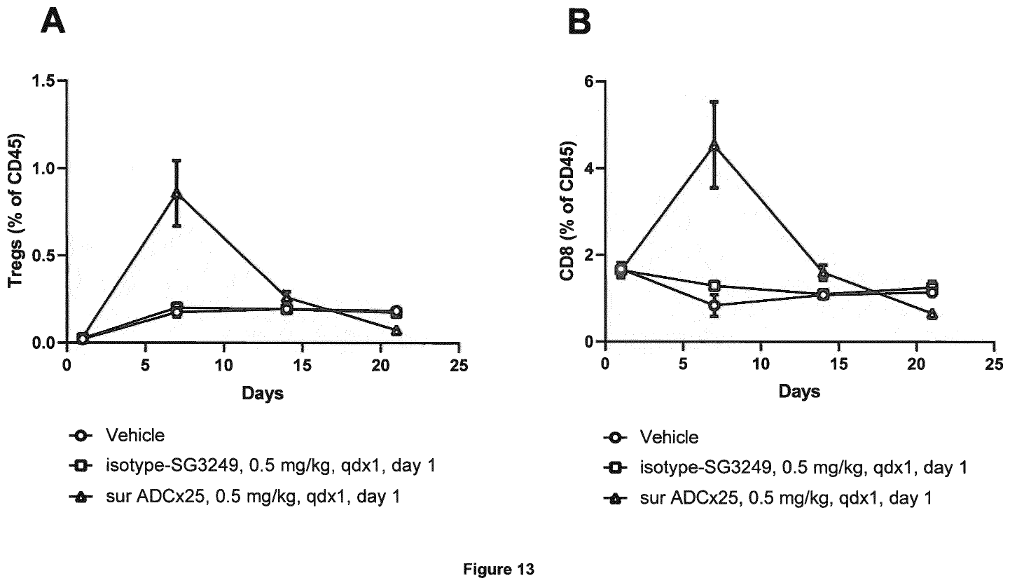

Thymus:
a clear increase of thymus Tregs & CD8+ Teffs was observed at 7 days post surADCx25 administration, with Treg & CD8+ Teff levels mostly recovering by day 14 (see FIGS. 13A & B)

Summary

A single dose of SurADCx25 caused significant depletion of Tregs in spleen, lymph nodes and blood (>95%).

There was a clear increase in the thymus in the amount of Tregs at 7 days post-dose.

There was no depletion of Teff cells from spleen, lymph nodes and blood caused by SurADCx25, however, an increase in thymus Teff cells was also observed 7 days post dosing of SurADCx25.

Around Day 15, Tregs levels in blood, spleen, thymus and lymph nodes are restored to normal (vehicle control).

### Example 8

T-Cells Immuno-Profiling after surADCx25 Dosing in CT26 Tumor-Bearing Immune-Competent Mice

Study Design

Female BALB/c mice were ten weeks old on Day 1 of the study.

Cultured CT26 cells were harvested during log phase growth and resuspended in phosphate buffered saline (PBS) at a concentration of 3×10⁶ cells/mL. Tumors were initiated by subcutaneously implanting 3×10⁵ CT26 cells into the right flank of each test animal. Fourteen days after tumor cell implantation, on Day 1 of the studies, animals were sorted into groups (n=24 or 18) with mean tumor volumes of 115-116 mm³.

SurADCx25 was administered intraperitoneally (i.p.) on Day 1. Anti-PD-1 was administered i.p. once daily on Days 2, 5, 8. Group 1 received the PBS vehicle and served as the control. Group 2 received anti-PD-1 at 5 mg/kg. Groups 3 received surADCx25 at 1 mg/kg. Group 4 received surADCx25 at 1 mg/kg in combination with anti-PD-1 at 5 mg/kg.

Samples were collected for analysis by flow cytometry on Days 1 (pre-dose; Group 1 only, n=6), 3 (Groups 1-4, n=6) and 9 (Groups 1-4, n=6). Full blood volume was collected from each animal via terminal cardiac puncture and was processed for flow cytometry.

Immediately following blood collection, the tumor and the spleen were harvested from each animal and processed for flow cytometry and CD4+ T-cells (CD45+CD3+CD4+CD8-), CD8+ T-cells (CD45+CD3+CD4− CD8+) and Treg cell (CD45+CD3+CD4+CD25+ FoxP3+) content determined.

Results

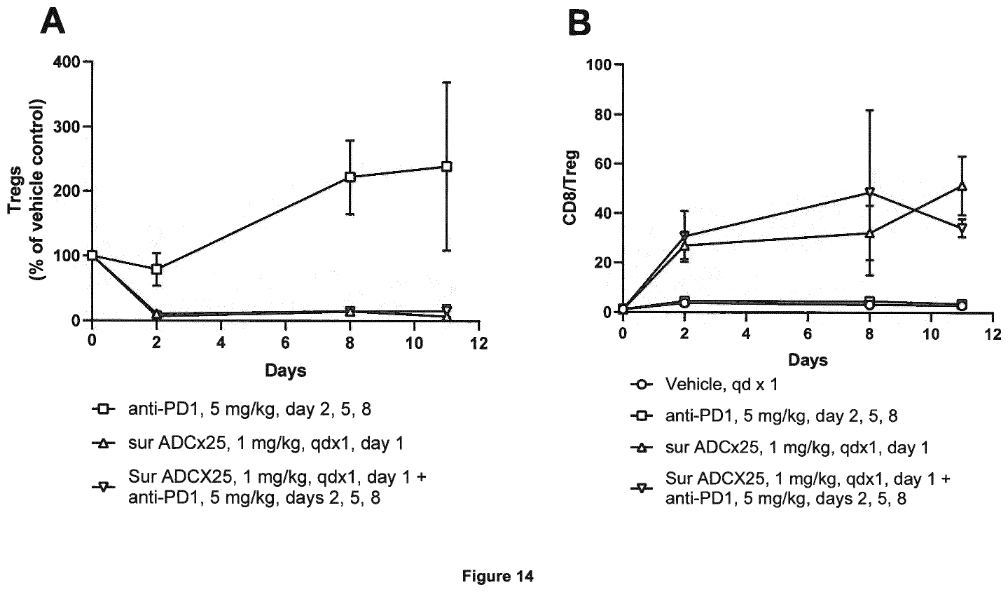

Tumour:
a significant and sustained depletion of tumour Tregs was observed from 2 days through 11 days post surADCx25 administration (see FIG. 14A)
an increased tumour CD8+ Teff/Tregs ratio was observed from 2 days through 11 days post surADCx25 administration (see FIG. 14B).

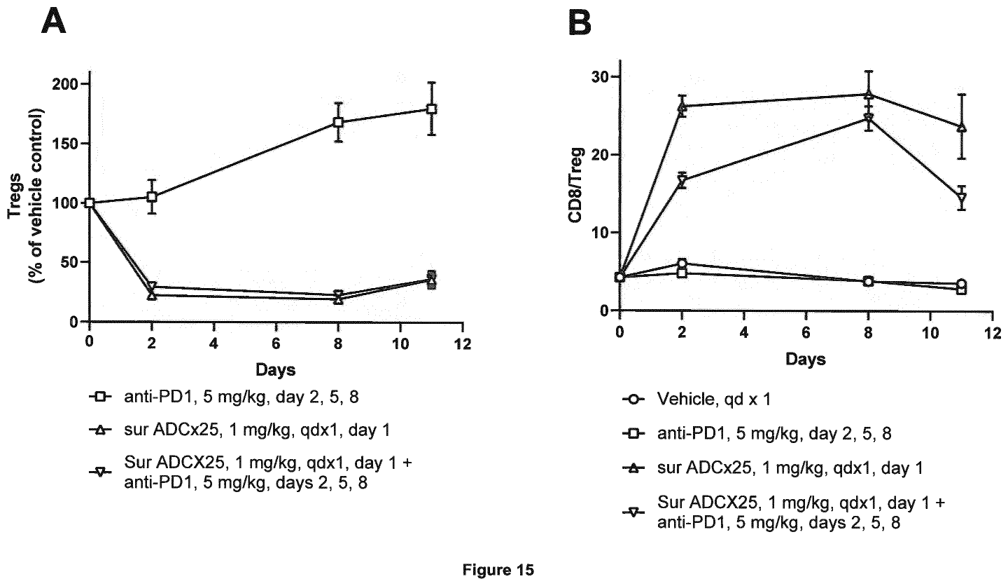

Spleen:
a significant and sustained depletion of spleen Tregs was observed from 2 days through 11 days post surADCx25 administration (see FIG. 15A)
an increased spleen CD8+ Teff/Tregs ratio was observed from 2 days through 11 days post surADCx25 administration (see FIG. 15B).

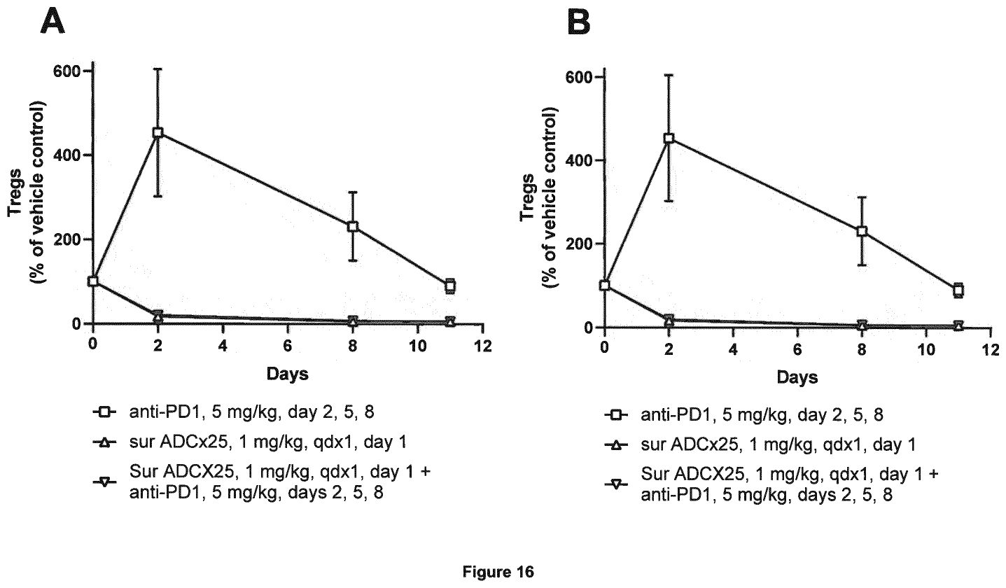

Blood:
a significant and sustained depletion of blood Tregs was observed from 2 days through 11 days post surADCx25 administration (see FIG. 16A)
an increased blood CD8+ Teff/Tregs ratio was observed from 2 days through 11 days post surADCx25 administration (see FIG. 16B).

Summary and Conclusions

A single dose of surADCx25 to immuno-competent mice bearing established CT26 tumors caused significant and sustained depletion of Tregs in tumors, blood, and spleen.

The simultaneous increase observed in CD8+ Teff/Tregs ratio in tumors, blood, and spleen indicates that surADCx25 did not negatively impact the overall Teff cell response.

Together, the data set out in Examples 6 to 8 indicate that SurADCx25 depletion of Tregs together with activation of the CD8+ Teff response is an important mode of action in surADCx25's anti-tumour activity.

### Example 9

Preliminary Data from a Phase 1b Dose-Escalation Study of Camidanlumab Tesirine in Patients with Advanced Solid Tumors

Background

This is a report of preliminary data from a Phase 1b trial of Camidanlumab Tesirine (NCT03621982; ADC Therapeutics) in patients (pt) with selected advanced solid tumors 35 (AST) who have failed existing therapies.

Methods

This multicenter, open-label, dose-escalation (part 1), and dose-expansion (part 2) study is currently enrolling subjects (aged≥18 years) with selected AST. The primary objective is to evaluate the safety and tolerability and to identify the recommended dose. Secondary objectives are to assess preliminary antitumor activity, pharmacokinetics (PK), and immunogenicity.

Subjects receive 1-hour intravenous infusions of Camidanlumab Tesirine every 3 weeks (Q3W) from a starting dose cohort of 20 μg/kg. Subsequent cohorts are enrolled at escalating doses according to a 3+3 design.

Results

As of Apr. 16, 2019, 6 subjects have been treated at doses of 20-30 μg/kg. Pathological subtypes treated include pancreatic (n=3) and breast, renal, and lung (n=1 each) cancer. The 45 μg/kg dose level is now under examination.

No dose-limiting toxicities have been reported within the 21-day reporting period. The most common (≥3 pts) adverse events (AE) regardless of relationship to Camidanlumab Tesirine were fatigue and nausea. Most AEs were grade 52 and not related to Camidanlumab Tesirine. There were no reported grade ?3 AEs or serious AEs considered related to Camidanlumab Tesirine.

Stable disease has been the best response recorded so far as per the response evaluation criteria in solid tumors (n=2 at 20 μg/kg and, reported a few days after the cutoff date, n=2 at 30 μg/kg). Two subjects remain on treatment at 30 μg/kg.

Conclusions

This continuing study will further refine the recommended dose of Camidanlumab Tesirine in selected advanced solid tumors.

1-35. (canceled)

36. A method of treating or preventing a CD25-ve solid tumor in a subject, the method comprising the step of administering a CD25-ADC to the subject;
wherein the CD25-ve solid tumor comprises CD25-ve neoplastic cells and CD25+ve regulatory T cells;
wherein the CD25-ADC is a conjugate of formula L—(D^{L})ₚ, where D^{L} is of formula I or II: 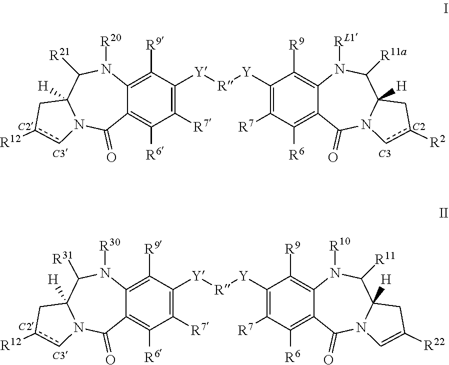
wherein:
p is from 1 to 8;
L is an antibody (Ab) which is an antibody that binds to CD25;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id) 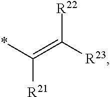
R², wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;
(ie) 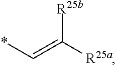
wherein one of R^{25a} and R^{25b} is H and the other is selected from: phenyl, which phenyl is optionally substituted by a group selected from halo, methyl, methoxy; pyridyl; and thiophenyl; and
(if) 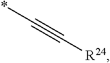
where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R¹² is 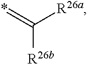
where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;
A. wherein, if D^{L} is of formula I:R^{L1′} is a linker for connection to the antibody (Ab);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id) 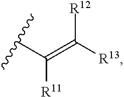
wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;
(ie) 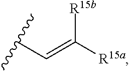
wherein one of R^{15a} and R^{15b} is H and the other is selected from: phenyl, which phenyl is optionally substituted by a group selected from halo, methyl, methoxy; pyridyl; and thiophenyl; and
(if) 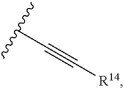
where R¹⁴, where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is 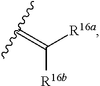
where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
B. wherein, if D^{L} is of formula II:
R²² is of formula IIIa, formula IIIb or formula IIIc:
(a) 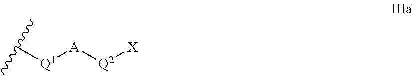
where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;
(b) 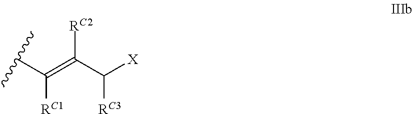
where;
R^{C1}, R^{C2} and R^{C3} are independently selected from H and unsubstituted C₁₋₂ alkyl;
(c) 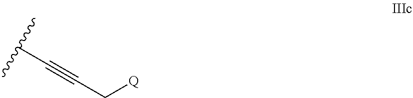
where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′}, 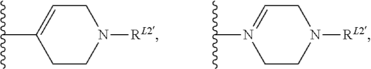
NR^{N}R^{L2′} wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M.

37. The method of claim 36, wherein the solid tumour is an established tumour or a metastatic tumour.

38. The method of claim 37, wherein the established tumour is a tumour diagnosed or identified in a naïve subject.

39. The method of claim 37, wherein the established tumour is a relapsed tumour.

40. The method of claim 36, wherein the CD25−ve solid tumor consists of CD25−ve neoplastic cells and CD25+ve regulatory T cells.

41. The method of claim 36, wherein the CD25−ve solid tumor consists of CD25−ve neoplastic cells.

42. The method of claim 36, wherein the solid tumour is selected from the group consisting of pancreatic cancer, breast cancer, colorectal cancer, gastric and oesophageal cancer, melanoma, non-small cell lung cancer, ovarian cancer, hepatocellular carcinoma, renal cell carcinoma, bladder, and head and neck cancer.

43. The method of claim 36, wherein the CD25-ADC is administered in combination with a cell therapy.

44. The method of claim 43, wherein the cell therapy comprises the administration of stem cells and/or immune cells;
optionally wherein the immune cells are CAR-T-cells, T-cells, Natural Killer (NK) cells, Natural Killer T-cell (NKT), Lymphokine-activated Killer (LAK) cells, or macrophages.

45. The method of claim 36, wherein the CD25-ADC is administered in combination with a checkpoint inhibitor or other immunostimulatory agent.

46. The method of claim 45, wherein the checkpoint inhibitor is a PD1 antagonist, a PD-L1 antagonist, a GITR agonist, an OX40 agonist, a CTLA-4 antagonist, or a bi-specific T-cell engager (BiTE).

47. The method of claim 36, wherein the CD25-ADC is administered in combination with radiotherapy.

48. The method of claim 36, wherein the anti-CD25-ADC has the chemical structure: 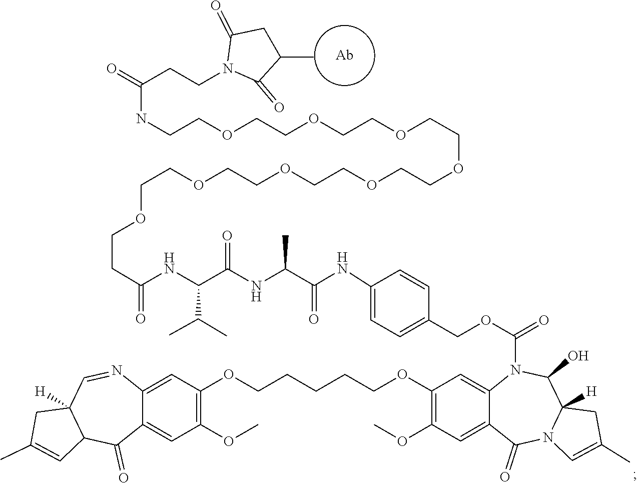
wherein the Ab is an anti-CD25 antibody having a VH domain comprising a VH CDR1 with the amino acid sequence of SEQ ID NO.3, a VH CDR2 with the amino acid sequence of SEQ ID NO.4, and a VH CDR3 with the amino acid sequence of SEQ ID NO.5; and a VL domain comprising a VL CDR1 with the amino acid sequence of SEQ ID NO.6, a VL CDR2 with the amino acid sequence of SEQ ID NO.7, and a VL CDR3 with the amino acid sequence of SEQ ID NO.8;
and wherein the drug to antibody ratio (DAR) is between 1 and 8.

49. The method of claim 36, wherein the CD25-ADC is Camidanlumab Tesirine.

50. A method of inducing or enhancing an immune response in a subject, the method comprising the step of administering a CD25-ADC and a disorder-associated antigen (DAA) to the subject;
wherein the CD25-ADC is a conjugate of formula L—(D^{L})ₚ, where D^{L} is of formula I or II: 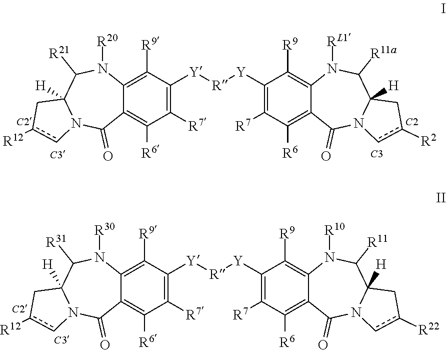
wherein:
p is from 1 to 8; L is an antibody (Ab) which is an antibody that binds to CD25;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id) 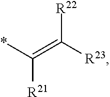
wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;
(ie) 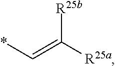
wherein one of R^{25a} and R^{25b} is H and the other is selected from: phenyl, which phenyl is optionally substituted by a group selected from halo, methyl, methoxy; pyridyl; and thiophenyl; and
(if) 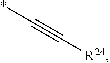
where R is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R¹² is 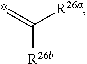
where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;
A. wherein, if D^{L} is of formula I,
R^{L1′} is a linker for connection to the antibody (Ab);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id) 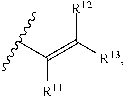
Wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;
(ie) 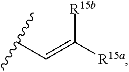
Wherein one of R^{15a} and R^{15b} is H and the other is selected from: phenyl, which phenyl is optionally substituted by a group selected from halo, methyl, methoxy; pyridyl; and thiophenyl; and
(if) 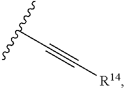
where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is 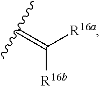
where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
B. wherein, if D^{L} is of formula II,
R²² is of formula IIIa, formula IIIb or formula IIIc:
(a) 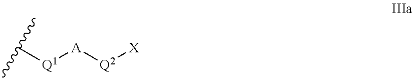
where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;
(b) 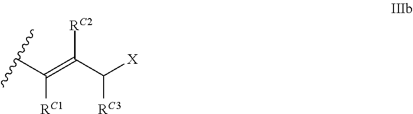
where;
R^{C1}, R^{C2} and R^{C3} are independently selected from H and unsubstituted C₁₋₂ alkyl;
(c) 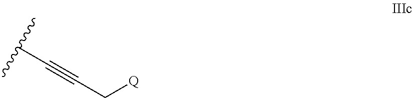
where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′}, 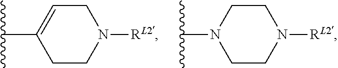
NR^{N}R^{L2′}, wherein R^{N} is selected from the group comprising H an 1-4 alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³¹ is H and R³¹ is selected from OH, OR^{A} and SO_{z}M.

51. The method of claim 50, wherein the DAA is a tumour-associated antigen (TAA) or pathogen-associated antigen (PAA).

52. The method of claim 50, wherein:
a) the DAA is administered as part of a vaccine composition; or
b) the DAA is administered as part of a vaccine composition, wherein the CD25-ADC is also part of the same vaccine composition.

53. The method of claim 50, wherein the anti-CD25-ADC has the chemical structure: 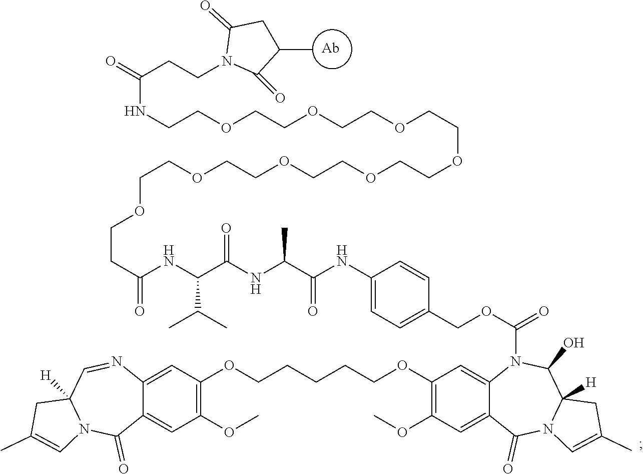
wherein the Ab is an anti-CD25 antibody having a VH domain comprising a VH CDR1 with the amino acid sequence of SEQ ID NO.3, a VH CDR2 with the amino acid sequence of SEQ ID NO.4, and a VH CDR3 with the amino acid sequence of SEQ ID NO.5; and a VL domain comprising a VL CDR1 with the amino acid sequence of SEQ ID NO.6, a VL CDR2 with the amino acid sequence of SEQ ID NO.7, and a VL CDR3 with the amino acid sequence of SEQ ID NO.8;
and wherein the drug to antibody ratio (DAR) is between 1 and 8.

54. The method of claim 50, wherein the CD25-ADC is Camidanlumab Tesirine.

55. A method of inducing or enhancing an immune response in a subject, the method comprising the step of administering a CD25-ADC to the subject;
wherein the CD25-ADC is a conjugate of formula L—(D^{L})ₚ, where D^{L} is of formula I or II: 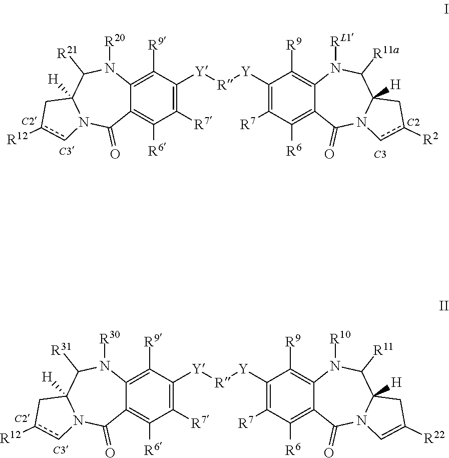
wherein:
p is from 1 to 8; L is an antibody (Ab) which is an antibody that binds to CD25;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id) 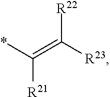
wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;
(ie) 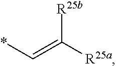
wherein one of R^{25a} and R^{25b} is H and the other is selected from: phenyl, which phenyl is optionally substituted by a group selected from halo, methyl, methoxy; pyridyl; and thiophenyl; and
(if) 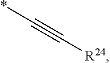
where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R¹² is 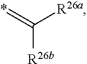
where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;
A. wherein, if D^{L} is of formula I,
R^{L1′} is a linker for connection to the antibody (Ab);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;
(id) 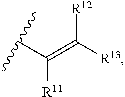
wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;
(ie) 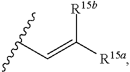
wherein one of R^{15a} and R^{15b} is H and the other is selected from: phenyl, which phenyl is optionally substituted by a group selected from halo, methyl, methoxy; pyridyl; and thiophenyl; and
(if) 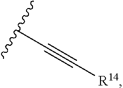
where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is 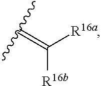
where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
B. wherein, if D^{L} is of formula II,
R²² is of formula IIIa, formula IIIb or formula IIIc:
(a) 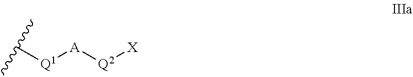
where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;
(b) 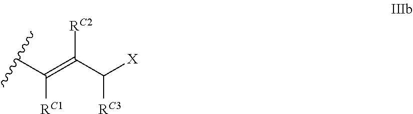
where;
R^{C1}, R^{C2} and R^{C3} are independently selected from H and unsubstituted C₁₋₂ alkyl;
(c) 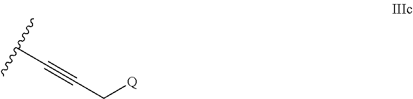
where Q is selected from OR^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′}, 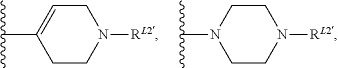
NR^{N}R^{L2′} wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M.
